SEQUENCE LISTING (103 sequences)

SEQ ID NO: 1 - DNA, 1509 bp, Arabidopsis thaliana
atggttatggagcaagctggaacgacatcgtattcggtcgtgtcagagtttgaaggaacaatactgaagaacgcagattcattctcttacttcatgctcgtagccttcgaagcagctggtctaattcgtttcgctatcttgttgtttctatggcccgtaatcacactccttgacgttttcagctacaaaaacgcagctctcaagctcaagatttttgtagccactgttggtctacgtgaaccggagatcgaatcagtggctagagccgttctgccaaaattctacatggacgacgtaagcatggacacgtggagggttttcagctcgtgtaagaagagggtcgtggtcacgagaatgcctcgagttatggtggagaggtttgctaaggagcatcttagagcagatgaggtcatcggtacggaactgattgtaaaccggttcggttttgtcaccggtttgattcgcgaaacggatgttgatcagtctgctttgaaccgtgtcgctaatttgtttgttggtcggaggcctcaactaggtcttggaaaaccggctttgaccgcctctacaaatttcttatcgttatgtgaggagcatattcatgcaccaatcccggagaactacaaccacggtgaccaacaacttcagctacgtccacttccggtgatatttcacgacggaagactagtgaagcggccaacgccggccaccgctctcatcatcctcctttggatcccatttggaatcattctcgccgtgatccggatctttcttggagccgtcctcccattgtgggccacaccttacgtctctcagatattcggtggccatatcatcgtcaaaggaaagcctcctcagccaccggcggctggaaaatccggcgtgctctttgtgtgtactcacagaaccctaatggaccctgtggtattatcttatgtcctcggacgtagcatcccagccgttacttactcaatctcgcgcttatcagagatcttatctcccattccaaccgtccgattgacaagaatccgagatgtggatgcggctaagatcaaacaacaactgtcaaaaggagatctagtggtttgtcctgagggaaccacttgtcgtgaaccgtttttgttaagattcagcgcgcttttcgctgagttaacggataggattgttccggttgcgatgaactacagagtcggattcttccacgcgactacagcgagaggctggaagggtttggacccaattttcttcttcatgaacccaagaccggtttacgagattacgttcttgaaccagcttcctatggaggcaacatgttcgtccgggaagagcccgcatgacgtggcgaactatgttcagagaatcttggcggctacgttagggtttgagtgcaccaacttcacaagaaaagataagtatagggttctcgctggaaacgatggaacggtgtcgtacttgtcgttgctagaccaattgaagaaggtggttagcactttcgagccttgtctccattga

SEQ ID NO: 2 - DNA, 2135 bp, Arabidopsis thaliana
ccaacacatttacttttcttcttcttctctctctacgccatagctatggttttaccagagcttctcgtgattctagcagaatgggttctttaccgtcttcttgctaaatcatgttacagagctgctagaaagctcagaggctatggtttccagttaaaaaatctcctaagcctctccaaaacacaatctctacacaacaattctcagcatcatcttcataaccatcatcaacaaaatcaccctaaccaaacccttcaagattctctagaccctctgttcccaagccttacaaaataccaagaacttcttctagacaaaaacagagcatgctctgtttcgtctgatcattatcgtgatacctttttctgcgatatcgatggtgttttattaagacaacattcgagcaaacacttccacacgttcttcccttacttcatgctcgtggcttttgaaggaggatcgatcatcagagcgatccttctcttgctctcctgttctttcctttggactctgcaacaagaaaccaaactcagagttctctctttcataaccttctctggtcttagagtcaaagacatggacaatgtttctagatcggttctaccaaaattcttcctcgagaacttgaatatccaagtttacgacatctgggctcgaactgagtactccaaagttgtgttcacgagcttgccgcaagtgctggtggagaggttcctcagagaacatctcaacgctgatgatgtgattgggactaaactgcaagaaataaaagtcatgggaaggaaattctacacaggcctagcttctggttctggtttcgtcctgaaacataaatcagctgaagactacttttttgatagcaagaagaagcctgcacttggaattggaagctcttcaagcccacaagatcacatcttcatctccatttgcaaagtaatttcctttccttcttaaagttactaactttcaattttgtttaagttttctacaaggtcatatctgttacttttcaaatttcacaaaaatagattaaagtttagaacgtcttcttttgttctgaatctcccacacaggaagcttatttttggaatgaagaagagtccatgagcaagaacaacgcattgcctagagaaagatacccaaagccactgatattccacgacgggagattggctttcttgccaacaccattagctacattagccatgttcatatggctccctatcggatttttactcgctgtcttcaggatctcggtaggagtctttcttccttaccatgtagccaatttcttggcttctatgtccggtgttaggatcactttcaagacccataatttgaataacggtcgtccagagaaaggtaatagtggagtcttgtatgtatgcaaccataggaccctcctcgacccagttttcctcaccacttcgctgggcaagcccttgaccgcggttacttacagcctaagcaagttctccgagttcatagcacctctcaagacggttagccttaaaagagataggaagaaagacggtgaggcaatgcagagactattaagcaaaggagatctagtggtttgtccggagggaaccacatgtagagagccttatctactaaggttcagtcctttgttcgctgagttaactgaagacattgttcccgtggctgtagatgctcgagtgagcatgttctatgggacaactgcgagcgggctcaagtgtttagaccctatcttcttcctcatgaaccctaggccggtttattgtcttgagattttaaagaagttgccaaaagagatgacttgtgcaggaggcaagtcaagctttgaagttgcgaatttcatacagggagaacttgctagggttttgggatttgaatgcactaatctcacaaggagggataagtacttggttcttgctggaaatgaaggcattgtcagataaactattccgaattttcattagtttttttgtttgtttgaaaagctaaatattattgttatttcaggacttagatgtttaattctatatggttgattcattggcatcatttctatttcttatcactcgggaccattgatatgtattgaaaatgtttgctttatatgattttgtttctggatttgtatagtccat

SEQ ID NO: 3 - DNA, 2440 bp, Arabidopsis thaliana
tcgacatgcagagtctagattccagcgacaccatataatatagttattagaaaacgagaggtcaaaaccatgtccggtaataagatctcgactcttcaagctcttgtcttcttcttgtaccggtttttcattctccgtcgttggtgtcatcgtagccctaaacaaaaataccaaaaatgcccttctcacggcctccaccaatatcaagacctatcgaatcacactttgatattcaacgtcgaaggagctctactcaaatcaaactctttattcccttacttcatggttgtggcattcgaagccggaggggtgataaggtcacttttcctcttagttctttatccatttataagcttgatgagctacgaaatgggcttgaagacgatggtgatgctgagcttctttggagttaaaaaggaaagcttccgagtggggaaatcagttttgcctaagtattttctagaagatgttgggctcgagatgttccaggttttgaaaagaggaggcaagagagttgctgtgagtgatttaccacaagttatgattgatgtattcttgcgagattacttggagatagaagttgtggtcggaagagacatgaaaatggtcggtggttactacctaggcatcgtggaggataagaagaaccttgaaattgcttttgataaagtggttcaagaagaaagacttggtagtggtcgtcgtcttattggcatcacttcctttaactcgccaagtcacagatctctcttctctcaattttgccaggtaaattatattctggagacgtatatacataatctaaaagtgctttagttgtgttattcttgcattaaaaaatttatgattctaaaagagacattggtaaccttataaaaaaacagacgtaactgtaattttttgggagacaaacaaagacaaagcttatgttactcttctatttttttttcccttacatttggtaacccgataataaatttatcaagtcccgaataatacttgcattctaattttgttaaataaatagtatacgtttaattggtttataggaacttaataataagaaatgataagattctttgaacttaataagaatccccaataggattcttttagttttaactgtggctctttggacgaatcgagatgcattgattttttattttttttatgttaaaacttaaaaccattgactaagacttgttgtaatctagtactgcgtcacaaatcaaactaatatatcaagtaaatgctattttgaaatttttggaaattaattatattataaagttctgatgcaataaattcaaagcatgcatagactacgtacgacttggaaccggtccacttcaactgatatatttcatttcctaatgataatctccccctaatcctttcttttttcttgtcataaacaggaaatttacttcgtcagaaattcagacaagaaaagttggcaaaccctaccaaagatcaataccctaaaccattgattttccacgatggtcgtttagccgttaagccaacacctttaaacacactcgtattattcatgtgggccccattcgccgccgtcttagccgctgcaagactcgtcttcggcctaaacttaccttactccctagccaatcccttcctcgccttttccggtatccaccttactctcactgtcaacaaccacaacgacctaatatccgccgacagaaaaagaggttgtctctttgtgtgtaaccatagaacgttattggacccactttacatttcatacgctctaagaaagaaaaacatgaaagccgtgacgtatagtctaagcagattatctgagcttctggctccgatcaagaccgttagattgactcgtgatcgagtcaaagatggtcaagccatggagaaattgctgagccagggagatctcgtggtttgtccggaagggactacgtgtagagagccttacttgcttcggtttagtccacttttctctgaggtttgtgacgtcatcgtacctgttgctattgactcacacgtgactttcttctatggcacgacggctagtggtcttaaggcatttgatcccattttcttccttttgaatcctttcccttcctacaccgtcaaattgcttgaccctgtctctggaagtagctcgtccacgtgtcgaggagtccctgacaatggaaaagttaacttcgaggtggctaatcacgtgcagcatgagatcgggaatgcgttggggtttgagtgcaccaacctcacgagaagagataagtacttgatcttggccggtaataacggagttgtcaagaaaaaataattgatttgagtagtttattttcttaattagggcctttttgtgtgacgtacactttatttcttataagattttttttctatgatgtatctatctttttttagttaaatgtaattatctaattgtgtgacc

SEQ ID NO: 4 - DNA, 2379 bp, Arabidopsis thaliana
gtctgaaaccaaacaaagaaaatacacgaaggaaggaaaagaaaagtaaaaagaaaagctttctgcaaaatccaaacatgtccgccaagatttcaatattccaagctcttgtctttctattctaccggtttatcctccggcgatatcggaactctaaaccaaaataccaaaatggcccttcttctctcctccaatccgacctatcacgccacacattgatcttcaacgtagaaggagctcttctcaaatccgactctctcttcccttacttcatgttagtagcatttgaggcgggaggcgtaataaggtcatttctcctcttcattctctatccattgataagcttgatgagccatgagatgggtgtcaaagtgatggtaatggtgagcttcttcgggatcaaaaaagaaggttttcgagcggggagagcggttttgcctaaatactttctagaagatgtcggactcgagatgttcgaagtgttgaagagaggagggaagaaaatcggagtgagtgatgatcttcctcaagttatgatcgaagggttcttgagagattacttggagattgacgttgtggtcgggagagaaatgaaagtcgttggaggttattatctaggtatcatggaggataaaaccaaacatgatcttgtctttgatgagttagttcgtaaagagagactaaacaccggtcgtgttattggcatcacttccttcaatacatctcttcaccgatatctattctctcagttttgccaggtcagtctcttttatcattattttgtttgcgtttacgtttacagattatacagtttttaattatatttggtaagagattttttggaaatactaaacgagaatgttttaactactaaatattgaagcagccaaaaaaagaaactactccatatatgttgatatctatattatgacttgttacagtttttatatatatgatgaccgataattagtaaaaagcaacatagcatattaaattgtgatagtagtgacggtgatgaaacacggacaacttgcttgattgcgaatatgaaatattaacaacataacgtcaaaaatattaacaacatatataaaaagggaaaacaaaaggaaagaacaaatcaaggcctatatcattaaatacgaataagaaacgacgacagatcctgtgtgcatagttattagtcatgacaatgaatatatctcgtcgagttgaatctcaatacctgtaattgatttttgttaggtattttcattcattgttttcatgttatactcaactgattttcttgtccttttctctctaataggaaatttatttcgtgaagaaatcagacaagcgaagctggcaaaccctaccacgaagccagtaccctaaaccattgattttccatgatggccgtctcgcgatcaaaccaaccctaatgaacactttggtcttgttcatgtggggtcctttcgcagccgcagccgcagcagccagactcttcgtctctctttgcatcccttactctttatcaatcccgatcctcgccttttccggttgcagactaaccgtcactaacgactacgtttcatctcaaaaacaaaaaccaagtcaacgcaaaggttgtctctttgtttgtaaccataggactttattggaccctctctatgttgcattcgctttgagaaagaaaaacatcaaaactgtaacgtatagtttgagtagggtatctgagattttggctccgatcaagacggtgagactgacccgtgatcgggtgagcgacggtcaagccatggagaaattgttaaccgaaggagatctcgttgtttgtcctgaaggaaccacttgtagagaaccttacctgcttaggtttagccctttgttcaccgaggttagtgatgtcatcgttcccgtggctgtgacggtacacgtgaccttcttctacggtacaacggcgagtggtcttaaggcacttgacccgcttttcttcctcttggatccttatcctacctacaccatccaatttctcgaccctgtctccggtgccacgtgccaagatcctgatggaaagttgaagtttgaggtggccaacaatgttcagagtgatattgggaaggcgttggatttcgagtgcacaagtctcactagaaaagacaagtatttgatcttggccggtaataatggagtagttaagaaaaattaaaatttgttttaggatgatgagtgagtgatgagttcggttctttgggtatgtaagtaaccaatcgattatttcatttgtttattaataatgtctacgtaaatgtaactacaatctttttttcttggtaatgtacacatatgttctatatcatatccaaaatctttgtcagacaattttgcctttcatgc

SEQ ID NO: 5 - DNA, 2625 bp, Arabidopsis thaliana
aaatctctctttatctctctttctctgtttactaaatcgccagtattcgccgcaagaaagatgtctccggcgaagaagagccgaagttttcctccgataagcgagtgtaaaagcagagagtatgattcgatcgctgcggatctcgacgggactctgcttctgtcaagaagctctttcccttacttcatgctggtggccattgaagcaggtagtctcttccgtggattgatccttcttctttctcttcccatcgtcatcatcgcttatctcttcgtctccgaatctctcggaatccaaatccttatcttcatctccttcgccggtatcaagatcaagaacatcgaactcgtctctcgcgccgttcttacacggtaaccacaatcaccggatctttattcttttcgattttggaaccctttcgatttttaatcttcgttttcaaatgttaggttttacgcggcggatgtgaggaaagatagttttgaggtgtttgataaatgtaagaagaggaaagtagtggtgactgcgaatccgatagtgatggttgagccattcgtcaaagactatttgggaggagataaagttttgggaacagagattgaagttaaccccaaaacgatgaaagctactggttttgtgaaaaagcctggtgttcttgttggtgatctcaagagattagccatcttgaaagagttcggcgatgactcaccggatcttggcctcggtgaccgcacctccgatcacgatttcatgtccatttgcaaggtttctctatctctctcctctctcgtttcattttgcatatccagtttacgttgcttaaacctgatttaggtggtaattaattaaagaactaactacttaaatgcggtgtggagaggattcacatgatctatgggatacacatgatttcataaaaatctagaataatgttttaggatgataaaatccatagtttcaaaacacatacacgtataagtgaatttaagtagaggaaagtaatcactgagagtgacaaaaagtagataacagaatagaataatagttttcataaatgcttccaacctatcaccaactaccatcaccacataagttggacggtccatttaatgaatgatccgatatgtcaatgtttgaaagttcaatagtattttacaacatttcttaccataataatcgtgaatttcagaatgattcttattatatgatatgttaacaggaaggttacatggtgcatgagaccaaatcagccacaacagtccctatagagagtctcaagaaccgcataatcttccatgatggccgtcttgtccaacgtccgaccccattaaacgccttaatcatttacctttggcttcctttcggcttcatgctctctgtcttccgcgtctacttcaacctccctttacccgaacgcttcgtccgctacacttacgagatcctcggcattcacctcacaatccgtggccaccgtcctccacctccttcccccggaaaacctggaaacctctacgttctcaaccaccgcaccgcccttgaccccattatcatcgccattgctctcggccgtaagatcacatgtgtcacttatagtgtctctcgcctctccctgatgctctcacccattcctgctgttgctctgacccgagaccgtgtcgctgacgcagcccgcatgaggcaactcctcgagaaaggtattttccatgctcaattatcgttatagaaaagcagagtatctctgtttttactctggttttctacacatttatttggtttcattattatcacattatctctgttctgaaaataaaagctatcacctgagttttgattttttatgtaacattaatattatagaatcatacacattccatctacagaaaacacaaatttttctgatctttatacacacataattcctatctttgtaactcttttttatttattttgcaacaggtgatttggtgatctgtcctgaaggcacaacgtgtagagaaccatatctactaaggttcagtgctctatttgcagagctgagcgaccggatcgtgcctgtggccatgaactgcaagcaagggatgttcaacgggacgacagttagaggtgtgaaattctgggatccgtacttcttcttcatgaacccccgacctagctacgaggccactttcttggaccgtttgcccgaagaaatgacggtaaacggtggtggcaagactccttttgaggtggctaattacgttcagaaggtgatcggtggagttttggggttcgaatgtactgaacttacaaggaaggataagtatctgttgcttggaggcaacgacggaaaggttgagtctatcaataagaccaagtccatggagtaataatccagctagtctggttgctgctattttgcttgggagtgaagttaggtctttcgggttttcccaattacttttttttttgttttttttcatgtattgttcgaggaaaatgttttgttgaatatgcactggataatgaaactacatttgaatcaaagtacgtacatgtagtgtaccatttccccttgccttaactatgaatgatttgtaaactcaccttgtattataagatatcatagagaaaaacacattctcgact

SEQ ID NO: 6 - DNA, 2888 bp, Arabidopsis thaliana
atatccattctcaccttccaaatctccccactctctctctctctctctctctctctctctctctctctctctctatctctctctatctctctctatcttctcttttctcctctctccaacttcttcactaatgggagctcaggagaaacggcgccgtttcgagcagatatcaaagtgcgatgttaaggaccggtccaaccataccgtggccgctgatctagacggaacactactaatctctcgtagcgccttcccttactatttcctcgtagccctcgaggcagggagcttgctccgagcgttgatcctacttgtgtccgtaccattcgtttatcttacgtacttgaccatctccgagactttagccatcaacgtatttgtcttcatcacgttcgcgggtctcaagatccgagacgttgagctagtggtccgttccgtcctcccgaggttctatgcggaggacgtgaggcccgatacctggcgtatcttcaacacgttcgggaaacggtacataataactgcgagccctcgaattatggtcgagccattcgtgaaaacattcctaggagttgataaagttcttggaacagagctagaggtctccaaatcgggtcgggcaaccgggttcaccagaaaaccaggtattctcgtcggtcagtacaaacgtgacgtcgttttgagagagtttggtggcctagcgtctgatttacctgatttggggctcggcgatagcaagacggaccacgacttcatgtccatctgcaaggttatttccgtcatcatctctttttaaatttggtttgtcgttttacattactttcttactatccaccgacattaaataatatcgatcgaggacgttatgagtatatacaactatgtgtctctggaacttgtccgttggtgcagcttctttggtataaataattattgtgttgtcctatggagtttactgcccttcctaataaatttgggccaatcatatttatacctttttgatgtgtagcagtaaatgacgataaaattaggtatataactattaaaggtattatatattttttaggaaaacaaaaaagtgggtgttattttcctagatatcttttgttataaaaaagcataatgttttattctagataacatgctattttacttagaagtgtaacactggaattaaatgttttttacgacattatttttttccaaataaaaaatagtcagccattacaaacttttatatcaacctaaatgaccatatcttaatttactttggtttttcatatgctaatataatttctagatttgttggaacctttcaagtgaatttagaaacttgggtcacttgacaatactctaatagcattttctttggttaaagaacagtatttatttttttccttttggaaactctcaagtgaataggttcctcatacacgaattaccggtttttattttcctcatgtaaaagctatgttcatctctctttttttgtttgttaaagatcatctctcttttttatttttcttttttcgtttttgactctcctttcattttgtaagcttcgaatagttatcctaataaactagctagtatatatacatatatgtaatctagataaatatttttctaaccatcaataaatttcatagtacacatatatatctctcgtgactttggtgtccactctcctatatacttagagaaaaatgaaacatgcatggtactaaacaattacgtacaaatcacaactgcttttgtactttgacccatagaaataatttacgtactttttttttctgttactgactgcaggaaggttacatggtgccacgtacgaaatgcgaaccattaccaagaaacaaactcttaagccccataatattccacgagggcagattagtccaacgcccaacgccgttagttgctctgttaactttcctctggcttcccgtcggtttcgtcctctctatcatccgcgtctacacgaatattccgttaccggaacgtatcgcccgttacaactacaagcttactggcatcaagctagtcgtcaacggccaccctcctccgccgccaaaacctggccagccaggccatcttttggtctgcaaccaccgcaccgttctcgatcctgtggtcacagctgtcgcactcggccggaaaatcagctgcgtcacttacagcatcagcaagttctctgagctaatctcaccaatcaaagccgttgcgttgactcgtcaacgtgagaaagacgcagcgaacatcaagcgtcttttggaggaaggcgatctcgtgatatgtcccgagggaaccacgtgccgtgagcctttccttctccggtttagtgctcttttcgctgagctcacggaccggatcgttcccgtggcgatcaacacaaagcagagcatgttcaatggtaccaccacacgtggatacaagcttcttgatccttactttgcgttcatgaacccgaggccgacgtatgagatcacgttcctcaaacagattccagctgagctgacgtgtaaaggaggcaaatctccgatagaggttgcgaattacatacagagggttttgggaggaaccttaggttttgagtgcaccaatttcacaagaaaggataagtacgcaatgcttgctggtactgacggtagggttccggtgaagaaggagaagacgtgattattgatcggaggatggagaaaacaaagaaataatacaaagattggaatctttttcatttggatggttaattatcaatacttaaggggcaaagaattgataagtttgtttttgttgttacctttatcttctctgcttcggtgaatgtttcttcaagttttacatttagtacaataacggtttaagaaaagaaagaaaaaagtatcggt

SEQ ID NO: 7 - DNA, 1616 bp, Arabidopsis thaliana
atggagtcatcaactacaacatcgtattcagttgtgtcggagctagaaggaacactactgaaaaacccaaaacccttcgcctacttcatgctcgtagctttcgaggcttcaggactaatccgttttgcgaccttgctgtttctctggcctatcatagcactccttgacgttttgggctacagaaacggtagccttaagttaatgatcttcgtcgcaacggccggtttgcacgaatcagagattgaatcagtggctcgagcggttcttcccaaattcttcatggacgatatcagcatggacgcttggagggcgtttggttcgtgcgataagagagtcgttgtgacaagaatgccacgagttatggtggagagattcgctaaggatcatcttagcgccgatgaggtcatcggtacagagattgtcgtaaaccgtttcggttatgctaccggtttgattcaggaaaccaatgtggaccaatctgttttcaacagtgtggctaacttgtttgtggatcggagacctcaactaggtctaggacgacatattatctcggattctccaactttcctatctttatgtgaggtaagtactaaagacaacataacgttaggaattcttttaggttgttttataatttatgaaagcgttactatcatttttctaatctgaatgattgcatatgttatgtacatgtaggagcaagttcatgcaccagttccatcgaactacaacggtcataaccagcggctacatgtgcagccgttaccagttatcttccacgatggacgacttgtgaagctaccaacaccagccaccgcactcatcatccttctttggatcccctttggaataattctagccatgattcggatattcgttgggttcttacttccattatgggccataccttacgtctcacggatattcaacactcgattcatcgtaaaaggaaagcctccggcacaagcaaccaccggaaacccaggcgtactattcgtatgcactcatagaactctaatggatccggtcgtattatcctatgtgctcggacgtagcattccagctgttacctactctatttctcgtttatccgagatcctatctccgattccaacctttaggttaactagaattcgagatgtagatgcggagatgatcaagaaagaactgtctaatggggacttagtggtatatcccgagggaaccacttgtcgcgagccgtttttgctgagatttagcgcgcttttcgcagaattaaccgacaatattgtgcccgtggcaatgaactacagagttggattctttcatgcgactaccgtagaggctggaaaggtttggacccgatcttctttttcatgaacccgagaccggtttatgaggtgacgttcttgaaccagctcgaggtggaggcgacgtgttcgtcagggaagagcccttatgacgtggctaactatgtacagagaatcttggcagctacgttaggctttgagtgtactaactttacgaggaaggataagtatagagttctcgcagggaacgacggaaccgtgtcgtatttgtcgtttctcgatcaagtcaagaaggtcgtcaccactttcaaaccttttttgcattaa

SEQ ID NO: 8 - DNA, 2766 bp, Arabidopsis thaliana
aaaacataacgttccctctcttgtcttcttatttttgctctctgttctttagaactgccattaacgagagtgagagagagagacacacacacagcgagtgaaaatgtctccggagaagaagagtcaaaacttccctccaataacggaatgcagagacggagagtacgattcgatagccgccgatcttgacgggactctgcttctctcaagaagctccttcccttacttcatgctcgtcgctgttgaagctggaagccttttacgtggactaatcctccttctctcgttaccattcgtcattatctcttacctattcgtatccgaatctcttggtatccagatcctcatcttcatctcattcgctggtctcaaaatccgcgatatcgaacttgtctctcgcgcagttcttccacggtacgagatctaacatagagtgttttcttgttagtagatcactttagtttttttctgatgaactttgtttaggttttacgcggcggatgtgaggaaagacagttttgaggtgtttgataagtgtaagagaaaagtggtagtgacggcgaatccgattgtgatggtggaggcgtttgtgaaggattatcttggaggtgataaagttttgggaacagagattgaagttaaccctaaaaccaatagagccactggatttgtgaagaagcctggtgttcttgttggtgatcttaagaggttagccattttaaaagagtttggtaacgaatcacctgatctcggcctcggtgatcgaacctctgatcatgatttcatgtctctctgcaaggtactgttccaatctttttatccaatcttggtcgattatagggtaaagttaagttgaattattgagtgttgattggtttagtgtaagatgtgttttggaggtaacttgagaatctaagcaaacgtttgatttgcttgttgattaagctagataatctaatttaacttgttgactggtttagtgtcattatgtctacgcgtgaatcttataaaaataatgaaatttttgatttggaagcaattagtaaggttcataaatgtatccaaccaatcacttcacgtgaccctctggattatttttctatccccaaccaccttactagttccaccaacattttgggctgtccatttaatgaacacatacttgaaatatcataaacaatcttctgttgtttaatgcttagttttttgaatcttgattacacaatttagagatatgttacattttcctagtacttttagttataagtttgaaatgaataacgtttttggtattataacagaaaggttacatggttcatgcgaccaagtcagccacaacgattccaaaagaacgcttaaagaaccgcatagtcttccatgatgggcgtttagcgcaacgtccaactccgttaaacgccattatcacatacctatggcttccttttggtttcatcctctccatcattcgcgtctacttcaacctccctttacctgaaagatttgtccgttacacttacgagatgctcgggatccacttaaccattcgtggtcatcgtcctccacctccttcccctggaactcttggcaacctctatgtccttaaccaccgtaccgcgcttgatcccatcatcgttgctattgctcttggacgtaagatctgttgcgtcacttacagtgtctctcgtctctcccttatgctttctcctattcctgctgttgccctcacccgtgaccgtgccaccgatgctgccaacatgagaaaacttctcgagaaaggtactttacacatcttctcatcatatattactagagcatacctataacaagtttcactgtccagggggttcaataggagtttctatttctttctttttatttttcgatgaatacatatatagtagcccatgcaaatctcaatatagtaaatattactttggtatggtctttgatgtttactttggtacctaattaacgcatcttctaaccaaatttatataaatgaacataaaataatattgtttacttcttttgaatattgacttacattcacattaatctttagttgagaaattaaaaatcaatggagttgttgagctaatgatatgtttttggtgtataacaggcgacttggtgatatgtccggaaggcacgacgtgtagagaagagtatctactgagatttagcgctctattcgcagagctaagcgaccggattgtgccagtagcgatgaactgtaaacaaggaatgttcaacgggaccacagttaggggtgtgaagttttgggacccttacttcttcttcatgaacccaagaccaagctatgaagccactttcttggatcgtttgcctgaagaaatgactgtcaacggtggtggcaagactcctatagaggtggctaattacgtccagaaagttatcggcgcggttttgggcttcgaatgcaccgaacttactcgcaaggataaatatcttttgcttggaggtaatgacggcaaggtggagtctatcaacaacaccaagaagtgaatatcttattcttgttataacttggcattgacttattgcaagtaaggttggatcatatacgaattttcatgtgtgtaattgcttgagctcttgtcatcattatggtacgttattgttatgtttattctattttgttcttaatgtcgaacgggtaactatatccttaaaaactattttgtgatcccgtttctatacggatgtgtatagagactttctatttaaagtgcaaaattttattgg

SEQ ID NO: 9 - protein, 502 aa, Arabidopsis thaliana
MVMEQAGTTSYSVVSEFEGTILKNADSFSYFMLVAFEAAGLIRFAILLFLWPVITLLDVFSYKNAALKLKIFVATVGLREPEIESVARAVLPKFYMDDVSMDTWRVFSSCKKRVVVTRMPRVMVERFAKEHLRADEVIGTELIVNRFGFVTGLIRETDVDQSALNRVANLFVGRRPQLGLGKPALTASTNFLSLCEEHIHAPIPENYNHGDQQLQLRPLPVIFHDGRLVKRPTPATALIILLWIPFGIILAVIRIFLGAVLPLWATPYVSQIFGGHIIVKGKPPQPPAAGKSGVLFVCTHRTLMDPVVLSYVLGRSIPAVTYSISRLSEILSPIPTVRLTRIRDVDAAKIKQQLSKGDLVVCPEGTTCREPFLLRFSALFAELTDRIVPVAMNYRVGFFHATTARGWKGLDPIFFFMNPRPVYEITFLNQLPMEATCSSGKSPHDVANYVQRILAATLGFECTNFTRKDKYRVLAGNDGTVSYLSLLDQLKKVVSTFEPCLH

SEQ ID NO: 10 - protein, 585 aa, Arabidopsis thaliana
MVLPELLVILAEWVLYRLLAKSCYRAARKLRGYGFQLKNLLSLSKTQSLHNNSQHHLHNHHQQNHPNQTLQDSLDPLFPSLTKYQELLLDKNRACSVSSDHYRDTFFCDIDGVLLRQHSSKHFHTFFPYFMLVAFEGGSIIRAILLLLSCSFLWTLQQETKLRVLSFITFSGLRVKDMDNVSRSVLPKFFLENLNIQVYDIWARTEYSKVVFTSLPQVLVERFLREHLNADDVIGTKLQEIKVMGRKFYTGLASGSGFVLKHKSAEDYFFDSKKKPALGIGSSSSPQDHIFISICKEAYFWNEEESMSKNNALPRERYPKPLIFHDGRLAFLPTPLATLAMFIWLPIGFLLAVFRISVGVFLPYHVANFLASMSGVRITFKTHNLNNGRPEKGNSGVLYVCNHRTLLDPVFLTTSLGKPLTAVTYSLSKFSEFIAPLKTVSLKRDRKKDGEAMQRLLSKGDLVVCPEGTTCREPYLLRFSPLFAELTEDIVPVAVDARVSMFYGTTASGLKCLDPIFFLMNPRPVYCLEILKKLPKEMTCAGGKSSFEVANFIQGELARVLGFECTNLTRRDKYLVLAGNEGIVR

SEQ ID NO: 11 - protein, 530 aa, Arabidopsis thaliana
MSGNKISTLQALVFFLYRFFILRRWCHRSPKQKYQKCPSHGLHQYQDLSNHTLIFNVEGALLKSNSLFPYFMVVAFEAGGVIRSLFLLVLYPFISLMSYEMGLKTMVMLSFFGVKKESFRVGKSVLPKYFLEDVGLEMFQVLKRGGKRVAVSDLPQVMIDVFLRDYLEIEVVVGRDMKMVGGYYLGIVEDKKNLEIAFDKVVQEERLGSGRRLIGITSFNSPSHRSLFSQFCQEIYFVRNSDKKSWQTLPQDQYPKPLIFHDGRLAVKPTPLNTLVLFMWAPFAAVLAAARLVFGLNLPYSLANPFLAFSGIHLTLTVNNHNDLISADRKRGCLFVCNHRTLLDPLYISYALRKKNMKAVTYSLSRLSELLAPIKTVRLTRDRVKDGQAMEKLLSQGDLVVCPEGTTCREPYLLRFSPLFSEVCDVIVPVAIDSHVTFFYGTTASGLKAFDPIFFLLNPFPSYTVKLLDPVSGSSSSTCRGVPDNGKVNFEVANHVQHEIGNALGFECTNLTRRDKYLILAGNNGVVKKK

SEQ ID NO: 12 - protein, 520 aa, Arabidopsis thaliana
MSAKISIFQALVFLFYRFILRRYRNSKPKYQNGPSSLLQSDLSRHTLIFNVEGALLKSDSLFPYFMLVAFEAGGVIRSFLLFILYPLISLMSHEMGVKVMVMVSFFGIKKEGFRAGRAVLPKYFLEDVGLEMFEVLKRGGKKIGVSDDLPQVMIEGFLRDYLEIDVVVGREMKVVGGYYLGIMEDKTKHDLVFDELVRKERLNTGRVIGITSFNTSLHRYLFSQFCQEIYFVKKSDKRSWQTLPRSQYPKPLIFHDGRLAIKPTLMNTLVLFMWGPFAAAAAAARLFVSLCIPYSLSIPILAFSGCRLTVTNDYVSSQKQKPSQRKGCLFVCNHRTLLDPLYVAFALRKKNIKTVTYSLSRVSEILAPIKTVRLTRDRVSDGQAMEKLLTEGDLVVCPEGTTCREPYLLRFSPLFTEVSDVIVPVAVTVHVTFFYGTTASGLKALDPLFFLLDPYPTYTIQFLDPVSGATCQDPDGKLKFEVANNVQSDIGKALDFECTSLTRKDKYLILAGNNGVVKKN

SEQ ID NO: 13 - protein, 503 aa, Arabidopsis thaliana
MSPAKKSRSFPPISECKSREYDSIAADLDGTLLLSRSSFPYFMLVAIEAGSLFRGLILLLSLPIVIIAYLFVSESLGIQILIFISFAGIKIKNIELVSRAVLTRFYAADVRKDSFEVFDKCKKRKVVVTANPIVMVEPFVKDYLGGDKVLGTEIEVNPKTMKATGFVKKPGVLVGDLKRLAILKEFGDDSPDLGLGDRTSDHDFMSICKEGYMVHETKSATTVPIESLKNRIIFHDGRLVQRPTPLNALIIYLWLPFGFMLSVFRVYFNLPLPERFVRYTYEILGIHLTIRGHRPPPPSPGKPGNLYVLNHRTALDPIIIAIALGRKITCVTYSVSRLSLMLSPIPAVALTRDRVADAARMRQLLEKGDLVICPEGTTCREPYLLRFSALFAELSDRIVPVAMNCKQGMFNGTTVRGVKFWDPYFFFMNPRPSYEATFLDRLPEEMTVNGGGKTPFEVANYVQKVIGGVLGFECTELTRKDKYLLLGGNDGKVESINKTKSME

SEQ ID NO: 14 - protein, 501 aa, Arabidopsis thaliana [sequence omitted]

SEQ ID NO: 15 - protein, 500 aa, Arabidopsis thaliana
MESSTTTSYSVVSELEGTLLKNPKPFAYFMLVAFEASGLIRFATLLFLWPIIALLDVLGYRNGSLKLMIFVATAGLHESEIESVARAVLPKFFMDDISMDAWRAFGSCDKRVVVTRMPRVMVERFAKDHLSADEVIGTEIVVNRFGYATGLIQETNVDQSVFNSVANLFVDRRPQLGLGRHIISDSPTFLSLCEEQVHAPVPSNYNGHNQRLHVQPLPVIFHDGRLVKLPTPATALIILLWIPFGIILAMIRIFVGFLLPLWAIPYVSRIFNTRFIVKGKPPAQATTGNPGVLFVCTHRTLMDPVVLSYVLGRSIPAVTYSISRLSEILSPIPTFRLTRIRDVDAEMIKKELSNGDLVVYPEGTTCREPFLLRFSALFAELTDNIVPVAMNYRVGFFHATTARGWKGLDPIFFFMNPRPVYEVTFLNQLEVEATCSSGKSPYDVANYVQRILAATLGFECTNFTRKDKYRVLAGNDGTVSYLSFLDQVKKVVTTFKPFLH

SEQ ID NO: 16 - protein, 500 aa, Arabidopsis thaliana
MSPEKKSQNFPPITECRDGEYDSIAADLDGTLLLSRSSFPYFMLVAVEAGSLLRGLILLLSLPFVIISYLFVSESLGIQILIFISFAGLKIRDIELVSRAVLPRFYAADVRKDSFEVFDKCKRKVVVTANPIVMVEAFVKDYLGGDKVLGTEIEVNPKTNRATGFVKKPGVLVGDLKRLAILKEFGNESPDLGLGDRTSDHDFMSLCKKGYMVHATKSATTIPKERLKNRIVFHDGRLAQRPTPLNAIITYLWLPFGFILSIIRVYFNLPLPERFVRYTYEMLGIHLTIRGHRPPPPSPGTLGNLYVLNHRTALDPIIVAIALGRKICCVTYSVSRLSLMLSPIPAVALTRDRATDAANMRKLLEKGDLVICPEGTTCREEYLLRFSALFAELSDRIVPVAMNCKQGMFNGTTVRGVKFWDPYFFFMNPRPSYEATFLDRLPEEMTVNGGGKTPIEVANYVQKVIGAVLGFECTELTRKDKYLLLGGNDGKVESINNTKK

SEQ ID NO: 17 - protein, 59 aa, Nicotiana tabacum
QVTFLNQLPVEATCSSGKGPHDVANYVQRILATTLGFECTNFTRKDKYRVLAGNDGIVS

SEQ ID NO: 18 - DNA, 512 bp, Nicotiana tabacum
caagttttaattaaatctatgtccaaacactagcttaattttatttatcttcttcatttatattttttctacaggtaacattcttgaatcagctaccagttgaagccacgtgttcatctgggaaaggtccacatgatgttgcaaattatgttcagaggatattggctacaacattagggtttgagtgtacaaactttacgagaaaggacaaatatagggttcttgctggaaatgatggaatagtttcacaaaattatgggacaaatggggttaacaagtttaatgaacacgtttaagcaagtggtgggcactttcaatccttttattcactgaaacaagtaaaagtttttgagtttttttttagtgatacgtttcacatactatataatttgtttttatcctatcattgtataaattacacagaaagaaaaaaaaactgagaaagcattctttgttttatatcttgtttcattaaataaattgttagagctattcagtgattaagaaaaagt

SEQ ID NO: 19 - protein, 523 aa, Medicago truncatula
MDSVVSELEGTLLKNSDVFSYFMLVAFEASGLIRFVLLLILWPVIRFLDMFGMEDISLKLMIFVAVAGIHKSEIESVSRAVLPKFFMDDLDMEAWKIFSSYAKSVVVTKMPGVMVKRFVEEHLRADEVIGSELVFNRFGLATGFVQSESITSVSERVAKLFNNQVPNLGMLRFSTTTSSNHSFSKLCKEQMQPPFRTSQKHNDSQLLRPHPVIFHDGRLVKRPTPFTSLLIILWIPIGILLAIIRLTLGAILPFWAIPHMSRLFGGKVIVKGNPPFPPTAGGPGVLFVCTHRTLMDPVVLSSVLQRKTPAVTYSISRLSEILSPIPTVRLTRMRNVDAERIKYELSKGDLVVCPEGTTCREPFLLRFSALFAELTDRIVPVAMNYRVGFFHATTARGWKGLDPVFFFMNPRPVYEVTFLNQLPVEATCSSGKSPHDVANYVQRILAATLGFECTNFTRKDKYRVLAGNDGIVSCTSLVDRVKKVVGSTKEASIKEERSGMIISRDKEKENVTVSDCNKNIIVL

SEQ ID NO: 20 - DNA, 1572 bp, Medicago truncatula
atggattctgttgtctctgaattagaaggcacacttctaaaaaactcagatgtattctcctacttcatgttagtagcatttgaggcctcaggtttgattcgcttcgttcttctgttaatcctatggcccgtgattcggttcctagacatgttcggcatggaggacataagtctcaagctaatgatctttgtagccgtagctggaattcataaatctgagattgaatcagtttcaagagctgttttaccaaaattcttcatggatgatcttgacatggaagcttggaagatttttagctcttacgcgaaaagcgttgtggtaacaaaaatgccaggagttatggtgaagaggtttgttgaggaacatttacgtgcagatgaagtaattggaagtgaattggttttcaataggtttggtttagctacaggatttgttcaaagtgaatcaattacttccgtttctgaaagagttgctaaacttttcaataaccaagttccaaatttgggtatgttaaggtttagtactactacatcaagcaatcattctttctccaaactttgcaaggaacaaatgcaaccaccatttaggacaagccaaaagcacaatgatagccaacttctccgacctcatccggtgatcttccacgacggccgattggtgaaacgaccaacaccgttcacctccctactaatcatcctttggatcccaataggtattttactagccataatcaggttaaccttgggagccattttacctttttgggccattcctcacatgtcaaggttatttggtggcaaagttattgtgaagggaaatccacctttccctcccaccgcaggcggtcccggtgtccttttcgtgtgtactcaccgcactctaatggaccctgttgtcctctcttccgtccttcaacgtaaaacccccgcggtaacatactctatttctcggttatcggaaatcctctcccccattcctaccgtgagactaaccaggatgcgaaatgtggacgctgagagaataaaatatgagttatccaaaggagatttagttgtctgtccagaaggaacaacatgtcgcgagcctttccttttacgatttagtgctctatttgccgagctaactgaccggatagtaccggtcgccatgaactatagagtagggttcttccatgcaaccactgcaaggggttggaaaggtttggacccggttttcttcttcatgaacccaagaccagtttatgaggttacattcttgaaccagttgccggttgaagcgacgtgttcgtccgggaagagtccacacgatgtggccaattatgttcagaggatattggctgcaactctagggtttgagtgcactaactttactcggaaagataaatatagggttttggctgggaatgatggcattgtgtcttgtacttcattggttgatcgagtaaagaaggtggtcggttctacaaaagaagcttccataaaagaggaaagatccgggatgataatctcaagggataaagagaaggagaatgtcaccgtatctgattgcaacaagaatatcattgttctataa

SEQ ID NO: 21 - protein, 519 aa, Oryza sativa
MSASAEAKSRRERRSVVAELEGALLRDAATFPYFMLVAFEASGLPRFAALLALWPLLWALERALGRGDLALRAAAFVATAGVPRAEVEAVARAVLPKFMADDVDPAAWAAFGSCGGRRVVVTRMPRVMVERFAREHLGAHEVVGCDLEYSRLRRSTGFVRGGGGGERAVAERVRALFADGDRPDVGIARSESATRSFLPFCKKQLRPPFCEDDGDDVAAAGEQHKCPPFRPVIFHDGRLVCRPTPLMSLVILLWLPLGALVAFVRIAVGISVPIQIIPRIAPYFGGAVVVHGAPPPAAASGPASGVLFVCTHRTLMDPVVLATVLGRRVAAVTYSISRLSEVLSPIPTVRLTRDRGVDAAQMRAELSRGDVAVCPEGTTCREPFLLRFSALFAELSDRIVPVAMNYRVGLFHPTTARGWKAMDPIFFFMNPRPVYEVTFLNQLPAEETCAAGKSAVDVANYVQRILAAKLGFECTTFTRKDKYRVLAGNDGIVNVKPPAAADPAPWQRRLKEVLGFLLH

SEQ ID NO: 22 - DNA, 1560 bp, Oryza sativa
atgtcggcgtcggcggaggcgaagagtcgacgggagaggcggtcggtggtggcggagctggagggggcgctgctgcgcgacgcggcgacgttcccgtacttcatgctggtggcgttcgaggcgtccgggctgccgcggttcgcggcgctgctggcgctgtggccgctgctgtgggcgctggagcgggcgctggggcggggcgacctggcgctgcgcgccgcggcgttcgtggcgacagcgggcgtgccgcgcgcggaggtggaggccgtggcgcgcgccgtgctgcccaagttcatggccgacgacgtcgacccggcggcgtgggcggcgttcgggtcctgcggcgggcggcgcgtcgtggtgacacgcatgccccgggtcatggtggagcggttcgccagggagcacctcggcgcgcacgaggtggtcggctgcgacctcgagtacagccgcctcaggcggtccaccggcttcgtcaggggcggcggcggcggcgagcgcgcggtcgccgaacgcgtgcgcgcgctcttcgccgatggcgaccggccggacgtcgggatcgcgcggtccgagtcggcgacgcgatccttcttgcccttctgcaagaagcaactccggccgccgttctgtgaggacgacggcgacgacgtcgccgccgccggcgagcagcacaaatgccctccgttccggccagtcatcttccacgacggccgcctcgtgtgccggcccacgccgctcatgtcgctcgtcatcctcctgtggcttccgcttggcgcgctcgtcgccttcgtccgcatcgccgtcggcatctccgtgcccatccagatcatcccccgcatcgcgccctacttcggcggcgccgtcgtcgtgcacggcgccccgccgcccgccgccgccagcgggccggcgtccggcgtcctcttcgtgtgcacgcaccggacgctgatggacccggtggtgctcgccaccgtgctcggccgccgcgtggccgccgtgacgtactccatctcccgcctctcggaggtcctctcgccgatcccgacggtgcggctgacgcgcgaccgcggcgtggacgcggcgcagatgcgcgccgagctgtcgcgcggcgacgtcgcggtgtgccccgaggggacgacgtgccgggagcccttcctgctccgcttctccgcgctgttcgccgagctcagcgaccggatcgtgccggtggcgatgaactaccgcgtcgggctcttccacccgacgacggcgagggggtggaaggccatggaccccatcttcttcttcatgaacccgaggccggtgtacgaggtgaccttcctcaaccagctccccgccgaggagacgtgcgccgccggcaagagcgccgtcgacgtcgccaactacgtgcagaggatactcgccgccaagctcgggttcgagtgcaccaccttcacgcgcaaggacaagtaccgcgtgctcgccggcaacgacggcatcgtcaacgtcaagccgccggcggcggcggatccggcgccgtggcagcgccgcctcaaggaggttctcggcttcttgctccactaa

SEQ ID NO: 23 - DNA, 711 bp, Brassica napus
cggggtctgcgttcacgttaacagtattcagcaaattctcttgagcctaatctctccaataaaagccgttgcgttgactcgtcagcgcgagaaagacgcagcgaacatcaagcgtctcttagaggaaggtgatctcgtgatatgtccggagggaaccacttgccgtgagcctttccttctccggtttagtgcactccttgccgagcttacggataggatcgttcccgtggcgattaatacgaaacaaagcgtgtttaatggtacaaccacacgtggctacaagtttcttgatccttacttcgcgttcatgaacccggttccgacgttcgagatcacgttccttaaacagcttccggctgagctgacgtgtaaaggaggcaagtcgccgatagaggtggcgaattacatacagagggttttagcaggaaccttagggtttgagtgcaccaacttcacaagaaaggataagtacgcaatgctagctggtaccgacggtagggttccggtgaagaaggagaagatatgattatatttgattggaggaaagagatgatgaagaaataatacaaatatttaaatcttttttttttcatttagcaagaattggtaagtttgttaatttatttgtcttctatgcttcgataattgtttctttgcaagttttttcgtgatattttcaagttttacttttagtataataaacgattaaga

SEQ ID NO: 24 - protein, 174 aa, Brassica napus
GVCVHVNSIQQILLSLISPIKAVALTRQREKDAANIKRLLEEGDLVICPEGTTCREPFLLRFSALLAELTDRIVPVAINTKQSVFNGTTTRGYKFLDPYFAFMNPVPTFEITFLKQLPAELTCKGGKSPIEVANYIQRVLAGTLGFECTNFTRKDKYAMLAGTDGRVPVKKEKI

SEQ ID NO: 25 - DNA, 398 bp, Nicotiana tabacum
atgaaaggaacatagggaatatccccgaggagtttgctcacttatttttcgctcctccacaggtataagttctcttgctacagtaaaattttagttgcttttcttatggttatatgctgcaactttatcgtatgagtgcactaattttactaggaaggataaatacagtgctttggctggaaatgatggaactgtgacaacaaaacctggacttgcggccaagaaaatgatgggctgctaatagagaaaaggttgtatggggtttaaaaattttcagaagtttttacttgtggtcatgttccaaattgtaaagagaattaaacagttaacagttgacaaaaaaacagaaataaaatacaaaacctctcattaaagtgattagtcgtttggcttcgaat

SEQ ID NO: 26 - protein, 78 aa, Nicotiana tabacum
LYAATLSYECTNFTRKDKYSALAGNDGTVTLLFLWLYAATLSYECTNFTRKDKYSALAGNDGTVTTKPGLAAKKMMGC

SEQ ID NO: 27 - protein, 543 aa, Oryza sativa
MPKKKLSHRLFSALVSLLLHGKPISRSSSNTNTTLPHPSLLHKSSSSFPPMEKLAAKTLVLDVEGGLLRSSSLFPYFMLVALEAGGFLRGLVLLLLYPLLCVMGSDMALKVMAMVSFCGLRASRFRAGRAVLPKWFLEDVGEEGFDVMRSAMRRVCVTKMPRIMVEGFLKEYLEVEVVSGREMKVIWGFFTGIMEEEEGGDQEEVLLEEKKMLVDVVGFSTSLEFLQHHLSHCCKEVYLVTREEKARWSALPRDKYPKPMVFHDGRLAFRPAAGDTLAMFTWLPFGAALAVARLAVALAVPYRYSTPILAATGLSWRLKGEAPTPLAGAGDGARRRGQLFVCNHRTLIDPVYVSVALDRPVRAVSYSLSRLSELISPIGRTVRLTRDRDSDGRAMARLLDGGDLVVVCPEGTTCREPCLLRFSPLFAELSDDVVPVGIAVDTAMFYATTAGGLKCLDPLYYIANPRTCYAVQFLERVDTSPARERRAPSTDVANLVQRRMGDALGYRCTMLTRKDKYLMLAGNDGVVNTTQDNHSAPGKKKMQ

SEQ ID NO: 28 - DNA, 1641 bp, Oryza sativa
atgcccaagaagaagctgtcacataggctgttctctgccttggtatccctactccttcatgggaagcctatctctagatcatcatccaacaccaacaccacattgccacacccttccttgctgcacaagtcctcctcaagcttccctcccatggagaagctcgccgcgaaaaccctagtactcgacgtcgaaggcggtttgcttagatcatcctccttgttcccctacttcatgcttgtggcactagaggcaggagggttcttgaggggccttgtgctcctcctcctctacccattgctatgtgtcatgggaagtgacatggccttgaaggtcatggcaatggtgtccttctgcggtttacgggcaagccggtttcgggccgggcgagccgtgcttcccaagtggttccttgaggatgttggggaggaagggtttgatgtgatgaggagtgccatgaggagggtgtgtgtgacaaagatgcctaggatcatggttgaagggttcttgaaggagtacttggaggttgaggttgtttcaggtagggagatgaaggtgatttgggggttcttcactggcatcatggaggaggaagaaggtggtgatcaagaggaggtgttgcttgaggagaagaaaatgttggttgatgttgtgggcttctctacctccttggagttcctccaacatcatctctcacattgttgcaagctagttcaggaggtgtacctggtgacacgtgaggagaaggcgaggtggtcggcgctgccgcgcgacaagtacccgaagccgatggtgttccacgacggccgcctcgcgttccggccagccgccggcgacacgctcgccatgttcacctggctccccttcggcgccgccctcgccgtcgcccgcctcgccgtcgcgctcgccgtgccgtacaggtactcgacgccgatcctggccgccacgggcttgtcgtggcgcctcaagggcgaggcccccacgccgctcgccggcgccggcgacggcgcgcgccggcgcgggcagctgttcgtgtgcaaccaccggacgctgatcgacccggtgtacgtgtcggtggcgctggaccgccccgtgcgcgccgtgtcgtacagcctgagccggctgtcggagctgatctcgccgatcgggcggacggtgcggctgacgcgggaccgcgacagcgacgggcgcgccatggcgcggctcctcgacggcggcgacctcgtcgtcgtctgccccgaggggacgacgtgccgggagccgtgcctgctccggttcagcccgctgttcgcggagctgagcgacgacgtggtccccgtcgggatcgccgtcgacacggcgatgttctacgcgacgacggccggcgggctcaagtgcctggacccgctctactacatcgccaacccgaggacgtgctacgccgtgcagttcctggagagggtggacacgtcgccggcgagggagcggagggcgccgtccaccgacgtggccaacctcgtgcagaggaggatgggcgacgcgctcggctaccgctgcaccatgctcaccaggaaggacaagtacctcatgctcgccggcaacgatggcgtcgtcaacaccacccaagacaaccactctgctccggggaagaagaagatgcaataa

SEQ ID NO: 29 - protein, 471 aa, Oryza sativa
MATMRNRFATRMLNLHRLVKSNLVAGLLRRRGHRPLDAPPPRIRVLIDGNEATVVDVDRLLLKPSSPSSATALFPPFFLLAVEAGGFVRGLVLLALYPALRLLTAEGARLKAMAMVCFLGLRRGEAARAGRAVLPRYFFREAADVEALMEAAASARKEVITVVAAASRLFPTVMVETFLKEYVGFDAVVGRETPRNGAGPRPLIFHDGRLAFTPTPAAALAMYVYLPLAVALSVLRIAIFTLLPRGVSGAAAALAGVRLRVAGAPRPPAAGDAAGGRLYACNHRTLLDAVAVSGALGRPVSSVTYSLGRLSELLSPIPLLPLAREREEDRRRMASLLSRGDVVVCPEGTTCREPYLLRFSPLFAELADEVNPVAVRAAAGMFYGTSTSPSAKCFDSVFFLMNPSPEYGVHFLEPVATAGAGAGSSIEVANRVQRVIAGALGYEATTLTRKAKYLLLAGNEGGVATNRSNNK

SEQ ID NO: 30 - DNA, 1416 bp, Oryza sativa
atggcgacgatgaggaatcggttcgccactcgaatgctcaacctccaccgactcgtcaagtccaatctcgtcgccggcctcctccgccgccgcggccaccgcccgcttgacgcgccgccgccgcggatacgcgtgctcatcgatggcaacgaggctacggtggtcgacgtcgaccggctgctgctcaagccgtcctcgccgtcctccgccaccgcgctgttcccgccattcttcctcctcgccgtcgaggccggcgggttcgtcaggggcctcgtgctgctcgcgctctacccggcgctgcgcctcctgacggcggagggggcccggttgaaggccatggccatggtctgcttccttgggctgcgccgcggcgaggcggccagggccggcagggctgtgctccccaggtatttcttccgggaggcggcggacgtcgaggcgctgatggaggcggcggcttctgcgcgcaaggaggtgatcacggtggtggcggcggcgagcaggttgttcccgacggtgatggtggagacgttcttgaaggagtacgtggggttcgacgcggtggtcgggagggagacaccgaggaatggagcagggccgcggccattgatcttccacgatggccggctcgcgttcacgccgacgcccgcggcggcgctcgccatgtacgtgtacctcccgttggccgtcgccctctccgtcctccgcatcgccatcttcaccctgctcccacggggcgtgtcgggcgccgcagccgcgctcgccggcgtgaggctgcgcgtcgccggcgcgcctaggcctccagcggcgggcgacgccgccggcggccggctctacgcctgcaaccaccgcacgctgctcgacgcggtcgccgtgtcgggcgcgctcgggaggcccgtgtcctccgtgacctacagcctgggccgcctgtcggagctgctctcgccgatcccgctgctgccgctggcgcgcgagcgcgaggaggaccggcgccgcatggcgtcgctgctgtcgcgcggcgacgtcgtggtgtgccccgaggggacgacgtgccgggagccgtacctgctccggttcagcccgctgttcgccgagctcgccgacgaggtgaaccccgtggccgtgcgcgccgccgcggggatgttctacggcacgtcgacgtcgccgtccgccaagtgcttcgactcggtgttcttcctgatgaacccgtcgccggagtacggcgtgcacttcttggagccggtggccacggccggcgccggcgccggcagcagcatcgaggtggcgaaccgggtgcagagggtgatcgccggcgcgctcgggtacgaggcgacgacactgacgaggaaggccaagtacttgctgcttgccggcaacgaaggtggcgtggcaaccaacagaagcaacaacaagtga

SEQ ID NO: 31 - protein, 254 aa, Lycopersicon esculentum
MPIGIILSILRVYLNIPLPEKIVRYNYIILGIKLIVKGTTPHHPPKNGQRGVLFVCNHRTILDPVVTAVALGRKISCVTYSISKFSEMISPIKAVALSREREKDAAHIKQLLEEGDLVICPEGTTCREPFLLRFSALFAELTDRIVPVAINTKQSVFNGTTVRGYKLLDPYFVFMNPRPTYEITFLNQLPPELNCTNGGKSAIEVANYIQKVLGGTLGFECTNLTRRDKYAILAGTDGRVAIKDKDKEKENEKK

SEQ ID NO: 32 - DNA, 762 bp, Lycopersicon esculentum
gccaataggaataattctatcaattcttagggtatatcttaatattcctttaccagaaaaaattgttagatataattatattatccttggtattaaacttattgtaaaaggtaccaccccccaccacccccctaaaaatggccaacgtggtgtactttttgtgtgcaaccatagaactatactagaccccgttgttactgctgttgcactaggtagaaaaataagttgtgttacgtatagtattagtaaattttctgagatgatatcgccgattaaagcggtcgcgttatctcgagagagggagaaagatgcagcacatattaaacaattacttgaagaaggtgatttggttatatgtcccgaaggtacaacatgtagggaaccgtttttattgagatttagcgcactttttgctgaattgactgataggattgtacctgtggcaatcaacactaagcaaagtgtgtttaatgggacaactgttagagggtacaaattattggatccttattttgttttcatgaatccaagaccgacttatgagattacattcctgaatcaactccctcctgaacttaattgtactaacggagggaaatccgcgattgaagttgctaattatattcaaaaggtacttggtggtacattgggatttgagtgtactaatttgacaagaagagataagtacgcaatacttgctggaactgacggtcgcgttgcaataaaggacaaggacaaggagaaggagaatgagaagaaata

SEQ ID NO: 33 - DNA, 505 bp, Lycopersicon esculentum
taaacgtccaacaccatcaattgctctattaattcttttatggattccatttggcattatattagcaacaatacgtattataataggtttaattctaccattatggattgtaccctatttagctccattatttggtggtaaagttattgtcaagggcaaaccaccaccaccagcgtcaatcactaactcgggcgtgctctttgtgtgcacacaccgaaccctactggaccctgtggtcctatccaccgtacttcaacgtaggataccggccgtgacgtactcaatctctcgattatcggagattttgtccccgataccgaccgttcgtctcacgaggattcgagaagtggacgcgcaaaaaatcaaacgtcaactcgagaaaggagatttagtcgtctgccccgaagggacgacatgtagggaaccatttttactgagatttagcgcgctattcgctgaattaaccgatcgaatcgtaccagtggcgatgaattatagagtaggg

SEQ ID NO: 34 - protein, 168 aa, Lycopersicon esculentum
KRPTPSIALLILLWIPFGIILATIRIIIGLILPLWIVPYLAPLFGGKVIVKGKPPPPASITNSGVLFVCTHRTLLDPVVLSTVLQRRIPAVTYSISRLSEILSPIPTVRLTRIREVDAQKIKRQLEKGDLVVCPEGTTCREPFLLRFSALFAELTDRIVPVAMNYRVG

SEQ ID NO: 35 - protein, 246 aa, Lycopersicon esculentum
MGAHPHIEPISKYNKEIDRSNQTIASDLDGTLLISTSSFPYFFLIALEGGSLIRALLLLLFVPIIYFIYIFISESIAIETLIFISFAGLKIKDIEIVSRAVLPKFYSEDVHPHTWKVFNSFGKRYIVTANPTIMVEHFAKNFLGVDKVLGTELHVTKSGRATGFVTKPGVLVGQYKRDALIKEFGTNVPHVGLGDRETDRDFISICKEGYMVPRSKIEPLPRNKLLSPVIFHEGRFVQRPTPIMAL

SEQ ID NO: 36 - DNA, 738 bp, Lycopersicon esculentum
atgggtgctcatccccatattgaaccaatctcaaaatataacaaagaaattgatagatcaaatcaaacaattgcatctgatttagatggaacactcttaatatcaacaagttcatttccatatttctttcttattgccttagaaggtggtagtctcataagagcattactactactactatttgttccaattatttacttcatttacatattcatctcagaatctattgcaattgaaaccctaatcttcatatcctttgctggattaaagattaaagacatcgaaatcgtctctagggctgttttacctaagttttattctgaagatgtacatccacatacttggaaagtgttcaattcttttggtaagagatatattgttaccgcgaatccaacaattatggttgaacattttgcaaagaatttcttaggtgttgataaagttcttggaactgaattacatgttacaaaatctggacgtgcaactggatttgtaacaaaacctggtgttcttgttggacaatataaaagggatgctcttattaaagaatttggaacaaatgtacctcatgtaggtcttggagatagagagactgatcgcgatttcatctccatatgcaaggagggttacatggttccaagatcaaaaatagaacctttaccaagaaataaacttctaagtccagtaatttttcatgaaggtcgatttgtacaaagacctacaccaattatggcactt

SEQ ID NO: 37 - protein, 88 aa, Lycopersicon esculentum
TPVRGVKFWDPYFLFMNPRPTYELTFLEPLPMEMTCKAGKTSIEVANHVQKVLGGVLGFECTQLTRKDKYMLLGGNDGKVESMYSKKA

SEQ ID NO: 38 - DNA, 267 bp, Lycopersicon esculentum
acaccagtccgcggagtgaagttttgggatccatatttccttttcatgaacccaaggccaacttatgaattgacatttcttgaaccattgccaatggaaatgacatgtaaggctgggaaaacatcaattgaggtagccaaccatgtgcagaaagtgttgggtggggttcttggatttgaatgcactcagttgacaaggaaggataagtatatgttgcttggtggaaatgatggcaaagtggagtctatgtactccaagaaagcctga

SEQ ID NO: 39 - DNA, 1191 bp, Lycopersicon esculentum
gtcacttccaaaatccaaaaaatccttcccgtcggtgacgacatgcgatacctccgccgttaaccaccactccgtggcggcggatctcgacggaactctgttgatttcccgttcatctttcccttacttcatgctagtagcaatcgaagccgggagccttttccgaggactgatcctccttctctctttccctctcatcgcgattgcgtacgtatttgtatccgaagcattagctattcagatgctgatttacatttccttcgccggactcaaagtccgggacattgagctcgcttcgcgagctgttctcccgagattttacgcgacggatgttaggaaggagagtttcgaggttttcgatcagtgtaagaggaaagttgtggttactgcaaatccgactattatggttgagccttttgtgaaggattttttaggaggtgataaagtacttggaacagaaatcgaggtgaatccgaagacgaaaaaggctactggatttgtgaagagtcctgggatttttggtagggaaatggaagaaattgtcaattttgaagggaatttggagaagaaatgcctgatattggattggggtgatcgagaatccgatcatgattttatgtccatttgcaaggagggctatatggtgctccctagtgaatcagcaaagccagtaccacttgatcgcttgaaaagcagattgatcttccatgacggtcgtttggtgcaacgcccaactccattcaacgcgttagtcacctacatttggttgccatttggatttgctcttggagttttccgtgtatacttcaacctccctctccctgagcgtattgttcgttatacatatgggatggtagggatcaacctcgtgatcaaaggccctcgaccacctccaccatcaccgggtaccccagggaacctctatgtgtgtaatcatcgttcagcacttgaccctatcgtcattgctattgcactagggaggaaagtgtccactgtcacatatagcgtgagcaagctctcgagattcttatcccccattccagccattgccttgacacgtgatcgtgaagctgatgctgcaatgattcagaaattgctcgagaaaggtgatctcgttgtatgccccgaggggactacatgtagggagcccgtttttgctgaaggtcagtgctctggttgcagaattaagtg

SEQ ID NO: 40 - protein, 397 aa, Lycopersicon esculentum
MSLPKSKKSFPSVTTCDTSAVNHHSVAADLDGTLLISRSSFPYFMLVAIEAGSLFRGLILLLSFPLIAIAYVFVSEALAIQMLIYISFAGLKVRDIELASRAVLPRFYATDVRKESFEVFDQCKRKVVVTANPTIMVEPFVKDFLGGDKVLGTEIEVNPKTKKATGFVKSPGIFGREMEEIVNFEGNLEKKCLILDWGDRESDHDFMSICKEGYMVLPSESAKPVPLDRLKSRLIFHDGRLVQRPTPFNALVTYIWLPFGFALGVFRVYFNLPLPERIVRYTYGMVGINLVIKGPRPPPPSPGTPGNLYVCNHRSALDPIVIAIALGRKVSTVTYSVSKLSRFLSPIPAIALTRDREADAAMIQKLLEKGDLVVCPEGTTCREPVFAEGQCSGCRIK

SEQ ID NO: 41 - protein, 505 aa, Medicago truncatula
MSPPKPKRSFPPISECNGKTSSSSPRESVAADLDGTLLISRSSFPYFMLVAVEAGSLLRGFILLLSLPLVIIAYLFISEALGIQILIFISFAGLKVRDIELASRAVLPRFYAADVRRESFEVFDKCKRKVVVTANPTVMVDAFVKDYLGGDKVLGTEIEVNPKTKKATGFVKKPGVLVGDLKRLAVVKEFGDEVPDIGLGDRKTDHDFMSICKEGYMVPPSKSAKPVPLERLKSRLIFHDGRFVQRPDPLNAIITFAWLPFGFILSIFRVYFNLPLPERIVRYTYELLGIKLVIRGNRPPPPSRGTPGNLYVCNHRTALDPIVIAIALGRKVSCVTYSVSKLSRFLSPIPAVALTRDRAADAARITEILEKGDLVVCPEGTTCREPFLLRFSALFAELSDRIVPVAVDCKQNMFFGTTVRGVKFWDPYFFFMNPRPVYEINFLDRLPEEMSVKAGGKSSIEVANHVQKVLGDVLGFECTGLTRKDKYLLLGGNDGKVESMYGGKK

SEQ ID NO: 42 - DNA, 1518 bp, Medicago truncatula
atgtcaccgccgaaaccgaaacgtagcttccctccgatatcggaatgcaacggaaaaacttcctcatcttcgccgcgtgaatctgttgccgctgatctcgatggcacgcttctgatttcgagaagctcttttccttacttcatgcttgtcgccgttgaagctggtagcctcctccgtggcttcatccttcttctctcccttccgttggttatcatcgcttatctcttcatttctgaagctctcggtattcagatacttatcttcatctctttcgccggtctcaaagttcgtgacattgaactcgcttcacgcgctgttcttccaaggttttatgctgcggatgtacggagggagagtttcgaggtgtttgataagtgtaaacggaaggtagtggtgactgcgaatccgacggtgatggtggatgcgtttgtgaaggattatctcggcggggataaggttttgggaacggagatcgaggtgaatccgaagacgaagaaggcgactggatttgtgaagaagcccggtgtgcttgttggagatttgaagcgtttggcggttgtgaaagagtttggtgatgaagtgcctgatattggacttggagatcgtaaaactgatcatgatttcatgtctatttgcaaggaaggttacatggtgccaccaagcaaatcagcaaaaccagtcccactagagcgcctcaaaagccgactcatcttccacgacggtcgctttgtacaacgtccagacccactcaacgccatcatcaccttcgcatggctccccttcggcttcatcctctccatcttcagagtctacttcaacctccctctccctgaacgcattgtccgctacacctacgaattacttggcatcaagctcgtaatccgtggcaaccgtcctccaccaccatcacgaggaacccctggcaacctctacgtctgcaaccaccgcaccgcccttgaccccattgtcatcgccattgcccttggccgtaaagtctcatgtgttacttacagtgtaagtaaactctcccgcttcctgtcaccaatccccgctgttgccctcacccgtgaccgtgctgccgatgctgccagaatcactgaaattctcgaaaagggtgatcttgttgtgtgtccagaaggcacaacttgtcgtgaaccgttcttgctacggttcagtgctctgtttgccgaactgagcgataggattgtgcctgtagctgttgattgtaagcagaacatgttctttggaaccactgttcgaggagtcaaattctgggatccttatttcttcttcatgaatcctaggcctgtttatgagattaatttccttgaccggttaccagaagagatgtctgtcaaggctggtgggaaatcttccattgaggttgctaatcatgtgcagaaggttttgggtgatgtgcttggttttgagtgtactgggttgacaaggaaagataagtatttgttgcttggaggaaatgatgggaaggtggagtccatgtatggtggcaaaaagtga

SEQ ID NO: 43 - protein, 633 aa, Zea mays
MEEAVEAVVAAGVEPFPTVDKCDASGRGAHAVVADLDGALLRSRSAFPYYALVAFEAGGVPRLLLLLLLAPLAAALRALASESACARVLVFAATAGARVRDIESAARAVLPRFYAADVHPAAWRVFSACARRRVVLTAAPRIMAEPFLRDCLGADAVAGTELATWRGRATGMVDSRRGVLVGWSRADALREILAGDDDDDAAPDVGLGNSRSDYPFMSMCKVVDECGYVVDMGEQSLLTNLGGEDDRVDTLDLYDEGIPNLMKPPTPKFMLEKYSYVSIMCFTFITKLHLFDLSDYGPCVIARLLNDSLNDGGVTSAMAASSAMCGLAGRLSHEAYIVPRAPVEPLPMDQLPRPVIFHDGRLVRRPTPLAALLVVLWFPVGFALACLRIAAGALLPMPLVYYAFWALGVRVHVRGAPPPPRAERATGRRGVLFACSHRTLLDPIFLSTALGRPVAAVTYSLSRLSEFLSPIRTVRLTRDRASDAAMIRDLLAEGDLVICPEGTTCREPFLLRFSALFAELTHEVVPVAMENRMSMFHGTTARGWKGMDPFYFFMNPSPAYVVTFLNKLPPELTCAGGRTSHEVANYIQRLIAATLSYECTSLTRKDKYLALAGNDGVVATPKPPAPAKKPNGC

SEQ ID NO: 44 - DNA, 1902 bp, Zea mays
atggaggaggcggtggaggccgtggtcgcggccggagtggagccgttcccgacggtggacaagtgcgacgcgtcgggccgcggcgcgcacgccgtggtggcggacctggacggcgcgctgctgcgctcccgcagcgcgttcccgtactacgcgctggtggccttcgaggccggcggcgtgccgcgcctgctgctgctgctgctgctggccccgctggccgcggcgctgcgggcgctggcctcggagtcggcgtgcgcgcgcgtgctggtgttcgcggccacggcgggcgcccgggtgcgcgacatcgagtcggcggcgcgcgccgtgctcccgcgcttctacgccgccgacgtgcacccggccgcgtggcgggtgttctcggcctgcgcacggaggcgcgtcgtgctcacggccgcgcccaggatcatggccgagccgttcctcagggactgcctcggcgccgacgccgtcgcgggcaccgagctcgccacgtggcgcggccgcgccacgggcatggtggacagccgcaggggcgtcctcgtcggctggagcagggccgacgcgctgcgggagatattggccggcgacgacgacgacgacgccgcgcccgacgtcggcctcgggaacagccgctccgactaccccttcatgagcatgtgcaaggtcgtggatgagtgtgggtatgtagtagacatgggtgagcagtcgttgctcaccaatcttggtggtgaagatgacagagtcgacacccttgatctctatgatgaaggcatcccaaacttgatgaagcctcccacaccaaagttcatgttagagaagtactcctacgtgtcgatcatgtgcttcaccttcatcaccaagctccacttgtttgatcttagtgactatgggccatgtgtcatcgctagactccttaacgatagtctaaatgatgggggagtcacgtctgccatggcagcatcgtcagccatgtgcggcctagcgggtcgtctcagccacgaggcgtacatcgtcccgcgcgcgccggtggagccattgcccatggaccagctgcctcgcccggtcatcttccacgacggccgcctggtgcgccggccgacgccgctggcggcgctgctggtcgtgctgtggttccccgtgggcttcgcgctcgcctgcctgcgcatcgcggcgggggcgctgctgcccatgccgctggtgtactacgccttctgggcgctgggcgtgcgcgtgcacgtccggggcgccccgccgccgccgcgcgccgagcgcgccacgggccggaggggcgtcctcttcgcctgctcgcaccggacgctgctggaccccatcttcctgtccaccgcgctggggcgccccgtggcggcggtcacctactcgctctcgcgcctctccgagttcctctcgcccatccggacggtgcgcctcacgcgggaccgcgcctccgacgccgccatgatccgggacctgctggcggagggcgacctcgtcatctgccccgaggggaccacgtgccgggagcccttcctgctgcgcttctcggcgctcttcgcggagctcacccacgaggtggtgcccgtggccatggagaaccggatgagcatgttccacggcacaacggccagggggtggaaggggatggacccattctacttcttcatgaaccccagcccggcatacgtcgtcaccttcctcaacaagctcccgcccgagctcacctgcgccggcggcaggacaagccacgaggtggccaactacatccagaggctaatcgccgccacgctctcctacgagtgcacaagcctcaccaggaaggacaagtacctggcgctcgcaggcaacgacggggtggtggccacccccaagccgccggcgccggccaagaaacccaatggctgctaa

SEQ ID NO: 45 - protein, 504 aa, Sorghum vulgare
MAAASHHRCTSVASELEGTLLISGSLFPYFFLVALEAGGPLRALLLLAAYPLVALLGAAFSSDDLPLLAMTFLSTAGLGVADVTAVARATLPKFFLADLRGTAFRALARHGDAASGGERYVVTRLPRLVVEPFVREYLGADVRVVGTELRVVAGSRFSGTLVASPGVVGGGDRSLGALVAVLGRDRIVDVGLYCSASGGDRQPAFLQVCQERRVVSAPEKAPATPLPRSEYLRPLVFHDGRLVCRPDPLVCLAIFLWIPLGVLLSVARLLFGFFPHGAGLLLAAATGFRIRGSLGGAAEPAGPGRGTLFACNHQTLMDPVILSTVLRRKVTAVTYSLSSFSELIAPIPTVRLTRDRGRDSRIMQGELTRGDLVVCPEGTTCREPYLLRFSPLFAEIAGEVTPTAVRAGGTMFHGSTVRGHKWLDSVFFLMNPAPWYEIRILTPVATCGSGGGASSLDVANGVQRVIGDALGFECTGLTRRDKYRMIAGHDGVDARSSPPKPPPS

SEQ ID NO: 46 - DNA, 1515 bp, Sorghum vulgare
atggccgctgcttctcaccaccggtgcaccagcgtcgcctccgagctggaaggcacgctgctcatctccggcagcctcttcccatacttcttcctcgtcgctctcgaggccggcggcccgctcagggcgctgctcctgctcgccgcgtaccctctcgtcgcgctgctcggcgccgccttctcgtcggacgacctcccgctgctcgccatgacgttcctgtccaccgccggcctcggcgtcgccgacgtgacggcggtggcgcgcgccacgctgcccaagttcttcctggccgacctccggggcaccgcgttccgggccttggcgcgccacggcgacgccgccagcggcggcgagaggtacgtcgtcacgcgcctgcccaggctcgtcgtggagccgttcgtcagggagtacctcggcgccgacgtccgcgtcgtcggcaccgagctgcgggtggtcgctgggagccggttctccggcactttggtagcctcgccgggcgtcgtgggtggcggtgaccggagcttgggtgctctggtggccgttcttgggcgcgaccggatcgtcgacgtgggactctactgctccgcctccggcggggaccggcagccggcgtttctgcaggtttgccaggagcgccgcgtggtgtccgcgccggagaaggcgccggcgacgcctctgccgaggagcgagtacctgcggccgctggtcttccacgacggccgcctcgtgtgccggccggacccgctcgtgtgcctcgccatcttcctgtggatcccgctcggcgtgctcctctccgtggcgcgcctcctcttcggcttcttcccccacggcgccggcctcctcctcgccgcggccacgggcttccggatacgcggcagcctcggcggagcagcggagccggcgggaccagggcgaggcacattgttcgcctgcaaccaccagacgctgatggacccggtgatcctctcgacggtgctgcgccggaaggtgaccgccgtgacgtacagcctctcgagcttctcggagctgatcgcgccgatcccgacggtccggctgacgcgcgaccgcggcagggacagccggatcatgcagggcgagctcacccgcggcgacctggtggtctgcccggagggcaccacctgccgggagccgtacctgctgcggttcagcccgctgttcgccgagatcgccggcgaggtcacgccgacggccgtccgggccggcggcaccatgttccatggatccacggtgcggggacacaagtggctcgactccgtcttcttcctcatgaacccggcgccgtggtacgagatccggatcctcacgccggtggccacctgcggcagtggcggcggcgcgtcgagcctcgacgtggcgaacggcgtccagcgtgtgatcggcgacgcgcttgggttcgagtgcacagggctcacgcgcagggacaagtaccggatgatcgccggacacgacggcgtcgacgcgaggtcgtcgccgccgaagccgccgccgtcgtag

SEQ ID NO: 47 - protein, 514 aa, Oryza sativa
MGDATMIASPFPAVEKCSSKDRGGDTIVADLDGTLLCGRSSFPYFAHMAFETGGVLRLLALILLAPLAGVLYYLVSESAGIQVLIFASMAGARVADVEAVARAVLPKFYCSDIHPESWRVFSACGRRCVLTANPRMMVEAFLKEYLGADIVVGTELVVWRGRATGLVRSPGVLVGENKAAALREAFGDASPEIGIGDRRTDYPFMRLCKEGYVVPVSPTPRPVPREELPKPVVFHDGRLVQKPSPALALLTVLWIPIGFVLACLRIAAGALLPMRVVYHAFRALGVRVTVKGTPPPPASRETGQSGVLFICSHRTLLDPIFLSTALGRPITAVTYSVSRLSEILSPIRTVRLTRDRAADAAMIRRLLAEGDLVICPEGTTCREPFLLRFSALFAELTDEIVPVAMENQMSMFHGTTARGWKALDPFYFFMNPSPGYVVTFLSKLPGELTCNGGGGGGGGRSSHEVANYIQRLIASTLSYECTSFTRKDKYRALAGNDGTVVSKPNIDEKKAMGC

SEQ ID NO: 48 - DNA, 1545 bp, Oryza sativa
atgggggacgccacgatgatcgcgtcgccgttcccggcggtggagaagtgttcgtccaaggaccgcggcggcgacacgatcgtcgccgacctcgacggcacgctgctgtgcggccggagctcgttcccgtacttcgcgcacatggcgttcgagacggggggcgtcctccggctgctggcgctgatcctgctggcgccgctcgccggcgtgctctactacctggtgtccgagtcggccggcatccaggtgctcatcttcgcgtccatggccggcgcgagggtcgccgacgttgaggcggtggcgcgcgccgtgctgcccaagttctactgctccgacatccacccggagtcgtggcgcgtgttctcggcgtgcgggcgccggtgcgtgctcaccgcgaacccgaggatgatggtggaggcgttcttgaaggagtacctcggcgccgacatcgtcgtgggcacggagctcgtggtctggcgtggccgcgccacggggctcgtccggagccccggcgtgctcgtcggcgagaacaaggcggccgcgctccgggaggcgttcggcgacgcctcgccggagatcggcatcggcgaccggaggacggactacccgttcatgaggctatgcaaggaggggtacgtggtgccggtgtcgccgacgccgaggcccgtgccgcgggaggagctcccgaagccggtggtgttccacgacggccgcctggtccagaagccgtcgccggcgctcgcgctgctcaccgtgctctggatccccatcggcttcgtgctcgcctgcctccgcatcgccgccggcgcgctcctcccgatgcgcgtggtgtaccacgccttccgcgccctcggcgtgcgcgtcaccgtcaagggcaccccgccgccaccggccagccgcgagaccggccagtccggcgtgctcttcatctgctcccaccgcaccctcctcgaccccatcttcctctccaccgccctcggccgccccatcaccgccgtcacctactcggtgtcgcggctgtcggagatattgtcgccgatcaggacggtgcggctgacgcgggacagggcggcggacgcggcgatgatccggcggctgctggcggagggcgacctggtgatctgcccggaggggacgacgtgccgggagccgttcctcctccggttctcggcgctgttcgcggagctcaccgacgagatcgtgccggtggcgatggagaaccagatgagcatgttccacggcacgacggcgagggggtggaaggcgctggaccccttctacttcttcatgaaccctagccccggatacgtggtcaccttcctcagcaagctccccggcgagctcacctgcaacggcggtggcggcggcggcggcggccggagcagccacgaggtggccaactacatccagaggctcatcgcctccacgctctcctacgagtgcaccagcttcaccaggaaggacaagtacagggcgctcgccggcaacgacggcaccgtcgtgtccaagcccaacatcgacgagaagaaggccatgggatgttaa

SEQ ID NO: 49 - protein, 264 aa, Oryza sativa
MFMWLPLGAPLAILRTAVFLLLPFSISVPLLAALGMHSRRISSSSLPPAAAAAHNLFVCNHRSLVDPLYVSAATGRTDLSAATYSISRLSEILAPIRTFRLTRDRATDRAAMQAHLSLPGGGGGGLVVCPEGTTCREPFLLRFSPLFTELGADVQPVALHSAVSMFHGTTAGGWKLLDPLYLLMNPTPAYVVQLLDPVAVGGEGGGGGPELANEVQRRIAEALGYTRTALTRRDKYLALTGNDGGVDRRRVAGSHHHQEGVLNG

SEQ ID NO: 50 - protein, 467 aa, Oryza sativa
MAFETGGVLRLLALILLAPLAGVLYYLVSESAGIQVLIFASMAGARVADVEAVARAVLPKFYCSDIHPESWRVFSACGRRCVLTANPRMMVEAFLKEYLGADIVVGTELVVWRGRATGLVRSPGVLVGENKAAALREAFGDASPEIGIGDRRTDYPFMRLCKEGYVVPVSPTPRPVPREELPKPVVFHDGRLVQKPSPALALLTVLWIPIGFVLACLRIAAGALLPMRVVYHAFRALGVRVTVKGTPPPPASRETGQSGVLFICSHRTLLDPIFLSTALGRPITAVTYSVSRLSEILSPIRTVRLTRDRAADAAMIRRLLAEGDLVICPEGTTCREPFLLRFSALFAELTDEIVPVAMENQMSMFHGTTARGWKALDPFYFFMNPSPGYVVTFLSKLPGELTCNGGGGGGGGRSSHEVANYIQRLIASTLSYECTSFTRKDKYRALAGNDGTVVSKPNIDEKKAMGC

SEQ ID NO: 51 - protein, 537 aa, Oryza sativa
MAKKPCEFPTAVLSFRRFLQRRFLGGRHHHRPTSTTATGAAIPPADKLHNQTVMIDLESWLLRSPMSTFPYFMIVAIEAGSFLRGLILLLIYPLLWLLLSHDMLLKAMVMVSFFGLPEKEVVRIGKAVLPKFFLEGMAMEGLEVVRNAKKVVVFSPLFPRVMVEGFLKEYIGVNAVIGREVIAVAGRYVGLLVDHIDMDDGGFVDEVMEETKRGKGDGAVGLAGVGSKMHHLFSRYCKETYVVCDADKKEWQPVPREKYPKPLIFHDGRLAFKLTPCAAVAMYTYLPWGIFLAVFRSLAFGLLPYRVSVPLAAFTGMRSRLIAGPSPDATRRNSGTAGGRLYVCNHRTLLDPITVAAVLNKPITAVTYSVSPVSELIAPIRTARLTRDRDEDRRRMEALLARGDLVVCPEGTTCREPYLLRFSPLFAELTGEVTPVALETRIDMFYGTSTKPAAKWLDPFYFMLNSRPEYHVEFLQPVSTAPVDGEAGGHGHSINAANRVQRVLGEALAFELTEQTRKDKYEMLAGNKGNVKGEAKM

SEQ ID NO: 52 - protein, 570 aa, Oryza sativa
MPTNLSSSYSSLFEPLLALSSLLLRRLSAAVRHHRGRNTPPASSDVPPPPPPSSSPLELDRLYGRTVVVDVDAWLLLPAVDAFTFFMVVAVEAGGFLRGLLLLLVYPLLCLLLGDGDGGGGARARAMATVALVGLEEREVARVGRAVMPRFLMAAAAAEGAEAVRAARRSVAVSATLPRVMVEAFLREHVGVDAVVGPELRSVAGVVAGIMDDADAARVAARRLRALLGDEMDQGEADGAGAAVGLVGEGRSGGTVHYLFSRYYCKETFTATEADKRRWRPLPPGGECGGVKPLVFHDGRLAFPPTPSAALAMYAYLPFGVALAVSRIIALSLLPYGRATFLVGALTGVHYRLVGAGHDAAAGGGGGRLYVCNHRTLLDPIVVAAALGKPVTAVTYSLSRVSEMIAPIRTARLTRDREEDRRSMAALLARGDLVVCPEGTTCREGYLLRFSPLFAELGADVNPVALDARVDMFHGTSTTPAAKWMDPFYFMMNPKPSYRVEFLPRAAPAPAAEDGGDSIRVANRVQRQIGEALGFELTGMTRKDKYMMLAGNEGVVAAAAAAAATIKASR

SEQ ID NO: 53 - protein, 291 aa, Oryza sativa
MGVSLEVYVVNRESTKTARLPRDRYPKPLIFHDGRLAFLPTPSAALAFFLFLPLGVILSVIRISIGILLPYKISFGAGALFGVRYRTSGLRAPEPGVKRRGVLYVCTHRTLVDPIMLTAALQKPVPAVTYSLSRLSEVIAPIKTVRLTRDRERDAETMSRLLEHGDLAVCPEGTTCREPYLLRFSPLFAELADDMEPVALDAQVTTLYGTTASGHKWLDPVVFFANPQPAYRVDFLGAVPREWTRAGGRAGAEVANWVQRRLGEALGYECTGLTRRDKYMMLAGNDGVVAK

SEQ ID NO: 54 - protein, 495 aa, Oryza sativa
MATHQRRHPSVAAELEGTLLISGDLFPYFLLVALEAGGPLRAAVLLAAYPVAALLGVALPDDDLAVRVMTFVSTAGLSVADVAAVARATLPRFFLADLSDAAFRAFARRDAAERYVVTRLPTAMVEPFVREYVAEGARVVGAELRVVGGRFTGAAVNGDRSLGALQAVLGRGRRVIDVGLCSGDGAAKRQPAFMKICQERHVVSTPEKAPAAPLPRSEYLRPLIFHDGRLVGRPDPLACLAVALWLPLGAALAVTRILIAFLPYSVGLLLAAATGFQIRAHLGGAPPRWRGGTLYACNHRTLLDPAVLSTVVHRKVTAVTYSLSGLSEMIAPIPTVRLTRDRGRDRVIMQSVLAGGDLAVCPEGTTCREPYLLRFSPLFAEIAGEVTPVAVRAGGAMFHGTTVRGYKGMDSFFFLMNPAPWYHLQLLDPVPSSSAAADGDGGGDGGGESSRDVANRVQRAIGDALGFECTALTRRDKYRMIAGHDGVDMRGNARL

SEQ ID NO: 55 - protein, 532 aa, Oryza sativa
MVSRRFKPVEECSSDGRSEQTVAADFDGTLVRSRSAFPYYLLVALEAGSVLRAVVLLLSVPFVYVTYIFFSESLAISTLVYISVAGLKVRNIEMVARSVLPKFYAEDVHPESWRVFNSFGKRYIITASPRIMVEHFAKTFLGADKVVGTELEVGKNGKATGFMVKPGVLVGDHKRQAVVKELRDAVPDVGLGDRETDFDFMSICKEIGRLARETSELVLAVPARSTAALAFPFFLPFFQPEAYLVTSRKYSAVPKNQLLSPLILHDGRLVQRPTPLVALVTFLWMPFGFALALLRVYVNLPLPERIVFYTYKLMGIRLIVKGNPPPPPKKGHPGVLFVCNHRTVLDPVEVAVALRRKVSCVTYSISKFSELISPIKAVALSREREKDAENIRRLLEEGDLVICPEGTTCREPFLLRFSALFAELTDRIVPVAINTKESMFHGSTVRGFKLMDPYFFFMNPRPTYEITFLNQLPKELTCSGGKSPIEVANYIQKTLSGQLGFECTAITRKEKYSILAGTDGRVPSKNKEKEKN

SEQ ID NO: 56 - protein, 558 aa, Oryza sativa
MAKTKLFPALFSLLLHGGAATLPPPRVPAVTVHGCTPPAARLSAGGEKTVTMVVDVEGALLRSSSSRSLFPYFMLVAVEAGSFLRGLLLLLLYPVISLLAGAGGGDVAVRAMAAVAFCGLRESRFRAGRTVLPRWLLDDVGKEAVDAIVTLTRRRSSPAATATVVWASSMPRVMVEPFLREYMAAAEGGGEVVVAAREMKVVWGFYTGVMEDGGEVAAASPEVRRAMEGVDDVVGFSGGSMDLLRSPLVSFCKEVYVVSHEEKSKWRPLPRRREYPRPLVFHDGRLAFLPTPLAAAAMLVWLPFGAALAATRLAVALALPYRHATLLLAATGQSWRLRGSPPPTPTPPPRRATGERRRGQLYVCNHRTLIDPVYVSIALDRPVRAVSYSLSRVSDLLSPIGATVRLARDRAHDGAAMARLLEAGAHVVVCPEGTTCREPYLLRFSPLFAELADGVVPVALAAEAAAFHGTTAGGWKSMDALCYLANPRMCYTVEFLPAVDASPVREGKAASTELANAVQRRVAEALGYESTMLTRKDKYLMLAGNDGVVRRRGDVGAK

SEQ ID NO: 57 - protein, 506 aa, Oryza sativa
MAAAAAAGRRRFFPPVTAYDAAAGARRTVAADLDGTLLVSSSAFPYYFLVALEAGSYLRALALLLAAPWLLALYVGVSEAAAIALLVFITFAGLRVRDVEAVARAVLPRHYAAGVRADTWAVFHGCAERRVVVTASPAVMVGEFVREFLGAEVAGTELETFASGKRFTGRIKAVLVGEKKREVVERLFAGGDMPDVGLGDRESDHDFMAICKEAYMVPKNKRAPRAAADELLSRAIFHDGRLVRRPEPASALFALAYLPVGFAVALLRVFLNLPVPARLVRHTYRLTGIRLAVRGAPPPPPRPGTPGSLLVCNHRTALDPIIVSIALGRPVTCVTYSVSRLSTAISPIRAAALTRDRAADAARIAALLEEGDVVVCPEGTTCREPYLLRFSALFAELTARIVPVAVEARQGTYYGSTARGWKFLDPYFFYMNPRPGYEVTFLPALRPEETCVAGGRSAVEVANHVQRVIAKELGFQCTTLTRKDKYMKLAGNDGRVAAAADKPKAN

SEQ ID NO: 58 - protein, 401 aa, Arabidopsis thaliana
MASAYILKNKDSFSYFVLLAFDASGLIRFTVMLFHWPITKLLDVLRYNNAALKLMIFIATVGLCEGEIEAVARTVLPKFYMDDVSMDTWRVFSSCKKRVVVTGMPRVMVEIRSPRSILEPIMSSIHALVMHSRKIELQPVPVIFNDGRLVKRPTPATALLILIWIPFGMVLSPIRILSGFILPMWIRTHAMSILGCQIIVKGKPSQPREAVKSGVLFVWSLLLYTPFHGFPNSSLPFPYIVRIQRIRDVDAKTIKLDLVICPEGSTCRQPFLLRFSALFAELTDMIVPVAVNYRVGFFHANTVRGWNCMDMIFFFMNPRPGYEVTFLNKLPVEATCLSGKKPYDVANHVQRILADTLGFECTNLTRKDKYKVLTGKDGTVSYVSFQDQAKKVVNNFNPFSH

SEQ ID NO: 59 - protein, 530 aa, Oryza sativa
MEAVAEAVAAAVAAGVEPFPAVDKCDASGREAHAVAADLEGTLLRSRSAFPYYALVAFECGGVPRLALLLLLAPLAAALRAAVSEAAAVRVLVFAATAGARVREIESAARAVLPRFYAADVHPGAWRVFAACARRRVVLTATPRVMAEPFLVDCLGADAVAGTELATWRGRATGLVDDRRGGVLVGRRKAQALLDMFAGGDVPDVGLGDRRSDYPFMSLCKEGYIVPRSPAVEAVPMDKLPRPVIFHDGRLARRPTPLAALLAVLWFPVGFALACVRIAAGALLPMPWVYYAFWALGVRVVVRGAPPPRAERAAGRRGVLFACSHRTLLDPIFLSAALGRPVAAVTYSLSRLSEFLSPIRTVRLTRDRAADAAMIGELLDEGDLAICPEGTTCREPFLLRFSALFAELTDEVVPVAMESRMGMFHGTTARGWKGMDPFYFFMNPSPAYVVTFLGKLPPEHTCGAGGRSSHEVANYIQRLIAATLSYECTSLTRKDKYRALAGNDGVVDNATGKLPSPATATANNSKDKAC

SEQ ID NO: 60 - skipped

SEQ ID NO: 61 - protein, 519 aa, Oryza sativa; identical to SEQ ID NO: 21

SEQ ID NO: 62 - protein, 453 aa, Oryza sativa
MEAGGFLRGFIMLCVHLVLWCLAPLLPEEVKLKVMVMVCFFGLKEKKVARVARAALPKHFLEGVGMEGLEAVRGVKSVVGVSRVIPRVMVKPFLEDYLGVDVVVGREVKMVRGFYVGLLENMSDGRLELADLEGEEMIGFGSSSGYSGHDHHHLFSWCKEVYLVTPEEKRKWSPLPRDQYPKPLVFHDGRLAFRPTFQATLAMLTWLPFSLPLTIFRTLIFVTLPYPVSVAIGSVFGVRTRVINSPVGQAKADHPRNPKGHLYVCNHRTLLDPVYIAAMLNKKVSAVTYSVSRLSEWISPIPTIRLTRDRDEDRRRMEEALRRGDLVVCPEGTTCREPYLLRFSPLSLELVDEVYLVALVNWSDMFYGNSTGRSKCLDSFYYFMNPRPAYDVEFMEKVPTRMVVDGKTCESKHVANMVQGEIGRVLGFECTKFTRENKYLALAGNRGVVDANQ

SEQ ID NO: 63 - protein, 541 aa, Oryza sativa
MSWKAFTKSLLFHSNKIVRRLKSFVLHAHPPPPPPHVVRRPPSSDELPAAADVTVCKVEGGLLMSPSTFPYFMLVALEAGGLFRGLLLLLLYPVLLLLGHDRATKLMVMVSFAGVRKEKDGSPSFRVGRAVMPKLFLEDVSAEVFDAAARRRRLVCVSSMPREMVEPFLKEYLAVDAVVAPELRAFRGYYLGLAESDGEVMQRLDMEEVIGMKERGGAGDGDGDGQVTVVGIAGLGNSFHQLFQNYCKEVYVASEWARRRWRPLHPRRYAKPLIFHDGRVAFRPTTSATLAMFVWLPLAVPLALLRVALIVVVLPFSLAAPIAAALGIHCRCIAASTLRAAAVLDLFVCNHRSLLDPLYVSAVAGRADLAAATYSISRLSEILAPIRTFRLTRDRAADRAAMQAHLSRSRRGGGGGGLVVCPEGTTCREPFLLRFSPLFTELGADVQPVALHSEVAMFHGTTAGGWKMLDPFFLLMNPSPAYVVHFLDPVAGGGGGPEVANEVQRRIAETLGYTCTALTRRDKYLVLAGNDGVVANNNKSN

SEQ ID NO: 64 - skipped

SEQ ID NO: 65 - protein, 486 aa, Oryza sativa
MSPFKPIEQCSTEGRSQQTVASDLDGTLLLSRSAFPYYLLVALEAGGPLRAVALLMSVPFVYLTYVTISESLAVRALLYIAVAGLEVRDVESVARSVLPRFYAGDVHPEGWRVFSSFGRRCVVTASPRVMVEPFARAFLGADRVIGTELEVGEDGRATGFVAKPGVLIREHKRNAVVREFGDALPDVGMGDRESDFDFMAICKDAYVVTTSRKHRPVPESQLLRTVVLHDGRLAQRPTAINTLLVFLWMPVGFALALLRACLSLLLPERVLSYAYKLTGVGLVVRGRPPPPDGSPGVLFVCNHRTVLDQVAVAAALGRKVICVTYSVPRKTYGMSSRLPEALTASPVKAAVALCRERDRDADRVRRLLEEGVDIVAFPEGTTCRGAFLLRFSSLFAELTDRIVPVAIATRETMFHGSTARGFKGMDPYFFFMNPRPAYEVTFLSQLPSELTSGGGGKSPVEVANYVQKALAGQLGSEHIGITRKEK

SEQ ID NO: 66 - protein, 15 aa, Arabidopsis thaliana
HRTLMDPVVLSYVLG

SEQ ID NO: 67 - protein, 21 aa, Arabidopsis thaliana
GDLVVYPEGTTCREPFLLRFS

SEQ ID NO: 68 - DNA, 28 bp, Artificial Sequence
  Synthetic
cacactctagaatggttatggagcaagc

SEQ ID NO: 69 - DNA, 26 bp, Artificial Sequence
  Synthetic
cacacgagctctcaatggagacaagg

SEQ ID NO: 70 - DNA, 1812 bp, Artificial Sequence
  Binary Vector pBI121
atgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatga

SEQ ID NO: 71 - DNA, 20 bp, Artificial Sequence
  Synthetic
ccccaaaacgatgaaagcta

SEQ ID NO: 72 - DNA, 20 bp, Artificial Sequence
  Synthetic
ttctcgaggagttgcctcat

SEQ ID NO: 73 - DNA, 17 bp, Artificial Sequence
  Synthetic
tcgattgcaaaatacaa

SEQ ID NO: 74 - DNA, 20 bp, Artificial Sequence
  Synthetic
caagttcgatatcgcggatt

SEQ ID NO: 75 - DNA, 22 bp, Artificial Sequence
  Synthetic
tggttcacgtagtgggccatcg

SEQ ID NO: 76-77 - skipped (2 entries)

SEQ ID NO: 78 - DNA, 1588 bp, Arabidopsis thaliana
gataagaaattgcataaagggagaggctttgtaaacacgcaagtttacgtcaacttgacacatataaactcgtatataggaattgtgtaaaatgttaatcaatatatgtttatgaatatagtccttttatttccttaatctatagtcatgttatcatcatatcggcaagaaaattactattacaacatatgtttccgtgcttcgatttgtcgatcaggtaggcaaccaaaccgggccagattagtcacaaatgaaattaggctccactttggcaagttttttcacaattaaaaattagaaagtctaaccaaactaatagaaactgaaccaaacaaaaatgttcacgcaattacgtagcacttctctatgaaatactgtggataattcgatttgagctaatctatggtaagtcgtttctctcaaactgaattaagctaaaccaaaccgaaaataaaccgttgtagaacacccataataaacatatatatgttctctactcttatttggctatttttccatttgcagatacgtgtgacaacattccaaatataggcacatgatacaaattacaaagccagaaattataatttataaactatacataataaggtcagtagcctaattacatccatatatttagttgggcataataaggatagcctaattttggtgcatcccgccaaaaattcggacaaatggtgaatttcaaaccatgaatttctttgttatacatacattacgtgcactattacatcaaagaaaaatagtgacgtgcataaacgtattttcttcacataatctatatataatatatacagccatgtacatttttcaacatagaaaaatcatgtatacaaatacggtaggtgcaatactgataaactgccattcccattgcattagacgctagtcaatccaaaccttataacataaaaagttatagtccatttctttcatatccaatcgagtcaaaataatattcgagcaaaaaacaaaagaaatggttatggagcaagctggaacgacatcgtattcggtcgtgtcagagtttgaaggaacaatactgaagaacgcagattcattctcttacttcatgctcgtagccttcgaagcagctggtctaattcgtttcgctatcttgttgtttctatggcccgtaatcacactccttgacgttttcagctacaaaaacgcagctctcaagctcaagatttttgtagccactgttggtctacgtgaaccggagatcgaatcagtggctagagccgttctgccaaaattctacatggacgacgtaagcatggacacgtggagggttttcagctcgtgtaagaagagggtcgtggtcacgagaatgcctcgagttatggtggagaggtttgctaaggagcatcttagagcagatgaggtcatcggtacggaactgattgtaaaccggttcggttttgtcaccggtttgattcgcgaaacggatgttgatcagtctgctttgaaccgtgtcgctaatttgtttgttggtcggaggcctcaactaggtcttggaaaaccggctttgaccgcctctacaaatttcttatcgttatgtgag

SEQ ID NO: 79 - DNA, 1183 bp, Arabidopsis thaliana
gatatgaccaaaatgattaacttgcattacagttgggaagtatcaagtaaacaacattttgtttttgtttgatatcgggaatctcaaaaccaaagtccacactagtttttggactatataatgataaaagtcagatatctactaatactagttgatcagtatattcgaaaacatgactttccaaatgtaagttatttactttttttttgctattataattaagatcaataaaaatgtctaagttttaaatctttatcattatatccaaacaatcataatcttattgttaatctctcatcaacacacagtttttaaaataaattaattaccctttgcatgataccgaagagaaacgaattcgttcaaataattttataacaggaaataaaatagataaccgaaataaacgatagaatgatttcttagtactaactcttaacaacagttttatttaaatgacttttgtaaaaaaaacaaagttaacttatacacgtacacgtgtcgaaaatattattgacaatggatagcatgattcttattagagtcatgtaaaagataaacacatgcaaatatatatatgaataatatgttgttaagataaactagacgattagaatatatagcacatctatagtttgtaaaataactatttctcaactagacttaagtcttcgaaatacataaataaacaaaactataaaaattcagaaaaaaacatgagagtacgttagtaaaatgtatttttttggtaaaataatcacttttcatcaggtcttttgtaaagcagttttcatgttagataaacgagattttaattttttttaaaaaaagaagtaaactaactatgttcctatctacacacctataattttgaacaattacaaaacaacaatgaaatgcaaagaagacgtagggcactgtcacactacaatacgattaataaatgtattttggtcgaattaataactttccatacgataaagttgaattaacatgtcaaacaaaagagatgagtggtcctatacatagttaggaattaggaacctctaaattaaatgagtacaaccaccaactactccttccctctataatctatcgcattcacaccacataacatatacgtacctactctatataacactcactccccaaactctcttcatcatccatcactacacacatc

SEQ ID NO: 80 - DNA, 1349 bp, Nicotiana tabacum
tgaacttattaaccttttgataacatgacgtcaacttaattttatcattgcgtcaagacttttctttcacttcctacccaaagaaaagaacaaatctctaactgggaaagattggaatagaaacaagtattggcttaccgagcaccgacagatggagtggtaggaaatagaaaggcggcaacggcaatattcattattgaggtagcacaacatgatgcaatctatcgctgatttggtcagactgtggaccttgtcttttgtctcctcctattagattgaatgctactgttttgctcaacaactagggaagagaagggggaaaaaactaaagtacaaaagttaatatatttgatctcgcaaaagaaaattattttccatgttgccaccagcagtgtatacacagtttataatctcgctgaccatgtcgaattttcgtaagcatttccgaaatcatttgtggcatttctatattttaaatattaaaccgtaaaacatggggttaaaaagcaatgaaaattgtttcaagaacctgatatgcactatacttcaaagcctaactactcctaaagaagcaaaaggtgtgcttcaatatggtgattcagaaatatacaactcctaattaagtaattactatagaggttcttgattttcaggcaataggcctaacaacgatagtaacaagttgagatgtggaacccaattgcttgtttaaataatgtaattgtgttaaaaaatttattacaaatatgtatatacatattaaattttgaatctatttattagccatttgaagtgattgttctaaaatacaaaattaataaagttaaaattctggttccacatctatagtagagactttcaccatacaactaatactccctccgttttaatttatgtgagagaagacttttgaacttgtgatgtaaaatgaggcacaaatattttgtgcggttataaatcattgcataaaggtaaattgttaccaaataagaaaataagtcattctttttgacatgactaaaaagaaaatatgatcacataaattgaaatcgagagagtatgtcaataaatataaatcatataaaataatcagactaatatgtcaaatttcttccctcaacacgagctgatacattaccctaatttcaaagcattgaatagaacccaactatcagataattaattctctcaatcaaatccaattttctccctataaaaaccctaaggtcctatagtgttctatatccaacactagctcctactccctaaagcatttattatatcttcccctagctagatacttcattccacaaatagtttgcagctttttcttttcctctaaaacaatg

SEQ ID NO: 81 - DNA, 1176 bp, Arabidopsis thaliana
tgtgaattatattttactcttcgatatcggttgttgacgattaaccatgcaaaaaagaaacattaattgcgaatgtaaataacaaaacatgtaactcttgtagatatacatgtatcgacatttaaacccgaatatatatgtatacctataatttctctgattttcacgctacctgccacgtacatgggtgataggtccaaactcacaagtaaaagtttacgtacagtgaattcgtctttttgggtataaacgtacatttaatttacacgtaagaaaggattaccaattctttcatttatggtaccagacagagttaaggcaaacaagagaaacatatagagttttgatatgttttcttggataaatattaaattgatgcaatatttagggatggacacaaggtaatatatgccttttaaggtatatgtgctatatgaatcgtttcgcatgggtactaaaattatttgtccttactttatataaacaaattccaacaaaatcaagtttttgctaaaactagtttatttgcgggttatttaattacctatcatattacttgtaatatcattcgtatgttaacgggtaaaccaaaccaaaccggatattgaactattaaaaatcttgtaaatttgacacaaactaatgaatatctaaattatgttactgctatgataacgaccatttttgtttttgagaaccataatataaattacaggtacgtgacaagtactaagtatttatatccacctttagtcacagtaccaatattgcgcctaccgggcaacgtgaacgtgatcatcaaatcaaagtagttaccaaacgctttgatctcgataaaactaaaagctgacacgtcttgctgtttcttaatttatttctcttacaacgacaattttgagaaatatgaaatttttatatcgaaagggaacagtccttatcatttgctcccatcacttgcttttgtctagttacaactggaaatcgaagagaagtattacaaaaacatttttctcgtcatttataaaaaaatgacaaaaaattaaatagagagcaaagcaagagcgttgggtgacgttggtctcttcattaactcctctcatctaccccttcctctgttcgcctttatatccttcaccttccctctctcatcttcattaactcatcttcaaaaatacc

SEQ ID NO: 82 - DNA, 1080 bp, Arabidopsis thaliana
aaaaaacaaagacaccacaatttaatgaaaatgtagtcaatggataatataaagataattcgtgacacaaaggaaaaaaaaatatagtcggatgaattcatgtttctgtcatatatttgtcttgctcttatcccgaatctcaagtaaaaatcaaaatcagaccaaatgtttcattacttaacttcatgccgcttccagatgttggcaacatatttttagtcaacaagggtttttatggttatggttaaccactatgtatggtatatatttctgataaagtgtttatttgtatattgattatattttaaaatgtcatatactcatatggcctctataatatttggttagcaatatattattactgtttttataagtcaaatctcaatattataagcaacgatcttacacttgtgtgctgtcaatagaaagtggtaatttaataatttgattttttttttttttttaattttcgtatagtacttaattacttagaggtctgggtcattagaaatatgtgactaacatgtacaccagttattagatcccaaaatgttaacatatcctaaaagcagtagaacaatgatacaagacccaatatatattttatgtttccacattaatttacatttaaatcaattgttatatcattcaaaatgttttaacaaatgtggagaaaagtcaaactttataagtttatatcactgtaactagtcgagaatagtcattctgatcataaaatacggaaaggtaataaaatcaaaagtgtattaacactttattatgtcttcttttttattttttgtcaaattatttccattttgttagttgataaatagacaaaatttaattagcttcattgagggaaaaaaaaataatatttaattttcttataaataccacccaatattgaggattggtcagctaccctcgtgtccttgaaatgttttgttcctcaatcatcattataacattcacaccccattttaaatttctctctacttgctccttttacatttcaacgaacaaaacacatgtatacatagaaagaaacgcaaatacgcagatataataaatggcgac

SEQ ID NO: 83 - DNA, 835 bp, Artificial Sequence
  Binary Vector pBI121
agattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacg

SEQ ID NO: 84 - DNA, 795 bp, Artificial Sequence
  Binary Vector pBI121
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 85 - protein, 264 aa, Artificial Sequence
  Binary Vector pBI121
MIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKARMPDGDDLVVTHGDACLPNIMVENGRFSGFIDCGRLGVADRYQDIALATRDIAEELGGEWADRFLVLYGIAAPDSQRIAFYRLLDEFF

SEQ ID NO: 86 - DNA, 1812 bp, Artificial Sequence; identical to SEQ ID NO: 70
  Binary Vector pBI121

SEQ ID NO: 87 - protein, 603 aa, Artificial Sequence
  Binary Vector pBI121
MLRPVETPTREIKKLDGLWAFSLDRENCGIDQRWWESALQESRAIAVPGSFNDQFADADIRNYAGNVWYQREVFIPKGWAGQRIVLRFDAVTHYGKVWVNNQEVMEHQGGYTPFEADVTPYVIAGKSVRITVCVNNELNWQTIPPGMVITDENGKKKQSYFHDFFNYAGIHRSVMLYTTPNTWVDDITVVTHVAQDCNHASVDWQVVANGDVSVELRDADQQVVATGQGTSGTLQVVNPHLWQPGEGYLYELCVTAKSQTECDIYPLRVGIRSVAVKGEQFLINHKPFYFTGFGRHEDADLRGKGFDNVLMVHDHALMDWIGANSYRTSHYPYAEEMLDWADEHGIVVIDETAAVGFNLSLGIGFEAGNKPKELYSEEAVNGETQQAHLQAIKELIARDKNHPSVVMWSIANEPDTRPQGAREYFAPLAEATRKLDPTRPITCVNVMFCDAHTDTISDLFDVLCLNRYYGWYVQSGDLETAEKVLEKELLAWQEKLHQPIIITEYGVDTLAGLHSMYTDMWSEEYQCAWLDMYHRVFDRVSAVVGEQVWNFADFATSQGILRVGGNKKGIFTRDRKPKSAAFLLQKRWTGMNFGEKPQQGGKQ

SEQ ID NO: 88 - DNA, 14758 bp, Artificial Sequence
  Binary Vector pBI121
tgagcgtcgcaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggcccggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagaggatccccgggtggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtgtctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccgg

SEQ ID NO: 89 - DNA, 25 bp, Artificial Sequence
  Synthetic
gctatttttccatttgcagatacgt

SEQ ID NO: 90 - DNA, 22 bp, Artificial Sequence
  Synthetic
acatctcggattcttgtcaatc

SEQ ID NO: 91 - DNA, 25 bp, Artificial Sequence
  Synthetic
ctaaggagcatcttagagcagatga

SEQ ID NO: 92 - DNA, 25 bp, Artificial Sequence
  Synthetic
tccagcgagaaccctatacttatct

SEQ ID NO: 93 - DNA, 22 bp, Artificial Sequence
  Synthetic
tggttcacgtagtgggccatcg

SEQ ID NO: 94 - DNA, 25 bp, Artificial Sequence
  Synthetic
ctaaggagcatcttagagcagatga

SEQ ID NO: 95 - DNA, 25 bp, Artificial Sequence
  Synthetic
tccagcgagaaccctatacttatct

SEQ ID NO: 96 - DNA, 20 bp, Artificial Sequence
  Synthetic
ccttcgcctacttcatgctc

SEQ ID NO: 97 - DNA, 20 bp, Artificial Sequence
  Synthetic
ggtctcgggttcatgaaaaa

SEQ ID NO: 98 - DNA, 24 bp, Artificial Sequence
  Synthetic
ccagaaggcacacagtttgatgca

SEQ ID NO: 99 - DNA, 24 bp, Artificial Sequence
  Synthetic
tcatcatcacgggtcacgaaattg

SEQ ID NO: 100 - DNA, 30 bp, Artificial Sequence
  Synthetic
cacacaagcttaaaaaagcgttttaattag

SEQ ID NO: 101 - DNA, 29 bp, Artificial Sequence
  Synthetic
cacactctagactcacataacgataagaa

SEQ ID NO: 102 - protein, 546 aa, Oryza sativa
MPKKKLSHRLFSALVSLLLHGKPISRSSSNTNTTLPHPSLLHKSSSSFPPMEKLAAKTLVLDVEGGLLRSSSLFPYFMLVALEAGGFLRGLVLLLLYPLLCVMGSDMALKVMAMVSFCGLRASRFRAGRAVLPKWFLEDVGEEGFDVMRSAMRRVCVTKMPRIMVEGFLKEYLEVEVVSGREMKVIWGFFTGIMEEEEGGDQEEVLLEEKKMLVDVVGFSTSLEFLQHHLSHCCKLVQEVYLVTREEKARWSALPRDKYPKPMVFHDGRLAFRPAAGDTLAMFTWLPFGAALAVARLAVALAVPYRYSTPILAATGLSWRLKGEAPTPLAGAGDGARRRGQLFVCNHRTLIDPVYVSVALDRPVRAVSYSLSRLSELISPIGRTVRLTRDRDSDGRAMARLLDGGDLVVVCPEGTTCREPCLLRFSPLFAELSDDVVPVGIAVDTAMFYATTAGGLKCLDPLYYIANPRTCYAVQFLERVDTSPARERRAPSTDVANLVQRRMGDALGYRCTMLTRKDKYLMLAGNDGVVNTTQDNHSAPGKKKMQ

SEQ ID NO: 103 - protein, 502 aa, Arabidopsis thaliana
MSPAKKSRSFPPISECKSREYDSIAADLDGTLLLSRSSFPYFMLVAIEAGSLFRGLILLLSLPIVIIAYLFVSESLGIQILIFISFAGIKIKNIELVSRAVLTRFYAADVRKDSFEVFDKCKKRKVVVTANPIVMVEPFVKDYLGGDKVLGTEIEVNPKTMKATGFVKKPGVLVGDLKRLAILKEFGDDSPDLGLGDTSDHDFMSICKEGYMVHETKSATTVPIESLKNRIIFHDGRLVQRPTPLNALIIYLWLPFGFMLSVFRVYFNLPLPERFVRYTYEILGIHLTIRGHRPPPPSPGKPGNLYVLNHRTALDPIIIAIALGRKITCVTYSVSRLSLMLSPIPAVALTRDRVADAARMRQLLEKGDLVICPEGTTCREPYLLRFSALFAELSDRIVPVAMNCKQGMFNGTTVRGVKFWDPYFFFMNPRPSYEATFLDRLPEEMTVNGGGKTPFEVANYVQKVIGGVLGFECTELTRKDKYLLLGGNDGKVESINKTKSME

(19) United States
(12) Patent Application Publication
(10) Pub. No.: US 2009/0163729 A1
(43) Pub. Date: Jun. 25, 2009

(54) Compositions and methods for using acyltransferases for altering lipid production on the surface of plants

(75) Inventors: Yonghua Li, Lansing, MI (US); Fred Beisson, Lansing, MI (US); Mike Pollard, Okemos, MI (US); John Ohlrogge, Okemos, MI (US)

Correspondence Address: MEDLEN & CARROLL, LLP, 101 HOWARD STREET, SUITE 350, SAN FRANCISCO, CA, 94105, US

(73) Assignee: The Board of Trustees Operating Michigan State University, East Lansing, MI (US)

(21) Appl. No.: 11/821,182

(22) Filed: Jun. 22, 2007

Related U.S. Application Data
(60) Provisional application No. 60/815,770, filed on Jun. 22, 2006.
(60) Provisional application No. 60/928,565, filed on May 10, 2007.

(51) Int. Cl. C11B 1/00; C12N 15/63; A01H 5/00; C12N 15/82

(52) U.S. Cl. 554/8; 435/320.1; 800/317.3; 800/317.2; 800/314; 800/322; 800/320.1; 800/320.2; 800/295; 554/227; 800/281; 252/182.12

(57) ABSTRACT

The present invention relates to compositions comprising acyltransferase nucleic acid molecules for altering lipids on the surface of plants, and related methods. In particular, the present invention provides compositions and methods for increasing the amount of free fatty acids, acylglycerols, and other lipids on the surface of a plant. In a preferred embodiment, the present invention relates to increasing activity of a GPAT acyltransferase for altering lipid on the plant surface, for increasing surface lipids, for enhancing environmental stress tolerance, increasing resistance to biotic stress, and providing novel plant lipids for commercial products. In further embodiments, the present invention relates to using an Arabidopsis thaliana GPAT acyltransferase for altering lipid compounds on the surface of a plant.

This application claims priority to provisional patent application Ser. No. 60/815,770 filed Jun. 22, 2006 and provisional patent application Ser. No. 60/928,565, filed May 10, 2007; which are herein incorporated by reference in their entirety.

This invention was made in part with government support under National Research Initiative Grant No: 98-35504-6190, Cooperative State Research, Education, and Extension Service HATCH No. MICL01533, National Research Initiative of the United States Department of Agriculture Cooperative State Research, Education, National Science Foundation MCB-0615563, and Extension Service (Grant 2005-35318-15419) from the United States Department of Agriculture. As such, the United States Government has certain rights in the invention.

### FIELD OF THE INVENTION

The present invention relates to compositions comprising acyltransferase nucleic acid molecules for altering lipids on the surface of plant, and related methods. In particular, the present invention provides compositions and methods for increasing the amount of free fatty acids, acylglycerols, and other lipids on the surface of a plant. In a preferred embodiment, the present invention relates to increasing activity of a GPAT acyltransferase for altering lipid on the plant surface, for increasing surface lipids, for enhancing environmental stress tolerance, increasing resistance to biotic stress, and providing novel plant lipids for commercial products. In further embodiments, the present invention relates to using an Arabidopsis thaliana GPAT acyltransferase for altering lipid compounds on the surface of a plant.

### BACKGROUND

Plant lipids and hydrocarbon chain derivatives including oils, free fatty acids and wax esters from roots, bark and seeds of plants are used to provide a wide variety of commercial products. Thus lipid extracts and exudates from these types of plant parts are used in a multitude of applications such as insecticides, pesticides, coolants, lubricants, inks, coatings, as food, oils, soap, cosmetics, and in medicine. For example, vegetable oils are used in cooking, in making margarine and other processed foods and further used in producing several non-food items such as soap, cosmetics, medicine, paint, pesticides, fungicides, and bio-fuel.

Plant breeders have long sought to develop crops with altered lipids for providing, for example, “designer oils” suited for specific purposes such as enhancing nutritional value or removing an undesirable component or enhancing a desirable component. Rapeseed oil, for example, naturally contains high amounts of nutritionally undesirable erucic acid so plant breeders have successfully bred rape plant varieties producing virtually no erucic acid. These practices are however very time consuming, taking numerous years to genetically perfect and then to develop as commercially acceptable cultivars.

Advances in plant genetics have revealed biochemical pathways by which plants make waxes and other oil components and these advances provide theoretical ways to influence fatty acid production in order to make “designer oils.” Recently, a new soybean has been developed for producing a less saturated, more heat stable oil while also providing a nutritionally healthier fatty acid composition. Thus genetic modification now makes it possible to improve the composition and properties of oils from different plants more quickly and precisely than with traditional breeding techniques.

However, commercial uses of these genetically modified plants is limited because these “designer oils” are primarily restricted to altering lipid expression inside of seeds, even when using promoters for expressing transgenes in nonseed parts. Further, these alterations in seed oils are limited to oils produced and harvested from ground whole seeds and seed tissue. Another limitation of these engineered plants is that the engineering primarily serves to modify lipids inside of cells which may lead to toxic or negative effects on the plant. Thus even with genetic engineering, providing an oil of specific compositions can lead to negative consequences for plant growth and metabolism that may require multiple modifications to overcome in order to provide a commercially valuable plant.

Therefore, it would be of considerable advantage to shift the production of the desired lipid or hydrocarbon derivative composition to the outside of the plant cell rather than to inside cells. Further, it would be desirable to express designer lipids in a wider range of plant parts and further to provide new designer lipid compositions.

### SUMMARY OF THE INVENTION

The present invention relates to compositions comprising acyltransferase nucleic acid molecules for altering lipids on the surface of plants, and related methods. In particular, the present invention provides compositions and methods for increasing the amount of free fatty acids, acylglycerols, and other lipids on the surface of a plant. In a preferred embodiment, the present invention relates to increasing activity of a GPAT acyltransferase for altering lipid on the plant surface, for increasing surface lipids, for enhancing environmental stress tolerance, increasing resistance to biotic stress, and providing novel plant lipids for commercial products. In further embodiments, the present invention relates to using an Arabidopsis thaliana GPAT acyltransferase for altering lipid compounds on the surface of a plant.

The present invention is not limited to any particular plant gene sequence encoding a protein comprising acyltransferase activity. Indeed, a variety of plant gene sequences encoding proteins with acyltransferase activity are contemplated. In some embodiments, the invention provides an isolated nucleic acid comprising a glycerol phosphate acyltransferase nucleic acid sequence or fragment thereof. In some embodiments, the invention provides an isolated nucleic acid comprising a sequence selected from the group consisting of SEQ ID NO:01, and sequences at least 59% identical to SEQ ID NO:01, wherein said sequence encodes a protein that alters lipids on the surface of plants. In other embodiments, the present invention provides isolated nucleotide sequences at least 59%, 60%, 70%, 80%, 90%, 95%, 98%, 99% (or more) identical to any of SEQ ID NO:01. In other embodiments, the invention further provides an isolated nucleic acid comprising a sequence selected from the group consisting of SEQ ID NOs: 2, 3, 4, 5, 6, 7, 8, homologes, orthologs and fragments thereof. The present invention is not limited to any particular plant polypeptide sequence comprising acyltransferase activity. Indeed, a variety of plant polypeptide sequences comprising acyltransferase activity are contemplated. In some embodiments, the invention provides a polypeptide comprising a sequence selected from the group consisting of SEQ ID NO:09, and sequences at least 38% identical to SEQ ID NO:09, wherein said sequence encodes a protein that alters plant surface lipids. In other embodiments, the present invention provides polypeptide sequences at least 38%, 39%, 40%, 50%, 60%, 70%, 80%, 90%, 95%, 98%, 99% (or more) identical to any of SEQ ID NO:09. The invention further provides a polypeptide sequence comprising a sequence selected from the group consisting of SEQ ID NOs: SEQ ID NOs: 10, 11, 12, 13, 14, 15, 16, and 17, homologes, orthologs and fragments thereof. In some embodiments, the plant polypeptide sequence further comprises SEQ ID NO:66. In some embodiments, the plant polypeptide sequence further comprises SEQ ID NO:67. In some embodiments, the invention provides a vector construct comprising an isolated plant acyltransferase nucleic acid molecule. In some embodiments, the invention provides a vector construct comprising an isolated nucleic acid molecule selected from the group consisting of SEQ ID NOs:01 and sequences at least 59% identical to SEQ ID NO:01, homologes, orthologs and fragments thereof. In other embodiments, the present invention provides nucleotide sequences at least 59%, 60%, 70%, 80%, 90%, 95%, 98%, 99% (or more) identical to any of SEQ ID NO:01. In other embodiments, the invention further provides a vector construct comprising an isolated nucleic acid molecule selected from the group consisting of SEQ ID NOs: 2, 3, 4, 5, 6, 7, 8, homologes, orthologs and fragments thereof. In some embodiments, the invention provides a vector construct comprising a nucleic acid sequence that encodes a polypeptide that is at least 38% identical to SEQ ID NO:09, wherein said polypeptide alters plant surface lipids. In other embodiments, the present invention provides amino acid sequences at least 38%, 39%, 40%, 50%, 60%, 70%, 80%, 90%, 95%, 98%, 99% (or more) identical to any of SEQ ID NO:09. In some embodiments, the plant polypeptide sequence of SEQ ID NO:09 further comprises SEQ ID NO:66. In some embodiments, the plant polypeptide sequence of SEQ ID NO:09 further comprises SEQ ID NO:67. The present invention is not limited to any particular plant acyltransferase molecule. Indeed, a variety of acyltransferase molecules are contemplated including but not limited to glycerol phosphate acyltransferase (GPAT) family molecules. In some embodiments, a glycerol phosphate acyltransferase nucleic acid family molecule is selected from the group consisting of a glycerol phosphate acyltransferase 1, glycerol phosphate acyltransferase 2, glycerol phosphate acyltransferase 3, glycerol phosphate acyltransferase 4, glycerol phosphate acyltransferase 5, glycerol phosphate acyltransferase 6, glycerol phosphate acyltransferase 7, glycerol phosphate acyltransferase 8, homologs, orthologs, and fragments retaining enzymatic activity for altering a lipid on the surface of a plant. In one embodiment, said glycerol phosphate acyltransferase is a glycerol phosphate acyltransferase 5. In one embodiment, said glycerol phosphate acyltransferase is a glycerol phosphate acyltransferase 7. In one embodiment, said glycerol phosphate acyltransferase is a glycerol phosphate acyltransferase 4. In one embodiment, said glycerol phosphate acyltransferase is a glycerol phosphate acyltransferase 8. The present invention is not limited to any particular plant as a source of the glycerol phosphate acyltransferase (GPAT) nucleic acid molecule. Indeed, a variety of plant sources are contemplated, including but not limited to a plant from one or more an Arabidopsis sp., Oryza sp., Nicotiana sp., a Lycopersicon sp., a Gossypium sp., and a Botryococcus sp., or any member of a Brassicaceae family, a Poaceae family, a Fabaceae family, a Solanaceae family, and a Malvaceae family.

In some embodiments, the invention provides an expression vector, comprising a nucleic acid sequence, wherein said nucleic acid sequence encodes a glycerol phosphate acyltransferase polypeptide, in operable combination with a promoter. In one embodiment, said promoter is operable in a plant. In one embodiment, said promoter is a plant promoter. In one embodiment, said promoter is selected from the group consisting of constitutive promoters, enzyme promoters, tissue specific promoters, inducible promoters, and temperature sensitive promoters. In one embodiment, said promoter is selected from the group consisting of a glycerol-3-phosphate acyltransferase 5 (GPAT5) promoter, a glycerol-3-phosphate acyltransferase 4, (GPAT4) promoter, a glycerol-3-phosphate acyltransferase 7 (GPAT7) promoter, a glycerol-3-phosphate acyltransferase 8 (GPAT8) promoter, a glycerol-3-phosphate acyltransferase 1 (GPAT1) promoter, a glycerol-3-phosphate acyltransferase (GPAT2) promoter, a glycerol-3-phosphate acyltransferase (GPAT3) promoter, a glycerol-3-phosphate acyltransferase 6 (GPAT6), a Lipid Transfer Protein 1 (LPT1) promoter, a CUTICULAR 1 (CUT1, eceriferum 6 (CER6)) promoter, a Long Chain Acyl-CoA Synthetase 2 (LACS2) promoter, a acyl-CoA synthetase long-chain family member 3 (ACSL3) promoter, FbL2A promoter, E6 promoter, patatin promoter, a potato multicystatin (PMC) promoter, a R929A promoter, a RCI2A promoter, a RCI2B promoter, a CBF promoter, and a potato α-amylase promoter. In one embodiment, said glycerol phosphate acyltransferase polypeptide is selected from the group consisting of SEQ ID NOs: 9, 10, 11, 12, 13, 14, 15, and 16, homologes, orthologs, and fragments thereof. In one embodiment, said glycerol phosphate acyltransferase polypeptide is at least 38% identical to SEQ ID NO:09. In one embodiment, said glycerol phosphate acyltransferase polypeptide is SEQ ID NO:09. In one embodiment, said plant is selected from the group consisting of a mustard, tobacco, potato, cotton, rice, and algae. In one embodiment, said glycerol phosphate acyltransferase polypeptide alters extracellular lipid on a plant part. In one embodiment, said promoter is operable in a plant, in operable combination with an antisense nucleic acid targeted to a nucleic acid sequence encoding a glycerol phosphate acyltransferase polypeptide or portion thereof. In one embodiment, said glycerol phosphate acyltransferase polypeptide is SEQ ID NO:09. In one embodiment, said plant is selected from the group consisting of a mustard, potato, and cotton. In one embodiment, said promoter is chosen from the group consisting of a constitutive promoter, a tissue specific promoter, an inducible promoter, and a temperature sensitive promoter.

In some embodiments, the invention provides a silencing expression vector, comprising a plant promoter, in operable combination with an antisense nucleic acid targeted to a nucleic acid sequence encoding a glycerol phosphate acyltransferase polypeptide or portion thereof. In one embodiment, said promoter is selected from the group consisting of constitutive promoters, enzyme promoters, tissue specific promoters, inducible promoters, and temperature sensitive promoters. In one embodiment, said promoter is selected from the group consisting of a glycerol-3-phosphate acyltransferase 5 (GPAT5) promoter, a glycerol-3-phosphate acyltransferase 4, (GPAT4) promoter, a glycerol-3-phosphate acyltransferase 7 (GPAT7) promoter, a glycerol-3-phosphate acyltransferase 8 (GPAT8) promoter, a glycerol-3-phosphate acyltransferase 1 (GPAT1) promoter, a glycerol-3-phosphate acyltransferase (GPAT2) promoter, a glycerol-3-phosphate acyltransferase (GPAT3) promoter, a glycerol-3-phosphate acyltransferase 6 (GPAT6), a Lipid Transfer Protein 1 (LPT1) promoter, a CUTICULAR 1 (CUT1, eceriferum 6 (CER6)) promoter, a Long Chain Acyl-CoA Synthetase 2 (LACS2) promoter, a acyl-CoA synthetase long-chain family member 3 (ACSL3) promoter, FbL2A promoter, E6 promoter, patatin promoter, a potato multicystatin (PMC) promoter, a R929A promoter, a RCI2A promoter, a RCI2B promoter, a CBF promoter, and a potato α-amylase promoter. In one embodiment, said glycerol phosphate acyltransferase polypeptide is selected from the group consisting of SEQ ID NOs: 9, 10, 11, 12, 13, 14, 15, and 16, homologes, orthologs, and fragments thereof. In one embodiment, said glycerol phosphate acyltransferase polypeptide is SEQ ID NO:09.

In some embodiments, the invention provides a transgenic plant having altered plant surface lipid expression, wherein the transgenic plant comprises a heterologous acyltransferase nucleic acid sequence molecule for altering the plant surface lipid molecules. It is not meant to limit the type of acyltransferase nucleic acid sequence molecule. In some embodiments, the acyltransferase nucleic acid sequence molecule is a plant glycerol phosphate acyltransferase molecule. In some embodiments, the acyltransferase nucleic acid molecule is selected from the group consisting of SEQ ID NOs:01, sequences at least 59% identical to SEQ ID NO:01, SEQ ID NOs: 2, 3, 4, 5, 7, 8, and homologs, orthologs, and fragments thereof. Accordingly in some embodiments, the nucleic acid sequence encodes a polypeptide that is at least 38% identical to SEQ ID NO:09. In other embodiments, the present invention provides a polypeptide sequence at least 38%, 39%, 40%, 50%, 60%, 70%, 80%, 90%, 95%, 98%, 99% (or more) identical to any of SEQ ID NO:09. In some embodiments, the acyltransferase nucleic acid sequence molecule encodes a polypeptide selected from the group consisting of SEQ ID NOs: 10, 11, 12, 13, 14, 15, 16, and 17. In some embodiments, the polypeptide sequence further comprises SEQ ID NO:66. In some embodiments, the polypeptide further comprises SEQ ID NO:67. The present invention is not limited to any particular type of altering of lipids on a plant surface. Indeed a variety of types of altering a plant surface lipid are contemplated, including, increasing total lipids on the surface of a plant, increasing a lipid on a plant surface, providing lipids novel to a plant surface, increasing a novel surface lipid in a plant exudate, and altering the structure of lipids on a plant surface. In yet other embodiments, said altering plant surface lipids is decreasing a wild-type lipid. In other embodiments, said alters plant surface lipids is increasing a novel surface lipid and decreasing a wild-type surface lipid. The present invention is not limited to any particular surface lipid. Indeed a variety of plant surface lipids are contemplated, including but not limited to C22-C30 saturated free fatty acids (FFA), tetracosanoic acid (lignoceric acid), monoacylglycerols (MAGs), α-monoacylglycerol, β-monoacylglycerol, wax esters, free fatty acids, very long chain fatty acids, polyester monomers, fatty dicarboxylic acids, and polyol fatty acids. In one embodiment, said free fatty acid comprises a tetracosanoic acid. In one embodiment, said monoacylglycerol is selected from the group consisting of α-monoacylglycerol and β-monoacylglycerol. In one embodiment, said monoacylglycerol is selected from the group consisting of 22-30 carbon chains and 32-60 carbon chains. In one embodiment, said very long chain fatty acid is selected from the group consisting of 22-30 carbon chain molecules and 32-60 carbon chain molecules. In one embodiment, said wax ester is selected from the group consisting of carbon chain lengths of C48 to C54 and C56 to C120. In some embodiments, the surface lipids comprise even carbon chain lengths and odd carbon chain lengths. In some embodiments, the surface lipids are even numbered chain lengths. In some embodiments, the surface lipids are odd numbered chain lengths. Accordingly, in other embodiments, plants of the present invention provide surface lipids with chain lengths at least 4, 6, 8, 10, 12, 14, 16, 18, 20, 22, 24, 26, 28, 30, 32, 34, 36, 39, 40, 42, 44, 46, 48, 50, 52, 54, 56, 58, 60 (or more) Carbons in length. In other embodiments, said heterologous nucleic acid molecule further comprises a plant vector construct. The present invention is not limited to any particular type of vector construct. Indeed, the use of a variety of vector constructs is contemplated. In some embodiments, the vector construct is a eukaryotic vector. In other embodiments, said eukaryotic vector is a plant vector. In other embodiments, said vector plant vector comprises a T-DNA vector. In other embodiments, said vector is a prokaryotic vector. In one embodiment, said vector construct comprises a heterologous promoter. In some embodiments, said acyltransferase nucleic acid sequence is in operable combination with a heterologous promoter. The present invention is not limited to any particular type of promoter. Indeed, the use of a variety of promoters is contemplated. In some embodiments, the promoter is a eukaryotic promoter. In further embodiments, the eukaryotic promoter is active in a plant. In some embodiments, said promoter is capable of expression in a plant. In some embodiments, said promoter sequence selected from the group consisting of a tissue specific promoter, a temperature inducible promoter, a constitutive promoter, a developmental promoter. In some embodiments, said promoter sequence is selected from the group consisting of a glycerol-3-phosphate acyltransferase 5 (GPAT5) promoter, a glycerol-3-phosphate acyltransferase 4, (GPAT4) promoter, a glycerol-3-phosphate acyltransferase 7 (GPAT7) promoter, a glycerol-3-phosphate acyltransferase 8 (GPAT8) promoter, a glycerol-3-phosphate acyltransferase 1 (GPAT1) promoter, a glycerol-3-phosphate acyltransferase (GPAT2) promoter, a a glycerol-3-phosphate acyltransferase (GPAT3) promoter, a glycerol-3-phosphate acyltransferase 6 (GPAT6) promoter, a Lipid Transfer Protein 1 (LPT1) promoter, a CUTICULAR 1 (CUT1, eceriferum 6 (CER6)) promoter, a Long Chain Acyl-CoA Synthetase 2 (LACS2) promoter, a acyl-CoA synthetase long-chain family member 3 (ACSL3) promoter, and a potato multicystatin (PMC) promoter. In one embodiment, said temperature sensitive promoter is chosen from the group consisting of R929A, RCI2A, RCI2B, CBF1, and potato α-amylase. In some embodiments, said constitutive promoter is a cauliflower mosaic virus 35S promoter. In some embodiments, said tissue specific promoter is active in the epidermis of plants. In some embodiments, said promoter is operable in green algal cells. In some embodiments, said promoter is operable is a unicellular green alga Chlamydomonas reinhardtii alternative oxidase (Aox1) promoter. In some embodiments, said promoter overexpresses the heterologous nucleic acid in a plant. In other embodiments, said plant surface comprises a plant exudate. In one embodiment, said plant exudate comprises one or more of a plant resin, a plant oil, a plant gum, and a plant wax. In one embodiment, the surface lipid is extracellular lipid. In a further embodiment, the extracellular lipid is a secretion from said plant. In one embodiment, the surface of a plant is a cuticle. In one embodiment, the surface of a plant is a lipid coating. In one embodiment, the plant surface comprises a plant cell wall. In one embodiment, the lipid comprises suberin. In one embodiment, the lipid comprises cutin. In one embodiment, the lipid comprises cuticle. The present invention is not limited to any particular plant. Indeed, a variety of plants are contemplated, including but not limited to a flowering plant, a vegetable plant, a crop plant, an herb plant, a shrub plant, and a tree plant. In one embodiment, said method provides a transgenic plant of the present inventions. In some embodiments, the plant is selected from the group consisting of a Brassica carinata, Crambe abyssinica, corn (Zea mays), canola (Brassica napus), alfalfa (Medicago sativa), rice (Oryza sativa), rye (Secale cereale), sorghum (Sorghum bicolor), millet (Pennisetum glaucum), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium hirsutum), sweet potato (Ipomoea batatus), cassaya (Manihot esculenta), coffee (Coffea spp.), coconut (Cocos nucifera), pineapple (Ananas comosus), citrus (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, Cork Oak (Quercus suber), Aspen (Populus tremula), Loblolly pine (Pinus taeda). In some embodiments, the plant is selected from the group consisting of a Brassicaceae species, Nicotiana species, a Solanum species, a Gossypium species, and a Botryococcus species. In some embodiments, the crop plant is selected from the group consisting of a mustard, tobacco, potato, cotton, sunflower, corn, safflower, rice, and algae. In some embodiments, the flowering plant is an Arabidopsis sp. plant. In some embodiments, the crop plant is a tobacco plant. In some embodiments, the crop plant is a potato plant. In some embodiments, the crop plant is a cotton plant. In one embodiment, the plant comprises a seed. In one embodiment, the plant is a seed. In one embodiment, a plant surface is a plant part surface. The present invention is not limited to any particular plant part surface. Indeed a variety of plant part surfaces are contemplated but not limited to a seed, root, stem, tuber, leaf, needle, shoot, bud, pod, fruit, rind, nut, bark, rhizome, bulb, boll, fiber, flower, flower, and a whole plant. In one embodiment, the surface of a plant is a cuticle. In one embodiment, the surface of a plant is a lipid coating of a plant part. In one embodiment, the surface of a plant is extracellular. In one embodiment, said surface of said plant is the surface of a seed.

The invention further provides a transgenic plant part, wherein said transgenic plant part comprising a heterologous glycerol phosphate acyltransferase nucleic acid sequence and altered extracellular lipid. In one embodiment, said altered extracellular lipid is increased as compared to a wild-type plant part. In one embodiment, said altered extracellular lipid comprises free fatty acid, monoacylglycerol, very long chain fatty acid, wax ester, polyester monomer, fatty dicarboxylic acid, polyol fatty acid, suberin, and cutin. In one embodiment, said heterologous glycerol phosphate acyltransferase nucleic acid is selected from the group consisting of SEQ ID NOs: 1 and 7. In one embodiment, said plant part comprises a seed, a tuber, a root, a stem, a leaf, a flower, and a whole plant. In one embodiment, said free fatty acid comprises C24, C26, and C28 chain lengths. In one embodiment, said monoacylglycerol comprises C24, C26, and C28 chain lengths. In one embodiment, said monoacylglycerol comprises alpha-monoacylglycerol and beta-monoacylglycerol. In one embodiment, said plant is selected from the group consisting of Arabidopsis, tobacco, potato, sunflower, corn, cotton, safflower, and rice. In one embodiment, said heterologous glycerol phosphate acyltransferase nucleic acid sequence is in operable combination with a promoter. In one embodiment, said promoter is chosen from the group consisting of a plant promoter. In one embodiment, said plant promoter is chosen from the group consisting of a constitutive promoter, a tissue specific promoter, and an inducible promoter. In one embodiment, said inducible promoter is chosen from the group consisting of a chemically induced promoter and a temperature sensitive promoter. In one embodiment, said temperature sensitive promoter is chosen from the group consisting of R929A, RCI2A, RCI2B, CBF1, and potato α-amylase. In one embodiment, said lipid surface is extracellular lipid. In one embodiment, said extracellular lipid comprises cell wall lipid. In one embodiment, said extracellular lipid comprises suberin and cuticle.

In one embodiment, the invention provides an isolated extracellular plant lipid. comprising a first lipid, wherein said first lipid is a monoacylglycerols at least 5% w/w and a second lipid. said second lipid comprises a free fatty acid, very long chain fatty acid, wax ester, polyester monomer, fatty dicarboxylic acid, polyol fatty acid and combinations thereof. In one embodiment, said free fatty acid comprises C24, C26, and C28 chain lengths. In one embodiment, said monoacylglycerol comprises alpha-monoacylglycerol and beta-monoacylglycerol. In one embodiment, said extracellular lipid comprises a monoacylglycerol. In one embodiment, said plant lipid further comprises α-monoacylglycerol and β-monoacylglycerol. In one embodiment, said plant lipid comprises monoacylglycerols further comprising 32-60 carbon chains. Accordingly, in one embodiment, said plant lipid comprises monoacylglycerols further comprising a 22, 24, 26, 28, and 30 carbon chain molecule. In one embodiment, said plant lipid comprises monoacylglycerols further comprising 32-60 carbon chains. Accordingly, in one embodiment, said plant lipid comprises monoacylglycerols further comprising a 32, 34, 36, 38, 40, 42, 44, 46, 48, 50, 52, 54, 56, 58, and 60 carbon chain molecule. In one embodiment, said plant lipid comprises a very long chain fatty acid molecule further comprising 22-30 carbon chain molecules. In one embodiment, said plant lipid comprises a very long chain fatty acid molecule further comprising a 32-60 carbon chain molecule. Accordingly, in one embodiment, said plant lipid comprises a very long chain fatty acid molecule further comprising 22, 24, 26, 28, and 30 carbon chain molecule. Accordingly, in one embodiment, said plant lipid comprises a very long chain fatty acid molecule further comprising a 32, 34, 36, 38, 40, 42, 44, 46, 48, 50, 52, 54, 56, 58, and 60 carbon chain molecule. In one embodiment, said plant lipid comprises a wax ester molecule is selected from the group consisting of carbon chain lengths of C48 to C54 and C56 to C120. Accordingly, in one embodiment, said plant lipid comprises a wax ester molecule 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120 carbon chain molecule. In one embodiment, said plant lipid comprises a saturated wax ester further comprising C40-C54 molecules. Accordingly, in one embodiment, said plant lipid comprises a wax ester molecule C46, C48, C50, C52, and C54 length molecule. In one embodiment, said plant lipid comprises a surface of a plant, wherein said plant comprises a heterologous glycerol phosphate acyltransferase nucleic acid molecule.

The invention also provides an isolated extracellular plant lipid, wherein said extracellular lipid comprises free fatty acids and monoacylglycerols.

In one embodiment, said altered extracellular lipid comprises free fatty acid, monoacylglycerol, very long chain fatty acid, wax ester, polyester monomer, fatty dicarboxylic acid, polyol fatty acid, suberin, and cutin. In one embodiment, said altered extracellular lipid is increased as compared to a wild-type plant part. In one embodiment, said increased extracellular lipid is a total extracellular wax at least 1000 ug/gfw. Accordingly an extracellular wax load is at least 1000 ug/gfw, 2000 ug/gfw, 3000 ug/gfw, 4000 ug/gfw, and more. In one embodiment, said increased extracellular lipid is a wax load at least 25 ug/gfw, 50 ug/gfw, 100 ug/gfw, 200 ug/gfw, 300 ug/gfw, 400 ug/gfw, 500 ug/gfw, 600 ug/gfw, and more. In one embodiment, said monoacylglycerols are at least 100 ug/gfw. In one embodiment, said monoacylglycerol is selected from the group consisting of α-monoacylglycerol and β-monoacylglycerol. In one embodiment, said wax ester is selected from the group consisting of carbon chain lengths of C48 to C54 and C56 to C120. In one embodiment, said altered extracellular lipid is decreased as compared to a wild-type plant part. In certain embodiments, the present invention provides methods for altering lipid expression on the surface of a plant. In some embodiments, plant surface lipid expression is altered by transfecting acyltransferase nucleic acid molecules into plants for purposes of generating transgenic plants with altered plant surface lipids.

Specifically, the present invention provides methods for altering lipids on the surface of a plant, comprising, a) providing, i) a heterologous acyltransferase nucleic acid molecule, wherein said nucleic acid molecule alters a lipid on the surface of a plant, ii) a plant, wherein said plant comprises a lipid surface, and, b) transfecting a heterologous acyltransferase nucleic acid into the plant, wherein said nucleic acid is expressed, for providing a plant with altered surface lipids. In some embodiments, the acyltransferase nucleic acid molecule is selected from the group consisting of SEQ ID NOs:01, sequences at least 59% identical to SEQ ID NO:01, SEQ ID NOs: 2, 3, 4, 5, 7, 8, and homologs, orthologs, and fragments thereof. Accordingly in some embodiments, the nucleic acid sequence encodes a polypeptide that is at least 38% identical to SEQ ID NO:09. In other embodiments, the present invention provides a polypeptide sequence at least 38%, 39%, 40%, 50%, 60%, 70%, 80%, 90%, 95%, 98%, 99% (or more) identical to any of SEQ ID NO:09. In some embodiments, the acyltransferase nucleic acid sequence molecule encodes a polypeptide selected from the group consisting of SEQ ID NOs: 10, 11, 12, 13, 14, 15, 16, and 17. In some embodiments, the polypeptide sequence further comprises SEQ ID NO:66. In some embodiments, the polypeptide further comprises SEQ ID NO:67. It is not meant to limit the type of transfecting. Indeed, a variety of types of transfecting are contemplated, including, but not limited to Agrobacterium mediated transfecting, electroporating, vacuum infiltration, particle bombarding, and the like. In one embodiment, said heterologous acyltransferase nucleic acid molecule further comprises a vector construct. In one embodiment, said method further comprises, c) collecting lipids from the surface of the plant. In one embodiment, said collecting comprises removal of extracellular lipids from a plant surface. In one embodiment, said collecting further comprising an extraction solution, wherein said extraction solution comprises an organic solvent. In one embodiment, said organic solvent is selected from the group consisting of a chloroform and a dichlromethane. The present invention is not limited to any particular type of collecting altered lipids from the surface of a transgenic plant. Indeed, a variety of types of collecting are contemplated, including, but not limited to dipping, washing, extraction, scraping, absorption, centrifugation, and incubating a plant part in an extraction solution. In one embodiment, said dipping is briefly emersing a plant part into an organic solvent (organic solvent dipping). In one embodiment, said method further comprises, provides, a lipid collection medium. In a further embodiment, said collecting comprises incubating a plant part in a lipid collection medium. In one embodiment, said lipid collection medium is selected from the group consisting of a culture medium, growth medium, and an incubation medium. In one embodiment, said collecting further comprises, drying the lipid extract. In one embodiment, said collecting further comprises saponification. In one embodiment, said collecting further comprises adding methyl esters to lipids. In one embodiment, said collecting further comprises, treating a lipid extract with a methylation reagent selected from the group consisting of an acidic or basic methanol and/or a hydrogenolysis reagent. In one embodiment, said reagent is NaOCH₃. In one embodiment, said hydrogenolysis reagent is LiAlH₄. In one embodiment, said collecting further comprises, separating lipids using gas chromatography. In one embodiment, said collecting further comprises, separating lipids using classical phase partitioning. In one embodiment, said phase partitioning is hexane partitioning. In a further embodiment, said collecting further comprises, lipid fractionation techniques following phase partitioning.

The present invention provides methods for altering lipids on the surface of transgenic plants, comprising, a) providing, i) a heterologous acyltransferase nucleic acid molecule, wherein said nucleic acid molecule alters a lipid on the surface of a plant, ii) a transgenic plant, wherein said transgenic plant comprises a surface, and, b) transfecting a heterologous acyltransferase nucleic acid into the transgenic plant, wherein said nucleic acid is expressed, for providing a transgenic plant with altered surface lipids. In one embodiment, the method further comprises, c) collecting altered lipids from the surface of a transgenic plant.

The invention also provides a method for increasing extracellular lipids secreted by a plant part, comprising; a) providing, i) a vector comprising a nucleic acid sequence, wherein said nucleic acid sequence encodes a glycerol phosphate acyltransferase polypeptide, or portion thereof, for increasing extracellular lipid, and ii) a plant part; and b) transfecting the plant tissue with the vector under conditions such that the glycerol phosphate acyltransferase polypeptide increases extracellular lipids; and c) collecting extracellular lipid from said plant part. In one embodiment, said glycerol phosphate acyltransferase polypeptide is selected from the group consisting of SEQ ID NOs: 9, 10, 11, 12, 13, 14, 15, and 16, homologes, orthologs, and fragments thereof.

The invention also provides a method for altering extracellular plant lipids, comprising: a) providing, i) a vector comprising a T-DNA insertion sequence, wherein said T-DNA insertion sequence targets a nucleic acid sequence encoding a polypeptide selected from the group consisting of SEQ ID NOs: 9, 10, 11, 12, 13, 14, 15, and 16; and ii) a plant tissue; and b) transfecting the plant tissue with the vector under conditions such that the T-DNA insertion sequence alters extracellular lipids; c) collecting said altered extracellular lipids. In one embodiment, said altered extracellular lipid is increased lipid. In one embodiment, said increased lipid is increased insoluble primary alcohol lipid molecules.

The invention also provides a method of altering plant surface lipid, comprising, providing, a) providing; i) a silencing expression vector encoding an antisense nucleic acid targeted to a nucleic acid sequence encoding a plant glycerol phosphate acyltransferase polypeptide, and ii) a plant tissue, wherein said plant tissue comprises a lipid surface; and b) transfecting the plant tissue with the vector under conditions such that the antisense sequence is expressed and the plant surface lipid is altered. In one embodiment, said antisense nucleic acid silences the plant glycerol phosphate acyltransferase polypeptide. In one embodiment, said antisense nucleic acid is an siRNA sequence. In one embodiment, said glycerol phosphate acyltransferase polypeptide is selected from the group consisting of SEQ ID NOs: 9, 10, 11, 12, 13, 14, 15, and 16, homologes, orthologs, and fragments thereof. In one embodiment, said nucleic acid sequence is in operable combination with a promoter. In one embodiment, said promoter is chosen from the group consisting of promoters operable in plants. In one embodiment, said promoter is chosen from the group consisting of a constitutive promoter, a tissue specific promoter, and an inducible promoter. In one embodiment, said inducible promoter is chosen from the group consisting of a chemically induced promoter and a temperature sensitive promoter. In one embodiment, said temperature sensitive promoter is chosen from the group consisting of R929A, RCI2A, RCI2B, CBF1, and potato α-amylase. In one embodiment, said lipid surface is extracellular lipid. In one embodiment, said extracellular lipid comprises cell wall lipid. In one embodiment, said extracellular lipid comprises suberin and cuticle. In one embodiment, said method further comprises transfecting the plant tissue with the vector. In one embodiment, said method further comprises regenerating a plant from said transfected plant tissue, under conditions such that the antisense sequence is expressed and the plant surface lipid is altered. The invention also provides a method of producing lipids comprising: a providing a transgenic plant comprising a heterologous acyltransferase nucleic acid sequence or a silencing expression vector encoding an antisense nucleic acid targeted to a nucleic acid sequence encoding a plant glycerol phosphate acyltransferase polypeptide; and b) growing said transgenic plant under conditions such that said plant produces lipids. In one embodiment, said method further comprises the step of isolating said lipids from said plant.

The invention also provides for a use of a nucleic acid sequence encoding an acyltransferase polypeptide for providing a transgenic plant, for providing an extracellular lipid comprising free fatty acids and monoacylglycerols, for altering the cell wall thickness of a plant, for altering suberin production of a potato tuber, or for decreasing the extracellular lipid load of a cotton fiber.

The invention also provides for a use of a vector of the present inventions, as described herein, for making a transgenic plant. In one embodiment, said use comprises an expression vector comprising a nucleic acid sequence encoding a glycerol phosphate acyltransferase polypeptide in operable combination with a plant promoter.

The invention also provides for a use of a comprises a silencing expression vector comprising a plant promoter in operable combination with an antisense nucleic acid targeted to a nucleic acid sequence encoding a glycerol phosphate acyltransferase polypeptide or portion thereof. for making a transgenic plant.

In some embodiments, the invention provides for a use of the transgenic plant parts of the present inventions, as described herein, to produce a desired lipid or group of lipids. In one embodiment said use provides a desired lipid. In one embodiment said use produces a group of lipids.

### DESCRIPTION OF THE FIGURES

FIG. 1 shows exemplary scanning electron microscopy of altered surfaces of stems and mature seeds from WT (wild-type) Arabidopsis plants compared to Arabidopsis plants overexpressing a GPAT family acyltransferase 5 (35S::GPAT5) gene. Stems were taken from the bottom section of 5-week-old Arabidopsis plants.

FIG. 2 shows exemplary stem wax loads from wild-type Arabidopsis (WT) plants vs. transgenic Arabidopsis plants overexpressing a GPAT family acyltransferase 5 (35S::GPAT5) (n=4, 5-week-old plants). Two independent transformant plant lines are shown, Line 1 and Line 2. (Error bars=±S.D., n=6). A) a comparison of total waxes and major classes of wax components. B) Loads of wax components sorted by major classes and chain length. 29 ALK: C₂₋₉ alkane; 29 SA: C29 secondary alcohol (15-hydroxy); 29 KET (15-oxo): C29 ketone; and C26 to C30 PA: C26 to C30 primary alcohol.

FIG. 3 demonstrates an exemplary inverse relationship between the accumulation of Very Long Chain Fatty Acids (VLCFA C22-C30 including MAGs and FFAs) and decrease in wild-type (WT) waxes in exemplary independent transgenic Arabidopsis plant lines (35S::GPAT5) that overexpressed GPAT5. Specifically, VLFAs were released by transmethylation from intact stems collected from 18 independent 35S::GPAT5 overexpression plant lines. OE-1 and OE-2 are high-lighted black and labeled on the graph. The negative correlation between the amount of standard waxes and VLCFAs is highly significant (Kendall rank correlation tau statistic=0.63, two-tailed p=0.0002 with normal approximation and correction for ties, n=18).

FIG. 4 shows an exemplary high temperature gas chromatography (GC) chromatogram of trimethylsilyl (TMS)—derivatives of chloroform dipping fractions prepared from the stems of Arabidopsis plants that overexpressed GPAT family acyltransferase 5 (GPAT5). Specifically, this chromatogram demonstrated expression of increased and novel C22-C30 fatty acids in the form of free fatty acids (FFAs). A) transgenic plants (35S::GPAT5) and B) wild-type (WT). IS: internal standard. IS1-6: IS1=C17 monoacylglycerol (MAG), IS2=C23 free fatty acid, IS3=C₂₋₈ alkane, IS4=C12:0 triacylglycerol (TAG), IS5=C14:0 triacylglycerol (TAG) and IS6=C17:0 triacylglycerol (TAG).

FIG. 5 demonstrates exemplary mass spectra of C24 α-monoacylglycerol (MAG) (A) and C24 β-monoacylglycerol (MAG) and (B) from transgenic Arabidopsis plants that overexpressed GPAT family acyltransferase 5 (GPAT5).

FIG. 6 demonstrates exemplary mass distribution of lipids, including fatty acids and derivatives in the chloroform dipping lipid fraction (“extracellular”, i.e. from the stem surface) vs. the remaining total lipid fraction of the same stem after chloroform dipping (“intracellular”) in Arabidopsis plants overexpressing GPAT family acyltransferase 5 (GPAT5). Fatty acids were analyzed by acid-catalyzed transmethylation allowing the detection of WT-type waxes and total fatty acids, i.e. free and esterified fatty acids (mean±SE, n=6, data from the average of two independent lines shown).

FIG. 7 shows an exemplary multiple protein sequence alignment of GPAT family acyltransferase homologues from Arabidopsis thaliana and Oryza sativa (japonica cultivar-group) prepared using CLUSTAL W (1.83) a Multiple Sequence Alignment Program.

FIG. 8 shows an exemplary pBI121_GPAT5 vector construct of the present invention.

FIG. 9 shows exemplary nucleic acid and amino acid sequences (SEQ ID NOs:1-48, exemplary promoters and exemplary vector sequences (SEQ ID NOs: 78-88).

FIG. 10 demonstrates an exemplary phylogenetic comparison (Dendogram) of Arabidopsis and Oryza GPAT protein sequences.

FIG. 11 shows an exemplary structure of the GPAT5 Gene with a T-DNA Insertion, and GPAT5 expression analyzed by RT-PCR. (A) Genomic organization of the gpat5-1 and gpat5-2 loci. Boxes represent exons. The T-DNA insertion point is indicated as a triangle, with L and R indicating left and right borders, respectively. (B) RT-PCR analysis of the GPAT5 transcript in wild-type and mutant (gpat5-1 and gpat5-2) flowers. Approximately 0.1 mg of total RNA was used in each PCR, and eIF4A-1 (At3g13920) was used as a control. (C) RT-PCR analysis of GPAT5 expression in roots, rosette leaves, stems, open flowers, and developing seeds. Approximately 0.1 mg of total RNA was used in each PCR, and eIF4A-1 (At3g13920) was used as a control.

FIG. 12 shows an exemplary Fatty Acid extraction from the Seed Coat/Endosperm Fraction of the Wild Type and gpat5 Mutants. Mature seeds were manually dissected, and total fatty acids of the membrane and storage lipids of the seed coat/endosperm fraction were analyzed as fatty acid methyl esters by gas chromatography. Values are means of six replicates. Error bars denote 95% CI (Confidence Interval).

FIG. 13 shows an exemplary lipid polyester monomers from seeds, roots, and flowers of Wild-Type and gpat5 plants. (A) Polyester monomers from mature seeds, (B) Polyester monomers from roots of 1-week-old seedlings grown on agar, and (C) Polyester monomers from opened flowers. The insoluble dry residue obtained after grinding and delipidation of tissues with organic solvents was depolymerized with sodium methoxide, and aliphatic and aromatic monomers released were analyzed by gas chromatography-mass spectrometry. Values are means of six data points (two independent experiments using different biological samples involving triplicate assays for the depolymerization reaction). Error bars denote 95% CI (Confidence Interval). DCAs, fatty dicarboxylic acids; FAs, fatty acids; fw, fresh weight; PAs, primary alcohols. Polyol fatty acids are 10,16-hydroxy 16:0 and 9,10,18-hydroxy 18:1.

FIG. 14 shows exemplary brown pigmentation of Wild-Type and gpat5 Seeds. (A) Batch color of wild-type versus gpat5-1 and gpat5-2 seeds and (B) amount of soluble and insoluble PAs in wild-type versus gpat5-1 and gpat5-2 seeds. Values are means of six data points (two independent experiments using different seed batches involving triplicate assays for the depolymerization reactions). Error bars denote 95% CI (Confidence Interval.

FIG. 15 shows exemplary germination of gpat5 Seeds under various conditions. (A) Rate of germination after harvest and increasing periods of dry storage. (B) Rate of germination on MS medium supplemented with increasing NaCl concentrations. (C) Rate of germination on MS medium supplemented with increasing KCl concentrations. (D) Rate of germination on MS medium supplemented with increasing K₂SO₄ concentrations. Seeds were germinated after cold treatment (except in [A]). Values are means of 9 data points (A) or 12 data points ([B] to [D]; i.e., from three or four independent experiments, respectively, which use different seed batches and involve three replicate lots of approximately 100 seeds for each seed batch). Error bars denote 95% CI (Confidence Interval. Similar results were obtained with gpat5-2.

FIG. 16 shows an exemplary Gene Tree and Gene Expression Profile of the Eight Putative GPATs of Arabidopsis. (A) The cladogram shows the branching order of Arabidopsis GPATs according to a phylogenetic tree of protein sequences of plant acyltransferases (Kim and Huang, 2004, Plant Physiol. 134: 1206-1216; herein incorporated by reference). The original tree was built using the neighbor-joining method with 1000 bootstrap replicates. Bootstrap values are percentages. (B) Microarray expression data derived from AtGenExpress (Schmid et al., 2005, Nat. Genet. 37: 501-506; herein incorporated by reference). Expression levels in each tissue (root, leaf, stem, flower, and seed) at different developmental stages were averaged (bars represent means±SE). The expression profile for GPAT7 was determined directly by the inventors as described herein via RT-PCR analysis because its expression profile was not available at AtGenExpress.

FIG. 17 shows exemplary Fatty Acids from intracellular lipids. (A) Rosette leaves, (B) Roots, and (C) Seeds. Error bars denote 95% CI (Confidence Interval).

FIG. 18 shows an exemplary wax composition of the Arabidopsis Seed Surface. ALK: alkane, PA: primary alcohol, SA: secondary alcohol, KET: ketone. Error bars denote 95% CI (Confidence Interval).

FIG. 19 shows exemplary Lipid Polyester Monomers from Roots of 3-Week-Old Seedlings Grown on Agar. Analysis was carried as indicated for FIG. 13. Values are means of six data points. Error bars denote 95% CI (Confidence Interval). FAs: fatty acids; DCAs: fatty dicarboxylic acids; and PAs: primary alcohols.

FIG. 20 shows exemplary lipid polyester monomers from leaves of wild-type and gpat5 mutant plants. Rosette leaves analyzed from 5-week-old plants as indicated for FIG. 13. Values are means of six data points. Error bars denote 95% CI (Confidence Interval). FAs: fatty acids; DCAs: fatty dicarboxylic acids; and PAs: primary alcohols.

FIG. 21 shows an exemplary analysis of plant surface lipids of plant stems from transgenic GPAT7 ectopic expressing Arabidopsis plants compared to stems from wild-type Arabidopsis plants. This analysis demonstrated a GPAT7 induced increase in production of free fatty acids (FFA) and monoacylglycerols (MAG) in addition to an increase in the production of long chain extracellular lipids.

FIG. 22 shows an exemplary analysis of plant surface lipids of plant seeds from transgenic GPAT7 ectopic expressing plants compared to seeds from wild-type plants. This analysis demonstrated a GPAT7 induced increase in production of free fatty acids (FFA) and monoacylglycerols (MAGs).

FIG. 23 shows exemplary GPAT5 ectopic overexpression produced surface MAG on tobacco leaf. GPAT5 over expression also produces surface MAG on tobacco leaf analysis of plant surface lipids of plant leaves from transgenic GPAT5 ectopic expressing tobacco plants compared to seeds from wild-type tobacco plants. This analysis demonstrated a GPAT5 induced increase in production of monoacylglycerols (MAGs) on the surface of leaves.

FIG. 24 shows exemplary increased FFA and DCA in Arabidopsis stem analysis of plant surface lipids of plant stems from transgenic GPAT8 over-expression Arabidopsis plants compared to stems from wild-type Arabidopsis plants. This analysis demonstrated GPAT8 ectopic expression increased C16:0 DCA in Arabidopsis stem cutin. Error bars=95% CI (Confidence Interval).

FIG. 25 shows an exemplary profiling of root waxes of 7-week-old WT Arabidopsis plants. A, Recovery of total waxes by chloroform dipping of roots vs stems. B, Extraction kinetics of individual root wax components. C, Composition and content of Arabidopsis root waxes (mean with 95% CI (Confidence Interval), n=4). PA, primary alcohol; FFA, free fatty acid; MAG, monoacylglycerol; VLCFFA, very long chain free fatty acid.

FIG. 26 shows an exemplary identification of MAGs present in Arabidopsis root waxes by GC-MS of their bis-trimethylsilyl derivatives: mass spectra of C24 α-MAG (A) and C24 β-MAG (B).

FIG. 27 shows exemplary changes in the quantity of FFAs and MAGs (sum of both isomers, α and β) in the root waxes prepared from the T-DNA insertional mutant lines (gpat5-1 and gpat5-2), the 35S::GPAT5 overexpression lines (OE-1 and OE-2) as compared to that of WT (7-week old soil grown roots). Bars=mean with 95% CI (Confidence Interval) (n=4). The difference between the mean of the WT and the mean of a transgenic line was significantly different from zero for each comparison (p<0.05, 2-sided t-test with unequal variances).

FIG. 28. Polyester analysis of 7-week-old roots of WT and 35S::GPAT5 overexpression line OE-2 (mean with 95% CI, n=3).

FIG. 29 shows an exemplary composition and content of chloroform-dipping fraction of epicuticular waxes collected from stems of 5-week-old WT and 35S::GPAT5 overexpression lines OE-1 and OE-2 (mean with 95% CI (Confidence Interval, n=4). ALK, alkane; OH, secondary hydroxy; KET, ketone; and PA, primary alcohol). Inset figure shows exemplary amounts of individual lipid classes.

FIG. 30. Polyester analysis of 6-week-old stems of WT and 35S::GPAT5 overexpression lines (mean with 95% CI, n=4).

FIG. 31 shows an exemplary extractability of stem wax components after stems collected from Arabidopsis plants overexpressing 35S::GPAT5 were rapid dipped in chloroform. A, The chloroform-extracted lipid and the residual tissue were transmethylated to release total fatty acids as methyl esters prior to silylation and GC (Gas Chromatographic) Analysis. C16-C20 fatty acids are derived mainly from polar membrane lipids while C22-C30 fatty acids are derived almost exclusively from FFAs and MAGs. B, Chain length distribution of novel MAGs and FFAs in the chloroform dipping and residual fractions. These lipids were analyzed without transmethylation (mean with 95% CI (Confidence Interval, n=4).

FIG. 32 shows an exemplary 35S::AtGPAT5 ectopic expression in transgenic tobacco plants produced surface MAGs (mean with 95% CI (Confidence Interval), n=3).

FIG. 33 shows exemplary GPAT7 ectopic expression that produced surface FFAs and MAGs. Data shown are average of three independent lines. Error bars=±SE.

FIG. 34 shows exemplary GPAT7 ectopic expression that increased surface FFAs and MAGs. Data shown are average of three independent lines. Error bars=±SE.

FIG. 35 shows exemplary GPAT8 ectopic expression that increased surface C16:0 DCA on the stem cutin. Error bars=95% CI (Confidence Interval).

FIG. 36 shows exemplary GPAT4 ectopic expression that increased C16:0 DCA on the stem cutin. Error bars=95% CI (Confidence Interval).

### DEFINITIONS

To facilitate an understanding of the present invention, a number of terms and phrases as used herein are defined below:

The use of the article “a” or “an” is intended to include one or more.

The use of the term “comprise” or “comprising” or “comprises” refers to the inclusion of a stated integer or step or group of integers or steps but not the exclusion of any other integer or step or group of integers or steps.

As used herein, terms defined in the singular are intended to include those terms defined in the plural and vice versa.

The term “plant” is used in it's broadest sense. Plant includes, but is not limited to, any species of crop plant, grass (e.g. turfgrass), bush, shrub, sedge, rush, ornamental or decorative, cereal, fodder, forage, fruit, vegetable, herb plant, woody plant, tree, and algae.

The terms “crop” and “crop plant” are used herein its broadest sense. The term includes, but is not limited to, any species of plant or alga edible by humans or used as a feed for animals or fish or marine animals, or consumed by humans, or used by humans, or viewed by humans (flowers) or any plant or alga used in industry or commerce or education, such as vegetable crop plants, fruit crop plants, tree crop plants, and the like.

The term “algae” is used in it's broadest sense. Algae includes any organism also called “Protist” or “Protista” comprising a photosynthetic pigment, such as a chlorophyll (green, for example, Chlorophyta, including sea lettuce), a carotenoid (yellow, orange, or brown, for example, Phaeophyta; Laminaria species, such as kelp, including Rockweed (Ascophyllum nodosum)), and an anthocyanin (red, for example, Rhodophyta including nori). Algae encompasses microalgae, such as single cell organisms, for example, Botryococcus species, and macroalge, such as seaweed and kelp.

The term “plant part” as used herein refers to a plant structure or a plant tissue. It is not meant to limit a plant part to any particular plant structure or plant tissue. Such plant parts include, but are not limited to a seed, a root, a rhizome, bark, a stem, a tiller, a sprig, a stolen, a plug, a shoot, stomata, a leaf, a flower petal, meristem, crown, a fruit, and the like.

The term “plant tissue” includes differentiated and undifferentiated tissues of plants including those present in roots, shoots, leaves, pollen, seeds and tumors, as well as cells or tissues in culture (e.g., single cells, guard cells, protoplasts, embryos, callus, seeds, etc.). Plant tissue may be in planta, in organ culture, tissue culture, or cell culture.

The terms “leaf” and “leaves” refer to a usually flat, green structure of a plant where photosynthesis and transpiration take place and attached to a stem or branch.

The term “shoot” refers to a portion of a plant consisting of a stem and its attached leaves.

The term “stem” refers to a main ascending axis of a plant. The vascularized above-ground supportive portion of a plant, and below-ground structures with the same anatomy and development.

The term “meristem” refers to undifferentiated tissue from which new cells are formed, e.g., the tips of roots or stems; the growing tip.

The term “rhizome” refers to an underground stem usually horizontally oriented and sometimes specialized for food storage.

The terms “tapetum” or “tapeta” refers to a special tissue surrounding the microsporocytes in the anthers of Anthophyta or flowering plants.

The term “seed” refers to a ripened ovule, consisting of the embryo and a casing.

The term “silique” refers to a dry elongated fruit divided by a partition between the two carpels dividing it into two sections.

The term “pod” as in “seed pod” refers to a case or fruit containing one to many seeds.

The term “cuticle” refers to an extracellular layer of cutin and waxes covering aerial portions of plants. Cuticular waxes are complex mixtures comprising very long-chain fatty acids, alkanes, primary and/or secondary alcohols, aldehydes, ketones, esters, triterpenes, sterols, and flavonoids. Wax compounds may be embedded within the cutin polymer framework and form “intracuticular wax” or loaded outside of the cutin polymer and form an “epicuticular wax” layer.

The term “cutin” refers to a fatty acid-derived polymer making up the cuticle. Examples of cutin monomers comprise C16-C18 omega-hydroxy and dicarboxylic fatty acids and other types of in-chain-hydroxy fatty acids, etc. Cutin is embedded with intracuticular waxes and covered by epicuticular waxes.

The term “epidermis” or “epidermal layer” in reference to a stem or leaf or seed surface refers to the outermost cell layer of a plant.

The term “cortex” refers to an outer part of a plant body just under and inside of the epidermis and outside the vascular tissue, such that it provides a layer of cells surrounding the vascular tissue in roots, stems, or leaves of many seed plants, wherein an endodermis is the innermost layer of the cortex.

The term “periderm” or “bark” refers to an outer bark layer or cork cambium layer comprising suberin, a waxy substance, for sealing the stem against water loss or invasion by insects, or infection by bacteria or fungal spores, and may provide insulation for the plant.

The term “cork” refers to cells produced by the cork cambium that have suberized cells walls and are dead at maturity, cork may also be referred to as suberized tissue separate from the cork cambium.

The term “suberin” refers to a plant-specific cell wall-associated hydrophobic polymer containing a fatty acid-derived domain and an aromatic domain, that is found in or secreted by various tissues of underground plant parts and some aerial organs, for example, suberin is secreted by cork cells for sealing a stem against water loss or by various plant tissues in response to biotic or abiotic stresses.

The term “casparian strip” refers to a suberized, thickened ribbon on the walls of endodermis.

The term “exudate” refers to a liquid, resinous or gelatinous substance secreted by organs or parts of a plant, or oozes out of a plant as a natural surface coating or when a plant is damaged, for example, gum, sap, milky sap, resin, mucilages, oils, required oils, waxes, latex and the like.

The term “latex” refers to a viscous fluid exuded from cut surfaces of leaves and stems, such as from laticifer plants and plants from the Sunflower Family (Asteraceae), Euphorbia Family (Euphorbiaceae), Mulberry Family (Moraceae), etc., comprising polymers made up of isoprene units in the cis-configuration. Conversely, Gutta-percha latex consists of 1,4-polyisoprene residues in trans-configuration as produced by the sapodilla family (Sapotaceae), the chicle or naseberry tree), et cetera.

The term “mucilages” refers to a slimy water-soluble polysaccharide material exuded by certain plants or plant organs, such as in mucilaginous gums, mucilaginous latex, mucilaginous oils, mucilaginous resins, mucilaginous waxes and the like.

The term “resin” refers to a lipid-soluble terpenes or phenols, such as resins also found in mucilages, latex, oils, resins, waxes, and et cetera.

The term “gums” refers to a complex water-soluble polysaccharide chains, also found in mucilages, latex, oils, resins, waxes.

The term “resin duct” refers to a tube-like extracellular space lined with resin-producing cells for secreting resin.

The term “oil” refers to a combination of fatty acid and glycerol, such as in any of numerous mineral, vegetable, and synthetic substances and animal and vegetable fats that comprise any one of the following characteristics, slippery, combustible, viscous, liquid or liquefiable at room temperatures, soluble in various organic solvents such as diethylether but not in water.

The term “plant oil” refers to any of various oils obtained from plants. A plant oil can be used in food products, medicinally, and industrially.

The term “cell wall” refers to a wall bounding the cells in plants.

The term “plasmalemma” refers to a cytoplasmic membrane that in a walled plant cell is located inside of the cell wall.

The term “surface” in reference to the “surface of a plant” refers to the external or outside area, such as the cuticular area, or extracellular structures, such as wax structures on leaves and stems, of a plant or plant part or plant tissue as opposed to the internal or inside area of a plant or plant part or plant tissue. A plant surface may also refer to the extracellular surface of a plant cell as opposed to the intracellular area of a plant cell. For the purposes of the present invention, the terms “surface lipid” or “surface acyl derivative” or “surface wax” as in a lipid or an acyl derivative or a wax that is found on the outside of a plant, such as comprising an epidermal coating or a cell wall coating or within a cell wall or a surface structure, such as a wax crystal, or a component of bark or cork, or on the outside surface of a seed or the epidermal surface of a seed pod, or a plant secretion, or an exudate, and the like.

As used herein, the term “plant surface component” refers to any portion of a plant located in the vicinity of the plant surface. Examples of plant surface components s include, but are not limited to, exudates, leaf, needle, stem, shoot, bud, pod, fruit, rind, nut, seed, bark, root, rhizome, bulb, and flower.

The term “extracellular” refers to the area outside of a cell, for example, the external surface of a cell wall, the cell wall, intercellular space, and the like.

The term “intracellular” refers to the area inside of a cell, for example, the cytosol, cytoplasm, endoplasmic reticulum, plastid, and the like, are located inside of a cell.

The term “plastid” refers to an intracellular organelle that is a site of synthesis of starch, fatty acids, chlorophyll, et cetera.

The term “propagation” refers to the process of producing new plants, either by vegetative means involving the rooting or grafting of pieces of a plant, or by sowing seeds. The terms “vegetative propagation” and “asexual reproduction” refer to the ability of plants to reproduce without sexual reproduction, by producing new plants from existing vegetative structures that are clones, i.e., plants that are genetically identical to the mother plant and to each another. For example, the division of a clump, rooting of proliferations, or cutting of mature crowns can produce a new plant. The terms “tissue culture” and “micropropagation” in reference to reproduction of a plant refer to a form of asexual propagation undertaken in specialized laboratories, in which clones of plants are produced from small cell clusters from very small plant parts (e.g. buds, nodes, leaf segments, root segments, etc.), grown aseptically (free from any microorganism) in a container where the environment and nutrition can be controlled.

As used herein, the term “culture” in reference to a cell or tissue refers to any in vitro growth or maintenance. Included within this term are continuous cell lines (e.g., with an immortal phenotype), primary cell cultures, finite cell lines (e.g., non-transformed cells), and any other cell population maintained in vitro, including, but not limited to plant (e.g., protoplast, meristem, etc.), mammalian, yeast, bacterial, and insect cells.

The term plant cell “compartments or organelles” is used in its broadest sense. The term includes but is not limited to, the endoplasmic reticulum, Golgi apparatus, trans Golgi network, plastids, sarcoplasmic reticulum, glyoxysomes, mitochondrial, chloroplast, thylakoid membranes and nuclear membranes, and the like.

The terms “F” or “filial” refer to different generations involved in breeding experiments. The terms “parental generation” or “P” or “F0” refer to the parent in a genealogy. The term “F1” or “first filial generation” refers to a son or daughter of a parent or F0. When members of the F1 generation are crossed, their offspring are called the F2 generation, et cetera.

The terms “allele” and “alleles” refer to each version of a gene for a same locus that has more than one sequence. For example, there are multiple alleles for eye color at the same locus. The terms “nucleic acid sequence,” “nucleotide sequence of interest” or “nucleic acid sequence of interest” refer to any nucleotide sequence (e.g., RNA or DNA), the manipulation of which may be deemed desirable for any reason (e.g., treat disease, confer improved qualities, etc.), by one of ordinary skill in the art. Such nucleotide sequences include, but are not limited to, coding sequences of structural genes (e.g., reporter genes, selection marker genes, oncogenes, disease resistance genes, growth factors, etc.), and non-coding regulatory sequences which do not encode an mRNA or protein product (e.g., promoter sequence, polyadenylation sequence, termination sequence, enhancer sequence, etc.).

The term “gene” encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region termed “exon” or “expressed regions” or “expressed sequences” interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage and polyadenylation.

The term “oligonucleotide” refers to a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

The term “polynucleotide” refers to refers to a molecule comprised of several deoxyribonucleotides or ribonucleotides, and is used interchangeably with oligonucleotide. Typically, oligonucleotide refers to shorter lengths, and polynucleotide refers to longer lengths, of nucleic acid sequences.

The term “heterologous gene” refers to a gene encoding a factor that is not in its natural environment (in other words, has been altered by the hand of man). For example, a heterologous gene includes a gene from one species introduced into another species. In a further example, a heterologous gene also includes a gene native to an organism that has been altered in some way (for example, added in multiple copies, capable of being expressed in novel tissues or cells of the organism, mutated, linked to a non-native promoter or enhancer sequence, etc.). Heterologous genes may comprise plant gene sequences that comprise cDNA forms of a plant gene; the cDNA sequences may be expressed in either a sense (to produce mRNA) or anti-sense orientation (to produce an anti-sense RNA transcript that is complementary to the mRNA transcript). Heterologous genes are distinguished from endogenous plant genes in that the heterologous gene sequences are typically joined to nucleotide sequences comprising regulatory elements such as promoters that are not found naturally associated with the gene for the protein encoded by the heterologous gene or with plant gene sequences in the chromosome, or are associated with portions of the chromosome not found in nature (for example, genes expressed in loci where the gene is not normally expressed).

The terms “promoter element,” “promoter,” or “promoter sequence” as used herein, refer to a DNA sequence that is located at the 5′ end (in other words precedes) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA. Promoters may be tissue specific or cell specific or organelle specific.

The term “tissue specific” as it applies to a promoter refers to a promoter that is capable of directing selective expression of a nucleotide sequence of interest to a specific type of tissue (for example, expression in seeds, tubers, roots, stems, leaves, etc.) in the relative absence of expression of the same nucleotide sequence of interest in a different type of tissue (for example, expressed in seeds but not leaves or expressed in seeds but at a lower amount in leaves). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (for example, detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected.

The term “cell type specific” as applied to a promoter refers to a promoter which is capable of directing selective expression of a nucleotide sequence of interest in a specific type of cell in the relative absence of expression of the same nucleotide sequence of interest in a different type of cell within the same tissue, (for example, epidermis of leaf and stem) in the relative absence of expression of the same nucleotide sequence of interest in a different type of tissue (for example, expressed in leaf or stem epidermis but not in other types of leaf or stem cells such as parenchymal cells). One exemplary cell type specific promoter is a lipid transfer protein (LTP) promoter of Brassica napus, (see, for example, Sohal et al. (1999) Plant Mol. Biol. September; 41(1):75-87; herein incorporated by reference in its entirety). Such promoters have been used successfully to direct the expression of heterologous nucleic acid sequences in transformed plant tissue.

The term “cell type specific” when applied to a promoter also means a promoter capable of promoting selective expression of a nucleotide sequence of interest in a region within a single tissue. Cell type specificity of a promoter may be assessed using methods well known in the art, for example, immunohistochemical staining. Briefly, tissue sections are embedded in paraffin, and paraffin sections are reacted with a primary antibody which is specific for the polypeptide product encoded by the nucleotide sequence of interest whose expression is controlled by the promoter. A labeled (for example, peroxidase conjugated) secondary antibody which is specific for the primary antibody is allowed to bind to the sectioned tissue and specific binding detected (for example, with avidin/biotin) by microscopy.

The term “organelle specific” or “tissue specific” as it applies to a promoter refers to a promoter that is capable of directing selective expression of a nucleotide sequence of interest to a specific type of organelle, such as to a plastid organelle or chloroplast organelle or an endoplasmic reticulum organelle, etc., or a tissue, such as an epidermis, cuticle, seed, flower, et cetera.

Promoters may be constitutive or regulatable. The term “constitutive” when made in reference to a promoter means that the promoter is capable of directing transcription of an operably linked nucleic acid sequence in the absence of a stimulus (for example, heat shock, chemicals, light, etc.). Typically, constitutive promoters are capable of directing expression of a transgene in substantially any cell and any tissue. Exemplary constitutive plant promoters include, but are not limited to 35 Cauliflower Mosaic Virus (CaMV 35; see, for example, U.S. Pat. No. 5,352,605, herein incorporated by reference in its entirety), mannopine synthase, octopine synthase (ocs), superpromoter (see, for example, WO 95/14098, herein incorporated by reference in its entirety)), and ubi3 (see, for example, Garbarino and Belknap (1994) Plant Mol. Biol. 24:119-127, herein incorporated by reference in its entirety) promoters. Such promoters have been used successfully to direct the expression of heterologous nucleic acid sequences in transformed plant tissue.

In contrast, a “regulatable” promoter is one which is capable of directing a level of transcription of an operably linked nuclei acid sequence in the presence of a stimulus (for example, cold, heat, heat shock, chemicals, light, etc.) which is different from the level of transcription of the operably linked nucleic acid sequence in the absence of the stimulus. The term “oligonucleotide” refers to a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

The term “polynucleotide” refers to refers to a molecule comprised of several deoxyribonucleotides or ribonucleotides, and is used interchangeably with oligonucleotide. Typically, oligonucleotide refers to shorter lengths, and polynucleotide refers to longer lengths, of nucleic acid sequences.

The term “an oligonucleotide (or polypeptide) having a nucleotide sequence encoding a gene” or “a nucleic acid sequence encoding” a specified polypeptide refers to a nucleic acid sequence comprising the coding region of a gene or in other words the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc., may be placed in close proximity to the coding region of the gene if needed to permit proper initiation of transcription and/or correct processing of the primary RNA transcript. Alternatively, the coding region utilized in the expression vectors of the present invention may contain endogenous enhancers, exogenous promoters, splice junctions, intervening sequences, polyadenylation signals, etc., or a combination of both endogenous and exogenous control elements.

The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

The term “Tₘ” refers to the “melting temperature” of a nucleic acid. Melting temperature Tₘ is the midpoint of the temperature range over which nucleic acids are denatured (e.g. DNA:DNA, DNA:RNA and RNA:RNA, etc.). Methods for calculating the T.sub.m of nucleic acids are well known in the art (see, for example, Sambrook, et al., Molecular Cloning: A Laboratory Manual, 2 ed., Cold Spring Harbor Laboratory Press, New York (1989) pp. 9.50-51, 11.48-49, and 11.2-11.3, herein incorporated by reference).

The term “stringency” refers to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “low” stringency are often required with nucleic acids that are derived from organisms that are genetically diverse, as the frequency of complementary sequences is usually less.

“Low stringency conditions” when used in reference to nucleic acid hybridization comprise conditions equivalent to binding or hybridization at 42 degree C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent (50×Denhardt's contains per 500 ml:05 g Ficoll (Type 400, Pharmacia):05 g BSA (Fraction V; Sigma)) and 100 μg/ml denatured salmon sperm DNA followed by washing in a solution comprising 5×SSPE, 0.1% SDS at 42 degree C. when a probe of about 500 nucleotides in length is employed.

“Medium stringency conditions” when used in reference to nucleic acid hybridization comprise conditions equivalent to binding or hybridization at 42 degree C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×. Denhardt's reagent and 100 μg/ml denatured salmon sperm DNA followed by washing in a solution comprising 1.0×SSPE, 1.0% SDS at 42 degree C. when a probe of about 500 nucleotides in length is employed.

“High stringency conditions” when used in reference to nucleic acid hybridization comprise conditions equivalent to binding or hybridization at 42 degree C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42 degree C. when a probe of about 500 nucleotides in length is employed.

It is well known that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

The term “recombinant” when made in reference to a nucleic acid molecule refers to a nucleic acid molecule that is comprised of segments of nucleic acid joined together by means of molecular biological techniques. The term “recombinant” when made in reference to a protein or a polypeptide refers to a protein molecule that is expressed using a recombinant nucleic acid molecule.

The term “selectable marker” refers to a gene which encodes an enzyme having an activity that confers resistance to an antibiotic or drug upon the cell in which the selectable marker is expressed, or which confers expression of a trait which can be detected for example, luminescence or fluorescence). Selectable markers may be “positive” or “negative.” Examples of positive selectable markers include the neomycin phosphotransferase (NPTII) gene which confers resistance to G418 and to kanamycin, and the bacterial hygromycin phosphotransferase gene (hyg), which confers resistance to the antibiotic hygromycin. Negative selectable markers encode an enzymatic activity whose expression is cytotoxic to the cell when grown in an appropriate selective medium. For example, the HSV-tk gene is commonly used as a negative selectable marker. Expression of the HSV-tk gene in cells grown in the presence of gancyclovir or acyclovir is cytotoxic; thus, growth of cells in selective medium containing gancyclovir or acyclovir selects against cells capable of expressing a functional HSV TK enzyme.

The terms “protein,” “polypeptide,” “peptide,” “encoded product,” “amino acid sequence,” are used interchangeably to refer to compounds comprising amino acids joined via peptide bonds and a “protein” encoded by a gene is not limited to the amino acid sequence encoded by the gene, but includes post-translational modifications of the protein. Where the term “amino acid sequence” is recited herein to refer to an amino acid sequence of a protein molecule, the term “amino acid sequence” and like terms, such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein. The deduced amino acid sequence from a coding nucleic acid sequence includes sequences which are derived from the deduced amino acid sequence and modified by post-translational processing, where modifications include but not limited to glycosylation, hydroxylations, phosphorylations, and amino acid deletions, substitutions, and additions. Thus, an amino acid sequence comprising a deduced amino acid sequence is understood to include post-translational modifications of the encoded and deduced amino acid sequence. The term “X” may represent any amino acid.

The term “reverse-transcriptase” or “RT-PCR” refers to a type of “PCR” and “polymerase chain reaction” where the starting material is mRNA. The starting mRNA is enzymatically converted to complementary DNA or “cDNA” using a reverse transcriptase enzyme. The cDNA is then used as a “template” for a “PCR” reaction.

The term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “isolated” when used in relation to a nucleic acid or polypeptide, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may single-stranded), but may contain both the sense and anti-sense strands (i.e., the oligonucleotide may be double-stranded).

The term “purified” refers to molecules, either nucleic or amino acid sequences that are removed from their natural environment isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

As used herein, the terms “purified” and “to purify” also refer to the removal of contaminants from a sample. The removal of contaminating proteins results in an increase in the percent of polypeptide of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “host cell” refers to any cell or plant capable of harboring or replicating and/or transcribing and/or translating a heterologous gene or a whole or portion of a pathogen. Thus, a “host cell” refers to any eukaryotic or prokaryotic cell (e.g., plant cells, stomatal cells, guard cells, algal cells whether located in vitro or in vivo.

The term “vector” refers to nucleic acid molecules that transfer DNA segment(s) from one cell to another. The term “vehicle” is sometimes used interchangeably with “vector.” Introduction of the vectors into plant cells is achieved by methods known to those skilled in the art, such as polyethylene glycol methods, electroporation, Agrobacterium-mediated methods, and particle gun methods.

The terms “expression vector” or “expression cassette” refer to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. The term “transfection” as in “transfecting a plant cell” or “transfecting a plant tissue,” refers to the introduction of foreign DNA into cells. Transfection may be accomplished by a variety of means known to the art including calcium phosphate-DNA co-precipitation, DEAE-dextran-mediated transfection, polybrene-mediated transfection, glass beads, electroporation, microinjection, liposome fusion, lipofection, protoplast fusion, viral infection, biolistics (i.e., particle bombardment) and the like.

The term “transgenic” when used in reference to a plant or fruit or seed (in other words, a “transgenic plant” or “transgenic fruit” or a “transgenic seed”) refers to a plant or fruit or seed that contains at least one heterologous gene in one or more of its cells. The term “transgenic plant material” refers broadly to a plant, a plant structure, a plant tissue, a plant seed or a plant cell that contains at least one heterologous gene in one or more of its cells.

The terms “transformants” or “transformed cells” include the primary transformed cell and cultures derived from that cell without regard to the number of transfers.

The term “recombinant” when made in reference to a nucleic acid molecule refers to a nucleic acid molecule which is comprised of segments of nucleic acid joined together by means of molecular biological techniques. The term “recombinant” when made in reference to a protein or a polypeptide refers to a protein molecule which is expressed using a recombinant nucleic acid molecule.

The term “overexpression” refers to the production of a gene product in transgenic organisms that exceeds levels of production in normal or non-transformed organisms.

The term “cosuppression” refers to the expression of a foreign gene which has substantial homology to an endogenous gene resulting in the suppression of expression of both the foreign and the endogenous gene. The term “altered levels” refers to the production of gene product(s) in transgenic organisms in amounts or proportions that differ from that of normal or non-transformed organisms.

The term “sample” is used in its broadest sense. In one sense it can refer to a plant cell or tissue. In another sense, it is meant to include a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

The terms “eukaryotic” and “eukaryote” are used in it broadest sense. It includes, but is not limited to, any organisms containing membrane bound nuclei and membrane bound organelles. Examples of eukaryotes include but are not limited to animals, plants, alga, diatoms, and fungi.

The terms “prokaryote” and “prokaryotic” are used in it broadest sense. It includes, but is not limited to, any organisms without a distinct nucleus. Examples of prokaryotes include but are not limited to bacteria, blue-green algae, archaebacteria, actinomycetes and mycoplasma.

The terms “infecting” and “infection” when used with a bacterium refer to co-incubation of a target biological sample, (e.g., cell, tissue, etc.) with the bacterium under conditions such that nucleic acid sequences contained within the bacterium are introduced into one or more cells of the target biological sample or proteins produced by a bacterium produce disease symptoms in the target biological sample.

The term “Agrobacterium” refers to a soil-borne, Gram-negative, rod-shaped phytopathogenic bacterium which causes crown gall. The term “Agrobacterium” includes, but is not limited to, the strains Agrobacterium tumefaciens, (which typically causes crown gall in infected plants), and Agrobacterium rhizogens (which causes hairy root disease in infected host plants). Infection of a plant cell with Agrobacterium generally results in the production of opines (e.g., nopaline, agropine, octopine etc.) by the infected cell. Thus, Agrobacterium strains which cause production of nopaline (e.g., strain LBA4301, C58, A208, GV3101) are referred to as “nopaline-type” Agrobacteria; Agrobacterium strains which cause production of octopine (e.g., strain LBA4404, Ach5, B6) are referred to as “octopine-type” Agrobacteria; and Agrobacterium strains which cause production of agropine (e.g., strain EHA105, EHA101, A281) are referred to as “agropine-type” Agrobacteria.

The terms “bombarding, “bombardment,” and “biolistic bombardment” refer to the process of accelerating particles towards a target biological sample (e.g., cell, tissue, etc.) to effect wounding of the cell membrane of a cell in the target biological sample and/or entry of the particles into the target biological sample. Methods for biolistic bombardment are known in the art (see, for example, U.S. Pat. No. 5,584,807, herein incorporated by reference), and are commercially available (e.g., the helium gas-driven microprojectile accelerator (PDS-1000/He, BioRad).

The term “microwounding” when made in reference to plant tissue refers to the introduction of microscopic wounds in that tissue. Microwounding may be achieved by, for example, particle bombardment as described herein.

As used herein, the term “pathogen” refers a biological agent that causes a disease state (e.g., infection, anthracnose, etc.) in a host. “Pathogens” include, but are not limited to, viruses, bacteria, archaea, fungi, protozoans, mycoplasma, prions, parasitic organisms and insects.

The term “phytopathogen” refer to an organism that is pathogenic to a plant.

The terms “bacteria” and “bacterium” refer to all prokaryotic organisms, including those within all of the phyla in the Kingdom Procaryotae. It is intended that the term encompass all microorganisms considered to be bacteria, for example, Pseudomonas sp. including Mycoplasma, Chlamydia, Actinomyces, Streptomyces, and Rickettsia. All forms of bacteria are included within this definition including cocci, bacilli, spirochetes, spheroplasts, protoplasts, et cetera. Also included within this term are prokaryotic organisms which are gram negative or gram positive. “Gram negative” and “gram positive” refer to staining patterns with the Gram-staining process which is well known in the art. (See e.g., Finegold and Martin, Diagnostic Microbiology, 6th Ed., CV Mosby St. Louis, pp. 13-15 [1982]). “Gram positive bacteria” are bacteria which retain the primary dye used in the Gram stain, causing the stained cells to appear dark blue to purple under the microscope. “Gram negative bacteria” do not retain the primary dye used in the Gram stain, but are stained by the counterstain. Thus, gram negative bacteria appear red.

As used herein, the term “microorganism” refers to any species or type of microorganism, including but not limited to, bacteria, archaea, fungi, protozoans, mycoplasma, and parasitic organisms. The present invention contemplates that a number of microorganisms encompassed therein will also be pathogenic to a subject.

As used herein, the term “fungi” is used in reference to eukaryotic organisms such as the molds and yeasts, including dimorphic fungi and any fungi found growing on a plant.

The term “wild-type” when made in reference to a plant refers to a plant that has the characteristics of plants isolated from a naturally occurring source. The term “wild-type” when made in reference to a plant also refers to a gene and a gene product, which has the characteristics of a gene and a gene product isolated from a naturally occurring plant. A wild-type plant is that which is most frequently observed in a population and is thus arbitrarily designated the “normal” or “wild-type” form of the plant and genes found within that plant. In contrast, the term “modified” or “mutant” when made in reference to a plant refers to a plant comprising a gene or to a gene product, respectively, to a gene or to a gene product which displays modifications in sequence and/or functional properties (i.e., altered characteristics) when compared to the wild-type gene or gene product expressed in wild-type plants. It is noted that naturally-occurring mutants can be isolated; these are identified by the fact that they have altered characteristics when compared to the wild-type plant and the expressed wild-type gene or gene product.

The terms “homolog,” “homologue,” “homologous,” and “homology” when used in reference to amino acid sequence or nucleic acid sequence or a protein or a polypeptide refers to a degree of sequence identity to a given sequence, or to a degree of similarity between conserved regions, or to a degree of similarity between three-dimensional structures or to a degree of similarity between the active site, or to a degree of similarity between the mechanism of action, or to a degree of similarity between functions. In some embodiments, a homolog has a greater than 20% sequence identity to a given sequence. In some embodiments, a homolog has a greater than 40% sequence identity to a given sequence. In some embodiments, a homolog has a greater than 60% sequence identity to a given sequence. In some embodiments, a homolog has a greater than 70% sequence identity to a given sequence. In some embodiments, a homolog has a greater than 90% sequence identity to a given sequence. In some embodiments, a homolog has a greater than 95% sequence identity to a given sequence. In some embodiments, homology is determined by comparing internal conserved sequences to a given sequence. In some embodiments, homology is determined by comparing designated conserved functional regions. In some embodiments, homology is determined by comparing designated conserved “motif” regions. In some embodiments, means of determining homology are described in the Experimental section (Example VI).

The term “homology” when used in relation to nucleic acids or proteins refers to a degree of identity. There may be partial homology or complete homology. The following terms are used to describe the sequence relationships between two or more polynucleotides and between two or more polypeptides: “identity,” “percentage identity,” “identical,” “reference sequence,” “sequence identity,” “percentage of sequence identity,” and “substantial identity.” “Sequence identity” refers to a measure of relatedness between two or more nucleic acids or proteins, and is described as a given as a percentage “of homology” with reference to the total comparison length. A “reference sequence” is a defined sequence used as a basis for a sequence comparison; a reference sequence may be a subset of a larger sequence, for example, the sequence that forms an active site of a protein or a segment of a full-length cDNA sequence or may comprise a complete gene sequence. Since two polynucleotides or polypeptides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window,” as used herein, refers to a conceptual segment of in internal region of a polypeptide. In one embodiment, a comparison window is at least 77 amino acids long. In another embodiment, a comparison window is at least 84 amino acids long. In another embodiment, conserved regions of proteins are comparison windows. In a further embodiment, an amino acid sequence for a conserved transmembrane domain is 24 amino acids. Calculations of identity may be performed by algorithms contained within computer programs such as the ClustalX algorithm, see, for example, Thompson, et al. (1997 Nucleic Acids Res. 24, 4876-4882; herein incorporated by reference); MEGA2 (version 2.1) (Kumar, et al. (2001) Bioinformatics 17:1244-1245; herein incorporated by reference); “GAP” (Genetics Computer Group, Madison, Wis.), “ALIGN” (DNAStar, Madison, Wis.; herein incorporated by reference), BLAST (National Center for Biotechnology Information; NCBI as described at http://, followed by, www.ncbi., followed by, nlm.nih.gov/BLAST/blast_help.shtml) and MultAlin (Multiple sequence alignment) program (Corpet, (1988) Nucl. Acids Res., 16(22):10881-10890 at http://, followed by, prodes.toulouse.inra.fr/multalin/multalin., followed by, html), all of which are herein incorporated by reference).

For comparisons of nucleic acids, 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (Smith and Waterman, (1981) Adv. Appl. Math. 2:482; herein incorporated by reference) by the homology alignment algorithm of Needleman and Wunsch (Needleman and Wunsch, (1970) J. Mol. Biol. 48:443, herein incorporated by reference), by the search for similarity method of Pearson and Lipman (Pearson and Lipman, (1988) Proc. Natl. Acad. Sci. (U.S.A.) 85:2444), herein incorporated by reference), by computerized implementations of these algorithms (GAP, BESTFIT, FASTA, and TFASTA in the Wisconsin Genetics Software Package Release 7.0, Genetics Computer Group, 575 Science Dr., Madison, Wis., herein incorporated by reference), or by inspection, and the best alignment (i.e., resulting in the highest percentage of homology over the comparison window) generated by the various methods is selected. The term “sequence identity” means that two polynucleotide or two polypeptide sequences are identical (i.e., on a nucleotide-by-nucleotide basis or amino acid basis) over the window of comparison.

The term “percentage of sequence identity” is calculated by comparing two optimally aligned sequences over the window of comparison, determining the number of positions at which the identical nucleic acid base (e.g., A, T, C, G, U, or I) or amino acid, in which often conserved amino acids are taken into account, occurs in both sequences to yield the number of matched positions, dividing the number of matched positions by the total number of positions in the window of comparison (i.e., the window size), and multiplying the result by 100 to yield the percentage of sequence identity.

The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention.

The term “ortholog” refers to a gene in different species that evolved from a common ancestral gene by speciation. In some embodiments, orthologs retain the same function.

The term “hydrocarbon” refers to a compound consisting of carbon and hydrogen only, such as an alkane.

The term “hydrocarbon derivative” refers to any straight or branched hydrocarbon chain from 2 to 60 carbons in length that may be modified by single or double bonds and/or oxo, epoxyl, carboxyl, hydroxyl groups or any other functional group of interest. Examples of hydrocarbon derivatives include fatty acids and their derivatives. The number after the “C” is the number of carbon atoms in the chain, e.g. C2, C16, C24, C26, etc. The number after the colon tells us the number of double bonds in the carbon chain. Where there are no double bonds between the carbon atoms; the designation is “:0,” where there is one double bond “:1,” etc., such that a C6:2 represents a carbon chain of 6 with 2 double bonds.

For the purposes of the present invention a “lipid” refers to fatty acids and their derivatives as well as to substances related biosynthetically or functionally to these compounds, such as fatty alcohols, dicarboxylic acids, acylglycerols, wax esters, etc. For the purposes of the present invention the term “lipid” also refers to “wax.”

The term “wax” refers to any various natural or artificial and oily or greasy substance that is a hydrocarbon derivative that may include a free fatty acid or an ester of a fatty acid that is insoluble in water but soluble in nonpolar organic solvents. A wax can be a composition comprising a mixture of hydrocarbon derivatives or ester of a fatty acid. Waxes may also comprise saturated and unsaturated hydrocarbons, without substitution by oxygen such as in alkanes or with substitution by oxygen, such as in fatty acids, such as found in some plant epicuticular wax, gums, mucilages, latex, oils, resins, et cetera. For the purposes of the present invention the term “wax” also refers to “lipid.”

For the purposes of the present invention, the term “plant waxes” refers to any subset of a lipid present on the surface of a plant including but not limited to free fatty acids, very long chain fatty acids, and monoacylglycerols. Examples of plant waxes include waxes derived from plants such as carnauba wax, obtained from the leaves of a Brazilian palm, and candelilla wax, produced by an Euphorbia antisyphilitica.

The term “wax ester” or “WE” refer to a class of wax components that are esters of fatty acids and fatty alcohols. As used herein, a wax ester may comprise a carbon chain length of C48 to C54 or C56 to C120.

The term “acyl” refers to any compound comprising an RCO—, where R is an organic group derived from an organic acid, such as a fatty acid.

The terms “fatty acid derivatives” or “acyl derivatives” refer to glycerol esters, wax esters, fatty alcohols, esters of alcohols and dicarboxylic acids, etc., including those products catalyzed by an GPAT family acyltransferase of the present invention.

The terms “monoacylglycerol” or “MAG” refer to a glycerol esterified at any one of its three hydroxyl groups by a fatty acid: α-monoacylglycerol (sn position 1 or 3) or (β-monoacylglycerol (sn position 2). As used herein, a MAG may comprise a chain length ranging from a 2-30 carbon chain or a 31-60 carbon chain.

The terms “diacylglycerol” or “DAG” refer to a glycerol that was esterified at two of its three hydroxyl groups by a fatty acid.

The terms “triacylglycerol” or “TAG” refer to a glycerol that was esterified at each of its three hydroxyl groups by a fatty acid.

The term “fatty acid” refers to a compound comprising hydrogen (H), oxygen (O), and carbon (C), such that a fatty part of a fatty acid is a chain of carbon atoms bonded together; each C is also bonded to one or more Hydrogen's (H):

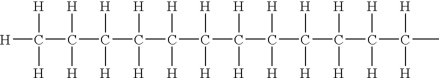

and an “acid” part of a fatty acid has one C, two O's and one H with a structure

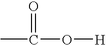

As used herein, a “free fatty acid” is a fatty acid that is not an ester or does not comprise an ester group.

As used herein, the term “fatty acid” comprises free fatty acids and fatty acids esterified to another compound.

As used herein, “two lines” represent “two bonds” or a “double bond” such as that shown for an acid structure, between a C and one of the O's.

The terms “very-long-chain fatty acids” or “VLCFA” refer to fatty acids with carbon chains ranging from C22 to C30 and C32 to C60 and higher, and including free fatty acids and esterified fatty acids that may be saturated or monounsaturated. Examples of a very long chain fatty acids (VLCFA's) are “lignoceric acid” or “tetracosanoic acid”; C24:0

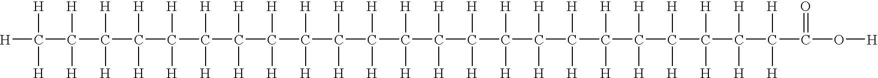

And hexacosanoic acid; C26:0

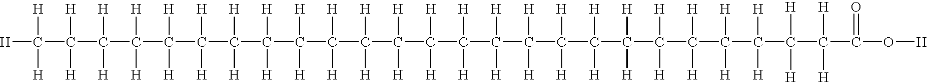

The term “ω-hydroxy fatty acid” refers to a fatty acid where the hydroxyl group is at the end (in the omega position. i.e. terminal position) of the hydrocarbon chain, where conversely, the carboxyl group is located at the beginning of the chain.

The terms “wax synthase” or “wax-ester synthase” or “long-chain-alcohol O-fatty-acyltransferase” refer to an enzyme, such as EC 2.3.1.75, for transferring saturated or unsaturated acyl residues of chain-length C₁₆ to C₃₀ to long-chain alcohols, forming waxes.

The term “acyltransferase” refers to a class of enzymes, such as EC 2.3.1 for transferring acyl groups from donor molecules onto acceptor molecules. For the purposes of the present inventions, acyltransferase encompasses genes and gene fragments encoding an enzyme and the enzyme including but not limited to active fragments, synthetic variants, mutants, et cetera.

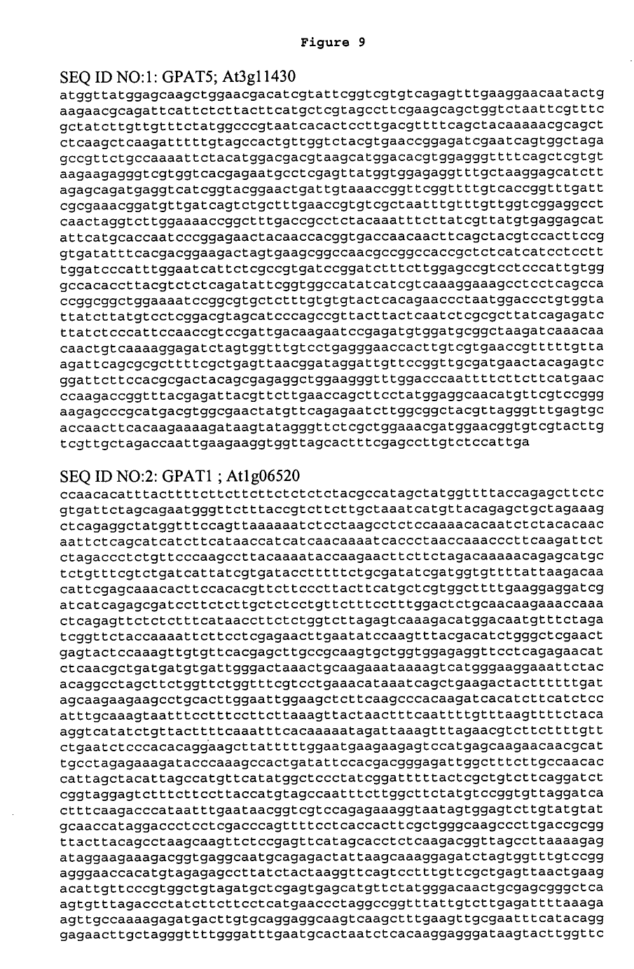

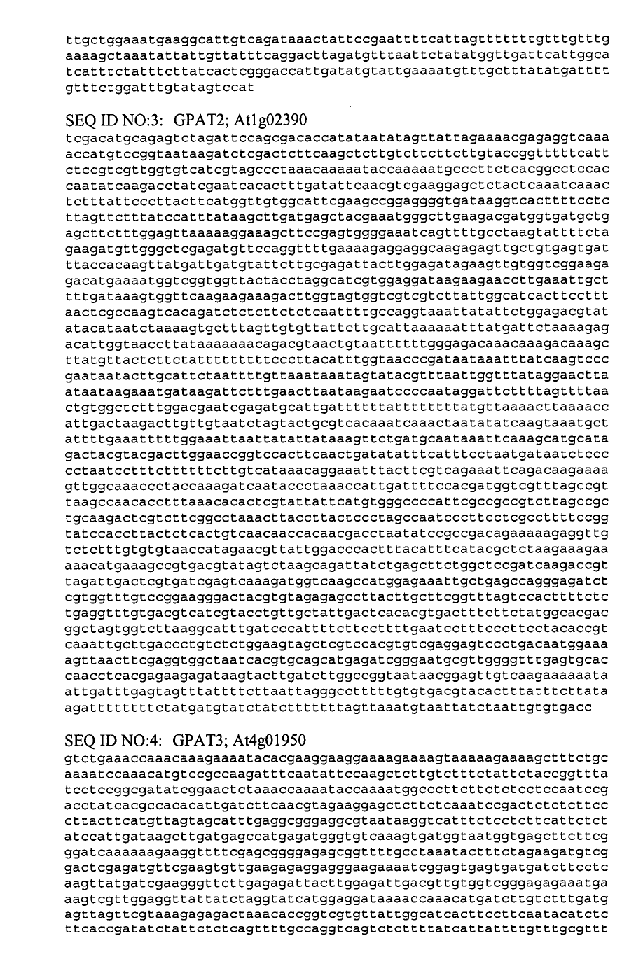

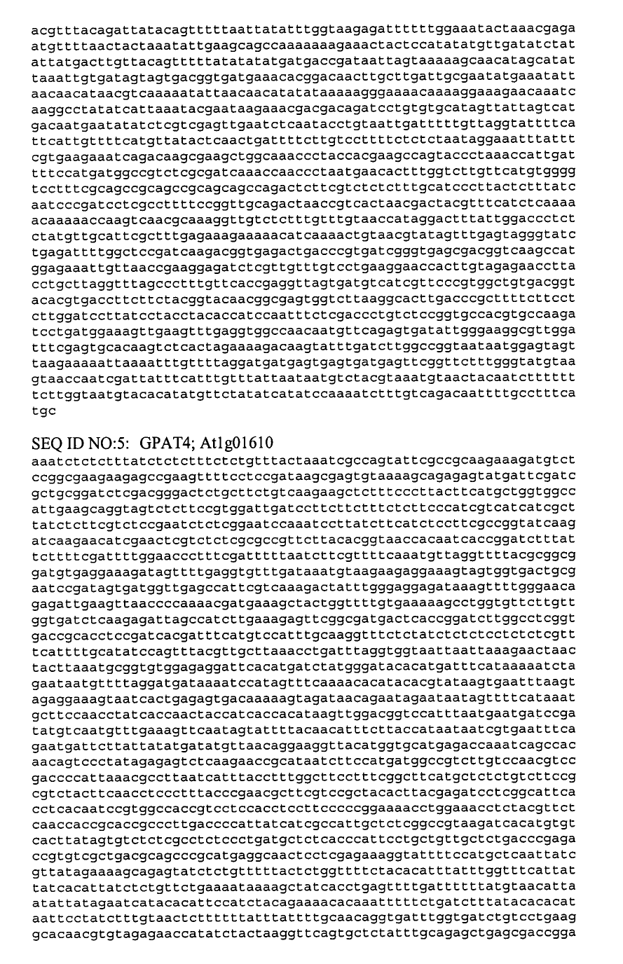

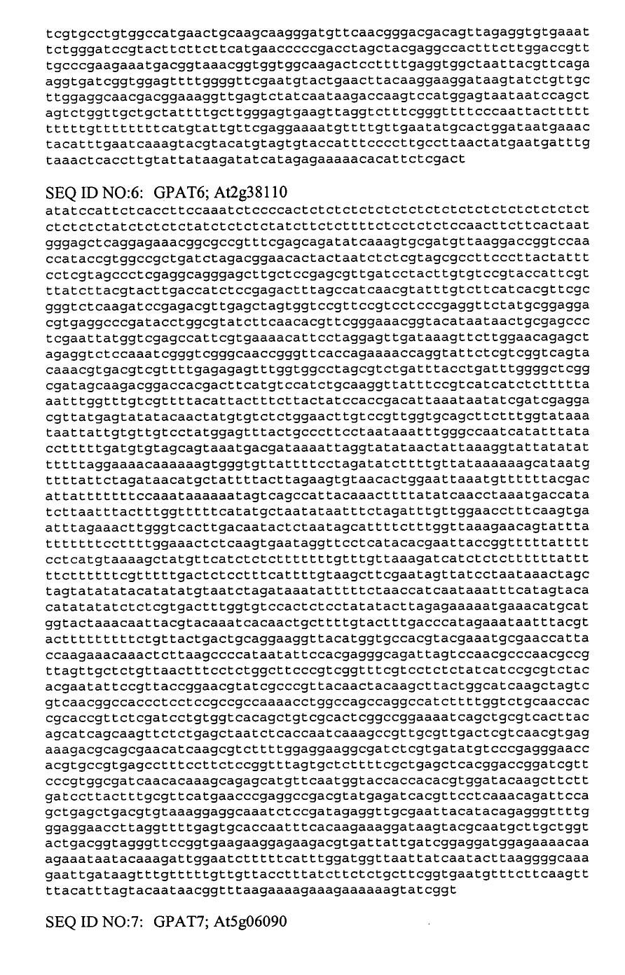

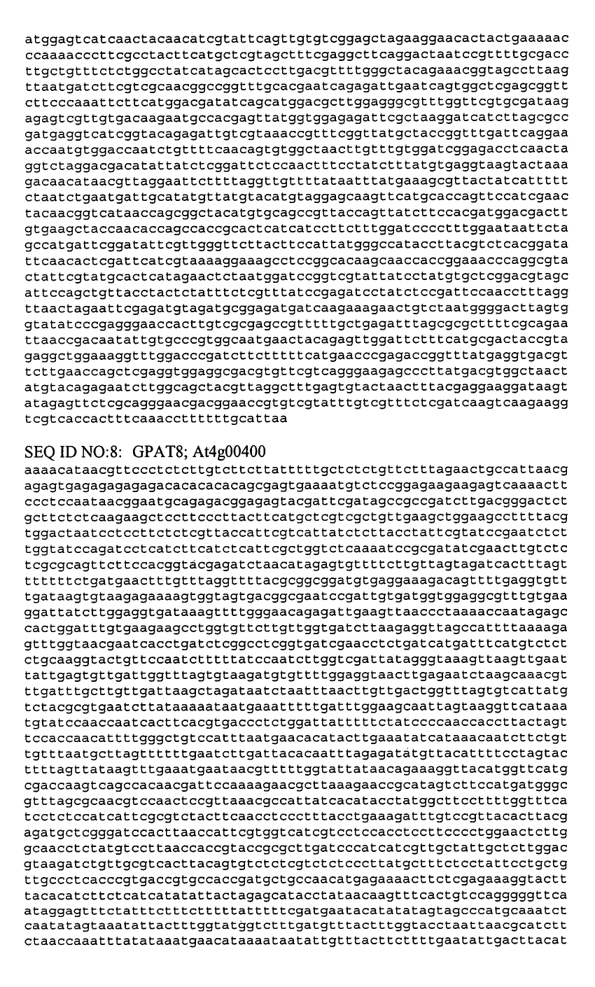

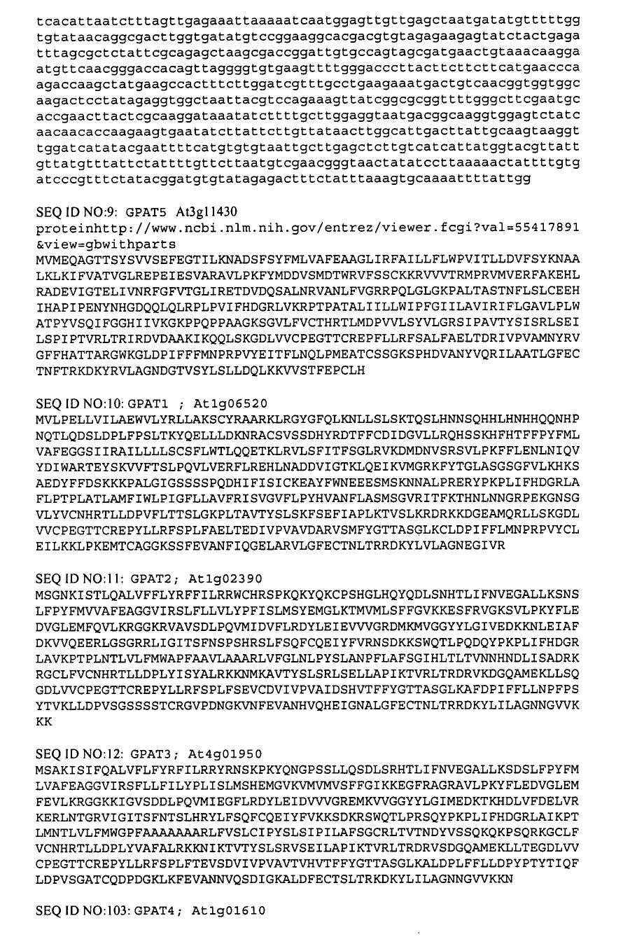

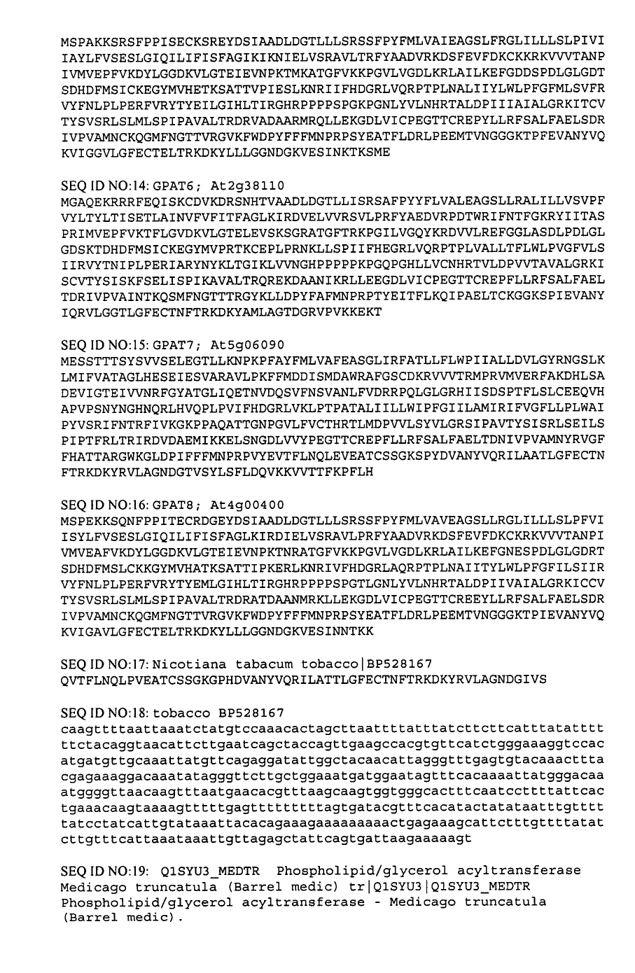

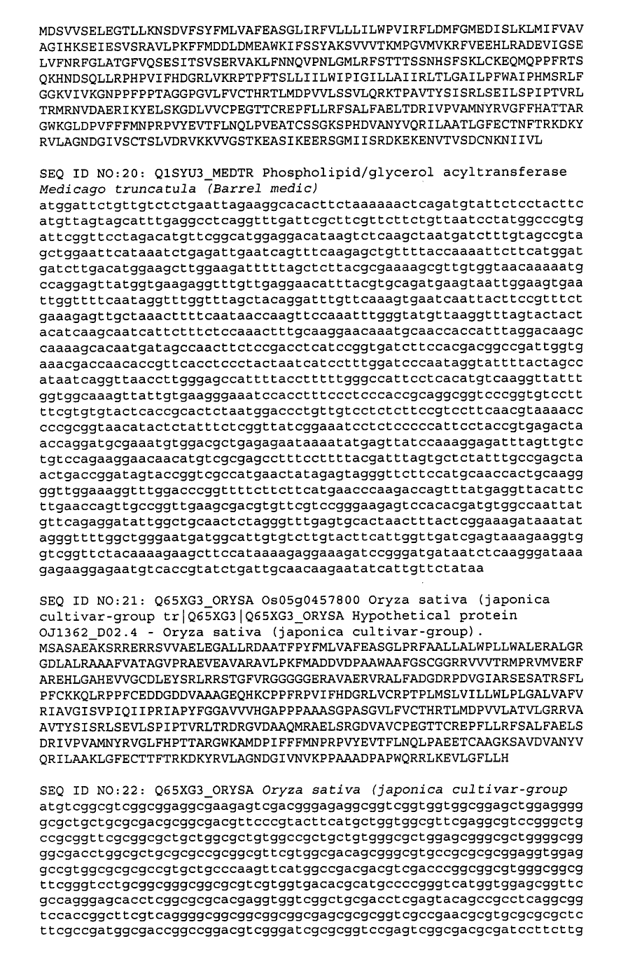

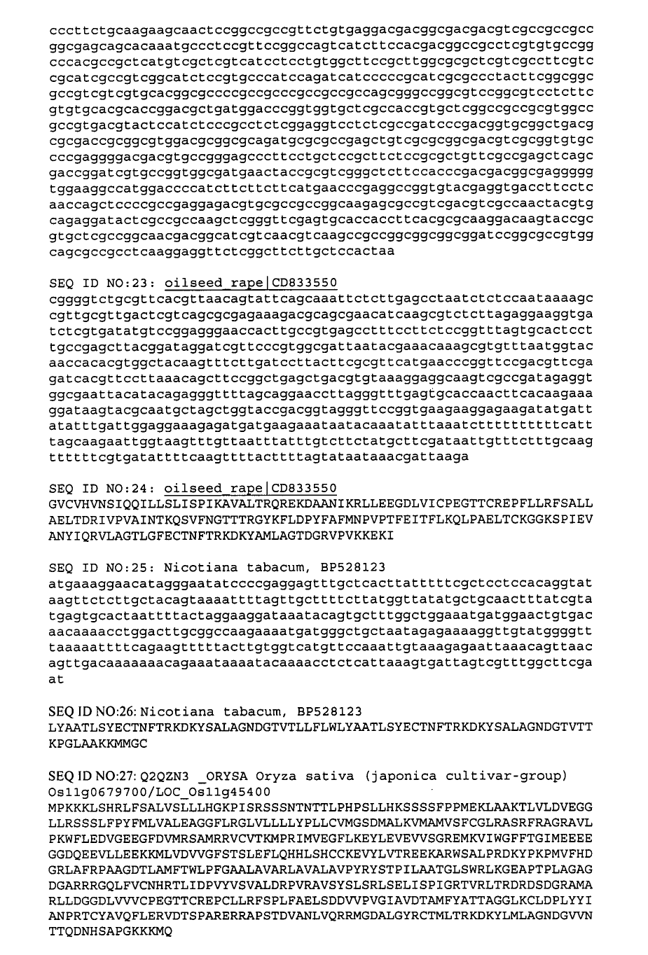

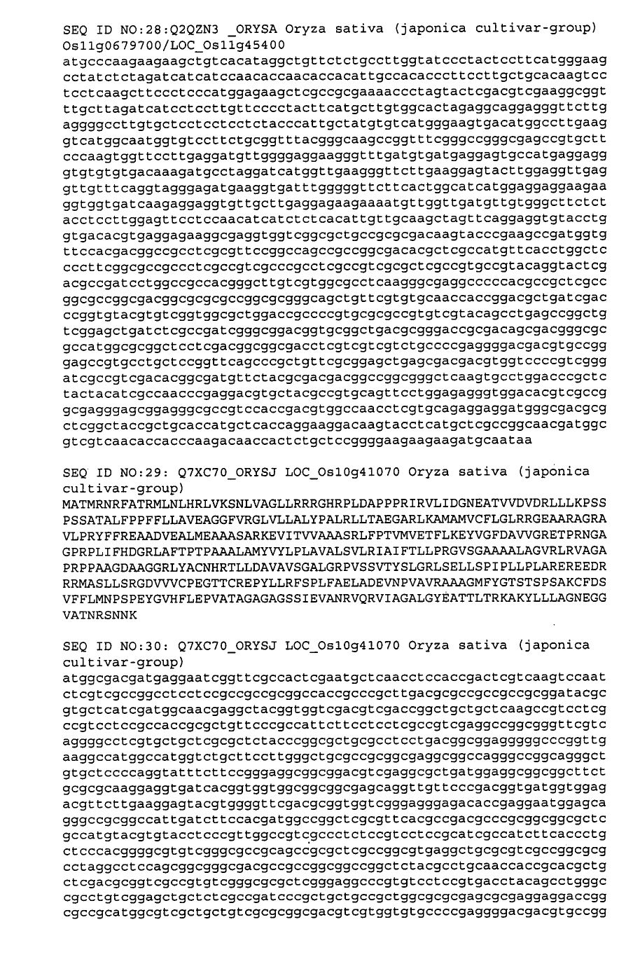

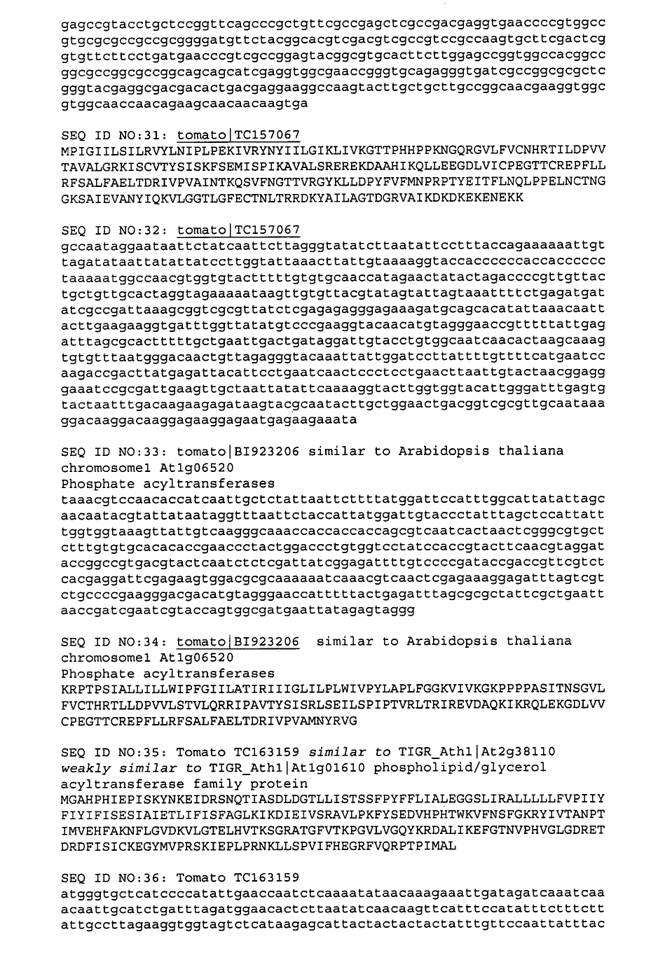

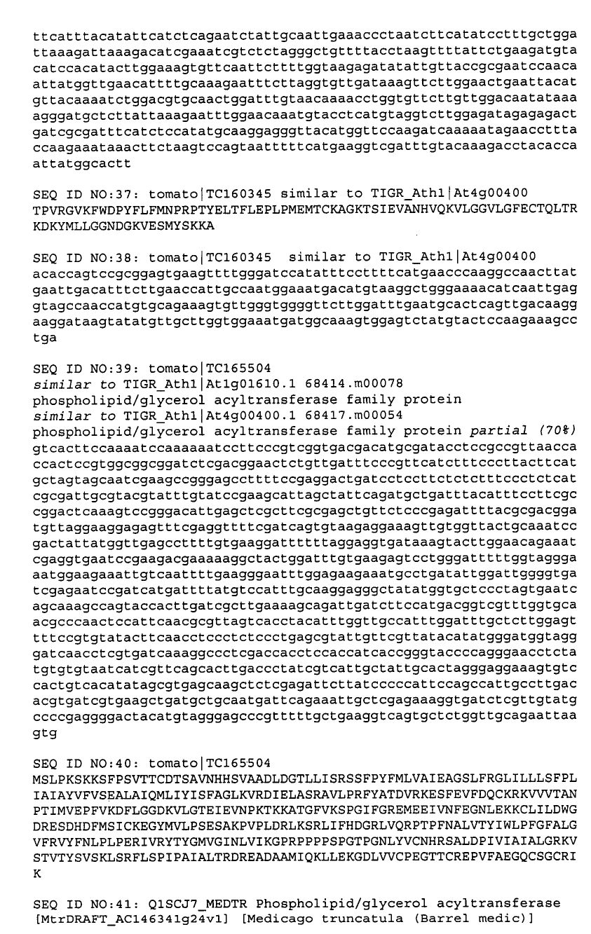

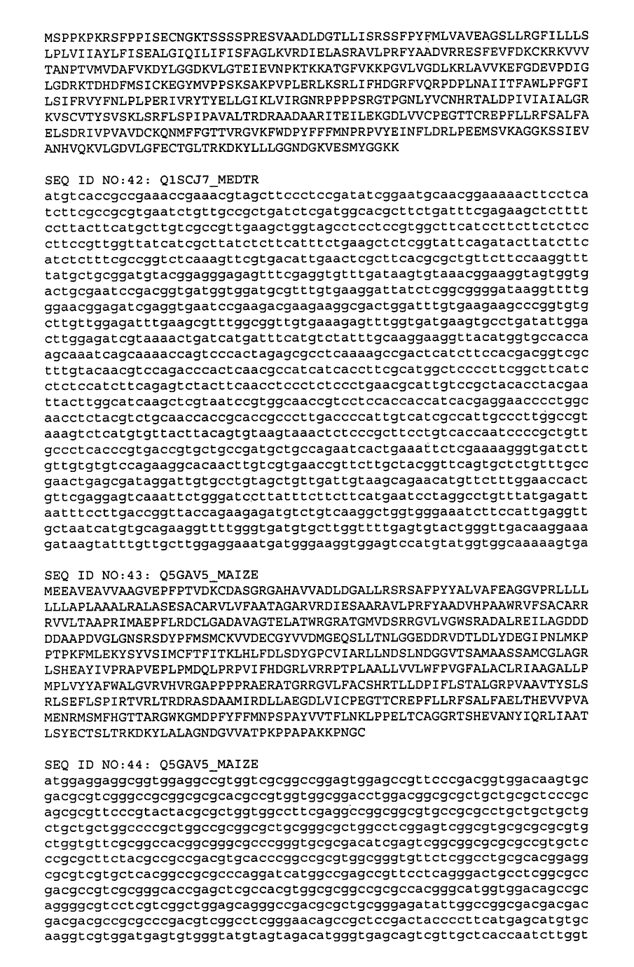

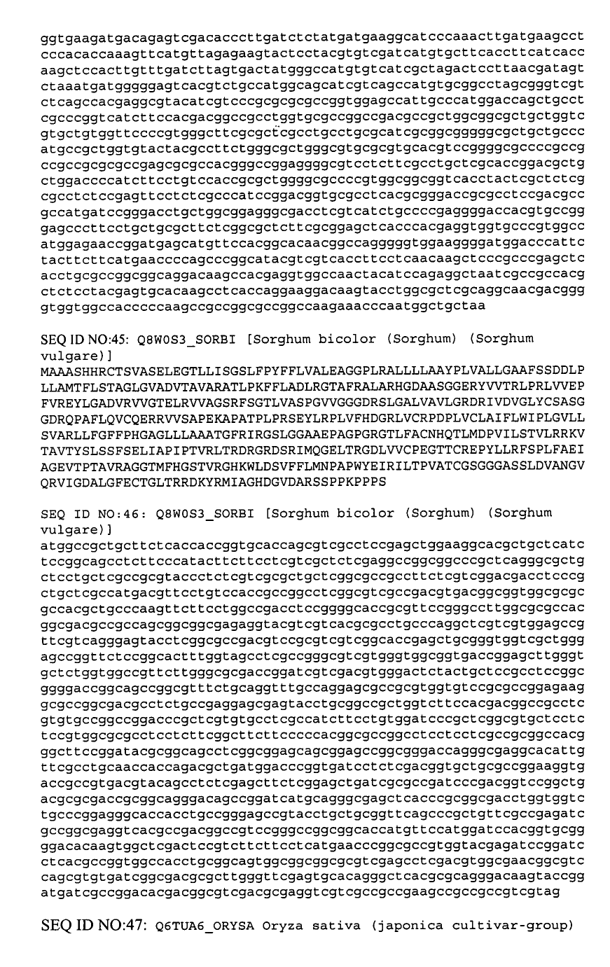

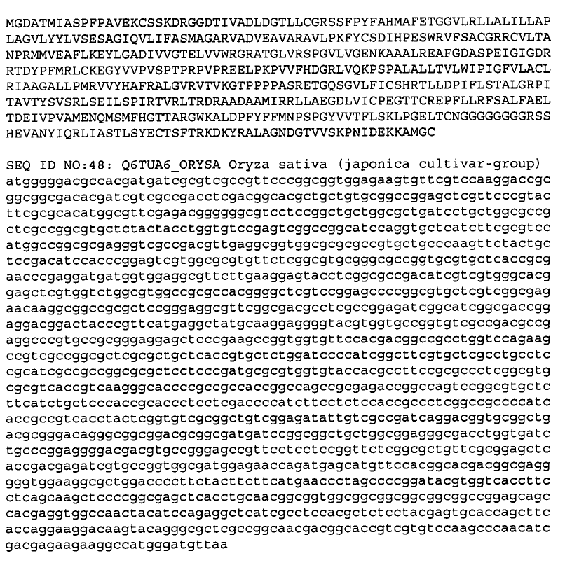

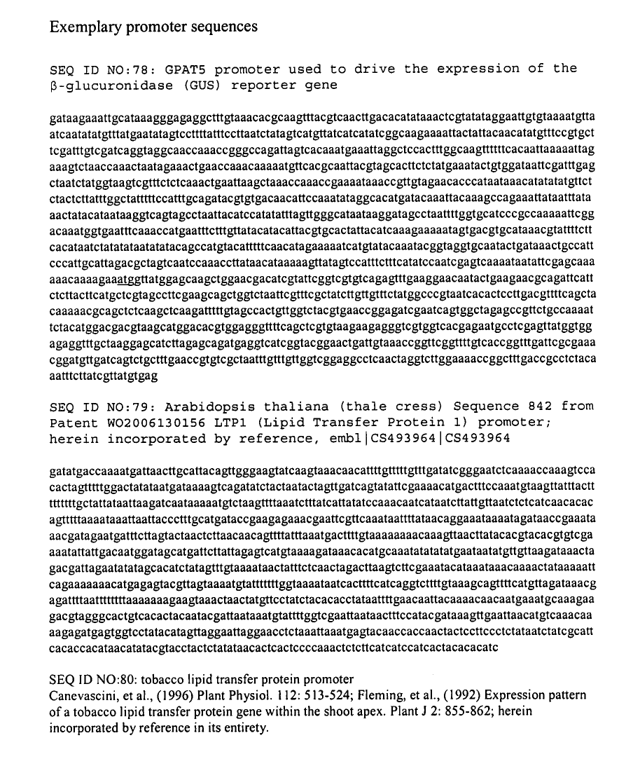

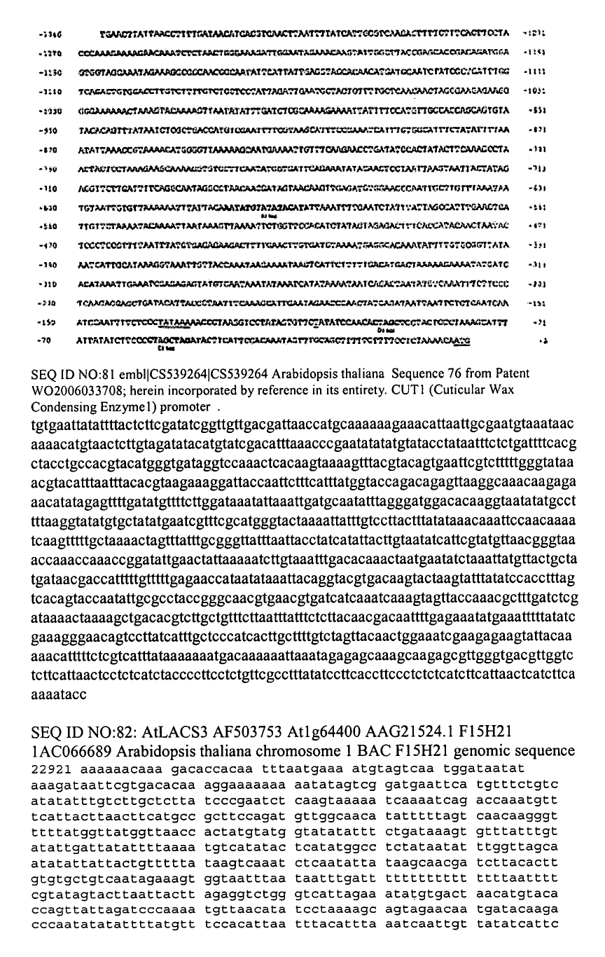

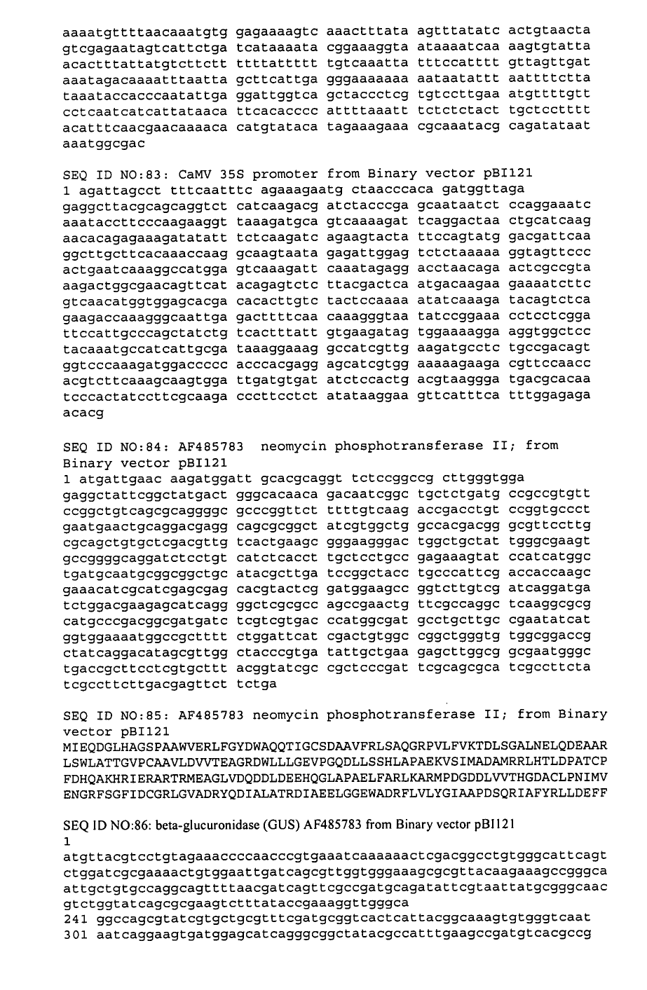

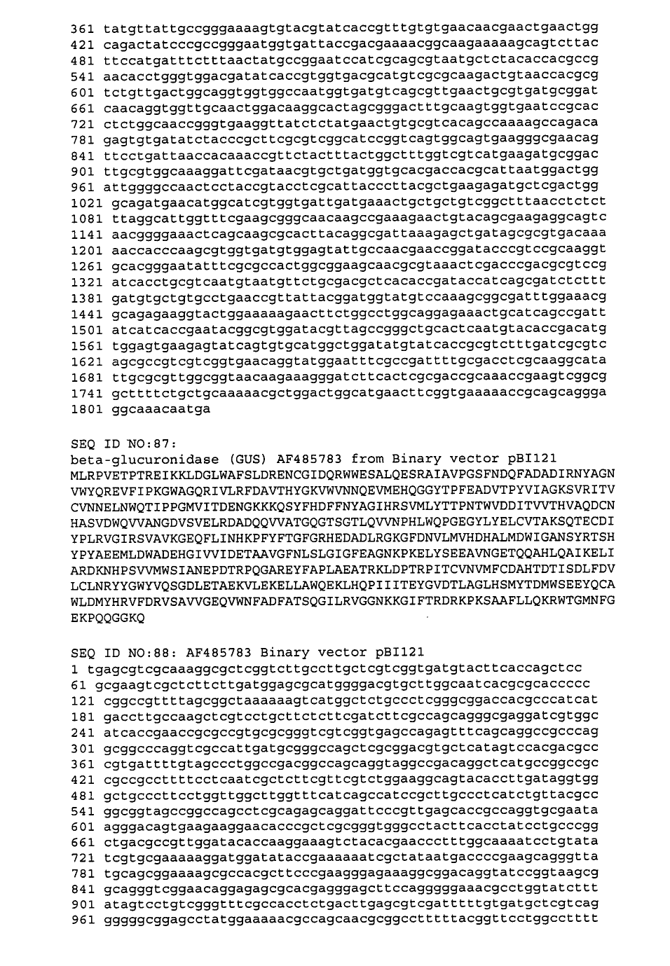

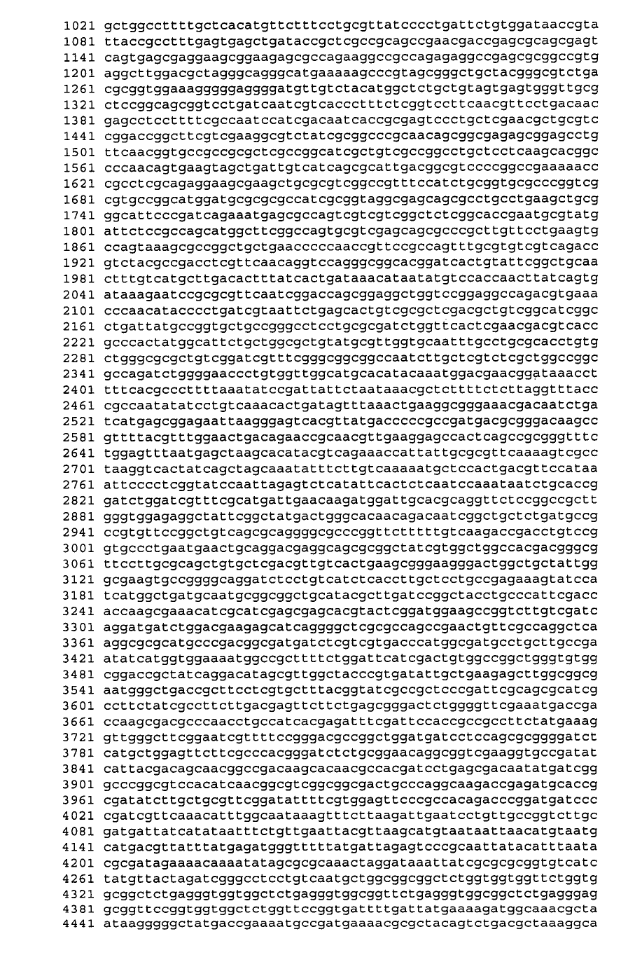

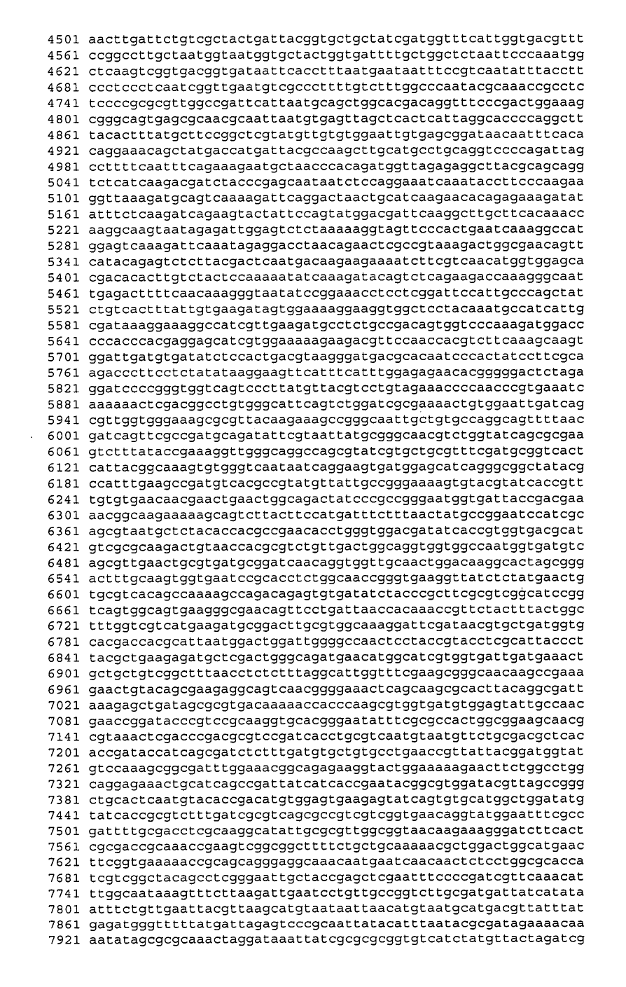

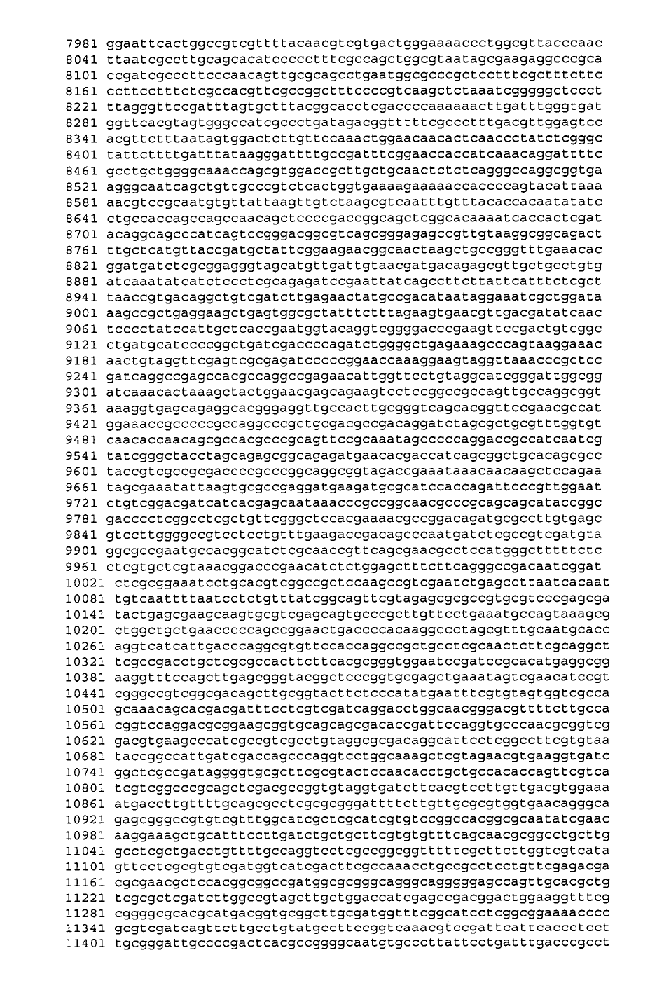

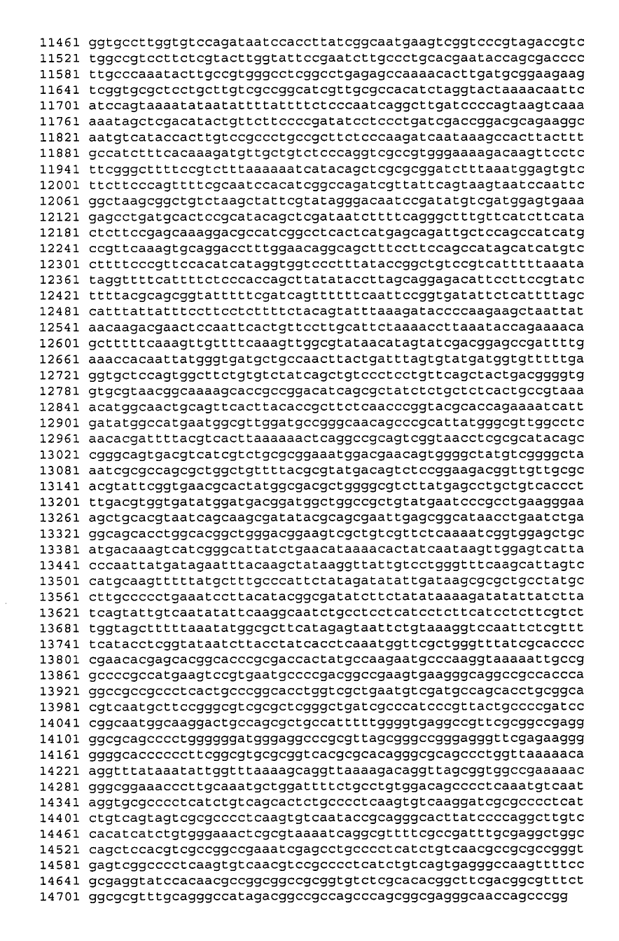

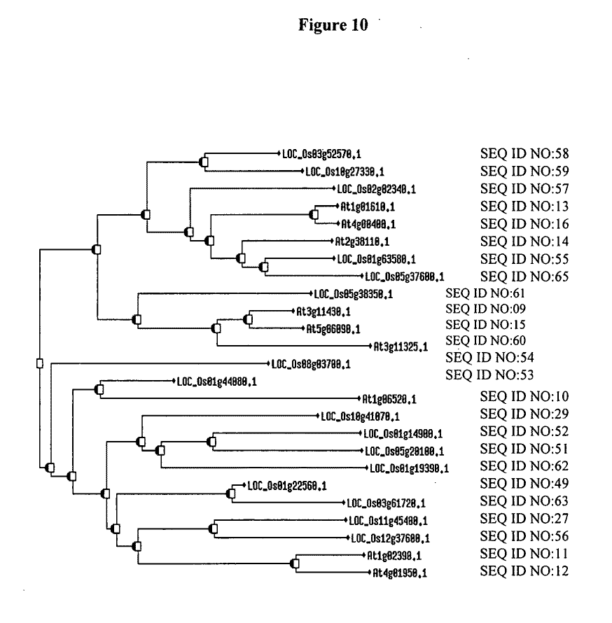

The term “Glycerol Phosphate Acyltransferase” or “GPAT” or “GPAT family acyltransferase” or “GPAT acyltransferase family” or “glycerol-3-phosphate O-acyltransferase” refer to a family of related enzymes designated E.C. 2.3.1.15, such as GPAT1-8 of Arabidopsis thaliana (for example, SEQ ID NOs:1-16), including but not limited to isoforms, homologs and orthologs in Plantae, active fragments, synthetic variants, mutants, and the like, wherein glycerol-3-phosphate O-acyltransferase further includes the term α-glycerophosphate acyltransferase” or “3-glycerophosphate acyltransferase” or “ACP:sn-glycerol-3-phosphate acyltransferase” or “glycerol 3-phosphate acyltransferase” or “glycerol phosphate acyltransferase” or “glycerol phosphate transacylase” or “glycerophosphate acyltransferase” or “glycerophosphate transacylase” or “sn-glycerol 3-phosphate acyltransferase” or “sn-glycerol-3-phosphate acyltransferase” or “acyl-CoA:sn-glycerol-3-phosphate 1-O-acyltransferase. Examples of Planta GPAT family members are shown in a dendogram of rice and Arabidopsis thaliana sequences (FIG. 10) with further examples shown in Table 4 and FIG. 9.

The terms “glycerol-3-phosphate-acyltransferase 5” and “GPAT5” refers to a GPAT family acyltransferase for transferring an acyl group from an acyl donor onto an acyl acceptor molecule for synthesizing a lipid molecule. For the purposes of the present inventions, GPAT5 encompasses genes and gene fragments encoding a polypeptide and the polypeptide including but not limited to active fragments, synthetic variants, mutants, etc., (for example, SEQ ID NOs: 1 and 9.

The term “isoform” refers to any one of several forms of the same protein that differs in its amino acid sequence; produced by different genes or alternative splicing of mRNA.

The term “antisense” when used in reference to DNA refers to a sequence that is complementary to a sense strand of a DNA duplex. A “sense strand” of a DNA duplex refers to a strand in a DNA duplex that is transcribed by a cell in its natural state into a “sense mRNA.” Thus an “antisense” sequence is a sequence having the same sequence as the non-coding strand in a DNA duplex. The term “antisense RNA” refers to a RNA transcript that is complementary to the whole or part of a target primary transcript or mRNA and that blocks the expression of a target gene by interfering with the processing, transport and/or translation of its primary transcript or mRNA. The complementarity of an antisense RNA may be with any part of the specific gene transcript, i.e., at the 5′ non-coding sequence, 3′ non-coding sequence, introns, or the coding sequence. In addition, as used herein, antisense RNA may contain regions of ribozyme sequences that increase the efficacy of antisense RNA to block gene expression. “Ribozyme” refers to a catalytic RNA and includes sequence-specific endoribonucleases. “Antisense inhibition” refers to the production of antisense RNA transcripts capable of preventing the expression of the target protein.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “target RNA molecule” refers to an RNA molecule to which at least one strand of the short double-stranded region of an siRNA is homologous or complementary. Typically, when such homology or complementary is about 100%, the siRNA is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of siRNA. Such targets include unprocessed mRNA, ribosomal RNA, and viral RNA genomes.

The term “ds siRNA” refers to a siRNA molecule that comprises two separate unlinked strands of RNA which form a duplex structure, such that the siRNA molecule comprises two RNA polynucleotides.

The term “hairpin siRNA” refers to a siRNA molecule that comprises at least one duplex region where the strands of the duplex are connected or contiguous at one or both ends, such that the siRNA molecule comprises a single RNA polynucleotide. The antisense sequence, or sequence which is complementary to a target RNA, is a part of the at least one double stranded region.

The term “full hairpin siRNA” refers to a hairpin siRNA that comprises a duplex or double stranded region of about 18-25 base pairs long, where the two strands are joined at one end by a linking sequence, or loop. At least one strand of the duplex region is an antisense strand, and either strand of the duplex region may be the antisense strand. The region linking the strands of the duplex, also referred to as a loop, comprises at least three nucleotides. The sequence of the loop may also a part of the antisense strand of the duplex region, and thus is itself complementary to a target RNA molecule.

The term “partial hairpin siRNA” refers to a hairpin siRNA which comprises an antisense sequence (or a region or strand complementary to a target RNA) of about 18-25 bases long, and which forms less than a full hairpin structure with the antisense sequence. In some embodiments, the antisense sequence itself forms a duplex structure of some or most of the antisense sequence. In other embodiments, the siRNA comprises at least one additional contiguous sequence or region, where at least part of the additional sequence(s) is complementary to part of the antisense sequence.

The term “mismatch” when used in reference to siRNAs refers to the presence of a base in one strand of a duplex region of which at least one strand of an siRNA is a member, where the mismatched base does not pair with the corresponding base in the complementary strand, where pairing is determined by the general base-pairing rules. The term “mismatch” also refers to the presence of at least one additional base in one strand of a duplex region of which at least one strand of an siRNA is a member, where the mismatched base does not pair with any base in the complementary strand, or to a deletion of at least one base in one strand of a duplex region which results in at least one base of the complementary strand being without a base pair. A mismatch may be present in either the sense strand, or antisense strand, or both strands, of an siRNA. If more than one mismatch is present in a duplex region, the mismatches may be immediately adjacent to each other, or they may be separated by from one to more than one nucleotide. Thus, in some embodiments, a mismatch is the presence of a base in the antisense strand of an siRNA which does not pair with the corresponding base in the complementary strand of the target siRNA. In other embodiments, a mismatch is the presence of a base in the sense strand, when present, which does not pair with the corresponding base in the antisense strand of the siRNA. In yet other embodiments, a mismatch is the presence of a base in the antisense strand that does not pair with the corresponding base in the same antisense strand in a foldback hairpin siRNA.

The terms “nucleotide” and “base” are used interchangeably when used in reference to a nucleic acid sequence.

The term “strand selectivity” refers to the presence of at least one mismatch in either an antisense or a sense strand of a siRNA molecule. The presence of at least one mismatch in an antisense strand results in decreased inhibition of target gene expression. The term “cellular destination signal” is a portion of an RNA molecule that directs the transport of an RNA molecule out of the nucleus, or that directs the retention of an RNA molecule in the nucleus; such signals may also direct an RNA molecule to a particular subcellular location. Such a signal may be an encoded signal, or it might be added post-transciptionally. The term “enhancing the function” when used in reference to an siRNA molecule means that the effectiveness of an siRNA molecule in silencing gene expression is increased. Such enhancements include but are not limited to increased rates of formation of an siRNA molecule, decreased susceptibility to degradation, and increased transport throughout the cell. An increased rate of formation might result from a transcript which possesses sequences that enhance folding or the formation of a duplex strand.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

### DESCRIPTION OF THE INVENTION

The present invention relates to compositions comprising acyltransferase nucleic acid molecules for altering lipids on the surface of plants, and related methods. In particular, the present invention provides compositions and methods for increasing the amount of free fatty acids, acylglycerols, and other lipids on the surface of a plant. In a preferred embodiment, the present invention relates to increasing activity of a GPAT acyltransferase for altering lipid on the plant surface, for use in solving a variety of problems, including but not limited to increasing environmental stress tolerance in plants, increasing plant resistance to biotic stress, including fungi, bacteria and insects, increasing storage tolerance of plant parts, such as tubers, and providing novel plant oils for a range of commercial products.

Eight members of the GPAT family have been identified in Arabidopsis of which certain GPATs, such as GPAT4 and GPAT 8, were shown to be upregulated in stem epidermis (see, for example, Suh et al. (2005), Plant Physiol 139: 1649-1665; herein incorporated by reference in its entirety) and expressed in leaves, as shown herein. Therefore, the inventors contemplated that GPATs are involved in formation of leaf and stem cutin and other polyester production thus providing variant chain length specificity (e.g. using acceptor molecules C16-C18). In experiments conducted during the course of the present invention, Arabidopsis double knock-out plants were created demonstrating that loss of GPAT4/GPAT8 related to alterations in leaf cuticle permeability, which is consistent with GPAT4 and/or GPAT8 providing a role in leaf surface lipid synthesis. Other plants, such as tobacco plants, are known to secrete branched chain fatty acid derivatives at their surface. Given the variety of lipid hydrocarbon chain derivatives present at the surface of various organs in various plant species, the overexpression of homologs, orthologs and even different isoforms of plant GPATs from various species is contemplated to provide enzyme activity on a wide range of straight and branched hydrocarbon chains and derivatives with lengths ranging from C2 to C60. Thus GPAT5 overexpressing plants described herein, demonstrate numerous exemplary characteristics of a model plant for providing useful lipid products.

In further embodiments, the present invention relates to using an Arabidopsis thaliana GPAT family acyltransferase for altering lipid compounds on the surface of a plant. In certain embodiments, the present invention relates to using an Arabidopsis thaliana GPAT5 acyltransferase (GPAT5) for altering wax compounds present on the surface of a plant. In certain embodiments, the present invention relates to using an Arabidopsis thaliana GPAT4 acyltransferase (GPAT4) for altering wax compounds present on the surface of a plant. In certain embodiments, the present invention relates to using an Arabidopsis thaliana GPAT7 acyltransferase (GPAT7) for altering wax compounds present on the surface of a plant. In certain embodiments, the present invention relates to using an Arabidopsis thaliana GPAT8 acyltransferase (GPAT8) for altering wax compounds present on the surface of a plant.

In a preferred embodiment, the present invention relates to decreasing Arabidopis thaliana GPAT family acyltransferase activity for altering lipid production on the plant surface, for providing novel surface lipids and structures for use in solving a variety of problems, including but not limited to altering environmental stress tolerance in plants, increasing storage tolerance of plant parts, such as tubers, increasing economic value of plants and plant parts, such as cotton fibers, and providing novel plant oils for a range of commercial products. In a further embodiment, the present invention relates to silencing an Arabidopsis thaliana GPAT family acyltransferase 5 (GPAT5) for altering wax compounds present on the surface of a plant. In certain embodiments, the present invention relates to silencing an Arabidopsis thaliana GPAT4 acyltransferase (GPAT4) for altering wax compounds present on the surface of a plant. In certain embodiments, the present invention relates to silencing an Arabidopsis thaliana GPAT7 acyltransferase (GPAT7) for altering wax compounds present on the surface of a plant. In certain embodiments, the present invention relates to silencing an Arabidopsis thaliana GPAT8 acyltransferase (GPAT8) for altering wax compounds present on the surface of a plant. In certain embodiments, the present invention relates to silencing an Arabidopsis thaliana GPAT7 and GPAT8 acyltransferase (GPAT7/8) for altering wax compounds present on the surface of a plant.

This invention provides compositions and methods for providing increased amounts, novel free fatty acids, such as acylglycerols, and other lipids on the surface of plants. As a result, the present inventions provide compositions and methods for providing transgenic plants that produce “designer lipids” and novel hydrocarbon derivatives on their extracellular surfaces. Thus transgenic plant parts provide high (economical feasible) amounts of specific types of lipid compounds that were merely collected (harvested) from the surface or easily extracted from surface plant tissues, such as from the surface of leaves, stems, silques, roots, and seeds. Further, the compositions and methods of the present inventions provide transgenic plants and plant parts with increased wax loads, such as changed (altered) wax composition, altered subrin, and the like, for commercial and pharmaceutical applications.

Wax loads vary between species of plants and under stressed environmental conditions. Sorghum and cotton (Gossypium hirsutum) leaf cuticular wax loads were in the range of 100 to 300 μg cm2 (sorghum: Premachandra et al., 1994, J Exp Bot 43:1569-1576; Bondada et al., 1996 Environ Exp Bot 36(1):61-69; all of which are herein incorporated by reference), while averages of less than 25 μg cm2 were reported for rice (Oryza sativa), oat (Avena sativa), willow (Salix spp.), hybrid poplar (Populus spp.), and Arabidopsis (Arabidopsis thaliana; Bengtson et al., 1978, Physiol Plant 44:319-324; O'Toole et al., 1979, Physiol Plant 47:239-244; Hietala et al., 1995, Phytochemistry 40:23-27; Jenks et al., 1995, Plant Physiol 108:369-377; Cameron et al., 2002, Phytochemistry 60:715-725; all of which are herein incorporated by reference). Increased wax loads were reported for stressed plants, such as where a mean wax load on well-watered, chamber-grown tree tobacco leaves (Nicotiana glauca L. Graham) was 10 μg c m² then increased 1.5- to 2.5-fold (26 μm²), on leaves periodically dried three times, however the composition of the wax was not altered (Cameron, et al., 2006, Plant Physiol. 140(1): 176-183; herein incorporated by reference). In contrast, increased and altered wax production was observed epicuticular waxes (EW) of leaves, bracts, and bolls of water stressed cotton (Gossypium hirsutum L.). Specifically, well-watered wax levels at 91.71 μg cm², 74.18 μg cm², and 152.58 μg cm² vs. water stress 154.60 μg cm², 108.91 μg cm², and 158.53 μg cm² in the leaf, bract, and boll, respectively, while the number and levels of long-chain, higher molecular weight alkanes in the leaf and bract wax were increased, (Bondada et al., 1996, Environ Exp Bot 36(1):61-69; herein incorporated by reference). However, unlike the morphology of altered waxes of the present inventions, these increased waxes had analogous wax morphology leaf, bract, and boll exterior surface had observed by scanning electron microscopy under both water-stressed and well-watered conditions.

The inventors contemplate that designing specific oil and wax production in transgenic plants will provide numerous advantages for farmers and benefits for consumers. Specifically, instead of producing undifferentiated commodity crops, farmers will be able to deliver genetically modified varieties of crops which produce lipids for specific uses, thus providing dietary benefits and a wider choice of less expensive products for consumers. Further, certain types of plant lipids would provide plant oils with a longer shelf life.

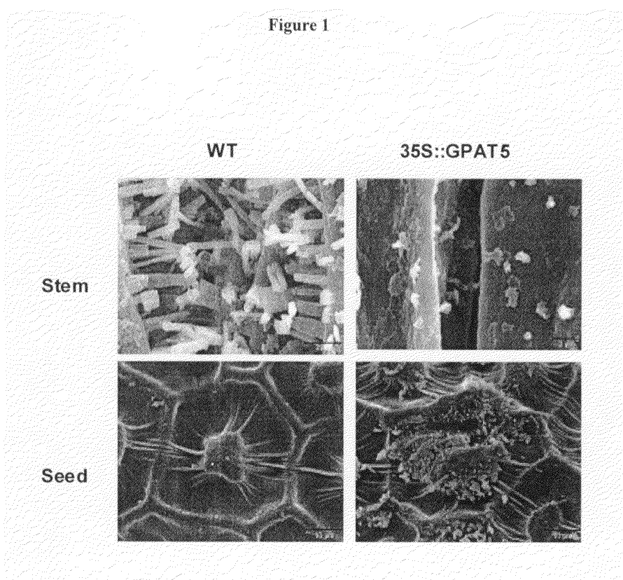

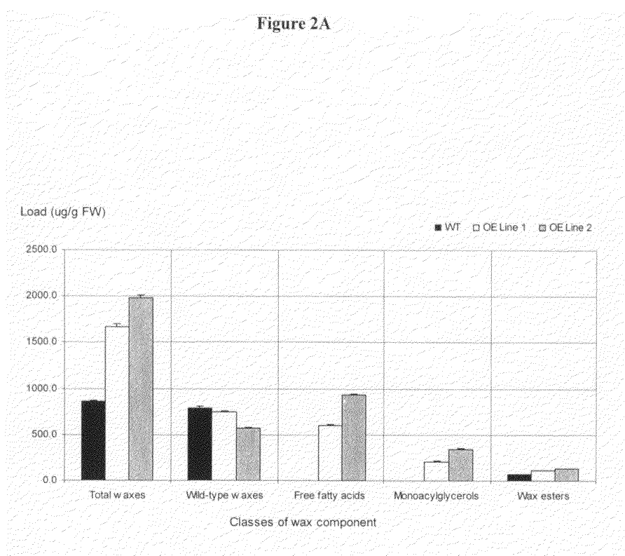

In experiments conducted during the course of the present invention, the epicuticular part of the plant surface waxes (e.g., the outermost layer and thus a primary site of interaction for pathogens), was shown to be modified by the overexpression of a plant GPAT as evidenced by the changes in epicuticular crystals of the GPAT5 overexpressor in Arabidopsis (see, FIG. 1). The discovery that overexpression of GPAT5 in plants leads to increased production of surface waxes with a high proportion of free fatty acids and modified epicuticular crystals, without observable harmful effects to the plant, indicates that the overexpression of various plant GPATs allows the production of plants for providing altered surface lipids for use in designer plant oils. The inventors further contemplate that such increased surface oils should provide increased resistance to various pathogens, including host-specific pathogens. Thus the use of GPAT family acyltransferases in engineering specific types of plant surfaces is further contemplated. An increase in surface waxes may provide disease resistance where increased surface wax layer contributes fungal pathogen resistance (see, for example, Ficke et al. (2004) Phytophatology 94:438-445; herein incorporated by reference in its entirety) and herbivorous insects (see, for example, Eigenbrode and Espelie (1995) Annual Rev Entomol 40:171-194; Sheperd et al. (1999), Phytochemistry 52:1239-1254; all of which are herein incorporated by reference). Further, altering surface wax would provide a physical deterrent for insect feeding and reproduction. Specifically, certain structure and compositions of epicuticular waxes in specific plant species- or cultivar-specific wax morphology provides a physical and/or chemical cue for proper orientation and choice of sites for feeding and ovideposition of insect specialists (Adati and Matsuda (1993), Appl. Entomol. Zool. 28:319-324; Muller and Hilker (2001), J Chem. Ecol. 5:985-94; all of which are herein incorporated by reference) as well as for induction of germination and appressorium formation of host-specific biotrophic fungi (see, for example, Podila et al. (1993) Plant Physiol 103:267-272; Hedge and Kolattukudy (1997) Physiol. Mol. Plant. Pathol 51:75-84; Gniwotta et al. (2005) Plant Physiol 139:519-530; all of which are herein incorporated by reference).

The inventors further contemplate the use of altered (designer) plant lipids for use as antimicrobials, such as in pesticides or in soaps. General microbicidal activity of free fatty acids and their derivatives is well known and there is evidence of significant activity against pathogens and insects attacking plants (see, for example, Ahmed et al. (1985), J. Am. Oil Chem. Soc 62: 1578-1580; Liu et al. (1996), J. Econ. Entomol. 89:1233-1239; Puterka et al. (2003), J. Econ. Entomol. 96:636-644; all of which are herein incorporated by reference). Fatty acid salts and derivatives (e.g. potassium salts of fatty acids) are used in agriculture as non-toxic to the user and environmentally safe active ingredients of commercially available pesticides (sold for example under trademarks SAFER INSECTICIDAL SOAP, De-Moss). Therefore the inventors contemplate the use of monoacylglycerols and derivatives, such as those produced by the transgenic plants of the present invention, as antimicrobials due to reported antimicrobial activity of other monoacylglycerols and derivatives, (see, for example, Kabara and Vrable (1977) Lipids 12:753-759; Wang and Johnson (1992) Appl Environ Microbiol. 58:624-9; all of which are herein incorporated by reference) while U.S. Pat. No. 4,002,775; herein incorporated by reference, claims a monoacylglycerol as microbicidal food additive.

The inventors discovered that in contrast to previous concerns that increasing lipid production in plants would inhibit plant growth or kill a plant, GPAT5 overexpressing plants that produced higher amounts of free fatty acids at their surface appeared healthy with few alterations in development or growth as compared to wild-type plants. Further, none of the phytotoxic effects were observed that were often reported for fatty acid and their salts applied exogenously at high doses (see, for example, U.S. Pat. No. 5,246,716 and No. 3,931,413, all of which are herein incorporated by reference). Further, the GPAT5 overexpressing plants demonstrated a large accumulation of C22 and C24 fatty acids and derivatives.

The inventors further discovered that decreasing certain types of surface lipids did not appear to severely reduce plant viability. Specifically, the inventors discovered that suberin synthesis was significantly reduced in silenced (knock-out) GPAT5 mutants required for suberin synthesis (Beisson et al, (2007) The Plant Cell 19:351-368; herein incorporated by reference). Thus the inventors contemplate that in some embodiments, alterations in surface wax compositions would decrease or increase suberin for altering cotton fiber properties. For example, certain types of cotton fibers have high levels of suberin and suberin waxes. These waxes can reduce the dye absorption properties of the fibers. Specific embodiments are contemplated for reducing suberin in cotton fibers for increasing dye absorption properties while reducing processing costs.

The inventors further contemplate the use of altered extracellular plant lipids for use in cellulosic biofuels. In particular, during conversion of crop residues, such as perennial grasses, and other plant material to biofuel, the carbohydrate from the plant must be digested, usually by enzymes. Grass leaves contain both cutin on the surface and suberin surrounding the bundle sheaths. These lipid materials restrict access of enzymes to the plant cell walls thus increasing the time and costs of processing such biomass. Therefore, the inventors contemplate that increased access of enzymes to cell-wall carbohydrates of grasses would be achieved by reducing the suberin and/or cutin levels by using antisense expression vectors for reducing GPAT, in particular GPAT5.

In some embodiments, alterations in surface wax compositions are contemplated for control of abscission. The term, “abscission” refers to the process by which a plant intentionally drops one or more of its parts, such as a leaf, fruit, flower or seed. Suberin is deposited as a seal and to control the time of separation of a plant part from a plant. Thus the inventors contemplate the use of promoters, such as inducible, developmental, to use control of GPAT expression for inhibited or enhanced abscission of a specific plant part.

In some embodiments, alterations in surface wax compositions for providing new and/or improved properties of resistance to biotic or abiotic stresses are provided. The inventorswidely documented that in various plant parts (leaves, stems, fruits, etc.) cuticular waxes (in particular epicuticular crystals) can influence significantly phytopathogen attacks by acting for example on colonization by epiphytic microorganisms, germination of phytopathogenic fungal spores, growth pattern of biotrophic fungi hyphae, host selection by herbivorous insects (see, for example, Kolattukudy et al. (1995) Proc Natl Acad Sci USA. 92:4080-4087; Muller and Riederer (2005), J Chem. Ecol. 31:2621-2651; all of which are herein incorporated by reference).

Previously, several types of transgenic plants were created in order to induce and/or shift fatty acid production, particularly in seeds, for providing designer oils. However, in contrast to the plants of the present invention, these other transgenic plants genetically modified for altering fatty acid synthesis have more limited uses. The first genetically modified vegetable oil, from Brassica napus (Canola), has a high proportion of lauric acid (high amounts of laurate (12:0) and myristate (14:0)) desirable for many food and non-food applications, such as, providing a critical ingredient in soaps, shampoos, detergents, and used in confectionery, icings, crackers and coffee whiteners. This oil was produced by a commercial variety of a genetically modified canola plant engineered for expressing a gene from the California bay laurel tree, a relative of oilseed rape, which codes for an enzyme involved in the synthesis of lauric acid, using a thioesterase encoding gene from the California bay laurel (Umbellularia californica). Other plants, Glycine max L. (Soybean) events G94-1, G94-19, and G1168 (DuPont Canada Agricultural Products), demonstrate high oleic acid soybean produced by inserting a second copy of the fatty acid desaturase (GmFad2-1) encoding gene from soybean, which resulted in “silencing” of the endogenous host gene and soybean event OT96-15 (Agriculture & Agri-Food Canada) demonstrating a low linolenic acid soybean produced through traditional cross-breeding to incorporate the novel trait from a naturally occurring fan1 gene mutant that was selected for low linolenic acid. However, when genes that lead to hydroxy fatty acid, or Lauric Acid (n-Dodecanoic Acid), or other unusual fatty acids are expressed with constitutive promoters in leaf, there is usually undetectable production, whereas in the same transgenic plants these fatty acids are found in the seeds. Thus these altered lipid traits are primarily expressed in seeds and the lack of production in leaves has been shown to be due to the rapid breakdown of the novel fatty acid (see, for example, Eccleston, et al., (1996) Planta 198:46-53; Eccleston and Ohlrogge (1998) Plant Cell. April; 10(4):613-621; all of which are herein incorporated by reference in its entirety). The present invention avoids this “futile cycle” of synthesis and breakdown by causing the export of the desired lipid structure to the plant surface. Furthermore, the invention has the important advantage of removing potentially detrimental fatty acyl structures from inside the cell where they may interfere with the growth, metabolism or physiology of the plant (see, for example, Millar et al (1998) Plant Cell. 10:1889-902; herein incorporated by reference in its entirety).

Thus further advantages of compositions and methods of the present invention include providing novel lipid compositions, such as external lipids comprising free fatty acids, acylglycerols and other hydrocarbon derivatives; lipids that are easily recovered from the surface of abundant plant parts, such as seeds, in contrast to using previous time-consuming and expensive procedures; methods of the present inventions are contemplated for providing an economic value for those plant parts that currently have no or low economical value, such as leaves and stems; external lipids can also be recovered from a mixture of plant surface lipophilic compounds where they represent a higher proportion than in the mixture of oil and other lipophilic components obtained from seeds; and further provide unique surface wax compositions, including higher amounts of free fatty acids and acyl glycerols. These products would be useful for a market of specialty fatty acids (lubricants, polymers, etc.) as well as markets interested in producing hydrocarbon derivatives and/or obtaining plants with specific types of cuticles.

Further, several types of plant acyltransferases have been used to alter or described as altering lipid production in plants, including, fatty alcohol acyltransferase (wax synthase) in U.S. Patent Application No. 20030228668; cholesterol acyltransferase in U.S. Patent Application No. 20020170091; sterol acyltransferase in U.S. Patent Application No. 20050102716; Arabidopsis GPATs for altering lipids in plants in Intl. Patent Publication No. WO03025165; and diacylglycerol acyltransferase from Brassica napus in U.S. Pat. No. 6,995,301, all of which are herein incorporated by reference. Additionally, WIN1, an Arabidopsis thaliana ethylene response factor-type transcription factor, that can activate wax deposition in overexpressing plants, induced leaf epidermal wax accumulation up to 4.5-fold higher in these plants than in control plants. Further a significant increase of wax was found in stems. However, approximately 50% of the additional wax required complete lipid extractions for collection, suggesting that the remaining wax was not extracellular, unlike the waxes of the present inventions (see, for example, Broun et al. (2004), Proc Natl Acad Sci USA. 101:4706-11; herein incorporated by reference in its entirety). Like WIN1, WXP1 (wax production 1), a homologue of Arabidopsis WIN1, overexpressed under the control of the CaMV35S promoter led to a significant increase in cuticular wax loading on leaves and conferred drought tolerance of transgenic alfalfa (Medicago sativa); see, for example, Zhang et al. (2005) Plant J. 42:689-707 and U.S. Patent Application No. 20060107349, all of which are herein incorporated by reference. However, these publications do not describe transgenic plants comprising a GAPT gene demonstrating the type of altered lipid production on the surface of plants as demonstrated herein. Additionally, lipids were altered in plants using fatty acid synthetases (elongases), such as fatty acid β-keto acyl synthases for producing very long chain fatty acids (VLCFA) in U.S. Patent Application No. 20060107350 and cyclopropane fatty acid synthase genes in U.S. Patent Application No. 20060053512; however these genes are not acyltransferases.

Arabidopsis GPATs were isolated by structural similarity to yeast glycerol-phosphate acyltransferases and many of the members of the GPAT family, including GPAT5, were shown to catalyze in vitro the transfer of acyl chains from acyl-CoA to glycerol-3-phosphate to form LPA (Lysophosphatidic acid) (see, for example, Zheng et al. 2003 Plant Cell 15:1872-87; herein incorporated by reference). In some embodiments, in vivo glycerol could be an acyl acceptor and hydroxy-acyl-CoA could be an acyl donor. Regardless of the exact details of the reaction catalyzed, the present invention describes compositions and methods comprising heterologous GPAT family acyltransferases for the production of and alteration in wild-type and novel lipids on the surface of plants. It was further contemplated to use GPAT family acyltransferases in combination with a variety of transport proteins, such as plant ABC transporter molecules, for example, an Arabidopsis CER5 (ECERIFERUM 5) gene, (NM_{—}104028, SEQ ID NO:76, and Arabidopsis CER6 (ECERIFERUM 6; CUT1 (CUTICULAR 1); acyltransferase SEQ ID NO:77) for further altering exportation of additional lipids to the plant surface for producing specific surface lipids (see, for example, Pighin et al., (2004) Science 306(5696):702-704; herein incorporated by reference in its entirety).

In experiments conducted during the course of developing the present invention, transgenic Arabidopsis plant lines were created and analyzed that secreted at and on their surface high amounts of free fatty acid molecules, high amounts of very long chain free fatty acids and very long chain-containing fatty acids and novel monoacylglycerols. In one embodiment, transformed tobacco plant leaves that ectopically overexpressed GPAT5 were created and analyzed. In one embodiment, novel monoacylglycerols were produced. In one embodiment, elevated levels of C22-C30 free fatty acids were produced. In one embodiment, the inventors contemplated tobacco plant lines with surface wax characteristics of the Arabidopsis plant lines secreting fatty acid derivatives with similar chain lengths and functional groups. In one embodiment, crop plant lines with surface wax characteristics of the Arabidopsis plant lines secreting fatty acid derivatives with similar chain lengths and functional groups was contemplated.

In another embodiment, the use of a promoter active in a plant epidermal cell was contemplated, for example, a CER6 promoter (see, for example, Hooker et al., (2002) Plant Physiol., 129, 1568-1580; herein incorporated by reference in its entirety. In another embodiment, the use of a lipid transfer protein (LTP) or other strong epidermal promoter for driving the expression of GPAT family acyltransferase genes (see, for example, such as those described in Thoma, et al., (1994) Plant Physiol. 105:35-45 and Sohal et al., (1999) Plant Mol. Biol. 41(1):75-87, all of which are herein incorporated by reference).

### I. Plant GPAT Family Acyltransferase Genes.

A. Arabidopsis GPATs.

Seven putative Arabidopsis GPATs, including Arabidopsis GPAT5, were previously identified via partial sequence homology search (see, for example, Zheng et al. (2003) Plant Cell 15:1872-87; herein incorporated by reference in its entirety). In experiments conducted during the course of the present invention, an eighth member (GPAT8, see SEQ ID NOs: 08 and 16) of the Arabidopsis GPAT family, which was previously not annotated as a GPAT, was identified. Within this Arabidopsis GPAT acyltransferase family, GPAT1 was previously characterized and linked to a cellular function (see, for example, Zheng et al. (2003) Plant Cell 15:1872-87; herein incorporated by reference in its entirety). Three isoforms (GPAT1, GPAT 2, and GPAT 3) were predicted to be located in the mitochondria while uptake of GPAT1 by mitochondria was demonstrated (see, for example, Zheng et al. (2003) Plant Cell 15:1872-87; herein incorporated by reference in its entirety). As demonstrated herein, (see, FIG. 10), five GPAT isoforms (GPAT4, GPAT5, GPAT6, GPAT7, GPAT8) are grouped together in a distinct subgroup while GPAT1, GPAT2, and GPAT3 are grouped together in another distinct subgroup. Unlike the GPAT1, GPAT2 and GPAT3 subgroup, the GPAT4, GPAT5, GPAT6, GPAT7, and GPAT8 subgroup showed no predicted targeting signal using website programs such as TargetP (see, for example, Nielsen et al., (1997) Protein Engineering, 10:1-6; herein incorporated by reference in its entirety) and PSORT (see, for example, Horton et al., (2006) Proceedings of the 4th Annual Asia Pacific Bioinformatics Conference APBC06, Taipei, Taiwan. pp. 39-48; herein incorporated by reference in its entirety). However transmembrane domains were predicted for GPATs1-8 (ARAMEMNON website database, Schwacke et al., (2003) Plant Physiol. 131(1): 16-26; herein incorporated by reference in its entirety). An additional protein (At3g11325) was found to be 57% similar to GPAT5 at the amino acid level. However, is the inventors determined that it was unlikely to be an active member of the GPAT family due to the absence of conserved residues (His and Asp residues in [HRTLMDPVVLSYVLG, SEQ ID NO:66], of the HXXXXD motif, and the Gly residue in [GDLVVYPEGTTCREPFLLRFS, SEQ ID NO:67]), which are considered catalytically important sites for functional GPAT activity (see, for example, Lewin et al. (1999) Biochemistry 38:5764-5771; herein incorporated by reference in its entirety). Several GPATs were expressed in leaves and stems and in experiments conducted during the course of the present inventions GPAT 4 and 8 were shown to be involved in cutin synthesis, see, Examples.

B. GPATs in Other Plants.

Sequences that are homologes and orthologs of GPATs can be found in rice (Oryza sativa), poplar (Populus spp), maize (Zea mays), lettuce (Lactuca sativa), and other species, yet not in other non-plant organisms indicating that GPATs are plant specific, which is consistent with a role in suberin and cutin synthesis. In addition, a phylogenetic tree constructed with the 8 Arabidopsis and the 16 rice GPAT sequences (Horan, et al. (2005) Plant Physiol. 138:47-54; http://bioweb ucr.edu/databaseWeb/indexjsp; herein incorporated by reference) indicates that for each Arabidopsis GPAT group (e.g., GPAT5, 7 group), there is at least one rice GPAT that clusters with it. Therefore, the subfamily organization of the different GPAT members observed in Arabidopsis is also found in rice, suggesting that the functional differences of various GPAT subfamilies were conserved over 100 million years.

The identification of acyltransferases involved in suberin and cutin synthesis is an important step toward understanding the biosynthesis of surface lipid polymers in plants and obtaining transgenic plants with a modified cuticle that may confer more resistance to pests or stresses. In addition, the main experimental model to date to study suberin deposition has been the potato (Solanum tuberosum) wounding system. Therefore compositions and methods comprising GPAT5 are contemplated for use in altering suberin deposition in the potato tuber and in other plant parts.

### II. Plant GPAT5, GPAT and GPAT Family Member Genes and Proteins.

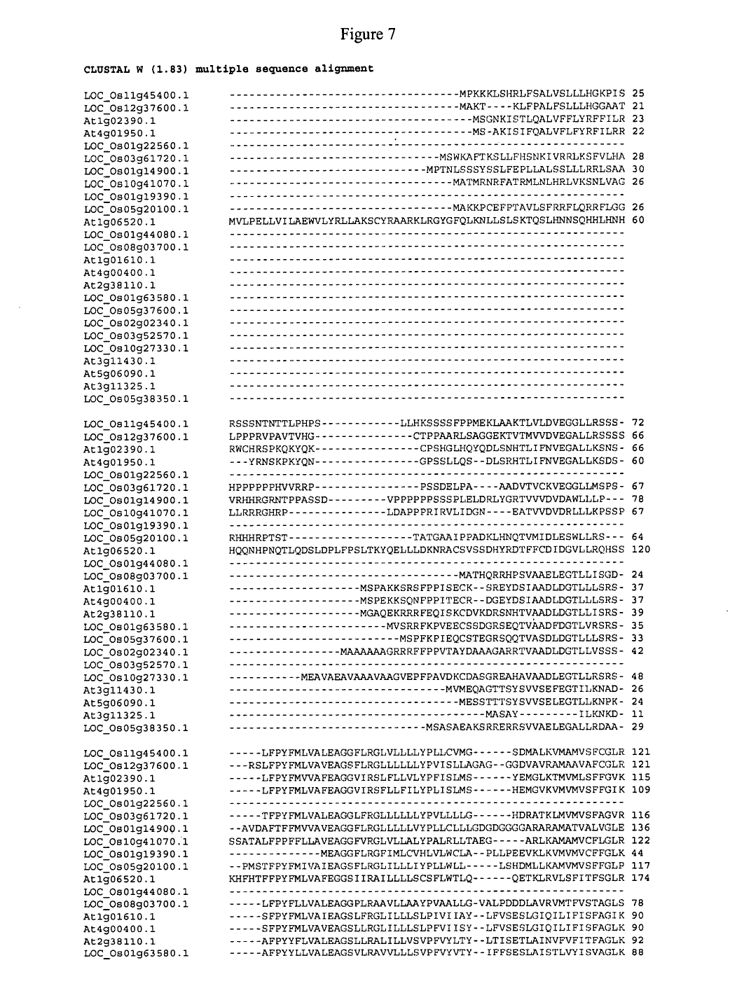

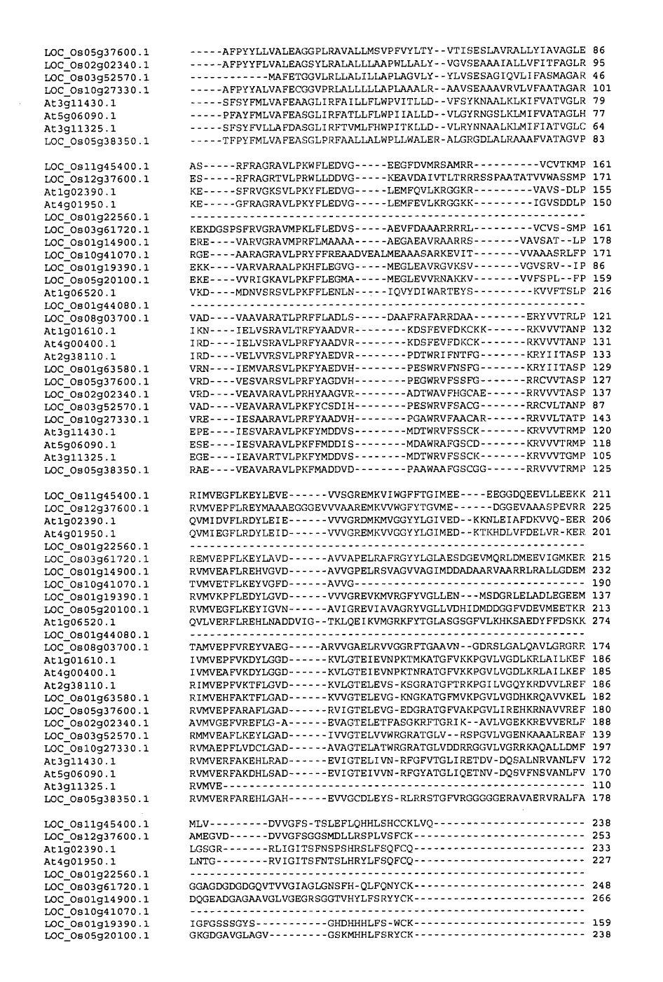

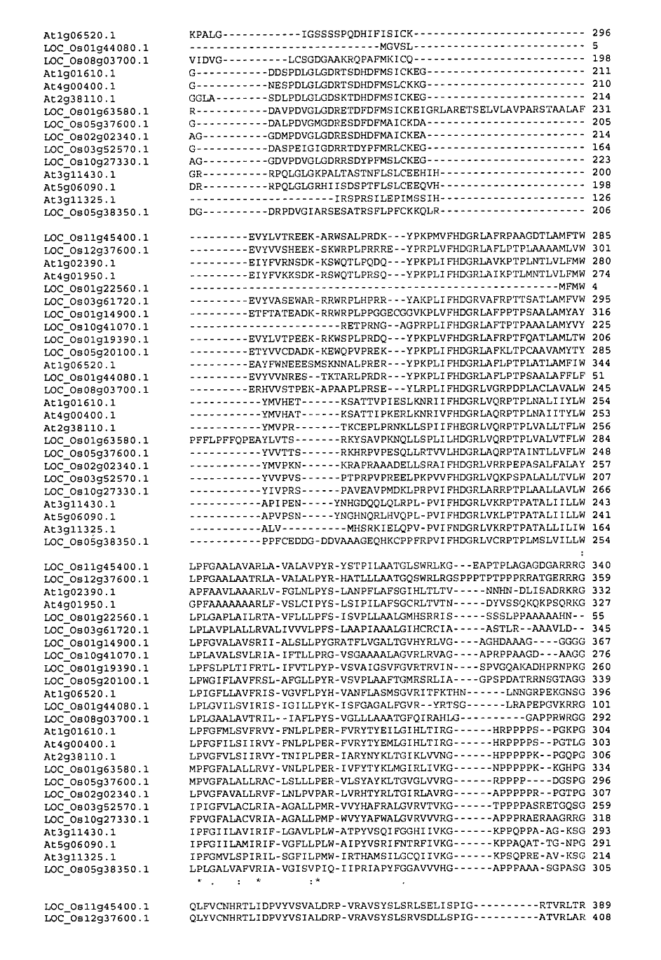

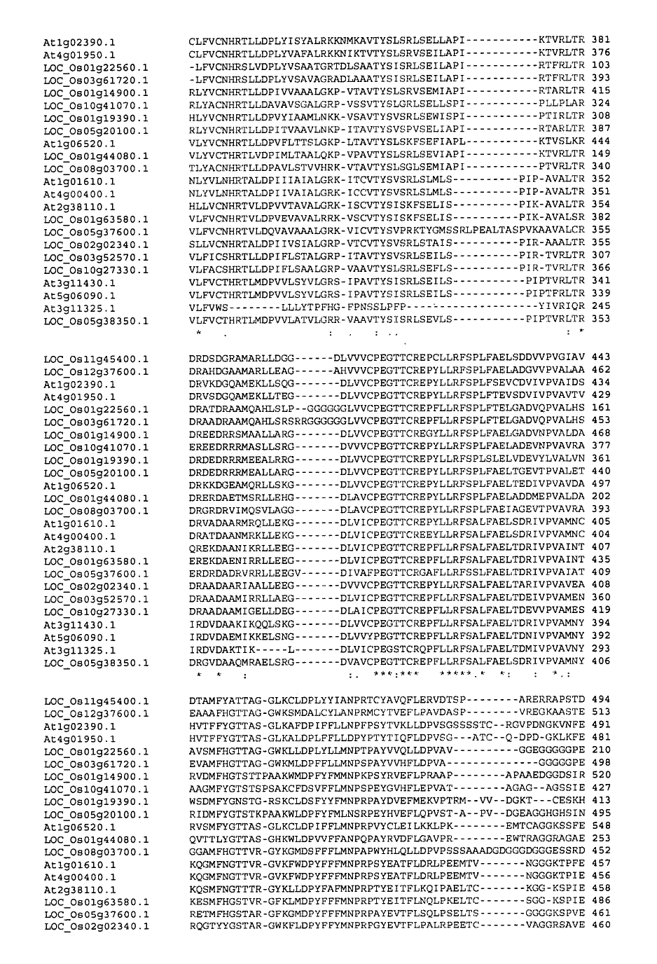

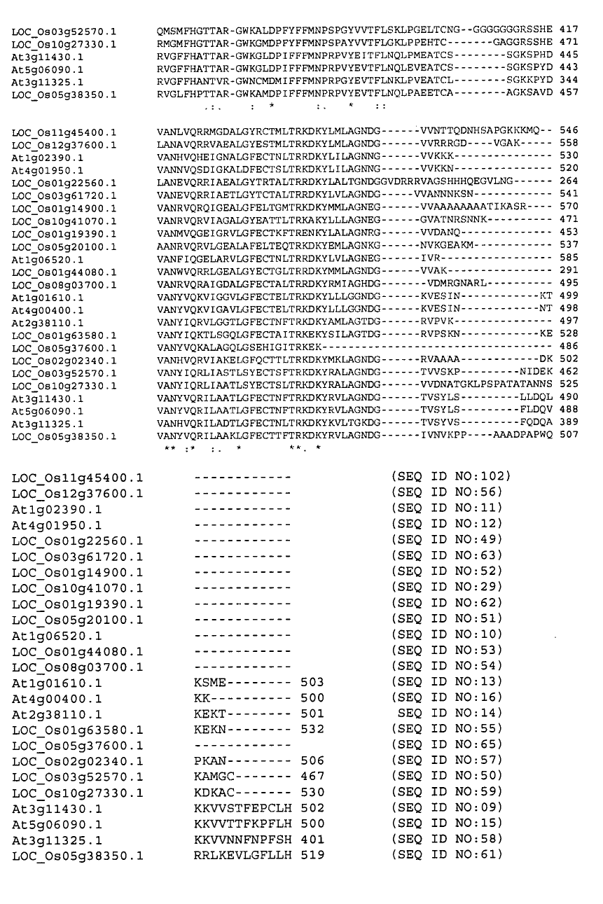

The present invention provides compositions comprising isolated nucleic acid sequences encoding plant GPAT5 or GPAT or GPAT acyltransferase family members. In some embodiments, the nucleic acid sequences encode an Arabidopsis GPAT5 or GPAT or GPAT acyltransferase family member protein. In other embodiments, the nucleic acid sequences encode a tobacco homolog of GPAT5 or GPAT or GPAT acyltransferase family member. In other embodiments, nucleic acid sequences encode a rice homolog of GPAT5 or GPAT or GPAT acyltransferase family member. In some embodiments, the sequences comprise a sequence shown in FIG. 9 (SEQ ID NO: 1). In other embodiments, the sequences encode the nucleic acid sequences shown in FIG. 9 (SEQ ID NOs:2-8). In yet other embodiments, the nucleic acid sequences encode proteins comprising at least one of the sequences shown in FIG. 9, FIG. 7, and Table 4. In other embodiments, the sequences encode at least one of the amino acid sequences shown in FIG. 9 (SEQ ID NOs:9-17). In preferred embodiments, the GPAT5 or GPAT or GPAT acyltransferase family member encoded by the nucleic acid sequences of the invention are functional and active as a GPAT.

In yet other embodiments, the present invention provides compositions comprising isolated nucleic acid sequences which encode a portion of a plant GPAT5 or GPAT or GPAT acyltransferase family member which retains some functional characteristic of a GPAT5 or GPAT or GPAT acyltransferase family member. Examples of functional characteristics include the ability to act as a plant GPAT family acyltransferase such as GPAT5 (see, for example, Examples 2 and 3). In preferred embodiments, the nucleic acid sequences encode the amino acid sequence shown in FIG. 9 (SEQ ID NOs:9-17).

The present invention provides isolated nucleic acid sequences encoding a plant GPAT5 or GPAT or GPAT acyltransferase family member and vectors comprising sequences encoding a plant GPAT5 or GPAT or GPAT acyltransferase family member. For example, some embodiments of the present invention provide isolated polynucleotide sequences that are capable of hybridizing to SEQ ID NOs:9-17 under conditions of low to high stringency as long as the polynucleotide sequence capable of hybridizing encodes a protein that retains a desired biological activity of a plant GPAT5. In preferred embodiments, hybridization conditions are based on the melting temperature (Tₘ) of the nucleic acid binding complex and confer a defined “stringency” as explained above (see, for example, Wahl et al. (1987) Meth. Enzymol., 152:399-407, incorporated herein by reference).

In other embodiments, an isolated nucleic acid sequence encoding a plant GPAT5 or GPAT or GPAT acyltransferase family member which is homologous to the Arabidopsis GPAT5 or GPAT or GPAT acyltransferase family member is provided. In some embodiments, the sequence is obtained from a plant from a family Brassicaceae, Apiaceae, Lauraceae, Leguminosae, Myrtaceae, Meliaceae, Rutaceae, Salicaceae, Santalaceae, and a Solanaceae family. In particular embodiments, such sequences are obtained from Arabidopsis; these sequences comprise at least one of SEQ ID NOs: 1-8.

In other embodiments of the present invention, alleles of a plant GPAT5 or GPAT or GPAT acyltransferase family member are provided. In preferred embodiments, alleles result from a mutation, (in other words, a change in the nucleic acid sequence) and generally produce altered mRNAs or polypeptides whose structure or function may or may not be altered. Any given gene may have none, one or many allelic forms. Common mutational changes that give rise to alleles are generally ascribed to deletions, additions or substitutions of nucleic acids. Each of these types of changes may occur alone, or in combination with the others, and at the rate of one or more times in a given sequence.

A. Variant Plant GPAT Family Acyltransferase Genes.

In some embodiments, the present invention provides isolated variants of the disclosed nucleic acid sequence encoding plant GPAT5 or GPAT or GPAT acyltransferase family, and the polypeptides encoded thereby; these variants include mutants, fragments, fusion proteins or functional equivalents of plant GPAT5 or GPAT or GPAT acyltransferase family. Thus, nucleotide sequences of the present invention are engineered in order to alter a plant GPAT5 or GPAT or GPAT acyltransferase family coding sequence for a variety of reasons, including but not limited to alterations that modify the cloning, processing and/or expression of the gene product (such alterations include inserting new restriction sites, altering glycosylation patterns, and changing codon preference) as well as varying the enzymatic activity (such changes include but are not limited to differing substrate affinities, differing substrate preferences and utilization, differing inhibitor affinities or effectiveness, differing reaction kinetics, varying subcellular localization, and varying protein processing and/or stability). For example, mutations are introduced which alter the substrate specificity, such that the preferred substrate is changed.

In other embodiments, the present invention provides isolated nucleic acid sequences encoding a plant GPAT5 or GPAT or GPAT acyltransferase family, where the encoded GPAT family acyltransferase competes for binding to a fatty acyl substrate with a protein comprising the amino acid sequence of SEQ ID NO:9.

B. Mutants of a Plant GPAT Family Acyltransferase Genes.

Some embodiments of the present invention provide mutant forms of a plant GPAT5 or GPAT or GPAT acyltransferase family (in other words, muteins). In preferred embodiments, variants result from mutation, (in other words, a change in the nucleic acid sequence) and generally produce altered mRNAs or polypeptides whose structure or function may or may not be altered. Any given gene may have none, one, or many mutant forms. Common mutational changes that give rise to variants are generally ascribed to deletions, additions or substitutions of nucleic acids. Each of these types of changes may occur alone, or in combination with the others, and at the rate of one or more times in a given sequence.

The inventors contemplated that it is the inventors possible to modify the structure of a peptide having an activity (for example, a plant GPAT5 or GPAT or GPAT acyltransferase family activity) for such purposes as increasing GPAT family acyltransferase activity or altering the affinity of the plant GPAT5 or GPAT or GPAT acyltransferase family for a particular fatty acyl substrate. Such modified peptides are considered functional equivalents of peptides having an activity of a plant GPAT5 or GPAT or GPAT acyltransferase family as defined herein. A modified peptide can be produced in which the nucleotide sequence encoding the polypeptide has been altered, such as by substitution, deletion, or addition. In some preferred embodiments of the present invention, the alteration increases GPAT5 or GPAT or GPAT acyltransferase family acyltransferase activity or alters the affinity of the plant GPAT5 or GPAT or GPAT acyltransferase family for a particular fatty acyl substrate. In particularly preferred embodiments, these modifications do not significantly reduce the synthetic activity of the modified enzyme. In other words, construct “X” can be evaluated in order to determine whether it is a member of the genus of modified or variant plant GPAT5 or GPAT or GPAT acyltransferase family of the present invention as defined functionally, rather than structurally. In preferred embodiments, the activity of variant plant GPAT5 or GPAT or GPAT acyltransferase family is evaluated by the methods described in Examples. Accordingly, in some embodiments the present invention provides nucleic acids encoding a plant GPAT5 or GPAT or GPAT acyltransferase family that complement the coding region of SEQ ID NO: 1. In other embodiments, the present invention provides nucleic acids encoding a plant GPAT5 or GPAT or GPAT acyltransferase family that compete for the binding of fatty acyl substrates with the protein encoded by SEQ ID NO: 1.

As described above, mutant forms of a plant GPAT5 or GPAT or GPAT acyltransferase family are also contemplated as being equivalent to those peptides and DNA molecules that are set forth in more detail herein. For example, it is contemplated that isolated replacement of a leucine with an isoleucine or valine, an aspartate with a glutamate, a threonine with a serine, or a similar replacement of an amino acid with a structurally related amino acid (in other words, conservative mutations) will not have a major effect on the biological activity of the resulting molecule. Accordingly, some embodiments of the present invention provide variants of a plant GPAT5 or GPAT or GPAT acyltransferase family disclosed herein containing conservative replacements. Conservative replacements are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur-containing (cysteine and methionine) (for example, Stryer ed. (1981) Biochemistry pg. 17-21, 2nd ed, WH Freeman and Co., herein incorporated by reference). Whether a change in the amino acid sequence of a peptide results in a functional homolog can be readily determined by assessing the ability of the variant peptide to function in a fashion similar to the wild-type protein. Peptides having more than one replacement can readily be tested in the same manner.

More rarely, a variant includes “nonconservative” changes (for example, replacement of a glycine with a tryptophan). Analogous minor variations can also include amino acid deletions or insertions, or both. Guidance in determining which amino acid residues can be substituted, inserted, or deleted without abolishing biological activity can be found using computer programs (for example, LASERGENE software, DNASTAR Inc., Madison, Wis.).

Mutants of a plant GPAT5 or GPAT or GPAT acyltransferase family can be generated by any suitable method well known in the art, including but not limited to site-directed mutagenesis, randomized “point” mutagenesis, and domain-swap mutagenesis in which portions of the Arabidopsis GPAT5 cDNA are “swapped” with the analogous portion of other plant GPAT5 or GPAT or GPAT acyltransferase family or yeast or bacterial GPAT-encoding cDNAs (Back and Chappell (1996) PNAS 93: 6841-6845, herein incorporated by reference).

Variants may be produced by methods such as directed evolution or other techniques for producing combinatorial libraries of variants. Thus, the present invention further contemplates a method of generating sets of combinatorial mutants of the present plant GPAT5 or GPAT or GPAT acyltransferase family proteins, as well as truncation mutants, and is especially useful for identifying potential variant sequences (in other words, homologs) that possess the biological activity of a GPAT5 or GPAT or GPAT acyltransferase family (for example, synthesis of GPAT5). In addition, screening such combinatorial libraries is used to generate, for example, novel plant GPAT5 or GPAT or GPAT acyltransferase family homologs that possess novel substrate specificities or other biological activities; examples of substrate specificities are described subsequently.

The inventors contemplate that the plant GPAT5 or GPAT or GPAT acyltransferase family nucleic acids (for example, SEQ ID NO: 1, and fragments and variants thereof) can be utilized as starting nucleic acids for directed evolution. These techniques can be utilized to develop plant GPAT5 or GPAT or GPAT acyltransferase family variants having desirable properties such as increased synthetic activity or altered affinity for a particular fatty acyl substrate, or increased protein stability.

In some embodiments, artificial evolution is performed by random mutagenesis (for example, by utilizing error-prone PCR to introduce random mutations into a given coding sequence). This method requires that the frequency of mutation be finely tuned. As a general rule, beneficial mutations are rare, while deleterious mutations are common. This is because the combination of a deleterious mutation and a beneficial mutation often results in an inactive enzyme. The ideal number of base substitutions for targeted gene is usually between 1.5 and 5 (see, for example, Moore and Arnold (1996) Nat. Biotech., 14:458-67; Leung et al. (1989) Technique, 1:11-15; Eckert and Kunkel (1991) PCR Methods Appl., 1:17-24; Caldwell and Joyce (1992) PCR Methods Appl., 2:28-33; and Zhao and Arnold (1997) Nuc. Acids. Res., 25:1307-08; all of which are herein incorporated by reference). After mutagenesis, the resulting clones are selected for desirable activity (for example, screened for GPAT5 activity as described subsequently). Successive rounds of mutagenesis and selection are often necessary to develop enzymes with desirable properties. It should be noted that only the useful mutations are carried over to the next round of mutagenesis.

In other embodiments of the present invention, the polynucleotides of the present invention are used in gene shuffling or sexual PCR procedures (see, for example, Smith (1994) Nature, 370:324-25; U.S. Pat. Nos. 5,837,458; 5,830,721; 5,811,238; 5,733,731, all of which are herein incorporated by reference). Gene shuffling involves random fragmentation of several mutant DNAs followed by their reassembly by PCR into full length molecules. Examples of various gene shuffling procedures include, but are not limited to, assembly following DNase treatment, the staggered extension process (STEP), and random priming in vitro recombination. In the DNase mediated method, DNA segments isolated from a pool of positive mutants are cleaved into random fragments with DnaseI and subjected to multiple rounds of PCR with no added primer. The lengths of random fragments approach that of the uncleaved segment as the PCR cycles proceed, resulting in mutations in present in different clones becoming mixed and accumulating in some of the resulting sequences. Multiple cycles of selection and shuffling have led to the functional enhancement of several enzymes (Stemmer (1994) Nature, 370:398-91; Stemmer (1994) Proc. Natl. Acad. Sci. USA, 91, 10747-10751; Crameri et al. (1996) Nat. Biotech., 14:315-319; Zhang et al. (1997) Proc. Natl. Acad. Sci. USA, 94:4504-09; and Crameri et al. (1997) Nat. Biotech., 15:436-38; all of which are herein incorporated by reference). Variants produced by directed evolution can be screened for GPAT5 or GPAT or GPAT acyltransferase family activity by the methods described subsequently (see Example II).

C. Homologs.

Still other embodiments of the present invention provide isolated nucleic acid sequence encoding plant GPAT5 or GPAT or GPAT acyltransferase family homologs, and the polypeptides encoded thereby. Some homologs of plant GPAT5 or GPAT or GPAT acyltransferase family 5 have intracellular half-lives dramatically different than the corresponding wild-type protein. For example, the altered protein are rendered either more stable or less stable to proteolytic degradation or other cellular process that result in destruction of, or otherwise inactivate plant GPAT5 or GPAT or GPAT acyltransferase family. Such homologs, and the genes that encode them, can be utilized to alter the activity of plant GPAT5 or GPAT or GPAT acyltransferase family by modulating the half-life of the protein. For instance, a short half-life can give rise to more transient plant GPAT5 or GPAT or GPAT acyltransferase family biological effects. Other homologs have characteristics which are either similar to wild-type plant GPAT5 or GPAT or GPAT acyltransferase family, or which differ in one or more respects from wild-type plant GPAT5 or GPAT or GPAT acyltransferase family.

The cDNA deduced amino acid sequence of Arabidopsis GPAT5 or GPAT or GPAT acyltransferase family was compared to the cDNA deduced amino acid sequences of other known plant GPAT5 or GPAT or GPAT acyltransferase family proteins, as shown in FIG. 7.

In some embodiments of the combinatorial mutagenesis approach of the present invention, the amino acid sequences for a population of plant GPAT5 or GPAT or GPAT acyltransferase family-like proteins are aligned, preferably to promote the highest homology possible, see, FIG. 7. Such a population of variants can include, for example, plant GPAT5 isoforms or homologs or orthologs from one or more species, or plant GPAT5 or GPAT or GPAT acyltransferase family isoforms or homologs or orthologs from the same species but which differ due to mutation. Amino acids that appear at each position of the aligned sequences are selected to create a degenerate set of combinatorial sequences.

### III. Expression of Cloned Plant GPAT Family Acyltransferase Genes.

In other embodiment of the present invention, nucleic acid sequences corresponding to the plant GPAT5 or GPAT or GPAT acyltransferase family genes, homologs and mutants as described above may be used to generate recombinant DNA molecules that direct the expression of the encoded protein product in appropriate host cells.

As will be understood by those of skill in the art, it may be advantageous to produce plant GPAT5 or GPAT or GPAT acyltransferase family-encoding nucleotide sequences possessing non-naturally occurring codons. Therefore, in some preferred embodiments, codons preferred by a particular prokaryotic or eukaryotic host (see, for example, Murray et al. (1989) Nucl. Acids Res., 17; herein incorporated by reference in its entirety) can be selected, for example, to increase the rate of plant GPAT5 or GPAT or GPAT acyltransferase family expression or to produce recombinant RNA transcripts having desirable properties, such as a longer half-life, than transcripts produced from naturally occurring sequence.

A. Vectors for Production of Plant GPAT Family Acyltransferase Genes.

The nucleic acid sequences of the present invention may be employed for producing polypeptides by recombinant techniques. Thus, for example, the nucleic acid sequence may be included in any one of a variety of expression vectors for expressing a polypeptide. In some embodiments of the present invention, vectors include, but are not limited to, chromosomal, nonchromosomal and synthetic DNA sequences (for example, plasmid vectors, binary agrobacterium vectors, T-DNA vectors, DNA viruses, RNA viruses, bicistronic vectors, bacterial plasmids, phage DNA; baculovirus, yeast plasmids, vectors derived from combinations of plasmids and phage DNA, and viral DNA such as cauliflower mosaic virus, Tomato leaf curl virus (TLCV) satellite DNA (sat-DNA) constructs (for example, L1, (2007), J Gen Virol 88:2073-2077; herein incorporated by reference) et cetera. The inventors contemplated that any vector may be used as long as it is the inventors replicable and viable in the host.

In particular, some embodiments of the present invention provide recombinant constructs comprising one or more of the nucleic sequences as broadly described above (for example, SEQ ID NO: 1). In some embodiments of the present invention, the constructs comprise a vector, such as a plasmid or viral vector, into which a nucleic acid sequence of the invention has been inserted, in a forward or reverse orientation. In preferred embodiments of the present invention, the appropriate nucleic acid sequence is inserted into the vector using any of a variety of procedures. In general, the nucleic acid sequence is inserted into an appropriate restriction endonuclease site(s) by procedures known in the art.

Large numbers of suitable vectors are known to those of skill in the art, and are commercially available. Such vectors include, but are not limited to, the following vectors: 1) Bacterial—pBI121, pQE70, pQE60, pQE-9 (Qiagen), pBS, pD10, phagescript, psiXI74, pbluescript SK, pBSKS, pNH8A, pNH16a, pNH18A, pNH46A (Stratagene); ptrc99a, pKK223-3, pKK233-3, pDR540, pRIT5 (Pharmacia); and 2) Eukaryotic—pWLNEO, pSV2CAT, pOG44, PXT1, pSG (Stratagene) pSVK3, pBPV, pMSG, and pSVL (Pharmacia). Any other plasmid or vector may be used as long as they are replicable and viable in the host. In some preferred embodiments of the present invention, plant expression vectors comprise an origin of replication, a suitable promoter and enhancer, and also any necessary ribosome binding sites, polyadenylation sites, splice donor and acceptor sites, transcriptional termination sequences, and 5′ flanking nontranscribed sequences. In other embodiments, DNA sequences derived from the SV40 splice, and polyadenylation sites may be used to provide the required nontranscribed genetic elements.

B. Promoters and Enhancers.

In certain embodiments of the present invention, a nucleic acid sequence of the present invention within an expression vector is operatively linked to an appropriate expression control sequence(s) (promoter) to direct mRNA synthesis. Promoters useful in the present invention include, but are not limited to, GPAT5 promoter, a GPAT4 promoter, a GPAT7 promoter, a GPAT8 promoter, a Lipid Transfer Protein 1 (LPT1) promoter, a CUTICULAR 1 (CUT 1, eceriferum 6 (CER6)) promoter, a Long Chain Acyl-CoA Synthetase 2 (LACS2) promoter, a acyl-CoA synthetase long-chain family member 3 (ACSL3) promoter, FbL2A promoter, E6 promoter, patatin promoter, a potato multicystatin (PMC) promoter, Cauliflower Mosaic Virus (CaMV) 35S Promoter (CaMV 35S promoter) (“35S promoter,” a chemically-inducible promoter from tobacco, Pathogenesis-Related 1 (PR1) (induced by salicylic acid and BTH (benzothiadiazole-7-carbothioic acid S-methyl ester)); a tomato proteinase inhibitor II promoter (PIN2) or LAP promoter (both inducible with methyl jasmonate); a heat shock promoter (e.g., U.S. Pat. No. 5,187,2675; herein incorporated by reference), a tetracycline-inducible promoter (e.g., U.S. Pat. No. 5,057,4225; herein incorporated by reference); seed-specific promoters, such as those for seed storage proteins (for example, phaseolin, napin, oleosin, and a promoter for soybean beta conglycin (e.g., Beachy et al. (1985) EMBO J. 4: 3047-3053; herein incorporated by reference) ABA Insensitive3 (ABI3), STIG1, TAP1, LAT52, TOBRB7, Petal Loss (PTL), Apetala3 (AP3), Apetala1 (AP1), Aymmetric Leaves1 (AS1), Kanadi4 (KAN4), Crabs Claw (CRC), Agamous (AG), ATML1, CLAVATA3 (CLV3), CLAVATA1 (CLV1), ANTINTEGUMENTA (ANT), Shoot Meristemless (STM), Chrorophyl A/B Binding Protein (CAB3), Agamous Like 1 (AGL1), Agamous Like 8 (AGL8), PHAVOLUTA (PHV), Revoluta (REV), Filamentous Flower (FIL), Cupshaped Cotyledons (CUC2), Pinformed (PIN3), AtlPT1, AtlPT5, AtlPT7, AtRPS5, Cryptochrome2 (CRY2), a napin promoter, a 2S albumin promoter, and an oleosin promoter and other promoters known to control expression of a gene in plant cells or algal cells (for example, Chlamydomonas reinhardtii alternative oxidase (Aox1) promoter, Baurain, et al., 2003, Plant Physiol. 131(3):1418-30; herein incorporated by reference) or their viruses. In other embodiments of the present invention, recombinant expression vectors include origins of replication and selectable markers permitting transformation of the host cell (for example, dihydrofolate reductase or neomycin resistance for eukaryotic cell culture, or tetracycline or ampicillin resistance in E. coli). In some embodiments, promoters contemplated for use include but are not limited to cold-inducible and tuber-specific promoter sequence from potato α-amylase gene a cold-inducible promoter region and a tuber-specific promoter region from an α-amylase gene from Solanum tuberosum in U.S. Pat. No. 6,184,443; herein incorporated by reference in its entirety, cold-inducible promoters and cold-inducible transcription factors such as R929A, DREB1A (Kasuga Plant and Cell Physiology, 2004, Vol. 45, No. 3 346-350; herein incorporated by reference) CBF, CBF1, CRT/DRE regulatory elements, (Gilmour et al., 1998, Plant J. 16(4):433-42, Zarka et al., 2003, Plant Physiol. 133(2):910-8; herein incorporated by reference), RCI2A, RCI2B (Medina, et al., 2001, Plant Physiol, 125:1655-1666; herein incorporated by reference), patatin promoter (Stupar et al., 2006, Genetics 172: 1263-1275; herein incorporated by reference) plant seed specific promoters (United States Patent Application No. 20070022502; herein incorporated by reference), and epidermal promoters identified from highly expressed transcripts described by (Suh et al., 2005, Plant Physiology, 139:1649-1665; herein incorporated by reference); all of which are herein incorporated by reference in their entirety.

In some embodiments of the present invention, transcription of the DNA encoding polypeptides of the present invention by higher eukaryotes is increased by inserting an enhancer sequence into the vector. Enhancers are cis- or trans-acting elements of DNA, usually about from 10 to 300 bp that act on a promoter to increase its transcription. Examples of enhancer elements for plants are shown in Chen et al., (1986) PNAS 83:8560 and Chen, et al. (1988) Embo Journal 7:297-302; herein incorporated by reference in their entirety.

In other embodiments, the expression vector also contains a ribosome binding site for translation initiation and a transcription terminator. In still other embodiments of the present invention, the vector may also include appropriate sequences for amplifying expression.

### IV. Host Cells for Production of Plant Surface Lipids

In a further embodiment, the present invention provides host cells containing any of the above-described constructs. In some embodiments of the present invention, the host cell is a higher eukaryotic cell (for example, a plant cell). In other embodiments of the present invention, the host cell is a lower eukaryotic cell (for example, a yeast or algal cell). In still other embodiments of the present invention, the host cell can be a prokaryotic cell (for example, a bacterial cell). Specific examples of host cells include, but are not limited to, Escherichia coli, Salmonella typhimurium, Bacillus subtilis, and various species within the genera Botryococcus, Pseudomonas, Streptomyces, and Staphylococcus, as well as Saccharomyces cerivisiae, Schizosaccharomycees pombe, Drosophila S2 cells, Spodoptera Sf9 cells, Chinese hamster ovary (CHO) cells, COS-7 lines of monkey kidney fibroblasts, (Gluzman (1981) Cell 23:175), 293T, C127, 3T3, HeLa and BHK cell lines, NT-1 (tobacco cell culture line), root cell and cultured roots in rhizosecretion (see, for example, Gleba et al. (1999) Proc Natl Acad Sci USA 96: 5973-5977; herein incorporated by reference in its entirety). Other examples include microspore-derived cultures of oilseed rape (see, for example, Weselake R J and Taylor D C (1999) Prog. Lipid Res. 38:401; herein incorporated by reference in its entirety), and transformation of pollen and microspore culture systems. Further examples are described in the Examples.

The constructs in host cells can be used in a conventional manner to produce the gene product encoded by any of the recombinant sequences of the present invention described above.

In some embodiments, introduction of the construct into the host cell can be accomplished by calcium phosphate transfection, DEAE-Dextran mediated transfection, or electroporation (see, for example, Davis et al. (1986) Basic Methods in Molecular Biology; herein incorporated by reference in its entirety). Alternatively, in some embodiments of the present invention, a polypeptide of the invention can be synthetically produced by conventional peptide synthesizers.

In some embodiments of the present invention, following transformation of a suitable host strain and growth of the host strain to an appropriate cell density, the selected promoter is induced by appropriate means (for example, temperature shift or chemical induction) and cells are cultured for an additional period. In other embodiments of the present invention, cells are typically harvested by centrifugation, disrupted by physical or chemical means, and the resulting crude extract retained for further purification. In still other embodiments of the present invention, microbial cells employed in expression of proteins can be disrupted by any convenient method, including freeze-thaw cycling, sonication, mechanical disruption, or use of cell lysing agents.

### V. Production of Large Quantities of Surface Lipids

In one aspect of the present invention, methods are provided for producing large quantities of surface lipids. In some embodiments, the amount of any one type of surface lipid, such as a VLCFA, is greater than the amount of that type of lipid located inside of an epidermal cell. In some embodiments, surface lipids are produced in vivo, in organisms transformed with a heterologous gene encoding a polypeptide exhibiting GPAT5 or GPAT7 or GPAT8 or GPAT or GPAT acyltransferase family and grown under conditions sufficient to effect production of surface lipids. In other embodiments, surface lipids are produced in vitro, from either nucleic acid sequences encoding a plant GPAT5 or GPAT7 or GPAT8 or GPAT or GPAT acyltransferase family acyltransferase or from polypeptides exhibiting plant GPAT5 or GPAT7 or GPAT8 or GPAT or GPAT acyltransferase family activity.

A. In Vivo in Transgenic Organism

In some embodiments of the present invention, lipids are produced in vivo, by providing an organism transformed with a gene encoding a plant GPAT5 or GPAT7 or GPAT8 or GPAT or GPAT acyltransferase family acyltransferase and growing the transgenic organism under conditions sufficient to effect production of surface lipids. In other embodiments of the present invention, surface lipids are produced in vivo by transforming an organism with a heterologous gene encoding a plant GPAT5 or GPAT7 or GPAT8 or GPAT or GPAT acyltransferase family and growing the transgenic organism under conditions sufficient to effect production of surface lipids. Illustrative examples of transgenic organisms are provided in the Examples.

Organisms which are transformed with a heterologous gene encoding a plant GPAT5 or GPAT7 or GPAT8 or GPAT or GPAT acyltransferase family include preferably those which naturally synthesize and secrete in some manner surface lipids and those which are commercially feasible to grow and suitable for collecting large amounts of the lipid products. Such organisms include but are not limited to algae and plants. Examples of algae include Dunaliella salina Botryococcus and similar organisms which can be grown in commercial-scale fermenters, (such as in Apt and Behrens, (1999) Journal of Phycology, 35:2151; and The Mera Growth Module (MGM), Mera Pharmaceuticals Inc., all of which are herein incorporated by reference in its entirety). Examples of plants include preferably tobacco, potato, cotton, tomato, rapeseed, rice, Brassica species, et cetera. Many commercial cultivars can be transformed with heterologous genes. In cases where that is not possible to transform commercial cultivars, non-commercial cultivars of plants would be transformed, after which the trait for expression of surface lipids produced by GPAT5 or GPAT7 or GPAT8 or GPAT or GPAT acyltransferase family moved to commercial cultivars by breeding techniques well-known in the art.

A transgenic organism is grown under conditions sufficient to effect production of surface lipids. In some embodiments of the present invention, a transgenic organism is supplied with exogenous substrates of the plant GPAT5 or GPAT7 or GPAT8 or GPAT or GPAT acyltransferase family (as for example as in a fermenter). Such substrates comprise fatty acids; the number of double bonds is from zero to more than one, and the chain length of such saturated or unsaturated fatty acids is variable, however is preferably about 12 to 30 carbons in length. The fatty acyl substrate may also comprise additional functional groups, including but not limited to acetylenic bonds, conjugated acetylenic and ethylenic bonds, allenic groups, furan rings, and epoxy-, and keto-groups; two or more of these functional groups may be found in a single fatty acid. The substrates are either free fatty acids, or their salts. Substrates may be supplied in various forms as are well known in the art; such forms include aqueous suspensions prepared by sonication, aqueous suspensions prepared with detergents and other surfactants, dissolution of the substrate into a solvent, and dried powders of substrates. Such forms may be added to organisms or cultured cells or tissues grown in fermenters.

In yet other embodiments of the present invention, a transgenic organism comprises a heterologous gene encoding a plant GPAT5 or GPAT or GPAT acyltransferase family operably linked to an inducible promoter, and is grown either in the presence of an inducing agent, or is grown and then exposed to an inducing agent. In still other embodiments of the present invention, a transgenic organism comprises a heterologous gene encoding a plant GPAT5 or GPAT or GPAT acyltransferase family operably linked to a promoter which is either tissue specific or developmentally specific, and is grown to the point at which the tissue is developed or the developmental stage at which the developmentally-specific promoter is activated. Such promoters include epidermal specific promoters or inducible promoters (such as induced by a chemical or an abiotic stress). In other embodiments of the present invention, the methods for producing large quantities of surface lipids further comprise collecting the lipids produced. Such methods are known generally in the art, and include collecting the transgenic organisms and extracting lipids from a variety of surfaces and plant parts (see, Example I).

1. Transgenic Plants Seeds, and Plant Parts

Plants are transformed with a gene encoding a heterologous plant GPAT family acyltransferase, such as Arabidopsis GPAT 1-8, in particular GPAT5, or transformed with a homolog or ortholog of a GPAT family acyltransferase, or transformed with a fusion gene encoding a fusion polypeptide expressing a plant GPAT family acyltransferase according to procedures well known in the art. In one embodiment, it was contemplated that the heterologous GPAT family acyltransferase genes are utilized to increase the level of the enzyme activities encoded by the heterologous genes. In one embodiment, it was contemplated that the heterologous GPAT family acyltransferase genes are utilized to introduce the enzyme activities encoded by the heterologous genes.

a. Plants.

The methods of the present invention are not limited to any particular plant. Indeed, a variety of plants are contemplated, including but not limited to tobacco, tomato, rice, Brassica, Arabidopsis, potato, pepper, corn, barley, wheat, sunflower, and soybean. The group also includes non-agronomic species which are useful in developing appropriate expression vectors such as tobacco, rapid cycling Brassica species, and Arabidopsis thaliana, and wild species which may be a source of unique fatty acids.

b. Vectors.

The methods of the present invention contemplate the use of a heterologous gene encoding a plant GPAT5 or GPAT or GPAT acyltransferase family member, as described above. Heterologous genes encoding mutants and variants of GPAT family acyltransferases are prepared as described above for plant GPAT. Further, it was contemplated that expression cassettes comprising a GPAT5 or GPAT or GPAT acyltransferase family member further comprise one or more additional heterologous genes. For example, additional heterologous genes may encode a fusion GPAT5/lipid altering gene, such as a fatty acid desaturase gene.

Heterologous genes intended for expression in plants are first assembled in expression cassettes comprising a promoter. Methods which are well known to those skilled in the art may be used to construct expression vectors containing a heterologous gene and appropriate transcriptional and translational control elements. These methods include in vitro recombinant DNA techniques, synthetic techniques, and in vivo genetic recombination. Such techniques are widely described in the art (see, for example, Sambrook. et al. (1989) Molecular Cloning, A Laboratory Manual, Cold Spring Harbor Press, Plainview, N.Y., and Ausubel, et al. (1989) Current Protocols in Molecular Biology, John Wiley & Sons, New York, N.Y.; all of which are herein incorporated by reference in their entirety).

In general, these vectors comprise a nucleic acid sequence of the invention encoding a plant GPAT5 or GPAT or GPAT acyltransferase family member (as described above) operably linked to a promoter and other regulatory sequences (for example, enhancers, polyadenylation signals, etc.) required for expression in a plant.

Promoters include but are not limited to constitutive promoters, tissue-, organ-, and developmentally-specific promoters, and inducible promoters. Examples of promoters include but are not limited to: constitutive promoter 35S of cauliflower mosaic virus; a wound-inducible promoter from tomato, leucine amino peptidase (see, for example, “LAP,” Chao et al. (1999) Plant Physiol 120:979-992; herein incorporated by reference in its entirety); a chemically-inducible promoter from tobacco, Pathogenesis-Related 1 (PR1) (induced by salicylic acid and BTH (benzothiadiazole-7-carbothioic acid S-methyl ester)); a tomato proteinase inhibitor II promoter (PIN2) or LAP promoter (both inducible with methyl jasmonate); a heat shock promoter (see, for example, U.S. Pat. No. 5,187,2675, herein incorporated by reference); a tetracycline-inducible promoter (see, for example, U.S. Pat. No. 5,057,4225, herein incorporated by reference); and seed-specific promoters, such as those for seed storage proteins (for example, phaseolin, napin, oleosin, and a promoter for soybean beta conglycin (see, for example, Beachy et al. (1985) EMBO J. 4:3047-3053; herein incorporated by reference in its entirety). All references cited herein are incorporated in their entirety.

The expression cassettes may further comprise any sequences required for expression of mRNA. Such sequences include, but are not limited to transcription terminators, enhancers such as introns, viral sequences, and sequences intended for the targeting of the gene product to specific organelles and cell compartments.

A variety of transcriptional terminators are available for use in expression of sequences using the promoters of the present invention. Transcriptional terminators are responsible for the termination of transcription beyond the transcript and its correct polyadenylation. Appropriate transcriptional terminators and those which are known to function in plants include, but are not limited to, the CaMV 35S terminator, the tm1 terminator, the pea rbcS E9 terminator, and the nopaline and octopine synthase terminator (see, for example, Odell et al. (1985) Nature 313:810; Rosenberg et al. (1987) Gene, 56:125; Guerineau et al. (1991) Mol. Gen. Genet., 262:141; Proudfoot (1991) Cell, 64:671; Sanfacon et al. (1991) Genes Dev. 5:141; Mogen et al. (1990) Plant Cell, 2:1261; Munroe et al. (1990) Gene, 91:151; Ballad et al. (1989) Nucleic Acids Res. 17:7891; Joshi et al. (1987) Nucleic Acid Res., 15:9627; all of which are herein incorporated by reference). In addition, in some embodiments, constructs for expression of the gene of interest include one or more of sequences found to enhance gene expression from within the transcriptional unit. These sequences can be used in conjunction with the nucleic acid sequence of interest to increase expression in plants. Various intron sequences have been shown to enhance expression, particularly in monocotyledonous cells. For example, the introns of the maize AdhI gene have been found to significantly enhance the expression of the wild-type gene under its cognate promoter when introduced into maize cells (see, for example, Calais et al. (1987) Genes Develop. 1:1183; herein incorporated by reference in its entirety). Intron sequences have been routinely incorporated into plant transformation vectors, typically within the non-translated leader.

In some embodiments of the present invention, the construct for expression of the nucleic acid sequence of interest also includes a regulator such as a nuclear localization signal (see, for example, Calderone et al. (1984) Cell 39:499; Lassoer et al. (1991) Plant Molecular Biology 17:229, all of which are herein incorporated by reference), a plant translational consensus sequence (Joshi (1987) Nucleic Acids Research 15:6643, herein incorporated by reference), an intron (Luehrsen and Walbot (1991) Mol. Gen. Genet. 225:81, herein incorporated by reference), and the like, operably linked to the nucleic acid sequence encoding plant GPAT5.

In preparing the construct comprising a nucleic acid sequence encoding plant GPAT5 or GPAT or GPAT acyltransferase family, various DNA fragments can be manipulated, so as to provide for the DNA sequences in the desired orientation (for example, sense or antisense) orientation and, as appropriate, in the desired reading frame. For example, adapters or linkers can be employed to join the DNA fragments or other manipulations can be used to provide for convenient restriction sites, removal of superfluous DNA, removal of restriction sites, or the like. For this purpose, in vitro mutagenesis, primer repair, restriction, annealing, resection, ligation, or the like, is preferably employed, where insertions, deletions or substitutions (for example, transitions and transversions) are involved.

Numerous transformation vectors are available for plant transformation. The selection of a vector for use will depend upon the preferred transformation technique and the target species for transformation. For certain target species, different antibiotic or herbicide selection markers are preferred. Selection markers used routinely in transformation include the NPTII gene which confers resistance to kanamycin and related antibiotics (Messing and Vierra (1982) Gene 19: 259; Bevan et al. (1983) Nature 304:184; herein incorporated by reference in its entirety), the bar gene which confers resistance to the herbicide phosphinothricin (see, for example, White et al. (1990) Nucl Acids Res. 18: 1062; Spencer et al. (1990) Theor. Appl. Genet. 79: 625; all of which are herein incorporated by reference), the hph gene which confers resistance to the antibiotic hygromycin (see, for example, Blochlinger and Diggelmann (1984) Mol. Cell. Biol. 4:2929; herein incorporated by reference in its entirety), and the dhfr gene, which confers resistance to methotrexate (see, for example, Bourouis et al. (1983) EMBO J., 2:1099; herein incorporated by reference in its entirety).

In some preferred embodiments, the vector is adapted for use in an Agrobacterium mediated transfection process (see, for example, U.S. Pat. Nos. 5,981,839; 6,051,757; 5,981,840; 5,824,877; and 4,940,838; all of which are incorporated herein by reference). Construction of recombinant Ti and Ri plasmids in general follows methods typically used with the more common bacterial vectors, such as pBR322. Additional use can be made of accessory genetic elements sometimes found with the native plasmids and sometimes constructed from foreign sequences. These may include but are not limited to structural genes for antibiotic resistance as selection genes.

There are two systems of recombinant Ti and Ri plasmid vector systems now in use. The first system is called the “cointegrate” system. In this system, the shuttle vector containing the gene of interest is inserted by genetic recombination into a non-oncogenic Ti plasmid that contains both the cis-acting and trans-acting elements required for plant transformation as, for example, in the pMLJ1 shuttle vector and the non-oncogenic Ti plasmid pGV3850. The second system is called the “binary” system in which two plasmids are used; the gene of interest is inserted into a shuttle vector containing the cis-acting elements required for plant transformation. The other necessary functions are provided in trans by the non-oncogenic Ti plasmid as exemplified by the pBIN19 shuttle vector and the non-oncogenic Ti plasmid PALA404. Some of these vectors are commercially available.

It may be desirable to target the nucleic acid sequence of interest to a particular locus on the plant genome. Site-directed integration of the nucleic acid sequence of interest into the plant cell genome may be achieved by, for example, homologous recombination using Agrobacterium-derived sequences. Generally, plant cells are incubated with a strain of Agrobacterium which contains a targeting vector in which sequences that are homologous to a DNA sequence inside the target locus are flanked by Agrobacterium transfer-DNA (T-DNA) sequences, as previously described (see, for example, U.S. Pat. No. 5,501,967, the entire contents of which are herein incorporated by reference). One of skill in the art knows that homologous recombination may be achieved using targeting vectors which contain sequences that are homologous to any part of the targeted plant gene, whether belonging to the regulatory elements of the gene, or the coding regions of the gene. Homologous recombination may be achieved at any region of a plant gene so long as the nucleic acid sequence of regions flanking the site to be targeted is known.

In yet other embodiments, the nucleic acids of the present invention is utilized to construct vectors derived from plant (+) RNA viruses (for example, brome mosaic virus, tobacco mosaic virus, alfalfa mosaic virus, cucumber mosaic virus, tomato mosaic virus, and combinations and hybrids thereof). Generally, the inserted plant GPAT5 polynucleotide of the present invention can be expressed from these vectors as a fusion protein (for example, coat protein fusion protein) or from its own subgenomic promoter or other promoter. Methods for the construction and use of such viruses are described in, examples, U.S. Pat. Nos. 5,846,795; 5,500,360; 5,173,410; 5,965,794; 5,977,438; and 5,866,785, all of which are incorporated herein by reference.

In some embodiments of the present invention, where the nucleic acid sequence of interest is introduced directly into a plant. One vector useful for direct gene transfer techniques in combination with selection by the herbicide Basta (or phosphinothricin) is a modified version of the plasmid pCIB246, with a CaMV 35S promoter in operational fusion to the E. coli GUS gene and the CaMV 35S transcriptional terminator (WO 93/07278).

c. Transformation Techniques for Expressing GPATs in Plants.

Once a nucleic acid sequence encoding a plant GPAT5 is operatively linked to an appropriate promoter and inserted into a suitable vector for the particular transformation technique utilized (for example, one of the vectors described above), the recombinant DNA described above can be introduced into the plant cell in a number of art-recognized ways. Those skilled in the art will appreciate that the choice of method might depend on the type of plant targeted for transformation. In some embodiments, the vector is maintained episomally. In other embodiments, the vector is integrated into the genome. In some embodiments, direct transformation in the plastid genome is used to introduce the vector into the plant cell (See for example, U.S. Pat. Nos. 5,451,513; 5,545,817; 5,545,818; PCT application WO 95/16783; all of which are herein incorporated by reference.)

The basic technique for chloroplast transformation involves introducing regions of cloned plastid DNA flanking a selectable marker together with the nucleic acid encoding the RNA sequences of interest into a suitable target tissue (for example, using biolistics or protoplast transformation with calcium chloride or PEG). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (see, for example, Svab et al. (1990) PNAS, 87:8526; Staub and Maliga, (1992) Plant Cell, 4:39; all of which are herein incorporated by reference). The presence of cloning sites between these markers allowed creation of a plastid targeting vector introduction of foreign DNA molecules (Staub and Maliga (1993) EMBO J., 12:601, herein incorporated by reference). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab and Maliga (1993) PNAS, 90:913, herein incorporated by reference). Other selectable markers useful for plastid transformation are known in the art and encompassed within the scope of the present invention. Plants homoplasmic for plastid genomes containing the two nucleic acid sequences separated by a promoter of the present invention are obtained, and are preferentially capable of high expression of the RNAs encoded by the DNA molecule.

In some embodiments, the vectors comprising a nucleic acid sequence encoding a plant GPAT5 of the present invention are transferred using Agrobacterium-mediated transformation (see, for example, Hinchee et al. (1988) Biotechnology, 6:915; Ishida et al. (1996) Nature Biotechnology 14:745; all of which are herein incorporated by reference).

Agrobacterium is a representative genus of the bacterial gram-negative family Rhizobiaceae. Its species are responsible for plant tumors such as crown gall and hairy root disease. In the dedifferentiated tissue characteristic of the tumors, amino acid derivatives known as opines are produced and catabolized. The bacterial genes responsible for expression of opines are a convenient source of control elements for chimeric expression cassettes. For transformation with Agrobacterium, disarmed Agrobacterium cells are transformed with recombinant Ti plasmids of Agrobacterium tumefaciens or Ri plasmids of Agrobacterium rhizogenes (such as those described in U.S. Pat. No. 4,940,838, the entire contents of which are herein incorporated by reference). The nucleic acid sequence of interest is then stably integrated into the plant genome by infection with the transformed Agrobacterium strain. For example, heterologous nucleic acid sequences have been introduced into plant tissues using the natural DNA transfer system of Agrobacterium tumefaciens and Agrobacterium rhizogenes bacteria (for review, see, for example, Klee et al. (1987) Ann. Rev. Plant Phys. 38:467-486; herein incorporated by reference in its entirety).

Heterologous genetic sequences (for example, GPAT nucleic acid sequences operatively linked to a promoter of the present invention), can be introduced into appropriate plant cells, by means of the Ti plasmid of Agrobacterium tumefaciens. The Ti plasmid is transmitted to plant cells on infection by Agrobacterium tumefaciens, and is stably integrated into the plant genome (Schell (1987) Science, 237: 1176). Species which are susceptible infection by Agrobacterium may be transformed in vitro. Alternatively, plants may be transformed in vivo, such as by transformation of a whole plant by Agrobacteria infiltration of adult plants, as in a “floral dip” method (Bechtold N, Ellis J, Pelletier G (1993) Cr. Acad. Sci. III-Vie 316:1194-1199). There are three common methods to transform plant cells with Agrobacterium. The first method is co-cultivation of Agrobacterium with cultured isolated protoplasts. This method requires an established culture system that allows culturing protoplasts and plant regeneration from cultured protoplasts. The second method is transformation of cells or tissues with Agrobacterium. This method requires (a) that the plant cells or tissues can be transformed by Agrobacterium and (b) that the transformed cells or tissues can be induced to regenerate into whole plants. The third method is transformation of seeds, apices or meristems with Agrobacterium. This method requires micropropagation.

One of skill in the art knows that the efficiency of transformation by Agrobacterium may be enhanced by using a number of methods known in the art. For example, the inclusion of a natural wound response molecule such as acetosyringone (AS) to the Agrobacterium culture has been shown to enhance transformation efficiency with Agrobacterium tumefaciens (see, for example, Shahla et al., (1987) Plant Molec. Biol. 8:291-298; herein incorporated by reference in its entirety). Alternatively, transformation efficiency may be enhanced by wounding the target tissue to be transformed. Wounding of plant tissue may be achieved, for example, by punching, maceration, bombardment with microprojectiles, etc. (see, for example, Bidney et al., (1992) Plant Molec. Biol. 18:301-313; herein incorporated by reference in its entirety).

In other embodiments, vectors useful in the practice of the present invention are microinjected directly into plant cells by use of micropipettes to mechanically transfer the recombinant DNA (Crossway (1985) Mol. Gen. Genet, 202:179). In still other embodiments, the vector is transferred into the plant cell by using polyethylene glycol (see, for example, Krens et al. (1982) Nature, 296:72; Crossway et al. (1986) BioTechniques, 4:320; all of which are herein incorporated by reference); fusion of protoplasts with other entities, either minicells, cells, lysosomes or other fusible lipid-surfaced bodies (see, for example, Fraley et al. (1982) Proc. Natl. Acad. Sci., USA, 79:1859; herein incorporated by reference in its entirety); protoplast transformation (EP 0 292 435); direct gene transfer (see, for example, Paszkowski et al. (1984) EMBO J., 3:2717; Hayashimoto et al. (1990) Plant Physiol. 93:857; all of which are herein incorporated by reference).

In still further embodiments, the vector may also be introduced into the plant cells by electroporation, (see, for example, Fromm, et al. (1985) Pro. Natl Acad. Sci. USA 82:5824; Riggs et al. (1986) Proc. Natl. Acad. Sci. USA 83:5602; all of which are herein incorporated by reference). In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus.

In yet other embodiments, the vector is introduced through ballistic particle acceleration using devices (for example, available from Agracetus, Inc., Madison, Wis. and Dupont, Inc., Wilmington, Del.). (See, for example, U.S. Pat. No. 4,945,050, and McCabe et al. (1988) Biotechnology 6:923; all of which are herein incorporated by reference). See also, Weissinger et al. (1988) Annual Rev. Genet. 22:421; Sanford et al. (1987) Particulate Science and Technology, 5:27 (onion); Svab et al. (1990) Proc. Natl. Acad. Sci. USA, 87:8526 (tobacco chloroplast); Christou et al. (1988) Plant Physiol., 87:671 (soybean); McCabe et al. (1988) Bio/Technology 6:923 (soybean); Klein et al. (1988) Proc. Natl. Acad. Sci. USA, 85:4305 (maize); Klein et al. (1988) Bio/Technology, 6:559 (maize); Klein et al. (1988) Plant Physiol., 91:4404 (maize); Fromm et al. (1990) Bio/Technology, 8:833; and Gordon-Kamm et al. (1990) Plant Cell, 2:603 (maize); Koziel et al. (1993) Biotechnology, 11:194 (maize); Hill et al. (1995) Euphytica, 85:119 and Koziel et al. (1996) Annals of the New York Academy of Sciences 792:164; Shimamoto et al. (1989) Nature 338: 274 (rice); Christou et al. (1991) Biotechnology, 9:957 (rice); Datta et al. (1990) Bio/Technology 8:736 (rice); European Patent Application EP 0 332 581, (orchardgrass and other Pooideae); Vasil et al. (1993) Biotechnology, 11: 1553 (wheat); Weeks et al. (1993) Plant Physiol., 102: 1077 (wheat); Wan et al. (1994) Plant Physiol. 104: 37 (barley); Jahne et al. (1994) Theor. Appl. Genet. 89:525 (barley); Knudsen and Muller (1991) Planta, 185:330 (barley); Umbeck et al. (1987) Bio/Technology 5: 263 (cotton); Casas et al (1993) Proc. Natl. Acad. Sci. USA 90:11212 (sorghum); Somers et al. (1992) Bio/Technology 10:1589 (oat); Torbert et al. (1995) Plant Cell Reports, 14:635 (oat); Weeks et al. (1993) Plant Physiol., 102:1077 (wheat); Chang et al., WO 94/13822 (wheat), and Nehra et al. (1994) The Plant Journal, 5:285 (wheat); all of which are herein incorporated by reference in their entirety.

d. Regeneration.

Whole plants were regenerated from transformed plant material selected for expression of the heterologous gene encoding a plant GPAT5 or GPAT4 or GPAT7 or other GPAT acyltransferase family gene. Plant regeneration from cultured protoplasts is described in Evans et al. (1983) Handbook of Plant Cell Cultures, Vol. 1: (MacMillan Publishing Co. New York) and Vasil I. R. (ed.), Cell Culture and Somatic Cell Genetics of Plants, Acad. Press, Orlando, Vol. I (1984), and Vol. III (1986), for e.g., all of which are herein incorporated by reference. It is known that many plants can be regenerated from cultured cells or tissues, including but not limited to major species of sugarcane, sugar beet, cotton, fruit and other trees, legumes and vegetables, and monocots (for example, the plants described above). Means for regeneration vary from species to species of plants, but generally a suspension of transformed protoplasts containing copies of the heterologous gene is first provided. Callus tissue is formed and shoots may be induced from callus and subsequently rooted.

Alternatively, embryo formation can be induced from the protoplast suspension. These embryos geminate and form mature plants. The culture media will generally contain various amino acids and hormones, such as auxin and cytokinins. Shoots and roots normally develop simultaneously. Efficient regeneration will depend on the medium, on the genotype, and on the history of the culture. The reproducibility of regeneration depends on the control of these variables.

e. Generation of Transgenic Lines and Cultivars.

Transgenic lines are established from transgenic plants by tissue culture propagation or other traditional methods for providing cultivars and lines comprising heterologous transgenes of the present invention.

The presence of nucleic acid sequences encoding exogenous a plant GPAT5 or GPAT or GPAT acyltransferase family of the present invention (including mutants or variants thereof may be transferred to related varieties by traditional plant breeding techniques.

These transgenic lines and/or cultivars are then utilized for evaluation of fatty acid and/or wax production and other agronomic traits.

VI. Production of Surface Lipid and/or Lipid Based Structures where not Normally Present.

In another aspect of the present invention, methods are provided for producing surface lipids in organisms and/or tissues where said surface lipids are not usually present or are present in very low levels in wild-type plants. In this aspect, surface lipids are produced in organisms transformed with a heterologous gene encoding a polypeptide exhibiting GPAT5 or any one of a GPAT or GPAT acyltransferase family activity and grown under conditions sufficient to effect production of surface lipids. In some embodiments, the methods comprise production of surface lipids in specific tissues or organs, such as in plant leaves or stems or seeds. In other embodiments, the methods comprise production of surface lipids at specific developmental phases. In yet other embodiments, the methods comprise production of surface lipids in specific tissues or organs and at specific developmental phases.

In this aspect, the surface lipids are contemplated to serve a physiological role. For example, the inventors contemplate that surface lipids may provide bacterial or fungal resistance in plant leaves. Thus, expression of surface lipids in plant leaves which normally do not possess free fatty acids and other fatty acid derivatives, or possess free fatty acids and other types of acyl fatty acid derivatives at insignificant levels, provides increased insect and/or bacteria and/or fungal resistance.

In some embodiments of the present invention, the methods comprise providing a transgenic organism comprising a heterologous gene encoding a plant GPAT5 or GPAT or GPAT acyltransferase family operably linked to an inducible promoter, and growing the transgenic organism either in the presence of the an inducing agent, or growing the organism and then exposing it to an inducing agent, thereby expressing GPAT5 or GPAT or GPAT acyltransferase family member resulting in the production of surface and/or novel fatty acid derivatives. In still other embodiments of the present invention, the methods comprise providing a transgenic organism comprising a heterologous gene encoding a plant GPAT5 or GPAT or GPAT acyltransferase family member operably linked to a promoter which is either tissue specific or developmentally specific, and growing the transgenic organism to the point at which the tissue is developed or the developmental stage at which the developmentally-specific promoter is activated, thereby expressing GPAT5 or GPAT or GPAT acyltransferase family member resulting in the production of surface and/or novel acyl fatty acid derivatives. Exemplary promoters include but are not limited to epidermal cell or leaf and stem specific promoters.

A heterologous gene encoding a plant GPAT5 or GPAT or GPAT acyltransferase family member, which includes mutants or variants of a plant GPAT5 or GPAT or GPAT acyltransferase family member, includes any suitable sequence of the invention as described above. Preferably, the heterologous gene is provided within an expression vector such that transformation with the vector results in expression of the polypeptide; suitable vectors are described above and following. Methods of producing transgenic organisms, and in particular transgenic plants, are described above.

### VII. Manipulation of Plant Fatty Acid Acyltransferase Activity in Plants.

As described above, certain fatty acids are considered to have negative consequences when expressed in transgenic plants. Examples are very long chain fatty acids which dramatically alter the morphology of plants when overproduced in plants using a constitutive promoter (see, for example, Millar et al (1998) Plant Cell 10:1889-902; herein incorporated by reference in its entirety). Therefore, the present invention provides methods to produce very long chain fatty acids and derivatives on the surface of the plant, thereby preventing their accumulation in the cell where consequences would be negative, as shown by Millar et al (1998) Plant Cell 10:1889-902; herein incorporated by reference in its entirety. These aspects of the invention have great utility in significantly expanding the range of fatty acid or fatty acid derivative structures that can be produced in transgenic plants.

A. Suberin and Cutin.

The inventors show herein, that a composition of Arabidopsis root waxes comprise MAGs with C22-C24 saturated acyl groups. Arabidopsis plants overexpressing an acyltransferase GPAT5 of the suberin biosynthesis pathway under the control of the 35S constitutive promoter demonstrated that (i) GPAT5 was involved in the synthesis of suberin polymer (see, Beisson et al. (2007) Plant Cell 19:351-368; herein incorporated by reference) and involved in the synthesis of the MAGs found in root waxes, (ii) that GPAT5 is an acyltransferase acting on a glycerol-based acceptor, and (iii) that the ectopic expression of this gene led to the production of MAGs in cuticular waxes of aerial organs.

Suberin and cutin are ubiquitous extracellular lipid polymers found in higher plants. Each polymer of these polymers was insoluble in organic solvents and often found in association with solvent extractable waxes. Suberin and its associated waxes form the suberin layer, which is often characterized by electron-translucent and electron-dense lamellae observed by TEM. Suberin is present in many external as well as internal tissues and has an important role in controlling water and solute fluxes. Its deposition is often induced by wounding or stress stimuli thereby providing a barrier against pathogen invasion (Lulai and Corsini, 1998, Physiol Mol Plant Pathol 53:209-222; Kolattukudy, 2001, Adv Biochem Eng/Biotechnol 71:1-49; Bernards, 2002, Can J Bot 80:227-240; all of which are herein incorporated by reference). Suberin was proposed to comprise of polyphenolic and poly-aliphatic domains (Bernards, 2002, Can J Bot 80:227-240; herein incorporated by reference). The term “aliphatic suberin” is used in this paper to describe the poly-aliphatic domain of the suberin. In contrast to cutin, depolymerization of suberin or suberin-rich tissues produces fatty acids, fatty alcohols, hydroxycinnamic acids and α,ω-dicarboxylic acids in addition to ω-hydroxy fatty acid monomers. Mid-chain functional groups are rare. Suberin often includes substantial amounts of saturated monomers with chain length >C20 (Kolattukudy, (1980) Cutin, suberin and waxes. In PK Stumpf, ed The Biochemistry of Plants—A Comprehensive Treatise. Vol 4, Academic Press, New York, pp 571-645; Kolattukudy (2001) Adv Biochem Eng/Biotechnol 71:1-49; Bernards, 2002, Can J Bot 80:227-240; Schreiber et al., 2005, Planta 220:520-530; all of which are herein incorporated by reference). Analyses of Arabidopsis tissues expected to be enriched in suberin, namely roots and seed coats, have shown typical suberin monomer compositions (Franke et al., 2006, Phytochemistry 66:2643-2658; Molina et al., 2006, Phytochemistry 67:2597-2610; Beisson et al., 2007, Plant Cell 19:351-368; all of which are herein incorporated by reference). A substantial portion of the hydroxycinnamic acids of suberin are believed to be crosslinked (Bernards et al., 1995, J Biol Chem 270:7382-7386; Bernards, 2002, Can J Bot 80:227-240; all of which are herein incorporated by reference). Besides aliphatic and aromatic components suberins contain a substantial proportion of glycerol (Holloway (1982) Structure and histochemistry of plant cuticular membranes: an overview. In D F Cutler, K F Alvin, C E Price, eds The Plant Cuticle. Academic Press, London, pp 1-32; Graça and Pereira, 1997, Holzforschung 51:225-234; Graca et al., 2002, Phytochemistry 61:205-215; all of which are herein incorporated by reference), the content of which is positively correlated to the suberization process (Moire et al., 1999, Plant Physiol 119:1137-1146; herein incorporated by reference). Partial chemical depolymerizations of bark and potato periderm suberins have yielded fragments which include monoacylglycerols (MAGs) of α,ω-dicarboxylic acids, ω-hydroxy fatty acids and fatty acids, diglycerol esters of α,ω-dicarboxylic acids, and an ω-ferulyloxy-acyl glycerol (Graça and Pereira, 1997, Holzforschung 51:225-234; 1999, Holzforschung 53:397-402, 2000, J Agr Food Chem 48:5476-5483; Graca and Santos, 2006, Biomacromolecules 7:2003-2010; Santos and Graça, 2006, Holzforschung 60:171-177; all of which are herein incorporated by reference). Unlike cuticular waxes and cutin, suberin waxes tend to reflect in part suberin polymer compositions.

Suberin-associated waxes have been studied mostly in the native and wound-healing periderm of plant subterranean storage organs, where the main constituents appear to be alkanes, primary alcohols, fatty acids and alkyl ferulates (Espelie et al., 1980; Planta 148:468-476; Bemards and Lewis, 1992, Phytochemistry 31:3409-3412; Schreiber et al., 2005, Planta 220:520-530; all of which are herein incorporated by reference). The suberin-associated waxes are considered a major contributor to the barrier for water diffusion across suberized cell walls (Soliday et al., 1979, Planta 146:607-614; herein incorporated by reference), but other factors controlling permeability remain to be identified (Schreiber et al., 2005, Planta 220:520-530; herein incorporated by reference). Green cotton fibers contain suberin and a significant amount of suberin-like waxes, including 1-(22-caffeyloxydocosanoyl)-glycerol as a major component (Schmutz et al., 1994, Phytochemistry 36:1343-1346; herein incorporated by reference).

A family of sn-glycerol-3-phosphate acyltransferase (GPAT) genes has been identified in Arabidopsis, five members of which gave detectable enzyme activity when expressed heterologously in a gat1Δ strain of yeast (Zheng et al., 2003, Plant Cell 15:1872-1887; herein incorporated by reference). The inventors recent characterization of a mutant gpat5, with a null-mutation in the GPAT5 acyltransferase gene (At3g11430), revealed its essential role in aliphatic suberin synthesis (Beisson et al., 2007, Plant Cell 19:351-368; herein incorporated by reference). The seeds of gpat5 plants have 50% of the wild type (WT) polyester load with large reductions in C20-C24 suberin-like aliphatic monomers, while roots from 1-week old seedlings have reductions in C20-C24 monomers. These findings are consistent with glycerol as a suberin monomer and offer the first clue as to how glycerol might be incorporated into the polyester network. However, the exact biochemical function of the GPAT5 enzyme remains unknown.

The inventors report herein on the characterization of root waxes in the model plant Arabidopsis thaliana and show that they have a distinct composition and contain MAGs. The inventors demonstrated that accumulation of MAGs is positively correlated with the expression of GPAT5, indicating a common pathway for the biosynthesis of aliphatic suberin and some of the suberin-associated waxes. Furthermore, the inventors show that ectopic expression of GPAT5 under the control of the CaMV 35S promoter leads to production of α- and β-isomers of MAGs as novel components of leaf and stem surface waxes, changing significantly the WT composition and morphology of aerial cuticle.

Cutin and its cuticular and epicuticular waxes form the cuticle layer covering all aerial organs of plants. The cuticle protects plants from biotic and abiotic stresses, limits gas and water exchange, and is likely involved in developmental processes during plant growth (Kolattukudy, 2001, Adv Biochem Eng/Biotechnol 71:1-49; Nawrath, 2006, Plant J 40:920-930; all of which are herein incorporated by reference). Usually, cutin polyesters are composed largely of C16 and C18 co-hydroxy fatty acid monomers, which often have mid-chain functionality such as epoxy, secondary hydroxyl or vicinal diol groups (Kolattukudy, 2001 Adv Biochem Eng/Biotechnol 71:1-49; Nawrath, 2002, Curr Opin Plant Biol 9:281-287; all of which are herein incorporated by reference). Glycerol is a monomer and was shown to become esterified to co-hydroxy fatty acids (Graça et al., 2002, Phytochemistry 61:205-215; herein incorporated by reference). In Arabidopsis and Brassica napus the polyesters of leaf and stem epidermis and isolated cuticles are unusual in that they contain high proportions of α,ω-dicarboxylic acids, and particularly α,ω-dicarboxylic acid derived from linoleic acid (Bonaventure et al., 2004, Phytochemistry 61:205-215; Franke et al., 2006, Phytochemistry 66:2643-2658; all of which are herein incorporated by reference). The cuticular waxes are derived from very-long-chain (C22-C34) saturated fatty acids. Alkanes are common in cuticular waxes in addition to a wide variety of neutral lipids (Kolattukudy, 1980, Cutin, suberin and waxes. In PK Stumpf, ed The Biochemistry of Plants—A Comprehensive Treatise. Vol 4, Academic Press, New York, pp 571-645; Jetter et al., 2006, Composition of plant cuticular waxes. In M Riederer, C Müller, eds, Biology of the Plant Cuticle, Annual Plant Reviews Vol 23 Blackwell Publishing Ltd, Oxford, pp 145-175; all of which are herein incorporated by reference). Chemical genetics of plant cuticular waxes was extensively studied as shown in Kunst and Samuels, 2003, Prog Lipid Res 42:51-80; herein incorporated by reference in its entirety). In particular, WT Arabidopsis stem epicuticular waxes were dominated by nonacosane and its 14-hydroxy, 15-hydroxy and 15-oxo-derivatives and contain smaller amounts of free fatty acids (FFA), aldehydes, primary alcohols and wax esters (Rashotte et al., 2001, Phytochemistry 57:115-123; herein incorporated by reference).

Thus, despite their co-occurrence there are few structural similarities between cuticular waxes and cutin monomers.

B. Altering Surface Lipids.

The inventors further contemplated that the nucleic acids encoding a plant acyltransferase, such as GPAT5, GPAT or GPAT acyltransferase family member, of the present invention may be utilized to either increase or decrease the level of plant GPAT5 or GPAT or GPAT acyltransferase family member mRNA and/or protein in transfected cells as compared to the levels in wild-type cells. Such transgenic cells have great utility, including but not limited to providing designer oils, designer plant surfaces, increasing pathogen resistance, controlling water loss, increasing economic utility, and for further research as to the effects of the overexpression of plant GPAT5 or GPAT or GPAT acyltransferase family member.

The inventors demonstrate herein, some effects from overexpressing a plant acyltransferase, for example, GPAT5, GPAT4, GPAT7, and GPAT8. Accordingly, in some embodiments, expression in plants by the methods described above leads to the overexpression of GPAT5 or GPAT7 or GPAT8 in plants, plant tissues, and plant cells. The inventors further contemplate overexpression of other GPAT or GPAT acyltransferase family member in transgenic plants, plant tissues, and plant cells. Such over-expression of a GPAT5 or GPAT or GPAT acyltransferase family member provides extracellular lipids as described by exemplary examples herein.

The inventors demonstrate herein, some effects from underexpression or lack of GPAT5 or GPAT4 or GPAT8 in plants. The inventors further contemplate underexpression or lack of other GPAT or GPAT acyltransferase family members in plants. Accordingly, in other embodiments of the present invention, the inventors contemplate expression of gene silencing vectors, comprising antisense sequences comprising GPAT5 or GPAT or GPAT acyltransferase family member in plants by methods described herein, leading to the underexpression or lack of plant GPAT5 or GPAT or GPAT acyltransferase family member polypeptides in transgenic plants, plant tissues, or plant cells. Specifically, exemplary methods are provided for silencing a plant GPAT5 or GPAT or GPAT acyltransferase family member for altering extracellular lipids in a seed, root, stem, or tuber. In particular, the inventions provide compositions and methods for altering extracellular lipid in a tuber, otherwise known as an “underground” stem. For example, the inventors contemplate that it would be desirable to increase or reduce the thickness of a potato skin (examples of suberin content described in Yu et al., 2006, Biomacromolecules 7:937-944; herein incorporated by reference), such that the suberin content, for example is increased or reduced. As another example, the inventors contemplate that it would be desirable to increase or reduce suberin layers in cotton (Moire et al., 1999, Plant Physiology, 119:1137-1146; herein incorporated by reference), such that the suberin content (for example, is increased or reduced). Thus, in one embodiment, expression of a plant GPAT5 or GPAT or GPAT acyltransferase family member is lowered for providing a thinner skinned potato (such as by antisense methods, for example, Diretto et al., 2006, BMC Plant Biology 6:13; herein incorporated by reference). In other embodiments, expression of a plant GPAT5 or GPAT or GPAT acyltransferase family member is increased for providing a thicker skinned potato. In yet other embodiments, the suberin layer is altered for providing increased protection of the potato during harvest. In yet other embodiments, the suberin layer is altered for providing increased protection of the potato during storage.

### VIII. Functions of GPAT5 in Lipid Metabolism.

Analysis of the fatty acid derivatives found in seeds, roots, and flowers showed that GPAT5 was required for the synthesis of lipid polyesters. Moreover, in these organs and tissues, the expression of the gene was restricted (e.g., in the root the gene was expressed in the differentiation zone but not in the division and elongation zones that must actively synthesize membrane lipids). These results indicated that GPAT5 is not likely to be a housekeeping gene of fatty acid metabolism that indirectly affects lipid polyester composition, like, for example, the FatB gene (Bonaventure, et al. (2004) Plant J. 40:920-930; herein incorporated by reference). A contemplated role for a GPAT family member in lipid polyester synthesis is further supported by two other observations, described as follows. First, a GPAT (GPAT6; At2g38110) was among the few genes upregulated by the overexpression of the transcription factor WIN1 that is specifically required for cuticle formation (Broun (2004) Proc. Natl. Acad. Sci. USA 101: 4706-4711; herein incorporated by reference). Second, the inventors found that at least two other GPATs were required for cutin synthesis and normal cuticle impermeability in leaves.

The pathways of the synthesis, transport, and assembly of plant extracellular lipid polymers are poorly understood. Central to polyester synthesis is fatty acid ω-oxidation to form fatty hydroxy and dicarboxylic acids. The reactions of fatty acid oxidation and some partially characterized enzymes (cytochrome P450 monooxygenases, etc.) that may be involved in these oxidation processes have been described (Kolattukudy, et al. (2001) Adv. Biochem. Eng. Biotechnol. 71:1-49; herein incorporated by reference), but the in vivo substrates of the oxidation enzymes are not known and might be acyl-CoAs, free fatty acids, etc. After the oxidation steps, the monomers are assembled by the polyester synthase(s) that is, the enzyme(s) responsible for the synthesis of the primary ester bonds in the polyester chain. The presence of glycerol in cutin and suberin raises the possibility that acylglycerols are substrates for oxidation and/or polymerization reactions. Glycerol 3-phosphate acyltransferase (EC 2.3.1.15) catalyzes the transfer of an acyl group to glycerol 3-phosphate to form lysophosphatidic acid (Murata, et al. (1997) Biochim. Biophys. Acta 1348: 10-16; herein incorporated by reference). Because Arabidopsis GPATs were isolated by structural similarity to yeast glycerol-phosphate acyltransferases and because most of the members, including GPAT5, have been shown to be able to catalyze the transfer of acyl chains from acyl-CoA to glycerol-3-phosphate to form lysophosphatidic acid (Zheng, et al. (2003) Plant Cell 15: 1872-1888; herein incorporated by reference), the inventors further contemplate that GPAT5 functions to provide lysophosphatidic acid for the oxidation or assembly machinery of suberin synthesis. However, because other acyl acceptors than glycerol-3-phosphate and other acyl donors than acyl-CoAs were not tested GPAT5 activity, it cannot be excluded that in vivo glycerol could be an acyl acceptor and hydroxy acyl-CoA could be an acyl donor. Direct acylation of glycerol has indeed been observed in animal cells (Hanel, et al. (1995) Biochemistry 34: 7807-7818; Lee, et al. (2001) J. Lipid Res. 42: 1979-1985, all of which are herein incorporated by reference). Thus, the reaction catalyzed by GPAT5 could occur before and/or after the oxidation steps. Other possibilities are less likely but cannot be ruled out (e.g., GPAT5 may be a polyester synthase). Therefore, based on the previous biochemical characterization of GPAT5 and the results described herein, the contemplated molecular function of GPAT5 is summarized as follows: (1) formation of unsubstituted fatty acid-containing acylglycerols to feed the oxidation machinery; (2) formation of oxidized fatty acid-containing acylglycerols that may be used as acyl carriers and/or substrates for the polymerization reactions; and (3) addition of acyl chains to a growing glycerol-containing lipid polyester network. These possibilities can be fitted into the tentative metabolic pathway for suberin biosynthesis described by Bernards ((2002) Can. J. Bot. 80:227-240; herein incorporated by reference).

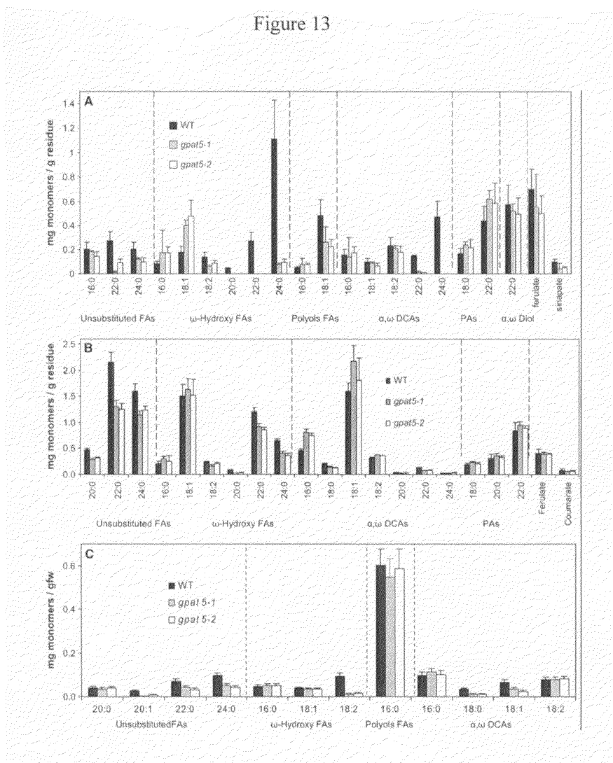

Regarding a contemplated acyl chain specificity of GPAT5, the inventors observed a common feature of the polyester monomer profiles in seeds, roots, and flowers of the gpat5 mutants of a strong reduction (at least twofold) observed in 22:0/24:0 fatty acids and derivatives (FIG. 13). Therefore, the inventors further contemplate that a role of GPAT5 is to provide a polyester synthesis pathway with acylglycerols containing 22:0 and 24:0 fatty acids and oxidized derivatives. The inventors' contemplate a specificity of GPAT5 for C22-C24 acyl chains is strongly supported by the observation that ectopic overexpression of GPAT5 in Arabidopsis results in the accumulation of saturated very long chain fatty acids attached to glycerol.

The inventors observed decreased amounts of very long chain fatty acids in lipids isolated from gpat5 mutant plant parts. Corresponding to this decrease were observed increased and decreased amounts of C16 and C18 monomers in different plant parts, such as in roots, seeds, and flowers. Indeed, the aliphatic monomers that were found are not part of independent simple lipid molecules but are structurally linked to each other in a complex manner, possibly a network in which glycerol and other monomers such as dicarboxylic acids allow cross-linking. Therefore, the observed decrease in very long chain fatty acids resulting from the loss of GPAT5 activity is contemplated to influence the incorporation of C16 and C18 monomers in different ways in seeds, roots, and flowers, depending on the following conditions: (1) the respective structures and compositions of aliphatic suberin in these organs, (2) the necessity of incorporation of GPAT5-provided long chain monomers before some C16-C18 chain monomers (sequential order requirement), and (3) the over incorporation of C16-C18 monomers wherever possible to maintain some structural features of the polymer (compensatory mechanism). Complex effects on polymer composition involving a sequential order requirement and a compensatory mechanism have been observed in xyloglucan chains of mutants affected in the incorporation of a specific sugar monomer (Madson, et al. (2003) Plant Cell 15:1662-1670; herein incorporated by reference).

### EXPERIMENTAL

The following examples serve to illustrate certain embodiments and aspects of the present invention and are not to be construed as liming the scope thereof.

In the experimental disclosures which follow, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); pg (picograms); L and l (liters); ml (milliliters); μl (microliters); cm (centimeters); mm (millimeters); μm (micrometers); nm (nanometers); U (units); d (day); h (hour); min (minute); s and sec (second); k (kilometer); deg (degree); ° C. (degrees Centigrade/Celsius); RT (room temperature); v/v (volume/volume); w/v (weight/volume); fw (fresh weight); gfw (gram fresh weight) eV (electron Volts); and amu (atomic mass unit).

### Example I

This example describes general materials and methods used in the development of the present inventions.

Plant Materials and Growth Conditions. Arabidopsis thaliana plants were ecotype Columbia-0 (Col-0), GPAT overexpression Arabidopsis thaliana plant lines (OE-1 and OE-2) were on the Col-0 background, and gpat T-DNA knockout lines (gpat5-1 and gpat5-2) were on the Columbia-0 background. The gpat5-1 and gpat5-2 mutants were isolated and characterized as described herein and in Beisson et al. ((2007) Plant Cell 19: 351-368; herein incorporated by reference).

Arabidopsis plants were grown in a controlled growth chamber at 21-22° C., 40-60% relative humidity (R^{H}), under a 16/8 h light/dark cycle (photoperiod) a light intensity of 80-100 μmol m⁻² s⁻¹ provided by fluorescent bulbs. Plants were grown in pots on a soil mixture (1:1:1 mixture of peat moss-enriched soil:vermiculite:perlite) or on solidified agar sucrose medium containing MS salts (Murashige and Skoog, 1962, Physiol Plant 15: 473-497, herein incorporated by reference)), 1% (w/v) sucrose, and 0.8% (w/v) Phytablend agar (Caisson Laboratories) adjusted to pH 5.7 using KOH.

Seeds grown on agar sucrose plates were surface-sterilized for 5 min in 70% (v/v) ethanol and rinsed three times with sterile water. For observation of root growth, seeds were grown on vertical agar plates.

Tobacco (Nicotiana tabacum var SR1). Tobacco plants were grown in an air-conditioned greenhouse with natural light.

Mutant Isolation. T-DNA insertional lines (SALK_{—}018117 and SALK_{—}142456) were identified using the SIGnAL T-DNA Express Arabidopsis Gene Mapping Tool (http://signal.salk.edu/cgi-bin/tdnaexpress; herein incorporated by reference) provided by the Salk Institute Genomic Analysis Laboratory (Alonso, et al. (2003) Science 301: 653-657; herein incorporated by reference). SALK_{—}018117 has a T-DNA insertion at the first exon, with the left border of the T-DNA pointing toward the 5′ end of the gene; SALK_{—}142456 has a T-DNA insertion in the first and only intron, with the T-DNA left border pointing to the 3′ end of the gene. Individual seeds for these lines were obtained from the ABRC at Ohio State University. Plants were grown from these seeds, and for each line DNA was prepared and used for genotype screening. The gene specific primers used for the screening of insertions into the GPAT5 gene were 5′-GCTATTTTTCCATTTGCAGATACGT-3′ (forward, SEQ ID NO:89) and 5′-ACATCTCGGATTCTTGTCAATC-3′ (reverse, SEQ ID NO:90) for SALK_{—}018117 and 5′-CTAAGGAGCATCTTAGAGCAGATGA-3′ (forward, SEQ ID NO:91) and 5′-TCCAGCGAGAACCCTATACTTATCT-3′ (reverse, SEQ ID NO:92) for SALK_{—}142456. These primers were used together with the T-DNA left border primer LBa1 (5′-TGGTTCACGTAGTGGGCCATCG-3′, SEQ ID NO:93) to check for the presence of a wild-type or T-DNA mutant allele, respectively.

RNA Isolation and Gene Transcript Analysis by RT-PCR. Total RNA was prepared from rosette leaves, stems, open flowers, roots, and developing seeds of Arabidopsis plants by grinding these tissues in liquid nitrogen followed by RNA extraction with the Plant RNeasy Mini kit from Qiagen. Extracted RNA was quantified by spectrophotometer, and its integrity was verified by separating 1 mg of total RNA on a denaturing formaldehyde gel. RNA was stored at 80° C. until use in reverse transcription reactions. To eliminate any contaminating DNA, RNA preparations were treated with DNase using the DNA-free kit from Ambion, and the treated RNA (1 to 5 mg) was subjected to reverse transcription using the SuperScript III first-strand synthesis system for RT-PCR (Invitrogen). The GPAT5 transcript was amplified using the gene-specific primers 5′-CTAAGGAGCATCTTAGAGCAGATGA-3′ (forward, SEQ ID NO:94) and 59-TCCAGCGAGAACCCTATACTTATCT-3′ (reverse, SEQ ID NO:95), and the GPAT7 transcript was amplified using the gene-specific primers 5′-CCTTCGCCTACTTCATGCTC-3′ (forward, SEQ ID NO:96) and 5′-GGTCTCGGGTTCATGAAAAA-3′ (reverse, SEQ ID NO:97), with the initiation factor eIF4A-1 (At3g13920, SEQ ID NO:94) as a control (forward, SEQ ID NO:98) 5′-CCAGAAGGCACACAGTTTGATGCA-3′; (reverse, SEQ ID NO:99) 5′-TCATCATCACGGGTCACGAAATTG-3′.

GPAT5 Promoter-GUS Fusion Analysis. A 1-kb sequence upstream of the first ATG in the GPAT5 cDNA, and 588 bp of the GPAT5 coding region, were cloned in-frame as a HindIII-XbaI fragment into the binary vector pBI101.1 carrying the GUS gene downstream of the inserted promoter. The primers used for amplification of the fragment from genomic DNA were 5′-CACACAAGCTTAAAAAAGCGTTTTAATTAG-3′ (forward, SEQ ID NO:100) and 5′-CACACTCTAGACTCACATAACGATAAGAA-3′ (reverse, SEQ ID NO: 101) (inserted restriction sites are underlined). The construct (ProGPAT5:GUS) was used to transform Arabidopsis wild-type plants by Agrobacterium tumefaciens vacuum infiltration (Bechtold et al., 1993, C. R. Acad. Sci. 316: 1194-1199; herein incorporated by reference). T1 seeds of ProGPAT5:GUS transformants were selected on sterile plates (50 mg/mL kanamycin) from surface-sterilized seeds. Resistant seedlings were transferred to soil for continued growth and seed collection. The T3 progeny from several individual kanamycin-resistant plants were analyzed for GUS gene expression. Briefly, fresh tissues were suspended and vacuum-infiltrated in a staining solution (0.1 M NaH₂PO₄, pH 7.0, 10 mM Na₂-EDTA, 0.5 mM K-ferricyanide, 0.5 mM K-ferrocyanide, 1.0 mM X-glucuronide prepared freshly each time in DMSO, and 0.1% Triton X-100 for 30 min to 1 h depending on the tissue) (Jefferson, et al. (1987) Plant Mol. Biol. Rep. 5: 385-405; herein incorporated by reference). The samples were then incubated at 37° C. for 2 to 18 h until staining was visible. Stained tissues were fixed in a mixture of ethanol:acetic acid:formaldehyde (50:5:3.7, v/v) for 10 min at 65° C. and destained in 80% (v/v) ethanol until removal of chlorophyll. Images of whole mount tissues were taken with a Leica MZ 12.5 microscope coupled with a digital camera.

Seed Germination and Dormancy Tests. Germination rates of wild-type and gpat5 seeds were compared for both soil-grown seeds and seeds grown on agar plates supplemented with increasing concentrations of salts. Plates were transferred to a controlled growth chamber (see Plant Materials and Growth Conditions) after cold treatment in the dark for 3 d at 4° C. Germination was scored at 7 d after transfer to the growth chamber (12 d in the case of added salts). Seeds that showed penetration of the radicle through the seed coat were counted as germinated seeds. Seedling establishment was scored by looking for the presence of two green cotyledons. The tests were repeated on at least three different batches of seeds harvested from plants grown at different times.

For dormancy release experiments, wild-type and gpat5 seeds harvested immediately after maturity from plants of the same age and grown under the same conditions were tested over a period of dry storage (0, 10, 17, 21, and 30 d at room temperature) (Bentsink, et al. (Apr. 4, 2002) Seed dormancy and germination. In The Arabidopsis Book. C. R. Somerville and E. M. Meyerowitz, eds (Rockville, Md.: American Society of Plant Biologists), doi/10.1199/tab.0050, www., followed by, aspb.org/, followed by, publications/arabidopsis/; herein incorporated by reference). Mature seeds were sown in triplicate in Petri dishes (100 to 150 seeds per plate), incubated in a controlled growth chamber, and scored for germination as described above. The tests were repeated on three different batches of seeds harvested from plants grown at different times.

Staining Procedures. For seed coat permeability tests, tetrazolium red assays were used (Debeaujon, et al. (2000) Plant Physiol. 122:403-414; herein incorporated by reference). Briefly, Arabidopsis dry seeds were incubated in the dark in an aqueous solution of 1% (w/v) tetrazolium red (2,3,5-triphenyltetrazolium) at 30° C. for 4 to 48 h. Mucilage of Arabidopsis mature seeds was stained in an aqueous solution of 0.03% (w/v) ruthenium red for 15 min at room temperature (Western et al., 2000, Plant Physiol. 122: 345-356; herein incorporated by reference). Seeds were rinsed in water before imaging.

For lipid polyester staining of roots, a solution of 1% (w/v) Sudan black in 75% ethanol was used. Roots were placed into this solution for 1 h, rinsed briefly in water, and imaged.

For lipid polyesters of seed coats, mature seeds were incubated for 24 h at room temperature in water containing 0.01% (w/v) Triton X-100 and 10% (v/v) commercial bleach to fade the seed coat pigments. After rinsing successively with distilled water and 100% ethanol, seeds were incubated for 30 min with chloroform:methanol (2:1, v/v), rinsed with 100% ethanol, and air-dried. Seeds were finally incubated at room temperature for 1 to 4 h with a solution of Sudan red 7B in polyethylene glycol 400:glycerol:water prepared as described by Bundrett et al. (1991) Biotech. Histochem. 66:111-116; herein incorporated by reference, rinsed in water, mounted between slide and cover slip, and observed with a Leica MZ12.5 light microscope coupled to a digital camera.

Analysis of Fatty Acids and Cuticular Waxes. Total fatty acid content and composition in Arabidopsis seeds were quantified according to L1, et al. (2006) Phytochemistry 67:904-915; herein incorporated by reference. The same method of direct methylation was used for total fatty acids of leaf, root, and flower. For cuticular waxes, the leaf tissue (1 g from 5-week-old plants) was dipped in chloroform (20 mL) for 30 s, with the addition of internal standards (20 mg/g n-octacosane, 10 mg/g docosanoic acid, and 10 mg/g fresh leaf 1-tricosanol). The epicuticular waxes were silylated to convert free alcohols and carboxylic acids to their trimethylsilyl ethers and esters, respectively, by heating the sample at 110° C. for 10 min in 100 mL of pyridine and 100 mL of N,O-bis(trimethylsilyl)trifluoroacetamide. After cooling, the solvent was evaporated under nitrogen and the product was resuspended in heptane:toluene (1:1, v/v) for gas chromatography-mass spectrometry analysis (Bonaventure, et al. (2003) Plant Cell 15: 1020-1033; herein incorporated by reference). For seed surface wax analysis, mature seeds (100 mg) were soaked in chloroform for 2 min, and internal standards (5 mg/g n-octacosane, 5 mg/g docosanoic acid, and 5 mg/g seed 1-tricosanol) were added and processed as described above for leaf cuticular wax analysis.

Seed surface areas were calculated assuming a prolate spheroid geometry and using semiaxes estimated from scanning electron microscopy images of seeds.

Analysis of Lipid Polyesters. Polyester monomers were obtained and analyzed according to Bonaventure et al. (2004) Plant J. 40:920-930; herein incorporated by reference. Briefly, the sodium methoxide depolymerization method was used with slight modifications described by Suh et al. (2005) Plant Physiol. 139:1649-1665; herein incorporated by reference. Polyester monomers were separated, identified, and quantified by gas chromatography-mass spectrometry. Splitless injection was used, and the mass spectrometer was run in scan mode over 40 to 500 atomic mass units (electron impact ionization), with peaks quantified on the basis of their total ion current. Polyester monomer amounts are expressed per surface area for seeds and per gram of dry residue depolymerized for roots. Further details of aliphatic and aromatic monomer identifications, analytical methods, and seed coat and embryo polyester monomer localization experiments are presented by Molina et al. (2006) Phytochemistry 67:2597-2610; herein incorporated by reference.

Analysis of PAs. Analysis was performed on samples of 15 mg of mature seeds using acid catalyzed depolymerization and spectrophotometric quantification as described previously (Routaboul, et al. (2006) Planta 224:96-107; herein incorporated by reference).

Scanning Electron Microscopy and Fluorescence Microscopy. Arabidopsis pollen and mature seeds were coated with gold particles during 3 min at a coating rate of 7 nm/min using an EMSCOPE SC500 sputtercoater (Ashford). Scanning electron microscopy images were taken with a JEOL JSM-6400V microscope. Assuming prolate spheroid seed geometry, semiaxes were estimated from scanning electron microscopy images using Analysis PRO software version 3.2 (Soft Imaging System). For seed coat autofluorescence, mature seeds were illuminated and observed with a Zeiss Axiophot microscope using epifluorescence interference and absorption filters (excitation filter, 365 nm; dichromatic beam splitter, 395 nm; long-pass emission filter, 435 nm) coupled to a digital camera.

Stem epidermal peels. Epidermis was collected as a thin transparent film (layer) under a dissecting microscope using sharp forceps to peel off the outer layer of epidermis (Suh et al., 2005, Plant Physiol 139:1649-1665; herein incorporated by reference).

Scanning Electron Microscopy Analysis: Epicuticular waxes were viewed using sections of stems treated in 1% (w/v) osmium tetroxide vapor for 24 h, air dried for 3 d, mounted onto standard aluminum stubs for JEOL SEM, and then sputter coated with around 30 nm of gold using an EMSCOPE SC-500 sputter coater (Kent, UK). Images were obtained with a JEOL 6400V scanning electron microscope (Tokyo, Japan).

Sudan Red 7B Staining and Microscopy: Sudan Red 7B (Sigma) was prepared as a 0.05% (w/v) solution in PEG400:glycerol (1:1, v/v) (Brundrett et al., 1991, Biotech Histochem 66:111-116; herein incorporated by reference in its entirety). Soil-grown Arabidopsis roots were stained in this solution for 1 h at RT, rinsed briefly in distilled water, and free-hand sections were made at the base of the roots with a razor blade. Images were taken with a Leica MZ 12.5 microscope.

### Example II

This example describes extraction and recovery of free fatty acids, acylglycerols and other hydrocarbon derivatives from transgenic plants and plant parts such as stems and seeds.

Cuticular Wax Analysis. Stems were dipped in chloroform for 30 s, the solvent evaporated under a stream of N₂ gas, and tricosane, tricosanoic acid, monoheptadecanoin and tridodecanoin added as internal standards. The waxes were derivatized by heating at 110° C. for 10 min in pyridine:BSTFA (N,O-bis-(trimethylsilyl)trifluoroacetamide) (1:1, v/v). The silylated sample was analyzed by GC using a 30 m DB5-ht capillary column temperature programmed at 10° C. min-1 to 370° Celcius. Eluate components were quantified based on uncorrected peaks areas resulting from an integrated FID ion current. For molecular identification, a Hewlett-Packard 5890 GC-coupled MSD 5972 mass analyzer was used, with the mass analyzer set in electron impact mode (70 eV) and scanning from 40-700 atomic mass units (amu).

Tobacco Leaf Epicuticular Wax Analysis. Waxes were collected as described above except that leaves were dipped in dichloromethane instead of chloroform (Severson et al., 1984, J Agric Food Chem 32: 566-570; herein incorporated by reference in its entirety).

Root Wax Analysis: Arabidopsis roots were carefully and thoroughly washed in distilled water, blotted then air dried at 50° C. for 30 min, and dipped in chloroform for 1 min, unless otherwise stated. The extracts were passed through a glass wool plugged column and evaporated to dryness under a stream of N₂ gas. The waxes were derivatized and analyzed as described above for cuticular waxes. Due to the complex architecture of the roots, it was not practical to calculate the root wax load based on surface area; therefore root wax load is reported as μg/g fresh weight. The inventors observed that after a comparison of root biomass between WT, mutant and 35S::GPAT5 overexpression lines there was no obvious differences observed in morphology, nor did the average epidermal root diameter (measured close to the crown) vary significantly between lines, suggesting that per g fresh weight units are approximately proportional to surface area units.

Additional Lipid Analyses. Fatty acid content and composition of Arabidopsis tissues was directly analyzed by acidic transmethylation according to (Li et al., 2006, Phytochemistry 67:904-915, herein incorporated by reference). For polyester analysis the NaOMe depolymerization protocol from Bonaventure et al., (2004, Plant J 40:920-930, herein incorporated by reference) was used with slight modifications as described in Suh et al., (2005, Plant Physiol 139:1649-1665, herein incorporated by reference). Polyester monomers were separated, identified and quantified by GC-MS. The mass spectrometer was run in scan mode (40-500 amu) with peaks quantified on the basis of their total ion current. Polyester monomer amounts were expressed per g of solvent-extracted dry residue.

Lipids collected from whole plants and whole plant parts. Plant Parts: Lipids were collected from extracellular surfaces of plants and extracellular surfaces of plant parts using the following methods. Plant parts: leaves, stems, siliques, etc. from overexpressor transgenic plants were dipped in chlororform or another organic solvent such as dichlromethane for 30 s at room temperature. The lipid extract was dried and treated with acidic methanol or other typical methylation reagents to form methyl esters of fatty acids or esters by transesterification. The fatty acid and fatty acid derivatives and hydrocarbon chains of interest were separated from glycerol and other compounds by classical phase partitioning (e.g. extraction with hexane) and fractionation techniques.

Whole Seeds: Seeds were ground with mortar and pestle followed by a soluble lipid extraction comprising using organic solvent for extracting the oil together with other soluble seed lipophilic compounds. The residue remaining after soluble lipid extraction was dried and treated with acidic methanol to form methyl esters of fatty acids or esters by transesterification. The hydrocarbon chain and lipids of interest were separated from glycerol and other compounds by classical phase partitioning (e.g. extraction with hexane) and fractionation techniques.

Statistics. Statistical tests were performed using Microsoft Excel and Analyze-it (version 1.73) software.

### Example III

This example describes the materials and methods used in generating transgenic plants of the present invention. This example further describes creating and analyzing plants with overexpressed GPAT5 using an exemplary constitutive promoter, CaMV35S.

### Cloning of GPAT5 and Construction of 35S::GPAT5 Overexpression Plants.

Genomic DNA was prepared from Arabidopsis leaf tissue using a Plant miniDNA kit according to manufacturer instructions (Qiagen). Genomic DNA sequences encoding a GPAT5 gene (see, genomic At3g11430 SEQ ID NO:1) were amplified by PCR using forward primer [5′-CACACTCTAGAATGGTTATGGAGCAAGC-3′ (underlined as added XbaI restriction site) SEQ ID NO:68] and reverse primer [CACACGAGCTCTCAATGGAGACAAGG (underlined as added SacI restriction site) SEQ ID NO:69]. The PCR product was initially cloned into pGEM-T easy vector (pGEM®-T Easy Vector Systems Technical Manual No. 042 Revised June 1999), and then subcloned as an XbaI-SacI fragment into binary vector pBI121 (accession number AF485783 SEQ ID NO:70 and 88 (contains a 35S promoter (AF485783 SEQ ID NO:83 (see, e.g., Chen et al, (2003) Molecular Breeding 11:287-293; herein incorporated by reference in its entirety), and a GUS gene) which replaced the GUS gene.

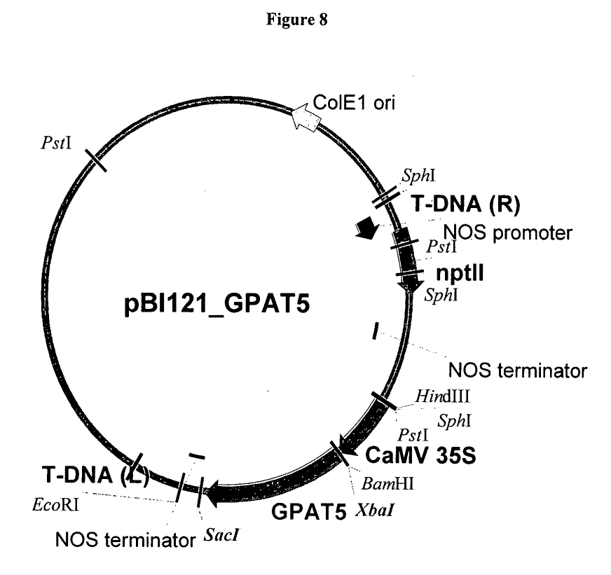

Arabidopsis plants: The construct (pBI121-GPAT5 otherwise known as 35S::GPAT5) (see, FIG. 8) was transfected into Agrobacterium tumefaciens strain C58C1 then transfected into Arabidopsis plant tissue by vacuum infiltration methods (for example, see, Bechtold et al. (1993) Life Sciences 316:1194-1199; herein incorporated by reference in its entirety) of Arabidopsis Col-0 WT plants.

Tobacco plants: For tobacco leaf disk transformation 35S::GPAT5 was transfected into Agrobacterium tumefaciens strain LBA4404 (for example, see, Rogers et al., 1986, Methods in Enzymology 118:627-640, herein incorporated by reference) then used to transform tobacco plant parts via Agrobacterium tumefaciens vacuum infiltration.

Transgenic (35S::GPAT5) plants were then selected on solidified agar sucrose medium containing MS salts Murashige and Skoog, 1962, Physiol Plant 15: 473-497, herein incorporated by reference, 1% (w/v) sucrose, 0.8% (w/v) Phytablend agar (Caisson Laboratories Inc., Rexburg, Id.) and adjusted to pH 5.7 using KOH containing 50 μg/ml-1 kanamycin.

The promoter used for driving GPAT5 overexpression, in other words a promoter in operable combination with GPAT5, was a CaMV 35S from the vector pBI121 (vector, including CaMV 35S promoter, purchased from ClonTech) (see, for example, Chen et al, (2003) Molecular Breeding 11, 287-293; herein incorporated by reference in its entirety).

Visual observations of the surface of GPAT5::35 S plants compared to WT plants showed significant differences by SEM (see, FIG. 1). In particular, the defined wax structures of wild-type plants were mostly absent on the stems of GPAT5::35 S plants. Further, seeds of transgenic GPAT5::35 S plants showed increased substances on their surface. The images showed that GPAT5 altered wax deposition on the surface of plants including stems and seeds.

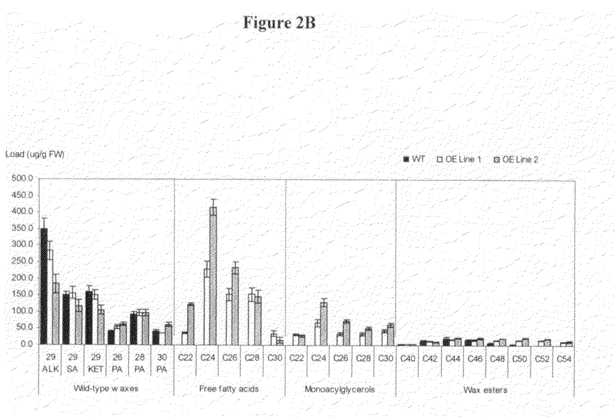

Analysis of surface lipid production in overexpressing GPAT5 plants compared to WT plants demonstrated an increase, of at least 2 fold, in total surface lipids of which C22-C30 free saturated fatty acids were increased (e.g., C24:0 free fatty acid and new products were expressed: C22-C30 α-(β-) monoacylglycerols such as where R=C21 to C29 alkyl group. (FIG. 2).

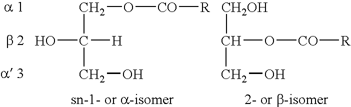

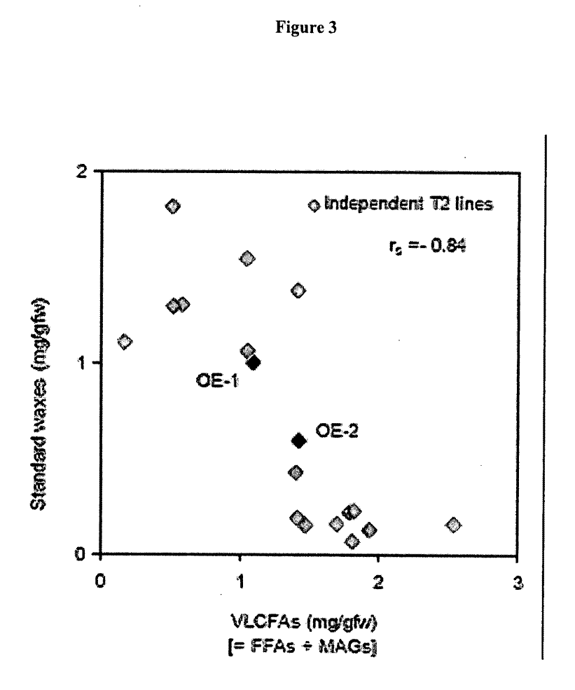

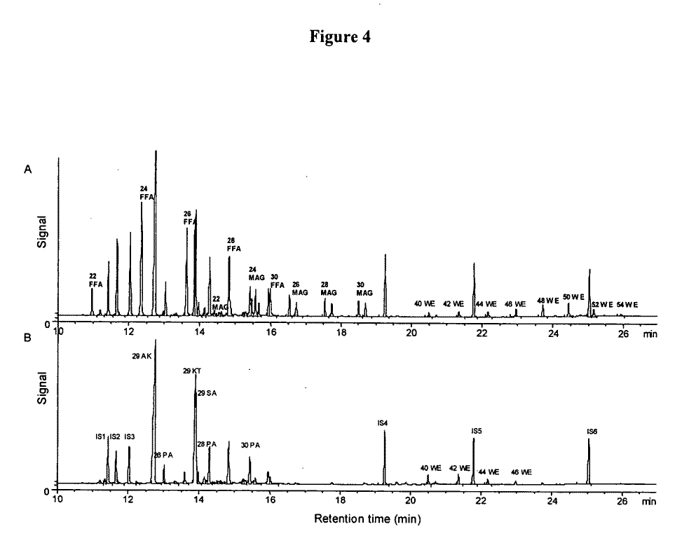

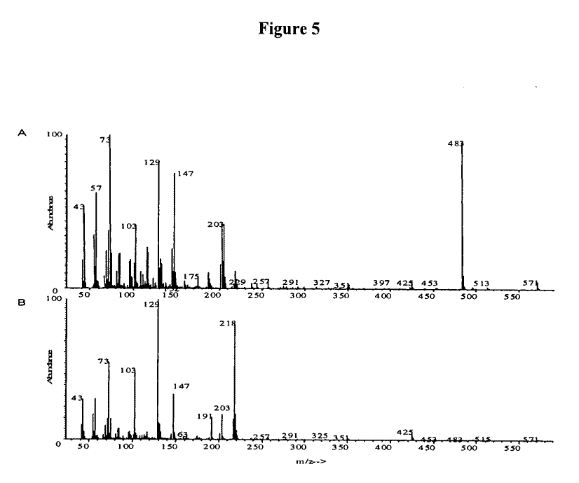

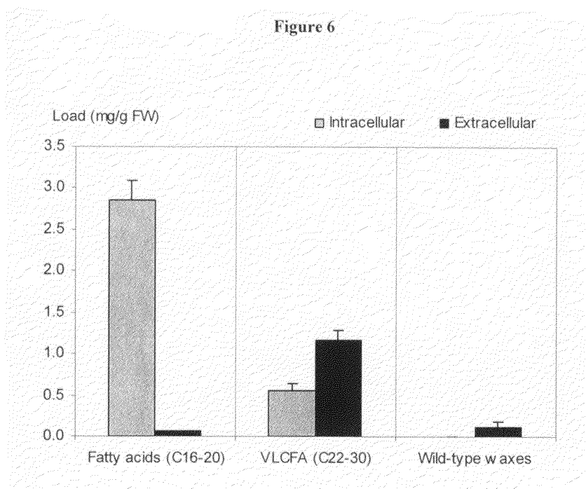

When the GPAT5 gene was ectopically expressed in Arabidopsis under control of the 35S promoter the stem cuticular wax composition was altered. The wax load increased 2 fold in leaves and in 5-week old stems, although the main components in wild-type waxes, namely nonacosane and its 15-oxo- and 15-hydroxy-derivatives, which are derived from C30 fatty acid, decreased. See, FIGS. 2-6, and Tables 1 and 2.

TABLE 1
Changes in total wax load in OE (two lines listed) comparing to
WT in various tissues and organs tested (mean ± S.E., n = 6,
each replicate was prepared from a mixture of plants).

| | | Stems | Leaves | Siliques | Seeds |
|---|---|---|---|---|---|
| Total | WT | 860 ± 60 | 80 ± 1 | 1500 ± 30 | 170 ± 10 |
| wax load | (Col-0) | | | | |
| (μg/g FW) | SF | 1660 ± 160 | 380 ± 5 | 1900 ± 70 | n.d. |
| | OE8 | 1960 ± 170 | 460 ± 10 | 2000 ± 80 | 860 ± 100 |

| | | | | |
|---|---|---|---|---|
| Fold increase | 2 | 5 | 2 | 5 |
| in wax load | | | | |
| FFA:MAG | 3:1 | 3:1 | 2:1 | 1:2 |
| a-MAG:β-MAG | 1:2 | 1:2 | 1:2 | 2:1 |

Associated with the reduction of the usual wax components was a large accumulation of C22-C30 saturated free fatty acids (FFA), with tetracosanoic acid as the major component. Particularly noteworthy was the accumulation of both 1- and 2-monoacylglycerols (MAGs) of the very long chain fatty acids. The ratio of FFA to MAG varied from 3:1 to 1:2, depending on the tissue examined. The presence of MAG indicates that GPAT5 functions in vivo as an acyltransferase to a glycerol-containing acceptor. The extracellular location of FFA and MAG products and the interception of the wax synthesis pathway suggest that ectopically-expressed GPAT5 has access to the same pool of acyl intermediates and/or may be targeted to the same membrane domain as that of epicuticular wax synthesis. In summary, these experiments showed that suberin and cuticular wax biosynthesis share the same subcellular localization.

Unlike other transgenic plants with altered internal lipid content, 35S::GPAT5 plants produce unusual and valuable lipid products such as very long chain fatty acids and monoacylglycerols on their surface. Furthermore, when combined with enzymes that modify the acyl structure, such as hydroxylases, GPAT family acyltransferases are contemplated to produce products such as ricinoleic acid (or lesqueroleate or lesquerolic acid, densipoleate, etc.) on their surface. In addition, WT plants and 35S::GPAT5 plants produced amounts of C16-C18 omega-hydroxy fatty acids and other types of in-chain-hydroxy fatty acids which are normal constituents of the cutin polymer.

TABLE 2
Chain length distribution and acid/alcohol composition for wax esters of wild-type (WT) stem and
overexpressing transgenic 35S::GPAT5 (OE) stems (line 1 listed). Major compounds are shaded.

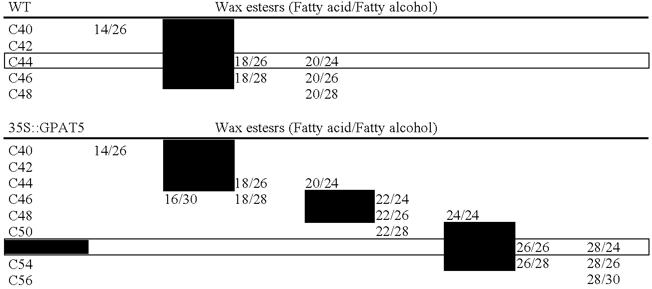

### Example IV

### This Example Describes Arabidopsis GPAT Genes

GPAT5 (At3g11430) (SEQ ID NO:01), is a member of a family of eight genes naturally found in Arabidopsis plant genomic libraries by the inventors. A few of the Arabidopsis GPATS were annotated and then functionally demonstrated glycerol-3-phosphate acyltransferase activity in vitro in yeast. In wild-type plants GPAT5 was found expressed primarily in seed coats, roots and anther. The following table shows the relationships of Arabidopsis GPAT5 to other Arabidopsis GPAT family members (GPAT 1-3 and GPAT 4, 6-8). Percent identity was determined following a Basic Local Alignment Search Tool (BLAST) at the European Molecular Biology Laboratory (EMBL)—The European Bioinformatics Institute (EBI) website using GPAT5 sequences (SEQ ID NOs:01 and 09).

TABLE 3
Relationship of Arabidopsis GPAT sequences - % identity.

| GPAT1 | GPAT2 | GPAT3 | GPAT4 | GPAT6 | GPAT7 | GPAT8 |
|---|---|---|---|---|---|---|
| At1g06520 | At1g02390 | At4g01950 | At1g01610 | At2g38110 | At5g06090 | At4g00400 |
| NA: 73% | NA: 61% | NA: 92% | NA: 59% | NA: 59% | NA: 83% | NA: 59% |
| partial | | partial | | | | |
| identity | | identity | | | | |
| AA: 44% | AA: 49% | AA: 39% | AA: 49% | AA: 49% | AA: 81% | AA: 49% |

### Example V

This example shows that disruption of the GPAT5 gene altered biochemical and physiological phenotypes, including altering compositions of extracellular lipid, such as a reduction in seed and root suberin aliphatic monomer content, increased seed coat permeability, and increased sensitivity of germinating seeds and roots to salts. The inventors contemplated the possible molecular functions of GPAT5 and other GPATs in the synthesis of aliphatic polyesters as well as the physiological roles of GPAT5 and aliphatic suberin monomers in specific organs.

Wild-Type GPAT5 Expression in Plant Parts and Identification of T-DNA Insertional Mutants.

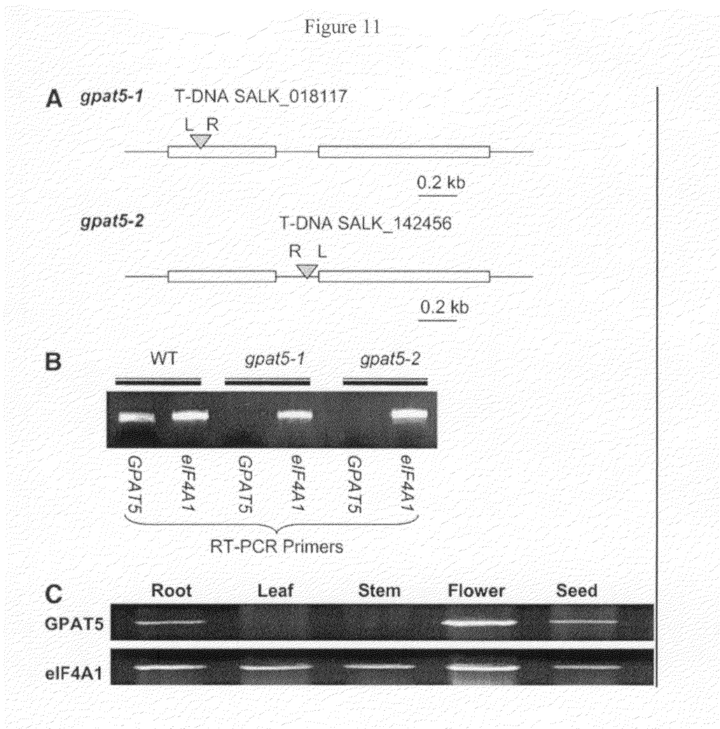

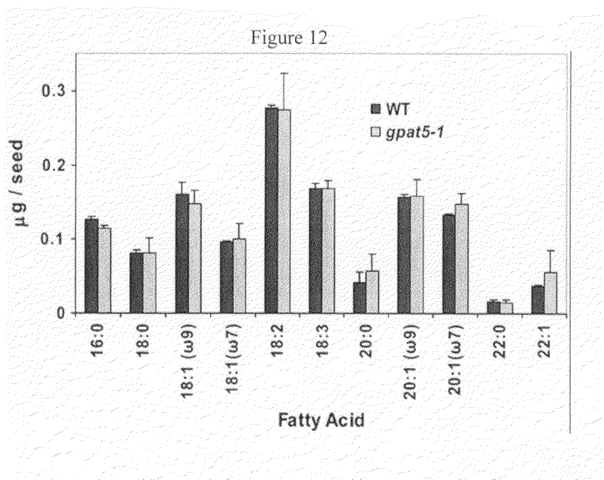

Two independent Arabidopsis T-DNA insertion lines, SALK_{—}018117 and SALK_{—}142456 (Alonso, et al., (2003) Science 301: 653-657; herein incorporated by reference), were selected and screened by the inventors for disruption of the GPAT5 gene using PCR analysis of GPAT5 using PCR primers described herein. As shown in FIG. 11A, SALK_{—}018117 has a T-DNA insertion in the first exon whereas SALK_{—}142456 has a T-DNA inserted in the intron of GPAT5.

Plant lines homozygous for a T-DNA insertion in GPAT5 were obtained for each of the independent insertion lines SALK_{—}018117 and SALK_{—}142456, named gpat5-1 and gpat5-2, respectively. The inventors tested for and did not detect a GPAT5 transcript from flowers of homozygous gpat5-1 and gpat5-2 plants, by RT-PCR (FIG. 11B). Further confirmation that both T-DNA gpat5-1 and gpat5-2 insertion lines generated a complete knockout of the GPAT5 gene. The phenotypes described herein for GPAT5 knock-out plants were observed in both gpat5-1 and gpat5-2 lines and their independent gpat alleles further supporting the observed phenotype resulting from disruption of the GPAT5 gene. See, FIG. 11, showing an exemplary structure of the GPAT5 Gene carrying a T-DNA Insertion, (11A and 11B) and analysis of GPAT5 expression by RT-PCR in wild-type plants, GPAT5 where mRNA was detected in flowers, roots, and seeds but not in stems and rosette leaves (FIG. 11C).

Tissue specific expression. The inventors identified tissue specificity of GPAT5 expression by ligating a fragment of an upstream region of a GPAT gene comprising a promoter region in combination with a marker gene. Thus a fragment comprising 1 kb of nucleic acids upstream of the first ATG of the GPAT5 cDNA together with the first exon (588 bp) of the gpat5gene, (for example, SEQ ID NO:78 ProGPAT5) was used to drive the expression of the β-glucuronidase (GUS) reporter gene in a transgenic Arabidopsis plant.

Consistent with the RT-PCR analysis, histochemical staining of transgenic plants expressing ProGPAT5:GUS showed GPAT5 expression (blue) in roots, flowers, and seeds but no blue color was observed in leaves and stems. GPAT5 expression in Arabidopsis wild-type plants identified by ProGPAT5:GUS expression at the beginning stages of seed desiccation showed that GUS-stained seeds, crushed for observations, was limited to the seed coat/endosperm, however not detected in the embryo. In contrast, staining in end staged seeds appeared limited to the funiculus attachment region. Further, dissection of GUS-stained seeds showed that at the beginning of the seed desiccation stage, GPAT5 expression was observed uniformly throughout the seed coat/endosperm fraction, whereas at the end of the desiccation stage, GUS staining was greater in the funiculus attachment region and possibly in some endosperm cells of this seed end stage.

GUS staining pattern in roots correlated with changes in development. In the seminal roots of 4- to 7-d-old seedlings grown on agar, staining was strongest at the junction of roots and hypocotyls, extended along the entire differentiated (specialization) zone where suberin deposition is known to occur, but blue color was not observed in the elongation zone or the root apical meristem. In 1- to 4-week-old roots grown on agar, where roots are still elongating but whose older root parts have entered the secondary state of growth (Dolan and Roberts, 1995 New Phytol. 131: 121-128; Baum et al., 2002, Am. J. Bot. 89: 908-920; all of which are herein incorporated by reference), GUS staining was present above but not observed in the division/elongation zones of the seminal root and the lateral roots, consistent with staining in 4-d old roots. Additional staining was seen in some older parts of the roots and in hypocotyls. Junctions to first and second order lateral roots were typically stained. GUS stained seminal root and a first-order lateral root of a 3-week-old seedling. Closer examination of 1- to 4-week old roots revealed that the additional staining along older parts of seminal and lateral roots was often made of small patches. In 6-week-old roots, which were grown on soil and clearly in their secondary state of growth, GUS staining was much weaker and was observed as patchy blue areas mostly at the periphery of older parts of the roots. GUS staining was not observed in stems and cauline leaves. GUS staining in 1- to 4-week-old roots of control Pro35S:GUS plants was not patchy but was strong and present throughout the root.

In Arabidopsis flowers, strong staining of GUS was observed in stamens but not observed in sepals, petals, and carpels. Microscopic examination of GUS-stained flowers showed that GUS activity was detected in the anthers and not observed in the filaments. Dissection of anthers showed that GUS staining was present in the developing pollen but not observed in mature pollen. This observed GUS and RT-PCR expression information was found to agree with results reported from AtGenExpress microarray data (Schmid et al., 2005 Nat. Genet. 37: 501-566; herein incorporated by reference) that showed GPAT5 mRNA significantly expressed in hypocotyls, roots, seeds, and stamens.

gpat5 Mutants are Affected in the Composition and Amount of Lipid Polyesters but not in Membrane and Storage Lipids.

Homozygous gpat5-1 and gpat5-2 mutant Arabidopsis plants were morphologically identical throughout development and reached similar sizes compared with wild-type Arabidopsis plants. No significant differences were observed in root growth when seeds were germinated on vertical agar plates. The fertility of gpat5 mutants was not affected (the number of seeds per silique was approximately 50, similar to that in the wild type). No differences in pollen grain size and shape were observed between the wild type and gpat5 under scanning electron microscopy.

GPAT1 was expressed in yeast was shown to have glycerol-3-phosphate acyltransferase activity in vitro (Zheng et al., 2003, Plant Cell 15: 1872-1888; herein incorporated by reference). Thus, to evaluate whether gpat5 mutants altered lipid production the inventors analyzed the fatty acids of lipid compounds in organs in which GPAT5 was expressed (seed, root, flower) compared to leaves as a control. Analysis was performed on intracellular lipids extractable and extracted into organic solvents (membrane and storage lipids) and on lipid polymers, which are nonextractable in organic solvents.

Fatty Acids from the Seed Coat/Endosperm Fraction of the Wild Type and gpat5 Mutants.

Mature seeds were manually dissected, and total fatty acids of the membrane and storage lipids of the seed coat/endosperm fraction were analyzed as fatty acid methyl esters by gas chromatography. Values are means of six replicates. Error bars denote 95% confidence intervals.

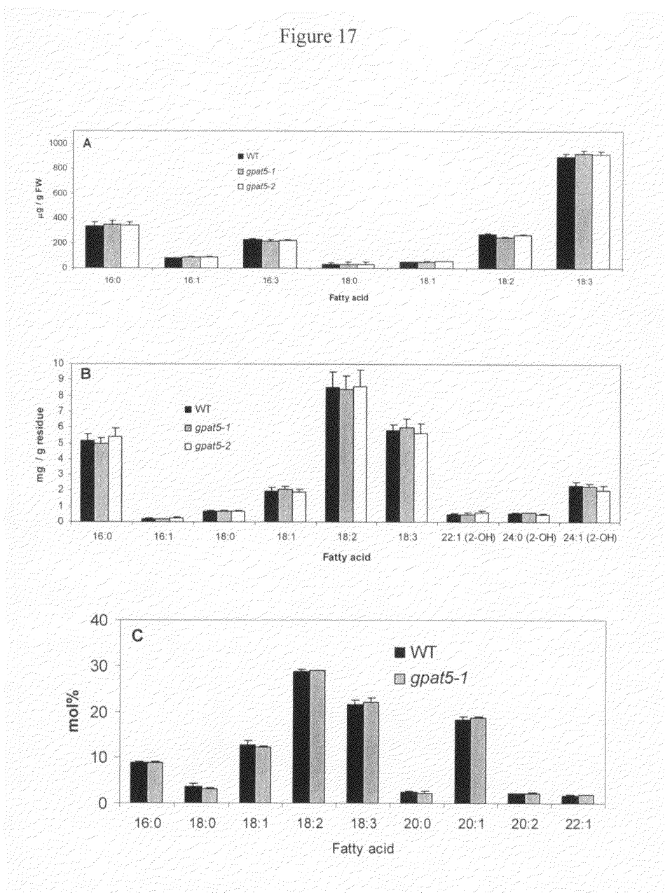

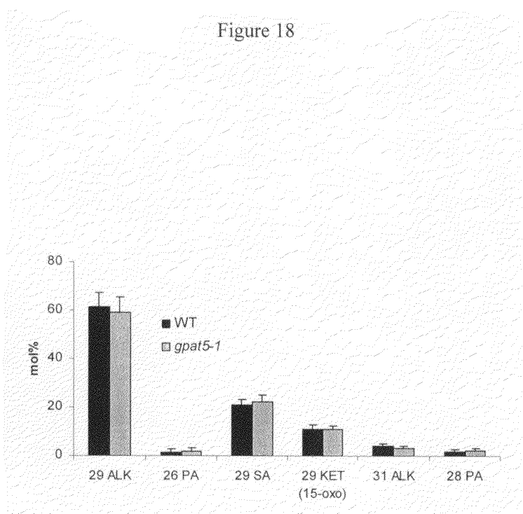

When soluble lipids of the manually dissected seed coat/endosperm fraction of mature seeds were analyzed, the amount of total fatty acids and the fatty acid composition were found to be the same in the wild type and gpat5 (FIG. 13). No differences in the amount of fatty acids derived from soluble lipids were detected in seeds, roots, and leaves of the mutants compared with the wild type (FIG. 17). The soluble lipids collected at the seed surface by chloroform dipping were typical Arabidopsis stem/leaf wax components, including alkane and fatty alcohols (see FIG. 18). The major component identified in seed coats was 29-carbon alkane, which is also the major wax component of Arabidopsis stems. Expressed per seed surface area, the wax coverage was approximately 0.3 mg/cm² for both the wild type and mutants. In contrast with soluble lipids, aliphatic monomers released from insoluble lipid polyesters were on average reduced by twofold in gpat5 seeds (Table 5).

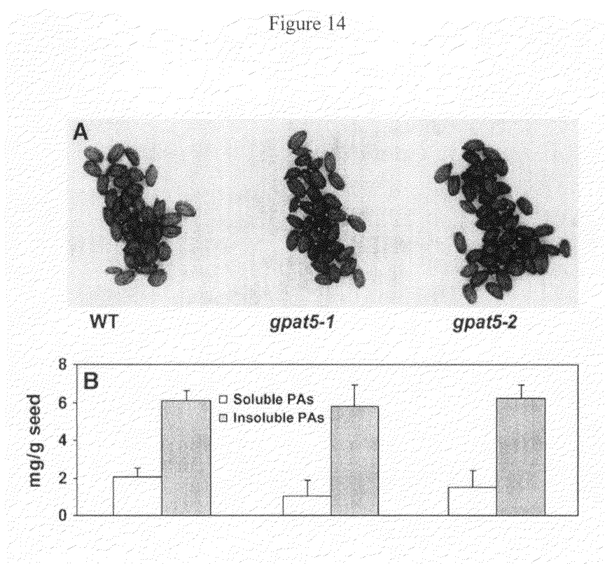

More than 90% of the total insoluble aliphatic polyester monomers found in seeds have been shown to come from the seed coat/endosperm fraction (Molina, et al. (2006) Phytochemistry 67: 2597-2610; herein incorporated by reference). The major aliphatic:monomer (24:0 α-hydroxy fatty acid), as well as 22:0 fatty acid, 22:0 α-hydroxy fatty acid, and 22:0/24:0 dicarboxylic acids, were reduced by at least twofold in the mutants compared with the wild type (FIG. 14A). These highly reduced monomers were typical monomers of aliphatic suberin (Kolattukudy, et al. (2001) Adv. Biochem. Eng. Biotechnol. 71:1-49; herein incorporated by reference). In contrast with the aliphatic monomers, the amount of aromatic monomers released from the polyester by depolymerization was not different between mutants and the wild type. However, depolymerization by transesterification presumably underestimates total hydroxycinnamate derivatives, which may be cross-linked by nonester bonds (Bernards, et al., (1995) J. Biol. Chem. 270:7382-7386; herein incorporated by reference).

Similar to seed coats, roots of 1-week-old seedlings grown on agar (primary state of growth), showed significant changes in the aliphatic monomers of suberin in gpat5 compared with the wild type (FIG. 13B). As with seeds, the change was not the same for all monomers. A 20 to 50% reduction was observed in C20-C24 fatty acid derivatives, whereas the C16-C18 fatty acid derivatives remained constant or increased.

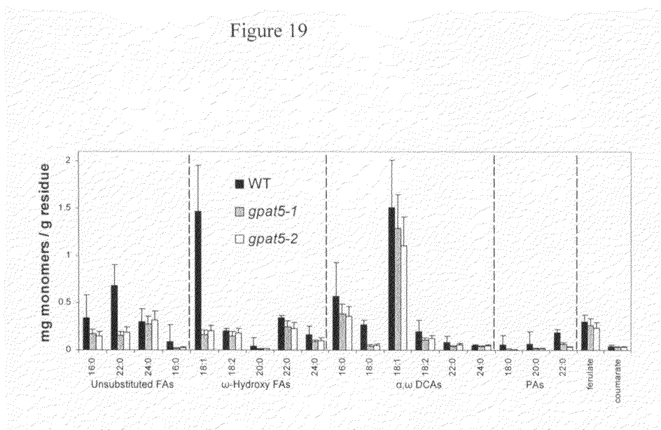

In 3-week-old roots grown on agar (beginning of the secondary state of growth), the suberin composition was different and the mutants showed a global 50% decrease in some monomers, including the 22:0 fatty acid and the major 18:1 α-hydroxy fatty acid (FIG. 19).

The reduction in total aliphatic suberin monomers in 3-week-old roots was also shown by the reduced staining intensity of some parts of the gpat5 roots when stained with Sudan black B, a lipophilic dye used for suberin histochemical detection (Robb, et al. (1991) Plant Physiol. 97: 528-537; herein incorporated by reference). In older wild-type roots grown on soil, in which the GPAT5 gene is weakly expressed (6 week old plants, secondary state of growth), the composition of suberin aliphatic monomers detected was similar to that reported previously (Franke, et al. (2005) Phytochemistry 66: 2643-2658; herein incorporated by reference), and no significant difference in the amount of each monomer was found between the wild type and the mutants.

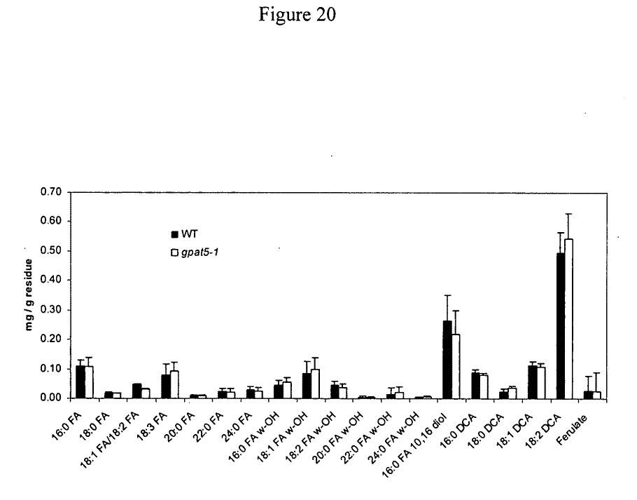

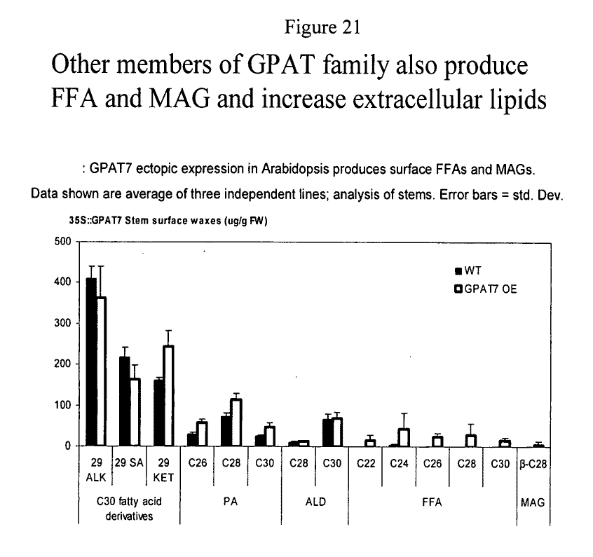

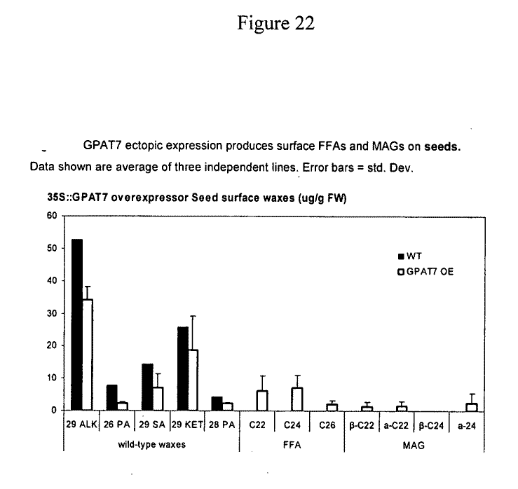

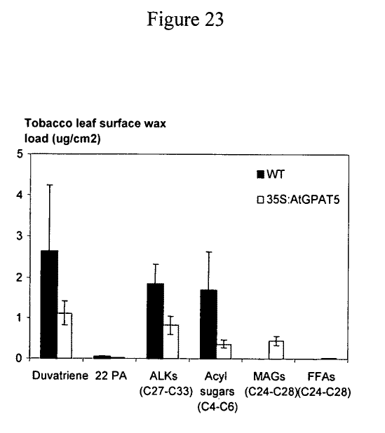

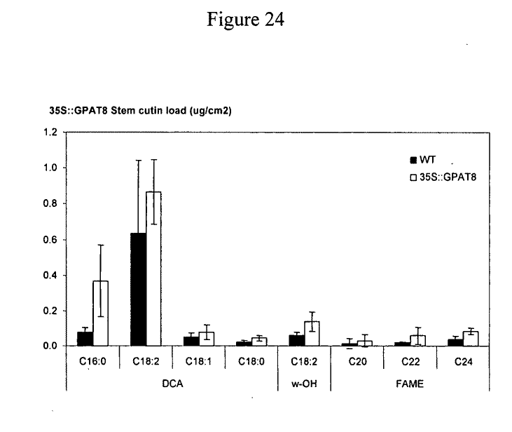

In flowers (FIG. 13C), a decrease in some lipid polyester monomers was also detected (e.g., 22:0 fatty acid and 18:2 co-hydroxy fatty acid) but was globally less strong than in roots and seeds, consistent with the fact that the gene is expressed in anthers. As expected, no difference in the lipid polyester monomers of leaves was detected (see, FIG. 20).

Together, these results show that the gpat5 mutants are altered (both quantitatively and qualitatively) in the synthesis of insoluble lipid polyesters but are not impaired in the synthesis of the bulk of storage and membrane lipids. A specific role of a GPAT family member in lipid polyester synthesis is strongly supported by other observations, including the fact that two other GPATs are required for lipid polyester synthesis in leaves.

Seed Coats of gpat5 Mutants Show Increased Permeability to Dyes and Darker Color.

The mutant gpat5 knock-out plants produced seeds of the same weight as wild type plants (17.0±0.7 compared with 16.9±0.1 mg/seed). Results similar to those of gpat5-1 were obtained with gpat5-2.

Scanning electron microscopy of the surface of wild-type versus gpat5-1 seeds. Close examination of the seeds via scanning electron microscopy showed that the mutant seeds are in the same range of size, have the same oblong shape, and show the same surface structure, with similar columella heaps, as wild-type seeds. Ruthenium red staining of seed mucilage in wild-type versus gpat5-1 seeds showed that mucilage production of the mutant seeds is also the same as that of wild-type seeds.

Tetrazolium salt staining (24 h) of wild-type versus gpat5-1 seeds. Permeability properties of the seed coat of the mutants were tested using tetrazolium red salt, a cationic dye that is normally excluded by the Arabidopsis seed coat but that is reduced to red products (formazans) by NADPH-dependent reductases after penetrating the embryo (Debeaujon, et al. (2000) Plant Physiol. 122:403-414; herein incorporated by reference). After staining for 24 h, gpat5-1 and gpat5-2 seed coats were much more permeable to tetrazolium red than were wild-type seed coats, suggesting that the seed coat is indeed affected in the mutants. Shorter incubation times with tetrazolium red showed that the staining first appeared in the region of the hilum and diffused outward from there. Control staining experiments run on embryos whose seed coats had been manually removed showed that wild-type and mutant embryos had the same kinetics and intensity of staining and that the red products appeared at the same time in other parts of the embryo surface. Therefore, these controls ruled out a possible difference in the capacity to metabolize tetrazolium red between mutant and wild-type embryos or between the parts of the embryo close to the hilum region and other parts.

Tetrazolium salt staining (4 h) of wild-type versus gpat5-1 seeds. Tetrazolium salt staining (24 h) of seeds resulting from the fertilization of wild-type plants by gpat5-1 pollen (left) and of gpat5-1 plants by wild-type pollen.

Differences observed by the inventors were attributed to a difference in the permeability of wild-type and mutant seed coats to tetrazolium red, especially in the hilum region. The hilum is the scar left on the seed coat after detachment from the funiculus. In mature seeds of Arabidopsis, the hilum is adjacent to the micropyle (where the radicle will emerge) and faces the chalazal pole. When excited at 365 nm, seed coats of the mutant showed a decrease in autofluorescence in the hilum region, suggesting a decrease in suberin content. Furthermore, the seed coat surface of gpat5 mutants was clearly less stained in the hilum region when using the lipophilic suberin dye Sudan red 7B (which proved to be more efficient than Sudan black B for staining polyesters of wild-type Arabidopsis seed coats). A localized area of strong Sudan red 7B staining was visible in the wild type, whereas in the mutant the staining was weak or not visible to the inventors in this region. The seed coats of the mutant were more fragile, as they almost always broke when mounted between slide and cover slip. The permeability of wild-type seeds remained unaffected after the removal of surface waxes by chloroform dipping, excluding the possibility that seed surface waxes contribute to seed coat impermeability to tetrazolium salts.

### Example VI

Characterization of a Suberin Mutant.

Genetic analysis of the seed coat permeability phenotype in gpat5 mutants indicated that this phenotype segregated as a single recessive allele and that its inheritance was consistent with the expression of GPAT5 in the seed coat (tissue of maternal origin). Indeed, F1 seeds (i.e., on the F0 mother plants) were nonpermeable when wild-type parental plants were fertilized by pollen from gpat5-1 knockout plants, whereas they were permeable when gpat5-1 knockout plants were fertilized by wild-type pollen. In addition, analysis of subsequent progeny of the F1 heterozygous plants showed that F2 seeds were nonpermeable. Segregation analysis of F2 plants demonstrated that the seed permeability phenotype segregated in F3 seeds fitted a 3:1 ratio for a Mendelian single recessive mutation (x²=0.17; P=0.68). As expected, F2 plants giving permeable F3 seeds were homozygous and the F2 plants giving nonpermeable F3 seeds were either wild type or heterozygous, as determined by PCR.

Furthermore, it was observed that gpat5-1 and gpat5-2 seeds had a darker appearance than wild-type seeds (FIG. 14A) and that in the F1, F2, and F3 seeds this darker seed coat color was always associated with the seed coat permeability phenotype and never with the nonpermeable phenotype. The segregation analysis thus demonstrated that seed coat permeability and color co-segregated with the T-DNA insertions and that these genetic lesions in GPAT5 segregated as single recessive alleles.

The inventors observed that Arabidopsis seed coat color is conferred by the brown pigments formed during seed desiccation by the oxidation of colorless proanthocyanidins (PAs; also called condensed tannins) (Lepiniec, et al. (2006) Annu. Rev. Plant Biol. 57: 405-430; herein incorporated by reference). Analysis of soluble PAs and the cell wall-bound insoluble PAs by an acid hydrolysis method optimized for Arabidopsis seeds (Routaboul, et al. (2006) Planta 224:96-107; herein incorporated by reference) showed that gpat5 mutant seeds have the same level of both types of PAs as wild-type seeds, ruling out an increase in PA amount (FIG. 14B). This result suggests that the darker color may result indirectly from a higher degree of oxidation of PAs, although the inventors are not excluding other alternative explanations.

gpat5 Mutants Showed an Increased After-Ripening Requirement of Seeds and Higher Sensitivity of Seedlings to Salinity.

The effect of the knockout of GPAT5 on seed physiology was further analyzed in terms of dormancy release, germination rate under various conditions, and seedling establishment. Seed dormancy is defined as the temporary failure of an intact viable seed to complete germination under favorable conditions (Bewley, et al. (1997) Plant Cell 9:1055-1067; herein incorporated by reference) and is controlled by environmental factors such as light, temperature, oxygen availability, and time of dry storage (after-ripening requirement) as well as by genetic factors (Bentsink, et al. (Apr. 4, 2002) Seed dormancy and germination. In The Arabidopsis Book. C. R. Somerville and E. M. Meyerowitz, eds (Rockville, Md.: American Society of Plant Biologists), doi/10.1199/tab.0050, www., followed by, aspb.org/publications/, followed by, arabidopsis/; herein incorporated by reference).

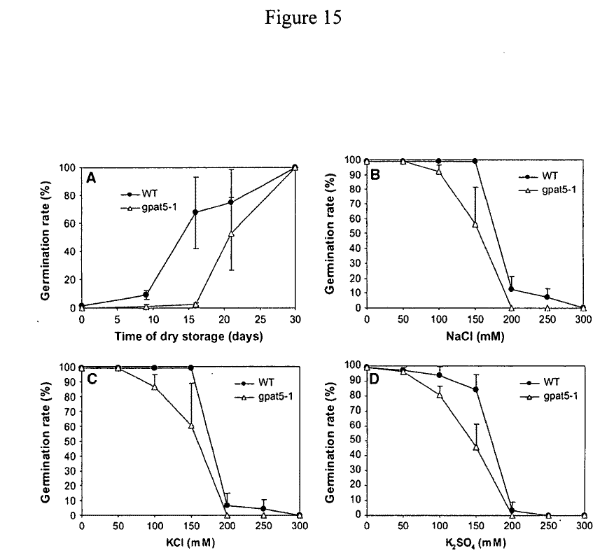

Dormancy release. The inventors compared the dormancy release of mutant seeds and wild-type seeds, which had been harvested at the same time from plants grown together under identical conditions. As shown in FIG. 15A, both wild-type and mutant seeds were dormant when harvested immediately after the end of seed maturation (no germination at day 0). Wild-type seeds increased germination approximately 60% after 17 d of dry storage; by contrast, gpat5 germination remained low (<10%). However, with increasing length of postharvest storage, dormancy was gradually released, with 100% germination observed after 30 d of storage for both wild-type and mutant seeds. Thus, the difference between wild-type and mutant seeds is the kinetics of the dormancy release. Germination of dormant mutant seeds in light or dark after cold treatment was almost 100% when scored at day 7 after transfer to a growth chamber. Moreover, the increased after-ripening requirement of the gpat5 mutants was observed in seeds resulting from the fertilization of gpat5-1 plants by wild-type pollen but not in the seeds of wild-type plants fertilized by gpat5-1 pollen, which showed that the phenotype was not attributable to the embryo.

Germination of gpat5 seeds under various conditions. When cold-treated seeds were germinated on Murashige and Skoog (MS) medium (Physiol. Plant. 15:473-497; herein incorporated by reference, with increasing salt concentrations, an average 50% reduction in the rate of germination of gpat5 seeds compared with wild-type seeds was observed at 150 mM NaCl (FIG. 15B). Similar results were obtained with other ions, ruling out a specific effect of sodium or chloride (FIGS. 15C and 15D). These data show that seed coat control of ion uptake is critical for proper germination.

Seedling establishment. Rate of seedling establishment under increasing salt concentrations and phenotypes of seedlings germinated on 150 mM NaCl. The inventors observed that more than half of the seeds that germinated at 150 mM NaCl showed a development arrest before the establishment of green cotyledons. Additionally, a sensitivity of gpat5 seedlings to salt (100 to 150 mM) was also observed with KCl and K₂SO₄. To test whether a defect in young developing roots could be at least partially responsible for this salt-sensitivity phenotype of the seedlings, seeds were germinated for 3 d on MS plates and transferred to MS plates supplemented with 200 mM NaCl, where they were grown for an additional 5 days. The percentage of gpat5 seedlings showing bleaching was significantly higher than that of wild-type seedlings after 5 d at 200 mM NaCl, suggesting that the roots of gpat5 seedlings were severely impaired in their ability to control ion uptake. No significant difference between the wild type and gpat5 was observed when seeds were germinated and seedlings were grown for 12 d on agar plates supplemented with up to 400 mM of the neutral organic osmoticum polyethylene glycol 8000 or mannitol.

GPAT5 and Seed Coat Permeability.

The polyesters of mature seed coats have been shown in several species to consist largely of cutin-type monomers, whereas typical suberin monomers have been found to be of lower abundance and thought to be restricted to a small region of the seed coat (Espelie, et al. (1979) Plant Physiol. 64:1089-1093; herein incorporated by reference). At the end of seed maturation, the import of nutrients from the mother plant via the funiculus ceases and the funiculus attachment region is sealed, leaving a scar on the seed coat, the hilum. In grapefruit (Citrus paradisi) seeds, it has been shown that the hilum region is indeed sealed by the deposition of aliphatic suberin in several cell layers (Espelie, et al. (1980) Planta 149:498-511; herein incorporated by reference). Unlike Arabidopsis seeds, grapefruit seeds can be dissected and enough material from the hilum/chalazal region of the seed coat can be obtained for polyester analysis. The polyester monomers in this seed coat region are typical suberin monomers, with 22- and 24-carbon α-hydroxy fatty acids and dicarboxylic acids representing approximately 37 mol % of the total composition (percentage based on the numbers of total moles) The rest of the grapefruit inner seed coat is enriched in cutin monomers. Therefore, based on the following lines of evidence, the inventors contemplate that a major role of GPAT5 in Arabidopsis seeds is to seal the hilum region by providing aliphatic monomers for suberin deposition: (1) the typical suberin very long chain monomers found in the grapefruit hilum are abundant in Arabidopsis seed coat polyester monomers, and these monomers are the ones that are specifically reduced several fold in gpat5 mutants (FIG. 14); (2) at the end of the desiccation stage, when suberin deposition in the hilum region is expected to occur, the expression of the GPAT5 gene is highest in this region; (3) the kinetics and pattern of appearance of the red staining in the gpat5 embryo suggest that the tetrazolium salts diffuse mostly or exclusively through the hilum region to the embryo; and (4) the hilum region of the mutant seed coat had a lower amount of insoluble lipophilic material. GPAT5 expression was also detected more broadly throughout the seed coat, especially during early seed desiccation. This observation suggests a role of GPAT5 in the synthesis of the polyesters in other parts of the seed coat than the hilum region (the extrahilar region), mostly by providing suberin-like monomers. However, the extrahilar region of the seed coat seemed to remain impermeable to tetrazolium salts, which suggests that a defect in GPAT5 may be compensated by functionally redundant GPATs.

The location of the lipid polyesters in the extrahilar region of Arabidopsis is unknown. At maturity, the Arabidopsis seed coat consists of dead cells corresponding to the five cell layers of epidermal origin differentiating from the ovule integuments (L1 to L5 from the outermost to the innermost). L1 (epidermis) has a preserved structure with the thick cell walls of the columella and the mucilage, whereas layers 2 to 5 are largely collapsed and crushed together and contain the brown pigments (Haughn, et al. (2005) Trends Plant Sci. 10:472-477; herein incorporated by reference). In mature Arabidopsis seeds, an electron-dense cutin-like layer of polyesters cannot be observed clearly on transmission electron micrographs in the L1 cell layer (epidermis) and may be located in the thick electron-dense crushed cell layers L2 to L5. Interestingly, in developing seeds, an electron dense layer bordering the inner cell wall of L5 (endothelium), which is in contact with the embryo sac, has been observed. This layer reacts positively with osmium tetroxide and therefore looks like a cuticle that is present throughout seed development until the mature embryo stage (Beeckman, et al. (2000) J. Plant Res. 113:139-148; herein incorporated by reference).

After-Ripening Requirement and Seed Coat Color in gpat5 Mutants.

The darker color of the gpat5 mutants is not attributable to an increased amount of PAs (FIG. 17B) but may result from a higher visibility of the brown pigments through the seed coat, to a fraction of the brown pigments that cannot be depolymerized or extracted (Routaboul, et al. (2006) Planta 224:96-107; herein incorporated by reference), or from a higher degree of oxidation of PAs. Although, at this time, the connection between seed coat polyesters and seed color is not understood, the inventors speculate that the crushed L2 to L5 layers of the final seed desiccating stage might be composed in part of a polyester network (the cuticle initially bordering L5) in close contact with oxidized polymerized PAs (brown complexes) and that disruption of the polyester network might cause some changes in the formation of these brown complexes or in their visibility.

The dormancy of the mutant seeds was released more slowly, as indicated by the time shift in germination rates measured under normal conditions (FIG. 15). The germination rate was always scored 7 d after inhibition, so the difference observed was not attributable to a delay in germination in mutant seeds. Reciprocal crossing of gpat5 and the wild type showed that the embryo of the mutants was not more dormant. In Arabidopsis, seed dormancy has been described as seed coat-enhanced dormancy (Bewley, 1997, Plant Cell 9:1055-1066; herein incorporated by reference). The seed coat exerts a germination restrictive action by being impermeable to water and/or oxygen, by producing germination-inhibiting compounds, and/or by its mechanical resistance to radicle protrusion. The increase in the after-ripening period and the darker seed color the inventors have observed in the gpat5 mutants are consistent with previous observations in Arabidopsis, in which a reduced dormancy phenotype has been observed in several seed coat mutants with reduced brown pigmentation (Leon-Kloosterziel, et al. (1994) Plant Cell 6:385-392; Debeaujon, et al. (2000) Plant Physiol. 122: 403-414, all of which are herein incorporated by reference), and in several other plant seeds in which it has been observed that the darker the seed coat, the more dormant the seeds (Kantar, et al. (1996) Ann. Appl. Biol. 128:85-93, herein incorporated by reference). The underlying mechanism connecting seed coat color and dormancy, however, is unclear, and a direct effect of seed coat polyesters on seed dormancy cannot be completely excluded. Indeed, the reduction in mutant seed coat polyesters could alter the kinetics of critical compounds leaking from the embryo, such as nitric oxide, the accumulation of which is important for the breaking of dormancy (Bethke, et al. (2006) J. Exp. Bot. 57:517-526; herein incorporated by reference). Because germination and dormancy are of agronomic significance, the identification of lipid polyesters from the hilum or extrahilar region as a factor regulating seed dormancy may provide additional insights into the control of this process and will be worth further investigation.

GPAT5 and Root Suberin.

The chemical analysis of 1- and 3-week-old roots grown on agar clearly shows that GPAT5 is required for suberin synthesis in roots. Thus, the expression of GPAT5 (as viewed by GUS staining) in the youngest part of the specialization zone (but not in the division and elongation zones) in 4-d-old roots and 1- to 4-week old roots still elongating is consistent with a role in the initial deposition of Casparian and lamellar suberin, which occurs in the primary state of growth for all roots investigated (Enstone, et al. (2003) J. Plant Growth Regul. 21:335-351; herein incorporated by reference), including Arabidopsis (Di Laurenzio, et al. (1996) Cell 86:423-433; Cheng, et al. (2000) Plant Physiol. 123:509-520; all of which are herein incorporated by reference. A transverse section of the differentiation zone of 4-d-old ProGPAT5:GUS roots showed that GUS staining was present in every cell layer observed. This pattern of expression of GPAT5 was also observed in microarray data from root tissues (Birnbaum, et al. (2003) Science 302:1956-1960; herein incorporated by reference). Histochemical staining of Arabidopsis young roots (Di Laurenzio et al., 1996, Cell 86:423-433; Cheng et al., 2000, Plant Physiol. 123:509-520; Franke et al., 2005, Phytochemistry 66:2643-2658; all of which are herein incorporated by reference) indicates an apparent restriction of suberized cell walls to the endodermis. The more widespread expression of the GPAT5 gene thus suggests that additional depositions of aliphatic lipid polyesters may occur in roots but might remain undetectable by the usual staining procedures, either because of a lower abundance than the endodermal suberin or a difference in structure (the usual dyes are thought to bind the aromatic domain of suberin, but their exact mode of action and their sensitivity/specificity for various lipophilic polymers remain unclear). Another possibility is that GPAT5 produces in the differentiation zone of roots a small pool of soluble glycerolipids.

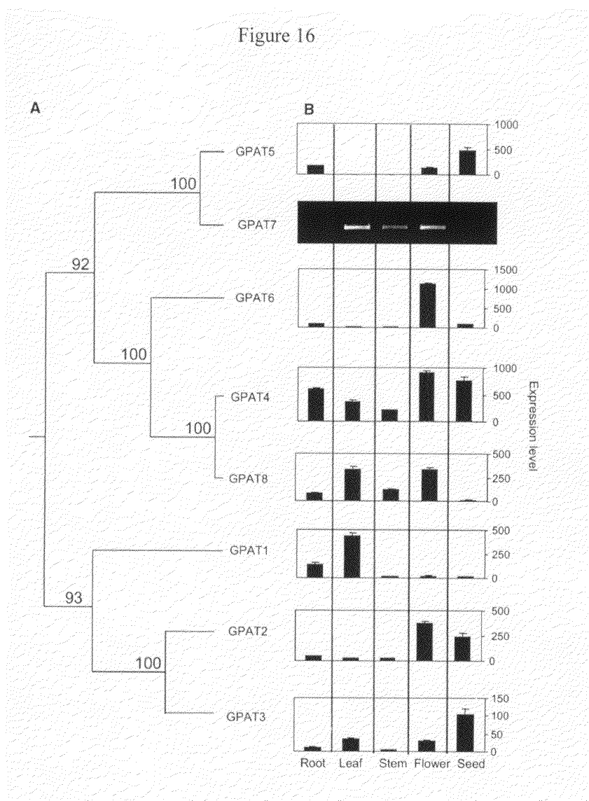

The patchy expression of GPAT5 in some of the older parts of 1- to 4-week-old roots, in cells in which the initial suberin deposition was laid down earlier during development, indicated that an additional deposition or a remodeling of suberin composition occurred later in root development before the formation of periderm. Thus explaining how the composition of aliphatic suberin in 3-week-old roots was changed compared with that in 1-week-old roots grown under the same conditions. The later deposition/remodeling of suberin in specific zones are contemplated to have several nonexclusive causes. First, in 1- to 4-week-old roots grown on agar, the endodermis divides periclinally according to a complex helicoidal pattern and features of secondary growth are observed, which may also give rise to new suberin deposition (Dolan, et al. (1995) New Phytol. 131: 121-128; Baum, et al. (2002) Am. J. Bot. 89:908-920, all of which are herein incorporated by reference). Second, there are at least six other GPATs expressed in roots (FIG. 16), and they could contribute to this additional suberin deposition/remodeling in other zones than GPAT5 (see below). Third, the formation of lateral roots could require the expression of GPAT5, as suggested by the results observed by the inventors. Fourth, the synthesis of suberin could be induced by environmental conditions. Concerning this latter point, it is not likely that the GPAT5 GUS staining pattern results from induction by pathogens (the roots are grown on sterile medium), but it could result from local gradients of water or nutrients on the agar plates. The understanding of the contribution of GPAT5 and the other GPAT isoforms expressed in roots to the building of the suberin depositions at various stages of Arabidopsis root development will require detailed studies of tissue gene expression in conjunction with histochemical studies of the deposition of Casparian and lamellar suberin.

### Example VI

This example describes identification, characterization, and comparisons of GPAT family acyltransferase genes from plants. Amino acid sequence and/or nucleic acid sequence of Arabidopsis GPAT5 (SEQ ID NO:09 and 01) were used to search The National Center for Biotechnology Information (NCBI) and The Institute for Genomic Research (TIGR) public internet databases. Sequences were used either directly or in a reverse search using amino acid sequences for obtaining nucleic acid sequences. Using the Basic Local Alignment Search Tool (BLAST), sequences from a variety of plants were identified. A sampling of sequences identified as homologs or orthologs for use in the present invention are shown in Table 4.

TABLE 4
Plant GPAT Family Acyltransferases and putative acyltransferase molecules.

| | Protein | | NA | |
|---|---|---|---|---|
| | SEQ ID | Percent | SEQ ID | Percent |
| Organism | NO: XX | Identity | NO: XX | Identity |
| Arabidopsis thaliana (Mouse- | 9 | 100% | 1 | 100% |
| ear cress) GPAT5_ARATHQ9CAY3 | | | | |
| GPAT5; At3g11430 | | | | |
| Nicotiana tabacum | 17 | 89% | 18 | 79% |
| tobacco\|BP528167, partial | | | | |
| Lycopersicon esculentum | 34 | 82% | 33 | 76% |
| tomato\|BI923206 | | | | |
| Arabidopsis thaliana | 15 | 81% | 7 | 83% |
| GPAT7_ARATH | | | | |
| GPAT7; At5g06090 | | | | |
| Nicotiana tabacum, | 26 | 76% | 25 | NS |
| BP528123, partial | | | | |
| Medicago truncatula (Barrel | 19 | 72% | 20 | partial |
| medic) Q1SYU3_MEDTR | | | | 74%/71% |
| Phospholipid/glycerol | | | | |
| acyltransferase | | | | |
| Brassica napus | 24 | 69% | 23 | partial |
| oilseed_rape\|CD833550 | | | | 97%/75% |
| Zea mays Q5GAV5_MAIZE | 43 | 63% | 44 | 80% |
| Lycopersicon esculentum | 31 | 62% | 32 | 79% |
| tomato\|TC157067 | | | | |
| Q65XG3_ORYSA | 21 | 61% | 22 | 74% |
| Oryza sativa (japonica | | | | |
| cultivar-group) | | | | |
| Q8W0S3_SORBI | 62 | 45% | 63 | 59% |
| [Sorghum bicolor (Sorghum) | | | | |
| (Sorghum vulgare)] | | | | |
| Q6TUA6_ORYSA | 64 | 52% | 65 | 67% |
| Oryza sativa (japonica | | | | |
| cultivar-group) | | | | |
| Arabidopsis thaliana | 11 | 49% | 3 | 61% |
| GPAT2_ARATH/Q9FZ22 | | | | |
| arab\|TC273270 | | | | |
| GPAT2; At1g02390 | | | | |
| Arabidopsis thaliana | 14 | 49% | 6 | 59% |
| GPAT6_ARATH/O80437 | | | | |
| GPAT6; At2g38110 | | | | |
| Arabidopsis thaliana | 13 | 49% | 5 | 59% |
| GPAT4_ARATH/Q9LMM0 | | | | |
| GPAT4; At1g01610 | | | | |
| Arabidopsis thaliana | 16 | 49% | 8 | 59% |
| GPAT8_ARATH | | | | |
| GPAT8; At4g00400 | | | | |
| Medicago truncatula | 19 | 47% | 41 | XX |
| (Barrel medic) | | | | |
| Q1SCJ7_MEDTR | | | | |
| Phospholipid/glycerol | | | | |
| acyltransferase | | | | |
| Arabidopsis thaliana | 10 | 44% | 2 | Identities = |
| GPAT1_ARATH/Q9SHJ5 | | | | 149/203 |
| GPAT1; At1g06520 | | | | (73%) |
| Lycopersicon esculentum | 40 | 41% | 39 | NS |
| tomato\|TC165504 similar | | | | |
| to TIGR_Ath1\|At1g01610 | | | | |
| similar to | | | | |
| TIGR_Ath1\|At4g00400 | | | | |
| Medicago truncatula (Barrel | 41 | 41% | 42 | NS |
| medic) Q1RU17_MEDTR | | | | |
| Arabidopsis thaliana | 12 | 39% | 4 | Identities = |
| GPAT3_ARATH/Q9SYJ2 | | | | 35/38 |
| GPAT3; At4g01950 | | | | (92%) |
| Lycopersicon esculentum | 35 | 38% | 36 | NS |
| Tomato TC163159 | | | | |

Partial in organism column = partial sequence.
Partial in identity column = high identity over a short area, no identity shown for the remainder of the sequence.
NS = no significant similarity.
XX = not calculated.

Gene Tree and Gene Expression Profiles of the Eight Putative GPATs of Arabidopsis.

A cladogram shows the branching order of Arabidopsis GPATs according to a phylogenetic tree of protein sequences of plant acyltransferases (Kim and Huang (2004) Plant Physiol. 134: 1206-1216; herein incorporated by reference). The original tree was built using the neighbor-joining method with 1000 bootstrap replicates. Bootstrap values are percentages. (B) Microarray expression data derived from AtGenExpress (Schmid, et al. (2005) Nat. Genet. 37:501-506; herein incorporated by reference). Expression levels in each tissue (root, leaf, stem, flower, and seed) at different developmental stages were averaged (bars represent means±SE). The expression profile for GPAT7 was determined in this study via RT-PCR analysis because its expression profile is not available at AtGenExpress. See, FIG. 16.

### Example VIII

### Identification and Characterization of Arabidopsis Knockout Mutants for a glycerol-3-phosphate acyltransferase5 Gene (GPAT5; At3g11430)

The inventors further show herein, that plants comprising knock-out mutants of gpat5, specifically gpat5 knock-out mutants gpat5-1 and gpat5-2, were altered in polyester synthesis in roots and in seed coats (such as the seed surface) of seeds from mutant gpat5 plants. Several phenotypic characteristics of the gpat5 mutants, such as enhanced seed coat permeability, decreased seed germination, and abnormal root growth under salt stress conditions, show that GPAT5 expression contributes to suberized cell wall biogenesis in seeds and roots and that gpat5 expression is required for normal seed and root function.

TABLE 5
Quantification of Waxes, Other Soluble Lipids,
and Polyesters in Wild-Type and gpat5-1 Seeds.

| | | Fatty Acid | | |
|---|---|---|---|---|
| | | Methyl Esters | | Aliphatic |
| | | from Extractable | | Polyester |
| | Seed Weight | Lipids | Surface Waxes | Monomers |
| Seed | (mg/Seed) | (ng/Seed) | (ng/Seed) | (ng/Seed) |
| Wild type | 16.9 ± 0.1 | 5230 ± 6 80 | 1.3 ± 0.05 | 68.9 ± 5.1 |
| gpat5-1 | 17.0 ± 0.7 | 4910 ± 370 | 1.4 ± 0.01 | 35.2 ± 1.2 |

Values shown are means ± SE from two independent experiments.

Transfer of 3-d-old germinated seedlings to a medium supplemented with 200 mM NaCl resulted on average in a fourfold higher rate of bleaching for gpat5 compared with the wild type. Thus gpat5 knock-out roots are indeed affected in their ability to restrict ion movements and prevent a massive entry into the cortex, a role that has been ascribed to suberin depositions of Casparian bands (Sattelmacher, et al. (2001) New Phytol. 149: 167-192; Enstone, et al. (2003) J. Plant Growth Regul. 21: 335-351; Ma, et al. (2003) Can. J. Bot. 81: 405-421, all of which are herein incorporated by reference). An effect attributable to osmotic stress could be ruled out by the absence of any difference in seed germination and seedling establishment between wild-type and mutant plants grown with up to 400 mM mannitol or polyethylene glycol 8000. The increased sensitivity to salt stress observed in a mutant affected in root suberin content thus confirms a role previously contemplated for suberin in roots and shows that it can be critical for seedling survival at moderate salt concentrations.

### Example IX

### MAGs are Components of Arabidopsis Root Waxes

Surface waxes from stem and leaf are lipid material extracted by chloroform. Using a chloroform dipping procedure, wherein the plant parts are quickly dipped into chloroform as described herein, the inventors examined the presence of surface lipids in roots of 7-week-old Arabidopsis plants. At this 7-week stage the roots had undergone considerable secondary growth and the epidermis, cortex and endodermis was cast off, leaving the newly-formed suberin-rich periderm as the root outer cell layers (Dolan et al. (1995) New Phytologist 131:121-128; Franke et al. (2006) Phytochemistry 66:2643-2658; all of which are herein incorporated by reference). The presence of suberized periderm at the periphery of the roots used in this study was confirmed by the inventors by stained root cross-sections with the lipophilic dye Sudan Red 7B.

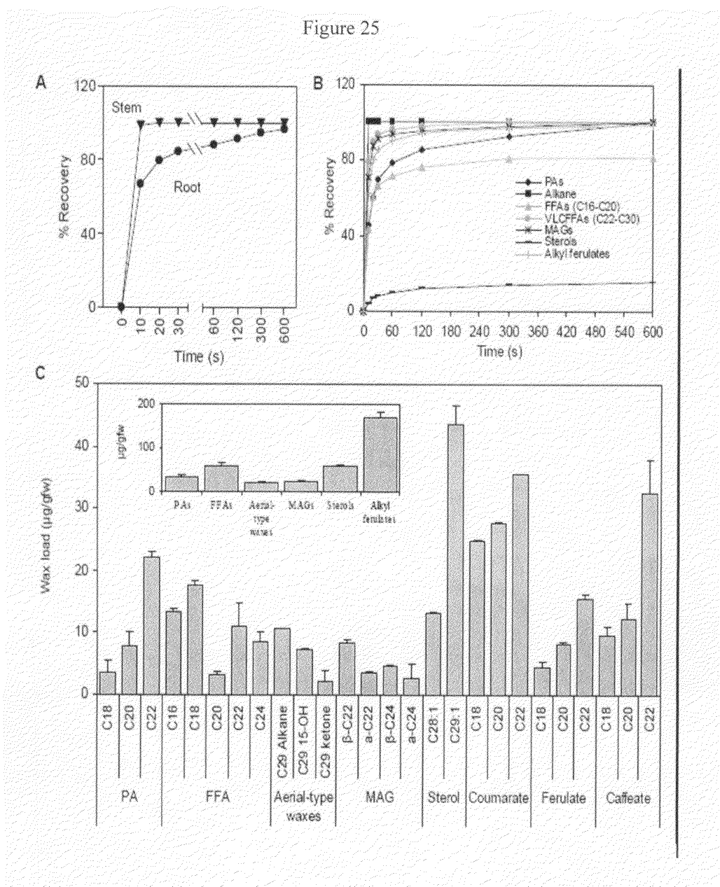

The inventors found that a 10 s dip in chloroform was sufficient to recover >98% (w/w) of total surface waxes from Arabidopsis stems. A 10 s dip also extracted significant amounts of lipid material from roots (FIGS. 25A and B). A 1 minute dip of roots into chloroform was found to dissolve approximately 90% (w/w) of extractable lipid material (FIG. 25A) without significant (<5% w/w) contamination from intracellular membrane lipids. This procedure was therefore used for subsequent root analyses with the lipid material recovered by this procedure collectively termed “root waxes.” At 7 weeks, Arabidopsis roots contained 360±32 μg/g fresh weight (fw) of root waxes (mean with 95% confidence interval (CI), n=4). This value compares with values of approximately 1000 μg/gfw for stem wax loads and approximately 100 μg/gfw for leaf wax loads.

The composition of waxes of Arabidopsis roots (FIG. 25C) was distinct from that of Arabidopsis aerial parts (Rashotte et al., 2001, Phytochemistry 57:115-123; herein incorporated by reference) and significantly different from that reported for the subterranean storage organs of seven species, including Crucifers (Espelie et al. (1980) Planta 148:468-476; herein incorporated by reference). In Arabidopsis roots, esters of p-coumaric, caffeic and ferulic acids with C18-C22 saturated fatty alcohols were the major component (47% w/w). Primary alcohols were present in significant amounts (10% w/w) with a chain length profile very similar to those esterified to hydroxycinnamic acids. This profile distinguishes them from the C26-C30 primary alcohols of Arabidopsis cuticular waxes in aerial organs (Rashotte et al. (2001) Phytochemistry 57:115-123; herein incorporated by reference). Substantial amounts of sterols and FFA were also observed (approximately 15% w/w each).

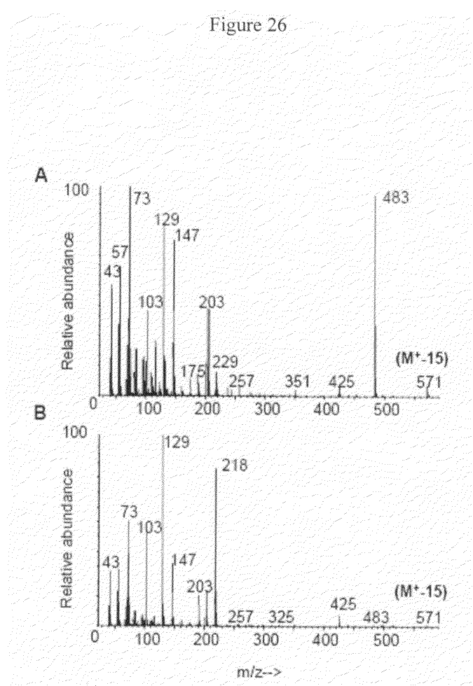

The inventors show herein that lipid components that dominate aerial waxes in WT Arabidposis plants, in particular nonacosane and its 15(14)-hydroxy and 15-oxo derivatives, were minor contributors to root waxes (approximately 5% w/w). The most unusual feature of Arabidopsis root waxes was the presence of both α- and β-isomers of MAGs (approximately 7% w/w). The acyl groups were C22>C24>C26-C30 saturates, with negligible C20, and in this particular characteristic the MAG distribution was similar to the longer-chain FFAs in roots. Identification of MAG isomers was accomplished primarily by use of GC-MS identification of their bis-trimethylsilyl derivatives (Murphy (1993) In Mass Spectroscopy of Lipids—Handbook of Lipid Research. Plenum Press, New York, pp 206; herein incorporated by reference), as shown in FIG. 26 (A and B) for the tetracosanoyl species. The inventors point out that although the α-MAG isomer is thermodynamically more stable than the β-MAG isomer (as described in Gunstone, (1967) In An Introduction to Chemistry and Biochemistry of Fatty Acids and Their Glycerides. Chapman Hall, London, pp. 141; herein incorporated by reference), the latter, β-MAG isomer, was found to be more prevalent in the root waxes than in stems. Specifically, the [M-15]+ ion at m/z=571 gives the MW=586. For α-MAG isomers cleavage of the C(β)-C(γ) bond of the glycerol backbone usually produces the base peak [M-CH2OTMSi]+. The m/z=483 ion in FIG. 26A corresponds to this cleavage. This [M-103]+ ion is very weak in the mass spectra of β-MAGs. Instead, their most diagnostic ions are [M-RCOOH]+ and [M-RCOOH-OTMSi]+, which correspond to m/z=218 and 129 respectively (FIG. 26B). The identification of these MAG isomers was consistent with their Rf values on silica TLC and their Rt values on GC analysis, when compared to standards.

Kinetics of chloroform-dipping extractions showed that after a 10s-dipping was sufficient to extract 100% of stem waxes, however a 10s dip recovered alkanes (FIG. 25B) whereas other root wax components required longer dipping times, most noticeably to recover primary alcohols. Kinetics of extraction of roots showed that increased dip time did not increase the collection of additional amounts of C16-C20 FFAs and sterols. However root tissue disruption by grinding increased these collected lipids. This indicated that there were distinct intracellular and wax fractions for C16-C20 FFAs and sterols.

The Accumulation of MAGs in Root Waxes Correlates with GPA T5 Expression.

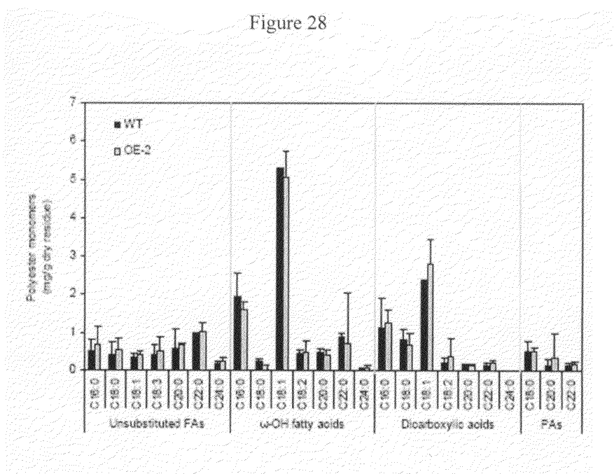

The inventors observed that the T-DNA knock-out mutant gpat5 showed large reductions in suberin-like aliphatic monomers released by transesterification of the residual fraction of seeds and roots remaining after extensive delipidation (Beisson et al. (2007) Plant Cell 19:351-368; herein incorporated by reference). Therefore the inventors expressed gpat5 in Arabidopsis under control of the CaMV 35S promoter as described herein. Ectopic expression of gpat5 was confirmed via RT-PCR transcript analysis of mRNA prepared from 7 independent lines using leaves, an organ where GPAT5 is not expressed in WT. Two lines, (designated OE-1 and OE-2), were used for subsequent analyses. Plants ectopically expressing GPAT5 displayed no obvious difference in growth or morphology compared to WT. There was no significant change in the suberin load and composition of 7-week-old roots of 35S::GPAT5-expressing plants (FIG. 28).

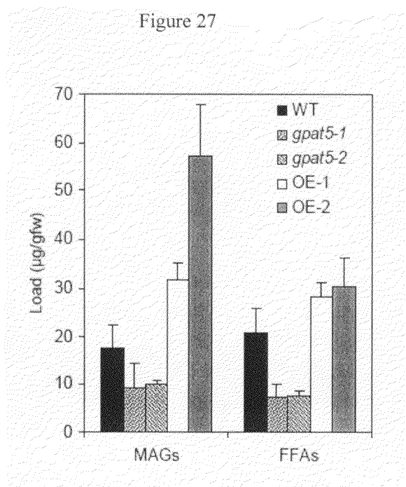

Analyses of the root waxes of WT, two 35S::GPAT5-expressing lines (OE-1 and OE-2) and two knock-out mutant lines (gpat5-1 and gpat5-2) revealed a strong positive correlation between GPAT5 expression and the concentration of MAGs (FIG. 27). Approximately a 50% (w/w) reduction was observed in the mutants compared to a 70% (w/w) increase in the 35S::GPAT5-expressing lines. Both positional isomers of MAG were affected to a similar degree. A positive correlation was also observed for C22-C30 FFAs (FIG. 27). Other root wax components were either not significantly changed with altered GPAT5 expression (primary alcohols, sterols) or weakly reduced by GPAT5 knock-out and not increased by over-expression (alkyl ferulates). One explanation for these results is that GPAT5 activity was limiting for the production of MAGs in waxes from WT roots. Thus null mutations of GPAT5 may not completely deplete MAGs from the suberin-associated waxes because of gene redundancy within the GPAT family. Indeed other GPATs are expressed in roots (Beisson et al. (2007) Plant Cell 19:351-368; herein incorporated by reference).

GPAT5 Overexpression Produces MAGs as Novel Components of Cuticular Waxes.

The inventors characterized the stem cuticular waxes of the 35S::GPAT5-expressing plants. SEM of the stem surface showed a large reduction in wax crystal density compared to WT. Stems of the WT plants were covered primarily with columnar-shaped crystals, although rods, tubes, vertical plates, and dendritic- and umbrella-like structures were also visible (Rashotte et al. (1998) Int J Plant Sci 159:773-779; Jetter et al. (2006) Composition of plant cuticular waxes. In M Riederer, C Müller, eds, Biology of the Plant Cuticle, Annual Plant Reviews Vol 23 Blackwell Publishing Ltd, Oxford, pp 145-175; all of which are herein incorporated by reference). In the stems of 35S::GPAT5 plants crystals that were still visible are mostly plate-like. These observations suggested changes of cuticular wax content and composition on stem surface of 35S::GPAT5-expressing plants. Paradoxically, despite a reduction in wax crystal densities chemical analysis revealed 1.8- and 2.2-fold increases in total cuticular wax load in OE-1 and OE-2 plants, respectively.

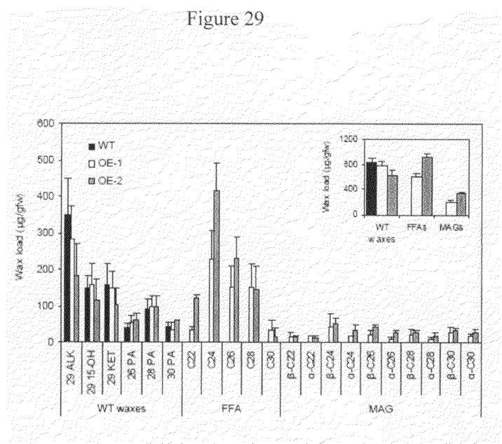

The inventor's analysis of wax demonstrated the presence of both α- and β-isomers of MAGs, with saturated C22-C30 acyl groups, as novel components of the cuticular waxes from stems of 35S::GPAT5-expressing plants. An exemplary analysis shows MAGs constituting up to 20% (w/w) of the total wax load (inset in FIG. 29).

Whereas FFAs were minor components of WT waxes (<5%), greatly elevated levels of C22-C30 FFAs (approximately 50% w/w) were measured. The accumulated MAGs have a slightly altered acyl chain length distribution when compared to those present in the root waxes of Arabidopsis plants (FIGS. 25 and 29). Tetracosanoic acid was the major FFA and acyl group in MAGs of stems. Another noticeable phenotype was a shift in distribution of the saturated wax esters, which constitute about 7-9% (w/w) of total wax load, from C40-C46 in WT to C40-C54 in the transgenes. In WT wax esters the predominant acyl groups was C16, but in the transgene there was an additional contribution largely from the C22 acyl group.

The novel MAG and FFA products observed in stem waxes for 35S::GPA T5-expressing plants were also observed in surface waxes from leaves, siliques and seeds (Table 6).

TABLE 6
Characteristics of the chloroform-dipping wax fraction from plant parts (organs)
of 35S::GPAT5 overexpression lines (mean with 95% CI, n = 4).

| Lipid | Organism | | Stems | Leaves | Siliques | Seeds | Roots |
|---|---|---|---|---|---|---|---|
| Total wax | WT | Standard | 860 ± 200 | 80 ± 5 | 1500 ± 100 | 170 ± 30 | 360 ± 30 |
| load | | waxes | | | | | |
| (μg/gfw) | | | | | | | |
| Total wax | WT | MAG + | n.d. | n.d. | n.d. | n.d. | n.d. |
| load | | FFA | | | | | |
| (μg/gfw) | | | | | | | |
| Total wax | OE-1 | Standard | 780 ± 320 | 68 ± 10 | 840 ± 200 | 150 ± 50 | 300 ± 120 |
| load | | waxes | | | | | |
| (μg/gfw)) | | | | | | | |
| Total wax | OE-1 | MAG + | 810 ± 280 | 310 ± 12 | 1100 ± 150 | n.d. | — |
| load | | FFA | | | | | |
| (μg/gfw) | | | | | | | |
| Total wax | OE-2 | Standard | 620 ± 300 | 80 ± 10 | 750 ± 100 | 400 ± 100 | 400 ± 150 |
| load | | waxes | | | | | |
| (μg/gfw) | | | | | | | |
| Total wax | OE-2 | MAG + | 1230 ± 400 | 380 ± 5 | 1250 ± 130 | 380 ± 50 | — |
| load | | FFA | | | | | |
| (μg/gfw) | | | | | | | |
| Fold increase | GPAT5 | | ~2 | ~5 | ~2 | ~5 | ~1 |
| in total wax | overexpression | | | | | | |
| load | lines over WT | | | | | | |
| FFA:MAG | | | 3:1 | 3:1 | 2:1 | 1:2 | 1:1 |
| (w/w) | | | | | | | |
| α-MAG:β- | | | 1:2 | 1:2 | 1:2 | 2:1 | 1:2 |
| MAG (w/w) | | | | | | | |
| Dominant | | | C24: 0 | C26: 0 | C24: 0 | C24: 0 | C22: 0 |
| acyl-chain | | | | | | | |
| length | | | | | | | |

n.d.: not detected,
~= approximately.

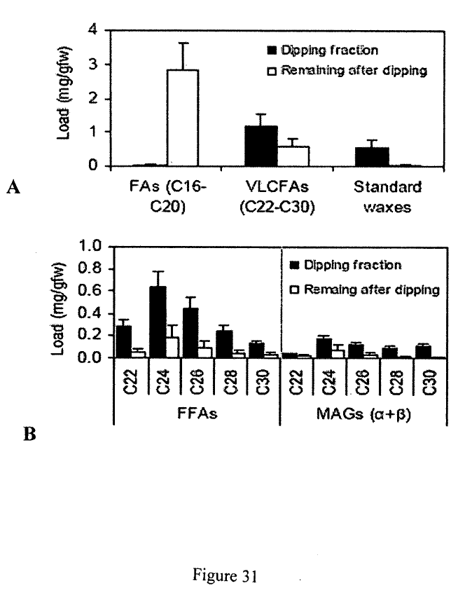

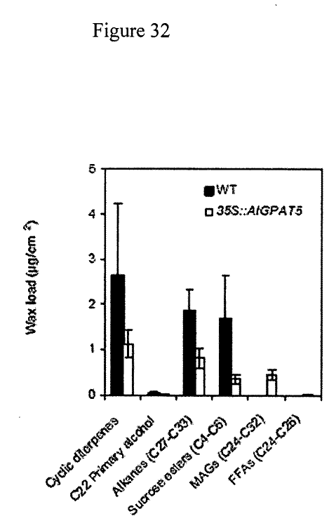

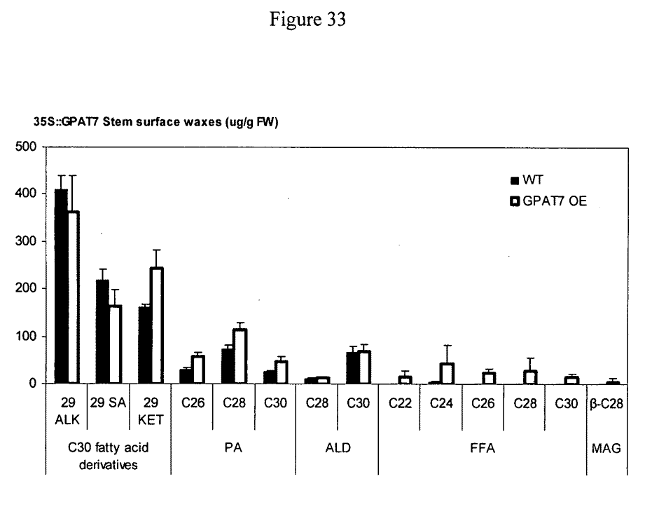

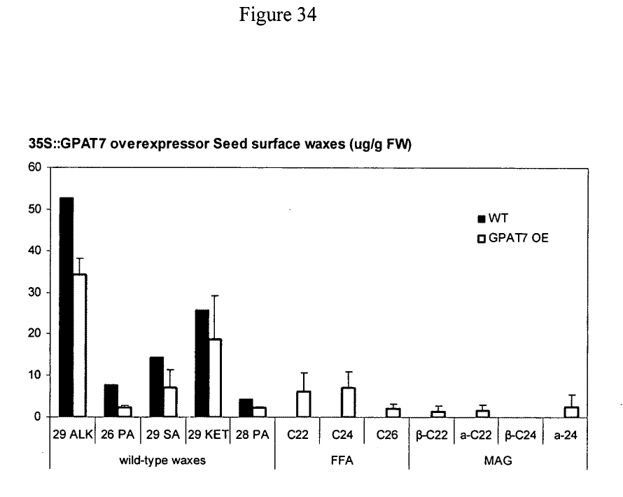

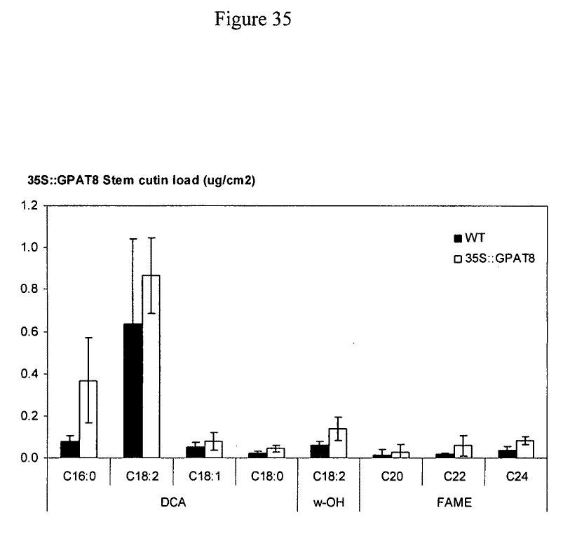

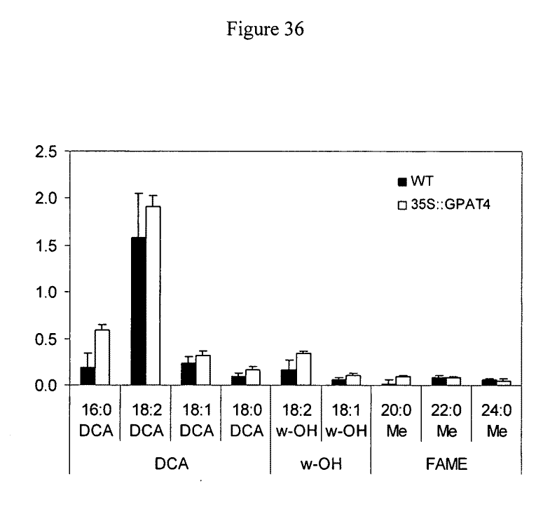

When the inventors analyzed the lipids in both the 30 s chloroform dip fraction and from the remaining stem tissue the inventors found that two thirds of VLCFAs (total acyl groups from MAGs and FFAs) were immediately extractable (FIG. 31A). This compares with the WT surface waxes, which are completely extracted, and with the intracellular lipids (i.e. C16-C20 fatty acids), which are not extracted. Furthermore, the chain length distribution of FFA and MAG in these two fractions was similar, although the longer chain MAG species did appear to be preferentially found on the surface (FIG. 31B). Epidermal peel experiments confirmed that the MAGs plus FFAs remaining after the short chloroform dip are largely epidermal (>90% w/w), although whether their site of deposition is extracellular, intracellular, or both, remains to be determined. Taken together, these findings show that ectopic expression of GPAT5 in the epidermis results in novel MAG, wax ester and FFA products, and that these products are largely secreted onto the plant surface, where they radically alter the crystal morphology of the epicuticular wax layer. Although lysophosphatidic acid might be expected to be the first product of the GPAT5 reaction (Zheng et al. (2003) Plant Cell 15: 1872-1887; herein incorporated by reference), analyses of the short chloroform dip and lipids extracted from residual stems failed to reveal any lysophosphatidic acid containing VLCFAs. Also, an analysis of the stem cutin showed that no additional long-chain C22-C30 fatty acids were incorporated into this polyester.

GPAT5 Overexpression Reduces the Accumulation of WT Cuticular Wax Components.

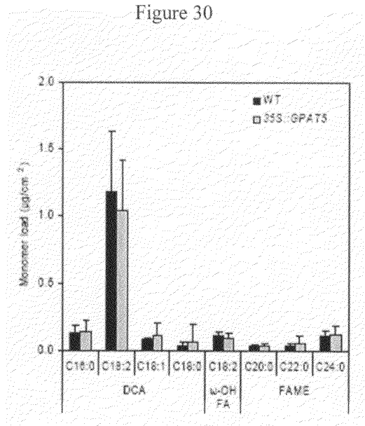

In addition to the appearance of novel components in 35S::GPAT5 overexpression lines there was a reduction in the load of some WT wax components, most dramatically for the C₂₋₉ alkane component of the decarboxylation/decarbonylation pathway, whereas the C26-C30 primary alcohols remained approximately constant (FIG. 28). To confirm the reduction in WT waxes the inventors analyzed the accumulation of C22-C30 fatty acids (from MAGs plus FFAs) in stems from 6-week-old plants for 18 independent T2 35S::GPAT5 lines. An inverse relationship was observed between the amount of WT waxes present in each line and the amount of newly formed VLCFAs due to GPAT5 expression (FIG. 30).

### Example XI

### Surface MAGs are Produced in 35S::GPAT5 Expressing Tobacco Plants

Experiments conducted using tobacco plant parts demonstrated similar results when compared to experiments using Arabidopsis plants. Thus the inventors contemplate that the results of the GPAT overexpression and underexpression, examples provided herein, are broadly applicable to other crop plants and algae, and further applicable to other plants comprising the plant kingdom. The results from tobacco overexpression are summarized as follows: First, WT tobacco roots from 8-week old plants were subjected to the rapid chloroform dip protocol for collection of lipids, then analyzed by GC-MS demonstrating trace amounts of docosanoyl- and tetracosanoyl-glycerols, wherein the majority were β-isomers; second, when tobacco plant parts were transformed with the same 35S::AtGPAT5 construct used for overexpression in Arabidopsis plants, significant amounts of MAGs (15% w/w of total leaf wax load) were produced that were novel components of tobacco cuticular waxes, as shown for lipids extracted from 4-week old plants. These MAGs contained saturated C24-C28 acyl groups, with C26 as the dominant acyl moiety. Both straight chain and iso- and anteiso-branched-chain acyl groups were present in the MAGs. This parallels the presence of iso- and anteiso-C29-C33 branched-chain hydrocarbons reported for tobacco epicuticular waxes (Severson et al., 1984, J Agric Food Chem 32: 566-570, herein incorporated by reference).

An exemplary molecular species distributions for the alkane and MAG fractions for tobacco plants transformed with the 35S::AtGPAT5 construct are shown in Table 7. The alkane distribution is very similar to that reported by Severson et al. (1984, J Agric Food Chem 32:566-570; herein incorporated by reference), and is annotated accordingly. The bis-TMSi derivatives of branched-chain β-MAC species have very similar mass spectra to their straight-chain isomers but elute ahead at distinctive fractional equivalent carbon numbers. Using this type of retention time analysis that the inventors previously used for the identification of branched chain components of Arabidopsis and Brassica seed polyesters (Molina et al., 2006, Phytochemistry 67:2597-2610; herein incorporated by reference), and with the comparison with the tobacco cuticular alkane composition, the inventors identified the novel MAGs as containing iso- and anteiso-branched-chain acyl groups synthesized from valine- or isoleucine-derived primers for fatty acid synthesis.

TABLE 7
Molecular distribution and identification of alkanes and
monoacylglycerols in the leaf waxes of tobacco plants
overexpressing 35S::AtGPAT5 (±SD, n = 3).

| | Rt | Cn | MAG Isomer | Configuration | % TIC |
|---|---|---|---|---|---|
| Alkanes | 28.02 | C27 | | n | 0.76 ± 0.04 |
| | 30.29 | C29 | | br (iso-) | 0.94 ± 0.03 |
| | 30.80 | C29 | | n | 1.52 ± 0.06 |
| | 31.79 | C30 | | br (anteiso-) | 2.71 ± 0.09 |
| | 32.11 | C30 | | br (iso-) | 0.31 ± 0.02 |
| | 32.95 | C31 | | br (iso-) | 4.14 ± 0.12 |
| | 33.45 | C31 | | n | 8.26 ± 0.11 |
| | 34.35 | C32 | | br (anteiso-) | 4.23 ± 0.1 |
| | 34.64 | C32 | | br (iso-) | 0.58 ± 0.03 |
| | 35.41 | C33 | | br (iso-) | 2.05 ± 0.05 |
| | 35.88 | C33 | | n | 4.67 ± 0.1 |
| Monoacyl- | 36.43 | C24 | β | n | 1.04 ± 0.02 |
| glycerols | 38.23 | C26 | β | br (iso-) | 1.14 ± 0.03 |
| | 38.64 | C26 | β | n | 3.24 ± 0.05 |
| | 39.08 | C26 | α | n | 0.2 ± 0.03 |
| | 39.44 | C27 | β | br (anteiso-) | 0.36 ± 0.06 |
| | 39.70 | C27 | β | n | 0.17 ± 0.03 |
| | 40.35 | C28 | β | br (iso-) | 1.19 ± 0.02 |
| | 40.75 | C28 | β | n | 3.36 ± 0.13 |
| | 41.16 | C28 | α | n | 0.16 ± 0.02 |
| | 41.50 | C29 | β | br (anteiso-) | 0.53 ± 0.03 |
| | 42.37 | C30 | β | br (iso-) | 0.81 ± 0.03 |
| | 42.74 | C30 | β | n | 0.57 ± 0.04 |
| | 43.49 | C31 | β | br (anteiso-) | 0.41 ± 0.03 |
| | 44.48 | C32 | β | br (iso-) | 0.21 ± 0.02 |
| | 44.87 | C32 | β | n | 0.74 ± 0.02 |

Rt: retention time;
Cn: carbon number;
TIC: total ion current;
br: branched;
n: normal.

### Example X

This example describes the creation and analysis of a T-DNA insertion GPAT4/GPAT8 double knock-out homozygous plant line.

Arabidopsis GPAT4×GPAT8 double homozygous knock-out plants such as those described herein, further demonstrated that GPAT4 and GPAT8 alter cuticle formation.

T-DNA insertional plant lines SALK_{—}106893 and SALK_{—}095122 for GPAT4 (WT At1g01610, SEQ ID NO:05), and GPAT8 (WT At4g00400, SEQ ID NO:08), respectively were identified using the SIGnAL “T-DNA Express” Arabidopsis Gene Mapping Tool (http:// followed by signal.salk.edu/cgi-bin/tdnaexpress) provided by the Salk Institute Genomic Analysis Laboratory (Alonso, et al., (2003) Science 301:653-657; herein incorporated by reference in its entirety in its entirety). Individual seeds for these lines were obtained from the Arabidopsis Resource Center at Ohio State University. Plants were grown from these seeds, and for each line DNA was prepared and used for genotype screening. The gene-specific primers used for the screening of insertions into the GPAT4 gene were: [5′-CCCCAAAACGATGAAAGCTA-3′ (forward) SEQ ID NO:71 and 5′-TTCTCGAGGAGTTGCCTCAT-3′ (reverse) SEQ ID NO: 72], and for GPAT8 [5′-TCGATTGCAAAATACAA-3′ (forward) SEQ ID NO: 73 and 5′-CAAGTTCGATATCGCGGATT-3′ (reverse) SEQ ID NO: 74]. These primers and the T-DNA left border primer LBa1 (5′-TGGTTCACGTAGTGGGCCATCG-3′; SEQ ID NO: 75) were used to check by PCR for the presence of a wild-type or T-DNA mutant allele. Homozygous plants for the T-DNA insertion at the GPAT4 locus and homozygous plants for the T-DNA insertion at the GPAT4 locus were isolated.

Homozygous plants gpat4 and gpat8 were crossed. F1 seeds were harvested, grown and allowed to self-pollinate. F2 seeds were harvested, then F2 plants were screened for double homozygotes (same primers used as for the screening of single homozygotes). Leaves from 3 week-old double homozygous plants were dipped for 2 minutes in toluidine blue dye (method adapted from Tanaka et al. (2004) Plant J. 37:139-46; herein incorporated by reference in its entirety).

Leaves of GPAT4×GPAT8 double homozygous knock-out plants showed increased permeability to Toluidine blue dye. Specifically, strong and uniform staining was obtained for the double homozygous T-DNA insertion line gpat4/gpat8 whereas WT leaves were not stained. These results indicated that the GPAT4 and GPAT8 genes are involved in cuticle formation in Arabidopsis leaves. These results indicate that GPAT5 prefers long-chain fatty acid structures (C20-C26), and that GPAT4 and GPAT8 prefer shorter chain lengths (C16-C18) as acceptor molecules. As such, the inventors contemplated that GPAT4 and/or GPAT8 provide useful embodiments for methods of the present invention for altering lipids on the surface of plants.

Segments from the apical 1 cm of stem were mounted onto cryo-scanning electron microscopy (SEM) stubs with 25% dextran and plunged into liquid nitrogen. Frozen samples were transferred into an Emitech K₁₂₅₀ cryo-system, where water was sublimed for 30 min at 277 degree C. and subsequently sputter coated with gold for 2.5 min at 35 mA. The coated samples were viewed with a Hitachi S4700 field emission SEM using an accelerating voltage of 2 kV and a working distance of 12 mm.

All publications and patents mentioned in the above specification are herein incorporated by reference. Reference to a cited document is not an acknowledgement that this teaching belongs to the common general knowledge in the art. Various modifications and variations of the described method and system of the invention will be apparent to those skilled in the art without departing from the scope and spirit of the invention Although the invention has been described in connection with specific preferred embodiments, it should be understood that the invention as claimed should not be unduly limited to such specific embodiments. Indeed, various modifications of the described modes for carrying out the invention that are obvious to those skilled in botany, biochemistry, chemistry, molecular biology, plant biology, plant disease, and plant pathogens, or related fields are intended to be within the scope of the following claims.

We claim:

1. An expression vector comprising a nucleic acid sequence encoding a glycerol phosphate acyltransferase polypeptide in operable combination with a plant promoter.

2. The expression vector of claim 1, wherein said promoter is selected from the group consisting of constitutive promoters, enzyme promoters, tissue specific promoters, inducible promoters, and temperature sensitive promoters.

3. The expression vector of claim 1, wherein said promoter is selected from the group consisting of a glycerol phosphate acyltransferase 5 (GPAT5) promoter, a glycerol phosphate acyltransferase 4, (GPAT4) promoter, a glycerol phosphate acyltransferase 7 (GPAT7) promoter, a glycerol phosphate acyltransferase 8 (GPAT8) promoter, a Lipid Transfer Protein 1 (LPT1) promoter, a CUTICULAR 1 (CUT1) promoter, a Long Chain Acyl-CoA Synthetase 2 (LACS2) promoter, an acyl-CoA synthetase long-chain family member 3 (ACSL3) promoter, a FbL2A promoter, an E6 promoter, a potato multicystatin (PMC) promoter, a R929A promoter, a RCI2A promoter, a RCI2B promoter, a CBF promoter, and a potato α-amylase promoter.

4. The expression vector of claim 1, wherein said glycerol phosphate acyltransferase polypeptide is selected from the group consisting of SEQ ID NOs: 9, 10, 11, 12, 13, 14, 15, and 16 and homologs, orthologs, and fragments thereof.

5. The expression vector of claim 1, wherein said glycerol phosphate acyltransferase polypeptide is at least 38% identical to SEQ ID NO:09.

6. The expression vector of claim 1, wherein said plant is selected from the group consisting of a mustard, tobacco, potato, cotton, rice, and algae.

7. The expression vector of claim 1, wherein said glycerol phosphate acyltransferase polypeptide alters extracellular lipid on a plant part.

8. A silencing expression vector comprising a plant promoter in operable combination with an antisense nucleic acid targeted to a nucleic acid sequence encoding a glycerol phosphate acyltransferase polypeptide or portion thereof.

9. The silencing expression vector of claim 8, wherein said glycerol phosphate acyltransferase polypeptide is SEQ ID NO:09.

10. The silencing expression vector of claim 8, wherein said plant is selected from the group consisting of a mustard, potato, and cotton.

11. The silencing expression vector of claim 8, wherein said promoter is chosen from the group consisting of a constitutive promoter, a tissue specific promoter, an inducible promoter, and a temperature sensitive promoter.

12. A transgenic plant part comprising a heterologous glycerol phosphate acyltransferase nucleic acid sequence and altered extracellular lipid.

13. The transgenic plant part of claim 12, wherein said altered extracellular lipid comprises free fatty acid, monoacylglycerol, very long chain fatty acid, wax ester, polyester monomer, fatty dicarboxylic acid, polyol fatty acid, suberin, and cutin.

14. The transgenic plant part of claim 13, wherein said altered extracellular lipid is increased as compared to a wild-type plant part.

15. The transgenic plant part of claim 14, wherein the increased extracellular lipid is a total extracellular wax at least 1000 ug/gfw.

16. The transgenic plant part of claim 14, wherein said increased extracellular lipid is a wax load at least 100 ug/gfw.

17. The transgenic plant part of claim 13, wherein said monoacylglycerols are at least 100 ug/gfw.

18. The transgenic plant part of claim 13, wherein said monoacylglycerol is selected from the group consisting of α-monoacylglycerol and β-monoacylglycerol.

19. The transgenic plant part of claim 13, wherein said wax ester is selected from the group consisting of carbon chain lengths of C48 to C54 and C56 to C120.

20. The transgenic plant part of claim 13, wherein said altered extracellular lipid is decreased as compared to a wild-type plant part.

21. The transgenic plant part of claim 12, wherein said polypeptide is selected from the group consisting of SEQ ID NOs:9, 10, 11, 12, 13, 14, 15, 16, and 17; homologous, orthologs, and fragments thereof.

22. The transgenic plant part of claim 12, wherein said glycerol phosphate acyltransferase polypeptide is SEQ ID NO:09.

23. The transgenic plant part of claim 12, wherein said plant part comprises a seed, root, stem, tuber, leaf, boll, fiber, needle, shoot, bud, pod, fruit, rind, nut, bark, rhizome, bulb, flower, and a whole plant.

24. The transgenic plant part of claim 12, wherein said plant part is a seed.

25. The transgenic plant part of claim 12, wherein said plant is selected from the group consisting of mustard, tobacco, potato, cotton, sunflower, corn, safflower, rice, and algae.

26. An isolated extracellular plant lipid comprising a first lipid, wherein said first lipid is a monoacylglycerols at least 5% w/w and a second lipid.

27. The isolated extracellular plant lipid of claim 26, wherein said second lipid comprises a free fatty acid, very long chain fatty acid, wax ester, polyester monomer, fatty dicarboxylic acid, polyol fatty acid and combinations thereof.

28. The isolated extracellular plant lipid of claim 27, wherein said free fatty acid comprises C24, C26, and C28 chain lengths.

29. The isolated extracellular plant lipid of claim 26, wherein said monoacylglycerol comprises alpha-monoacylglycerol and beta-monoacylglycerol.

30. A method of providing a transgenic plant having altered plant surface lipid expression, comprising:
a) providing,
i) a heterologous acyltransferase nucleic acid sequence for altering the plant surface lipid molecules, wherein said acyltransferase nucleic acid sequence molecule encodes a polypeptide selected from the group consisting of SEQ ID NOs:9, 10, 11, 12, 13, 14, 15, 16, and 17; homologous, orthologs, and fragments thereof, and
ii) a plant tissue,
b) transfecting said heterologous acyltransferase nucleic acid sequence molecule into said plant tissue; and
c) regenerating a plant from said transfected plant tissue, such that expression of said heterologous acyltransferase nucleic acid sequence molecule in said plant results in altered extracellular plant lipid.

31. A transgenic plant produced by the method of claim 30.

32. A method of altering plant surface lipid, comprising, comprising,
a) providing;
i) a silencing expression vector encoding an antisense nucleic acid targeted to a nucleic acid sequence encoding a plant glycerol phosphate acyltransferase polypeptide, wherein said polypeptide is selected from the group consisting of SEQ ID NOs: 9, 10, 11, 12, 13, 14, 15, and 16, homologes, orthologs, and fragments thereof, and
ii) a plant tissue, wherein said plant tissue comprises a lipid surface; and
b) transfecting the plant tissue with the vector,
c) regenerating a plant from said transfected plant tissue, under conditions such that the antisense sequence is expressed and the plant surface lipid is altered.

33. A method of producing lipids comprising:
a) providing a transgenic plant comprising a heterologous acyltransferase nucleic acid sequence or a silencing expression vector encoding an antisense nucleic acid targeted to a nucleic acid sequence encoding a plant glycerol phosphate acyltransferase polypeptide; and
b) growing said transgenic plant under conditions such that said plant produces lipids.

34. The method of claim 33, further comprising the step of isolating said lipids from said plant.
